(19) United States
(12) Patent Application Publication
(10) Pub. No.: US 2022/0402965 A1
(43) Pub. Date: Dec. 22, 2022

(54) STEROL ANALOGS AND USES THEREOF

(71) Applicant: ModernaTX, Inc., Cambridge, MA (US)

(72) Inventors: Kerry BENENATO, Lincoln, MA (US); Mark CORNEBISE, Arlington, MA (US); Edward J. HENNESSY, Westwood, MA (US)

(21) Appl. No.: 17/277,829

(22) PCT Filed: Sep. 19, 2019
(86) PCT No.: PCT/US19/51959

§ 371 Date: Mar. 19, 2021

Related U.S. Application Data
(60) Provisional application No. 62/733,540, filed on Sep. 19, 2018.
(60) Provisional application No. 62/798,752, filed on Jan. 30, 2019.

(51) Int. Cl. C07J 7/00; A61K 9/51; C07J 17/00; C07J 43/00; C07J 33/00; C07J 41/00; C07J 9/00; C07J 21/00; C07J 71/00; C07J 31/00; C07J 51/00; A61K 31/7115; A61K 38/46; A61K 31/7105; A61K 47/10; A61K 47/24; A61K 47/28; A61K 48/00

(52) U.S. Cl. CPC C07J 7/0005; A61K 48/0033; C07J 17/00; C07J 7/002; C07J 43/003; C07J 33/002; C07J 41/0061; C07J 9/005; C07J 41/0005; C07J 21/008; C07J 71/0005; C07J 31/006; C07J 51/00; A61K 31/7115; A61K 38/465; A61K 31/7105; A61K 47/10; A61K 47/24; A61K 47/28; A61K 9/5123

(57) ABSTRACT

The invention relates to compositions and methods for the preparation, manufacture, and therapeutic use of compositions comprising mRNA and a lipid nanoparticle comprising a compound of the invention and an ionizable lipid.

### BACKGROUND OF THE INVENTION

In recent years, nucleic acids have increasingly been looked to as possible therapeutic agents. Therapeutic uses of messenger ribonucleic acid (mRNA) are particularly sought as an mRNA could be designed to encode a wide variety of polypeptides for many applications. For example, many diseases, disorders, and conditions, including cystic fibrosis, are characterized by aberrant protein activity and/or protein deficiency. It is theorized that the introduction of an appropriate mRNA could be translated within a cell to generate a polypeptide to replace, subvert, or otherwise combat an aberrant species. mRNA delivery systems could also be used to regulate important polypeptides such as vascular endothelial growth factor (VEGF), the transient and targeted expression of which is posited to combat stenosis in renovascular structures. Disruption of translational machineries by the introduction of non-translatable mRNA may also be feasible. However, the delivery of therapeutic RNAs to cells is made difficult by the relative instability and low cell permeability of RNAs.

Accordingly, there exists a need to develop methods and lipid-containing compositions to facilitate the delivery of RNAs such as mRNA to cells, especially with regards to improvements in safety, efficacy, and specificity.

### SUMMARY OF THE INVENTION

This invention features sterol compounds which may be utilized in a lipid nanoparticle for delivering mRNA into cells. In an aspect, a lipid nanoparticle of the invention includes an ionizable lipid and a compound of the invention.

In an aspect, the invention features a compound having the structure of Formula I:

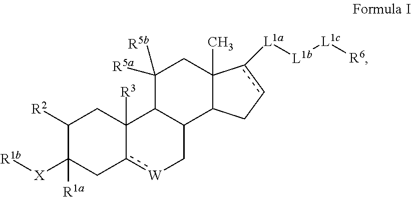

where

R^{1a} is H, optionally substituted C₁-C₆ alkyl, optionally substituted C₂-C₆ alkenyl, or optionally substituted C₂-C₆ alkynyl;

X is O or S;

R^{1b} is H, optionally substituted C₁-C₆ alkyl, or

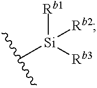

each of R^{b1}, R^{b2}, and R^{b3} is, independently, optionally substituted C₁-C₆ alkyl or optionally substituted C₆-C₁₀ aryl;

R² is H or OR^{A}, where R^{A} is H or optionally substituted C₁-C₆ alkyl;

R³ is H or

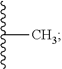

each [symbol] independently represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

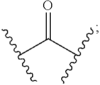 [SMILES: *C(*)=O]

L^{1a} is absent,

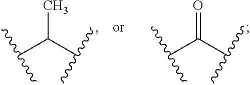

L^{1b} is absent,

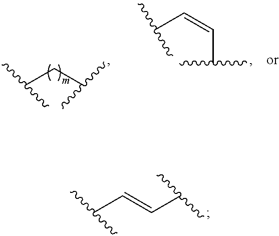

m is 1, 2, or 3;

L^{1c} is absent,

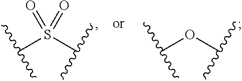

and

R⁶ is optionally substituted C₃-C₂₀ cycloalkyl, optionally substituted C₃-C₂₀ cycloalkenyl, optionally substituted C₆-C₂₀ aryl, optionally substituted C₂-C₁₉ heterocyclyl, or optionally substituted C₂-C₁₉ heteroaryl, or a pharmaceutically acceptable salt thereof.

In some embodiments, the compound has the structure of Formula Ia:

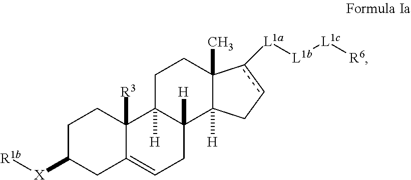

or a pharmaceutically acceptable salt thereof.

In some embodiments, the compound has the structure of Formula Ib:

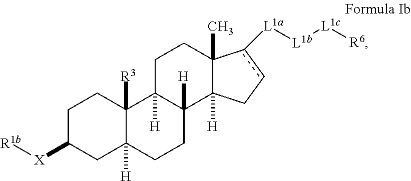

or a pharmaceutically acceptable salt thereof.

In some embodiments, the compound has the structure of Formula Ic:

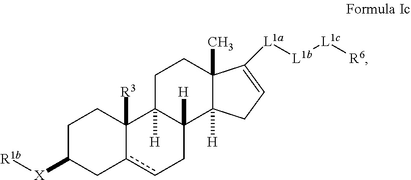

or a pharmaceutically acceptable salt thereof.

In some embodiments, the compound has the structure of Formula Id:

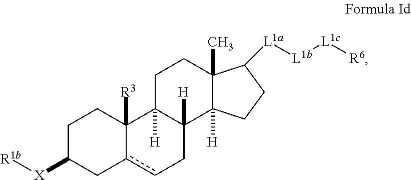

or a pharmaceutically acceptable salt thereof.

In some embodiments, L^{1a} is absent. In some embodiments, L^{1a} is

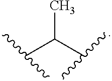 [SMILES: *C(*)C]

In some embodiments, L^{1a} is

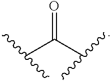 [SMILES: *C(*)=O]

In some embodiments, L^{1b} is absent. In some embodiments, L^{1b} is

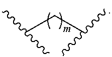

In some embodiments, L^{1b} is

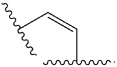 [SMILES: */C=C\*]

In some embodiments, L^{1b} is

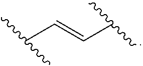 [SMILES: */C=C/*]

In some embodiments, m is 1 or 2. In some embodiments, m is 1. In some embodiments, m is 2.

In some embodiments, L^{1c} is absent. In some embodiments, L^{1c} is

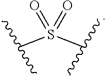 [SMILES: *S(*)(=O)=O]

In some embodiments, L^{1c} is

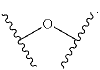 [SMILES: *O*]

In some embodiments, R⁶ is optionally substituted C₆-C₂₀ aryl. In some embodiments, R⁶ is optionally substituted C₆-C₁₂ aryl. In some embodiments, R⁶ is optionally substituted C₆-C₁₀ aryl.

In some embodiments, R⁶ is

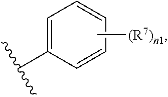

where

n1 is 0, 1, 2, 3, 4, or 5; and

each R⁷ is, independently, halo or optionally substituted C₁-C₆ alkyl.

In some embodiments, each R⁷ is, independently,

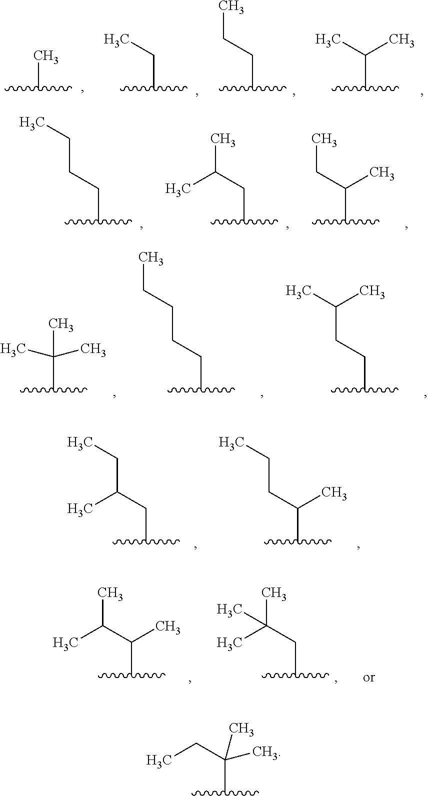

In some embodiments, n1 is 0, 1, or 2. In some embodiments, n is 0. In some embodiments, n1 is 1. In some embodiments, n1 is 2.

In some embodiments, R⁶ is

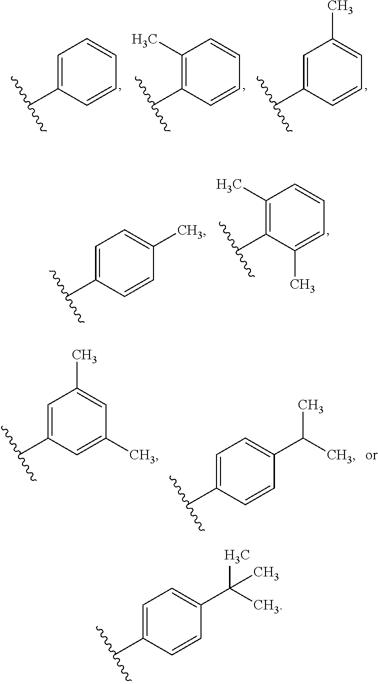

In some embodiments, R⁶ is optionally substituted C₃-C₂₀ cycloalkyl. In some embodiments, R⁶ is optionally substituted C₃-C₁₂ cycloalkyl.

In some embodiments, R⁶ is

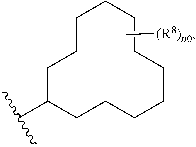

where

n0 is 0, 1, 2, 3, 4, 5, 6, 7, 8, 9, 10, 11, 12, 13, 14, 15, 16, 17, 18, 19, 20, 21, 22, or 23; and

each R⁸ is, independently, halo or optionally substituted C₁-C₆ alkyl.

In some embodiments, each R⁸ is, independently,

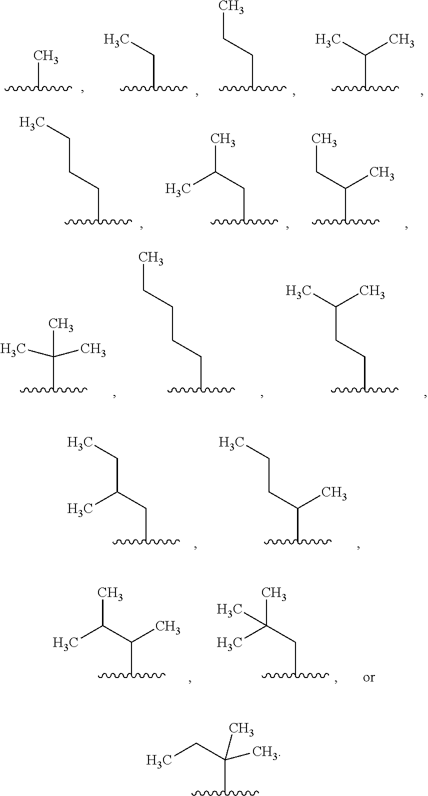

In some embodiments, n0 is 0, 1, 2, 3, 4, 5, or 6. In some embodiments, n0 is 0, 1, 2, or 3. In some embodiments, n0 is 0. In some embodiments, n0 is 1. In some embodiments, n0 is 2. In some embodiments, n0 is 3.

In some embodiments, R⁶ is

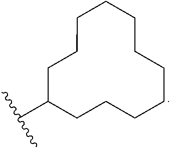 [SMILES: *C1CCCCCCCCCCC1]

In some embodiments, R⁶ is optionally substituted C₃-C₁₀ cycloalkyl.

In some embodiments, R⁶ is optionally substituted C₃-C₁₀ monocycloalkyl.

In some embodiments, R⁶ is

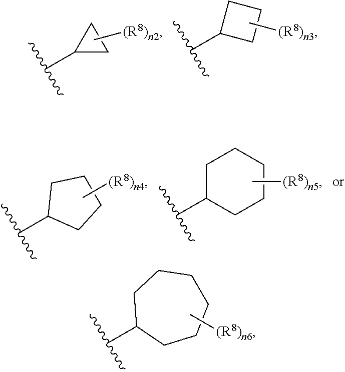

### Where

n2 is 0, 1, 2, 3, 4, or 5;

n3 is 0, 1, 2, 3, 4, 5, 6, or 7;

n4 is 0, 1, 2, 3, 4, 5, 6, 7, 8, or 9;

n5 is 0, 1, 2, 3, 4, 5, 6, 7, 8, 9, 10, or 11;

n6 is 0, 1, 2, 3, 4, 5, 6, 7, 8, 9, 10, 11, 12, or 13; and

each R⁸ is, independently, halo or optionally substituted C₁-C₆ alkyl.

In some embodiments, each R⁸ is, independently,

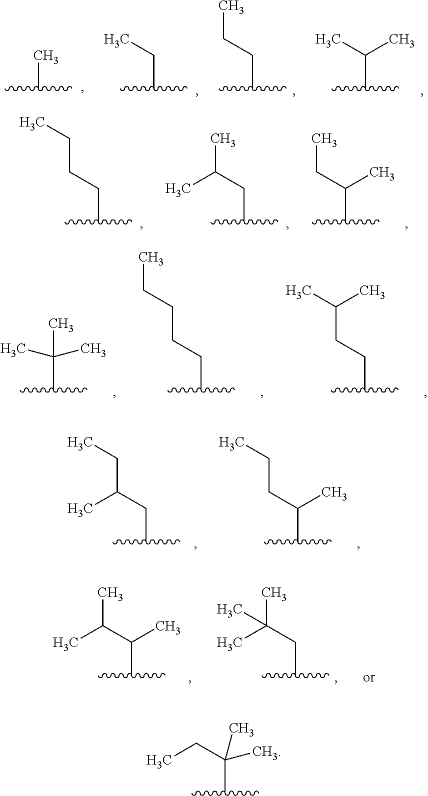

In some embodiments, n2 is 0 or 1. In some embodiments, n2 is 0. In some embodiments, n2 is 1.

In some embodiments, R⁶ is

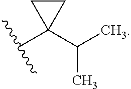 [SMILES: *C1(C(C)C)CC1]

In some embodiments, n3 is 0 or 1. In some embodiments, n3 is 1. In some embodiments, n3 is 2.

In some embodiments, R⁶ is

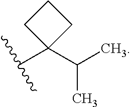 [SMILES: *C1(C(C)C)CCC1]

In some embodiments, n4 is 0, 1, or 2. In some embodiments, n4 is 0. In some embodiments, n4 is 1. In some embodiments, n4 is 2.

In some embodiments, R⁶ is

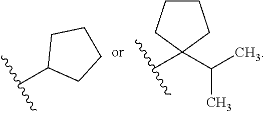

In some embodiments, n5 is 0, 1, 2, or 3. In some embodiments, n5 is 0. In some embodiments, n5 is 1. In some embodiments, n5 is 2. In some embodiments, n5 is 3.

In some embodiments, R⁶ is

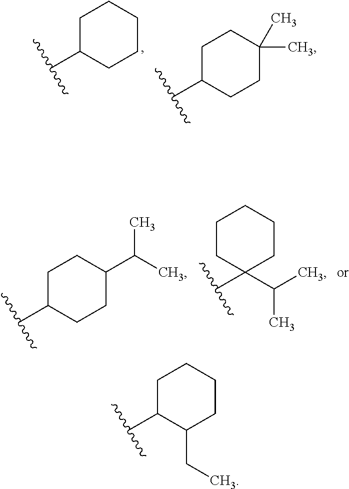

In some embodiments, n6 is 0, 1, 2, 3, or 4. In some embodiments, n6 is 0. In some embodiments, n63 is 1. In some embodiments, n6 is 2. In some embodiments, n6 is 3. In some 6embodiments, n6 is 4.

In some embodiments, R⁶ is

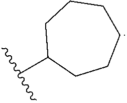 [SMILES: *C1CCCCCC1]

In some embodiments, R⁶ is optionally substituted C₃-C₁₀ polycycloalkyl.

In some embodiments, R⁶ is

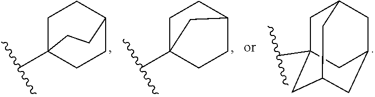

In some embodiments, R⁶ is optionally substituted C₃-C₂₀ cycloalkenyl. In some embodiments, R⁶ is optionally substituted C₃-C₁₂ cycloalkenyl. In some embodiments, R⁶ is optionally substituted C₃-C₁₀ cycloalkenyl.

In some embodiments, R⁶ is

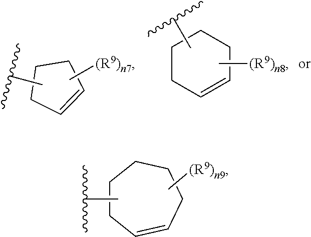

where

n7 is 0, 1, 2, 3, 4, 5, 6, or 7;

n8 is 0, 1, 2, 3, 4, 5, 6, 7, 8, or 9;

n9 is 0, 1, 2, 3, 4, 5, 6, 7, 8, 9, 10, or 11; and

each R⁹ is, independently, halo or optionally substituted C₁-C₆ alkyl.

In some embodiments, R⁶ is

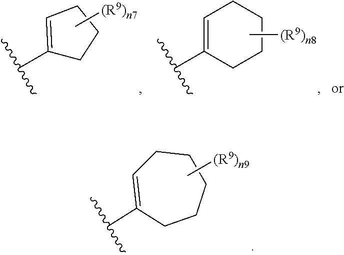

In some embodiments, each R⁹ is, independently,

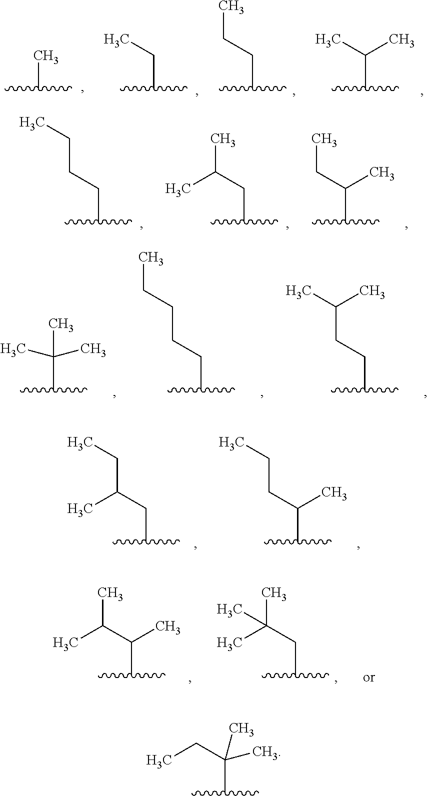

In some embodiments, n7 is 0, 1, or 2. In some embodiments, n7 is 0. In some embodiments, n7 is 1. In some embodiments, n7 is 2.

In some embodiments, R⁶ is

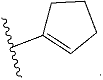 [SMILES: *C1=CCCC1]

In some embodiments, n8 is 0, 1, 2, or 3. In some embodiments, n8 is 0. In some embodiments, n8 is 1. In some embodiments, n8 is 2. In some embodiments, n8 is 3.

In some embodiments, R⁶ is

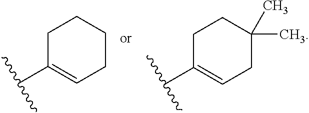

In some embodiments, n9 is 0, 1, 2, 3, or 4. In some embodiments, n9 is 0. In some embodiments, n9 is 1. In some embodiments, n9 is 2. In some embodiments, n9 is 3. In some embodiments, n9 is 4.

In some embodiments, R⁶ is

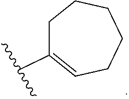 [SMILES: *C1=CCCCCC1]

In some embodiments, R⁶ is optionally substituted C₂-C₁₉ heterocyclyl. In some embodiments, R⁶ is optionally substituted C₂-C₁ heterocyclyl. In some embodiments, R⁶ is optionally substituted C₂-C₉ heterocyclyl.

In some embodiments, R⁶ is

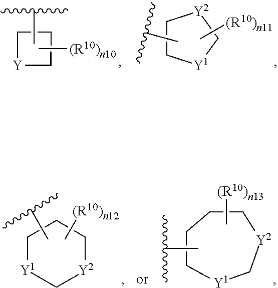

where

n10 is 0, 1, 2, 3, 4, or 5;

n11 is 0, 1, 2, 3, 4, 5, 6, or 7;

n12 is 0, 1, 2, 3, 4, 5, 6, 7, or 8;

n13 is 0, 1, 2, 3, 4, 5, 6, 7, 8, or 9;

each R¹⁰ is, independently, halo or optionally substituted C₁-C₆ alkyl; and

each of Y¹ and Y² is, independently, O, S, NR^{B}, or CR^{11a}R^{11b}, where R^{B} is H or optionally substituted C₁-C₆ alkyl;

each of R^{11a} and R^{11b} is, independently, H, halo, or optionally substituted C₁-C₆ alkyl; and

if Y² is CR^{11a}R^{11b}, then Y¹ is O, S, or NR^{B}.

In some embodiments, Y¹ is O. In some embodiments, Y¹ is S. In some embodiments, Y¹ is NR^{B}.

In some embodiments, Y² is O. In some embodiments, Y² is S. In some embodiments, Y² is NR⁸. In some embodiments, Y² is CR^{11a}R^{11b}.

In some embodiments, each R¹⁰ is, independently,

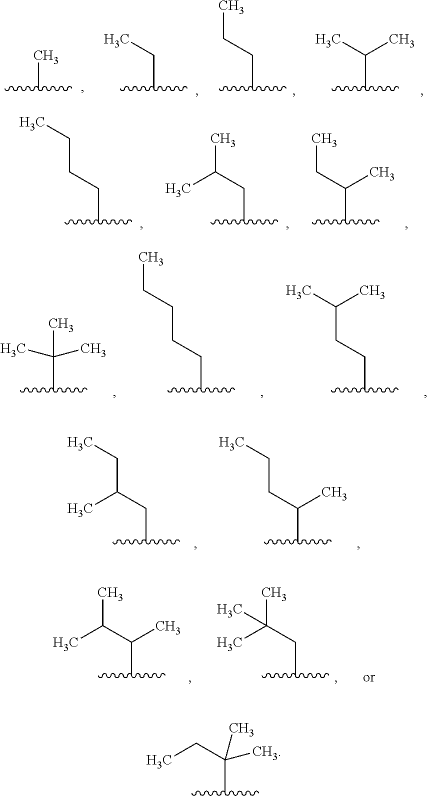

In some embodiments, n10 is 0 or 1. In some embodiments, n10 is 0. In some embodiments, n10 is 1.

In some embodiments, R⁶ is

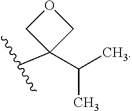 [SMILES: *C1(C(C)C)COC1]

In some embodiments, n11 is 0, 1, 2, 3, 4, or 5. In some embodiments, n11 is 0. In some embodiments, n11 is 1. In some embodiments, n11 is 2. In some embodiments, n11 is 3. In some embodiments, n11 is 4. In some embodiments, n11 is 5.

In some embodiments, R⁶ is

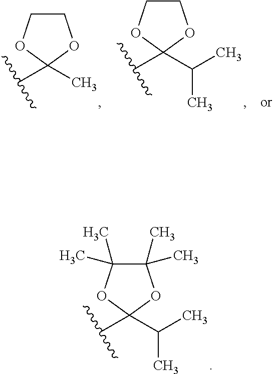

In some embodiments, n12 is 0, 1, 2, 3, 4, 5, or 6. In some embodiments, n12 is 0. In some embodiments, n12 is 1. In some embodiments, n12 is 2. In some embodiments, n12 is 3. In some embodiments, n12 is 4. In some embodiments, n12 is 5. In some embodiments, n12 is 6.

In some embodiments, R⁶ is

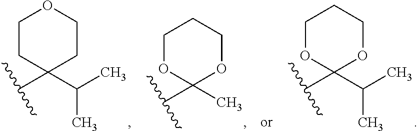

In some embodiments, R⁶ is optionally substituted C₂-C₁₉ heteroaryl. In some embodiments, R⁶ is optionally substituted C₂-C₁₁ heteroaryl. In some embodiments, R⁶ is optionally substituted C₂-C₉ heteroaryl.

In some embodiments, R⁶ is

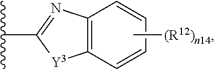

where

Y³ is NR^{C}, O, or S;

n14 is 0, 1, 2, 3, or 4;

R^{C} is H or optionally substituted C₁-C₆ alkyl; and

each R¹² is, independently, halo or optionally substituted C₁-C₆ alkyl.

In some embodiments, n14 is 0, 1, or 2. In some embodiments, n14 is 0. In some embodiments, n14 is 1. In some embodiments, n14 is 2.

In some embodiments, each R¹² is, independently,

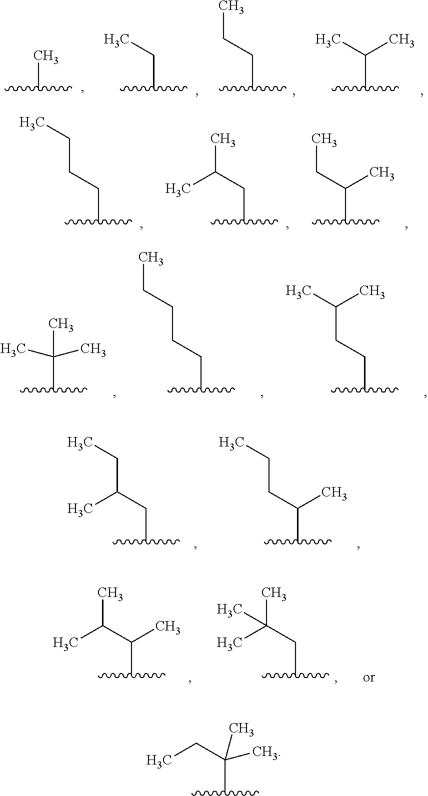

In some embodiments, Y³ is S. In some embodiments, Y³ is NR^{C}.

In some embodiments, R⁶ is

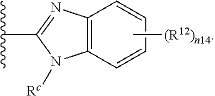

In some embodiments, R⁶ is

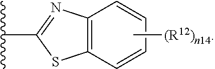

In some embodiments, R^{C} is H or

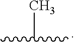

In some embodiments, R⁶ is

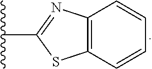 [SMILES: *c1nc2ccccc2s1]

In some embodiments, R⁶ is

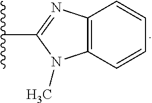 [SMILES: *c1nc2ccccc2n1C]

In an aspect, the invention features a compound having the structure of Formula II:

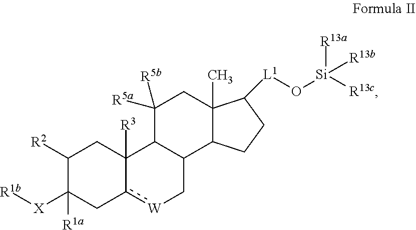

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

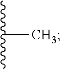

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

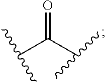 [SMILES: *C(*)=O]

L¹ is optionally substituted C₁-C₆ alkylene; and

each of R^{13a}, R^{13b}, and R^{13c} is, independently, optionally substituted C₁-C₆ alkyl or optionally substituted C₆-C₁₀ aryl,

or a pharmaceutically acceptable salt thereof.

In some embodiments, the compound has the structure of Formula IIa:

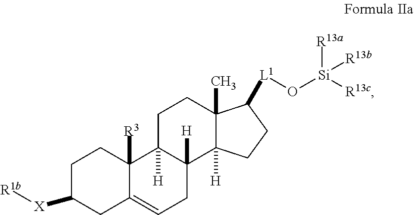

or a pharmaceutically acceptable salt thereof.

In some embodiments, the compound has the structure of Formula IIb:

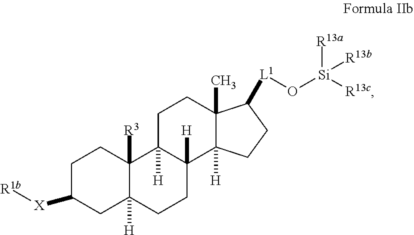

or a pharmaceutically acceptable salt thereof.

In some embodiments, L¹ is

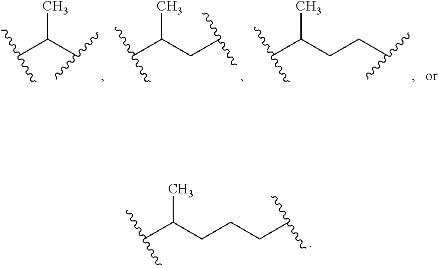

In some embodiments, each of R^{13a}, R^{13b}, and R^{13c} is, independently,

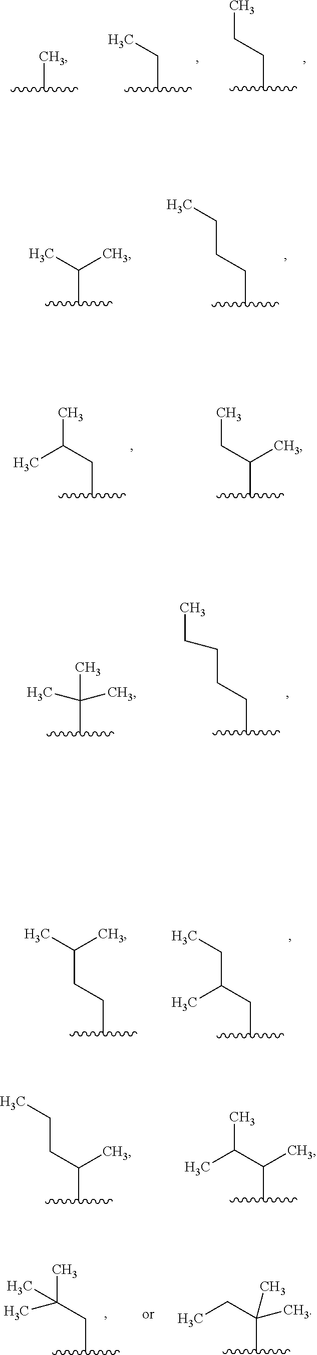

In an aspect, the invention features a compound having the structure of Formula III:

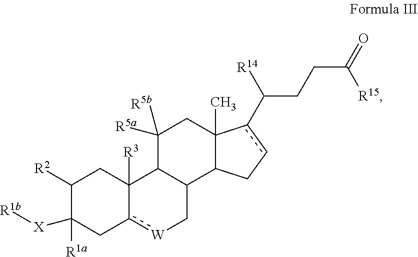

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

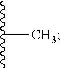

each [symbol] independently represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, hydroxyl, optionally substituted C₁-C₆ alkyl, —OS(O)₂R^{4c}, where R^{4c} is optionally substituted C₁-C₆ alkyl or optionally substituted C₆-C₁₀ aryl;

each of R^{5a} and R^{5b} is, independently, H or OR^{A}, or R^{5a} and R^{5b}, together with the atom to which each is attached, combine to form

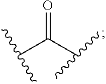 [SMILES: *C(*)=O]

R¹⁴ is H or C₁-C₆ alkyl; and

R¹⁵ is

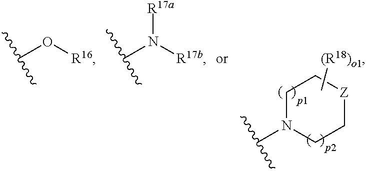

where

R¹⁶ is H or optionally substituted C₁-C₆ alkyl;

R^{17a} is H, optionally substituted C₆-C₁₀ aryl, or optionally substituted C₁-C₆ alkyl;

R^{17b} is H, OR^{17c}, optionally substituted C₆-C₁₀ aryl, or optionally substituted C₁-C₆ alkyl;

R^{17c} is H or optionally substituted C₁-C₆ alkyl;

o1 is 0, 1, 2, 3, 4, 5, 6, 7, or 8;

p1 is 0, 1, or 2;

p2 is 0, 1, or 2;

Z is CH₂, O, S, or NR^{D}, where R^{D} is H or optionally substituted C₁-C₆ alkyl; and

each R¹⁸ is, independently, halo or optionally substituted C₁-C₆ alkyl, or a pharmaceutically acceptable salt thereof.

In some embodiments, the compound has the structure of Formula IIIa:

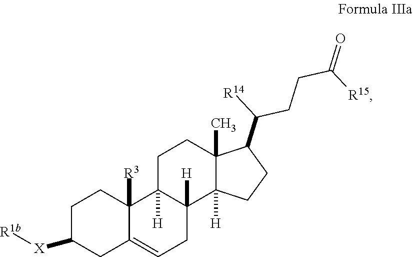

or a pharmaceutically acceptable salt thereof.

In some embodiments, the compound has the structure of Formula IIIb:

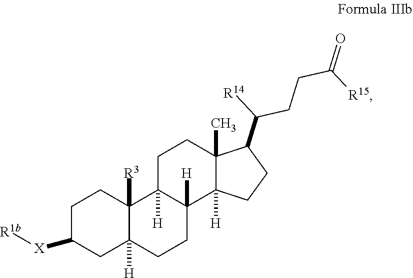

or a pharmaceutically acceptable salt thereof.

In some embodiments, R¹⁴ is

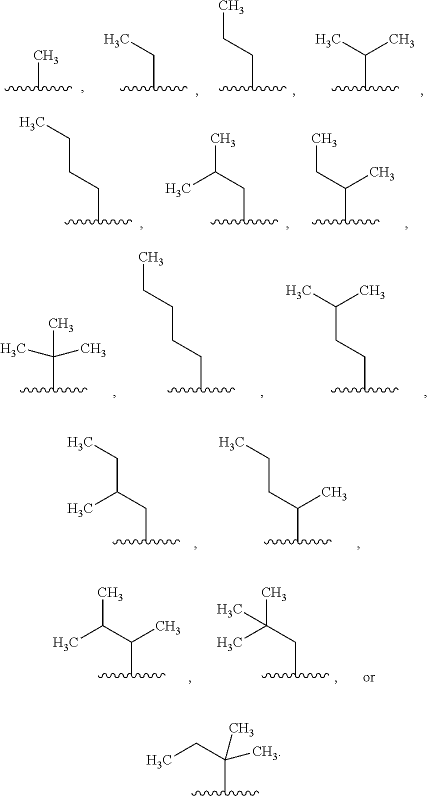

In some embodiments, R¹⁴ is

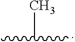

In some embodiments, R¹⁵ is

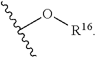

In some embodiments, R¹⁵ is

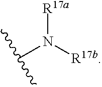

In some embodiments, R¹⁶ is H. In some embodiments, R¹⁶ is

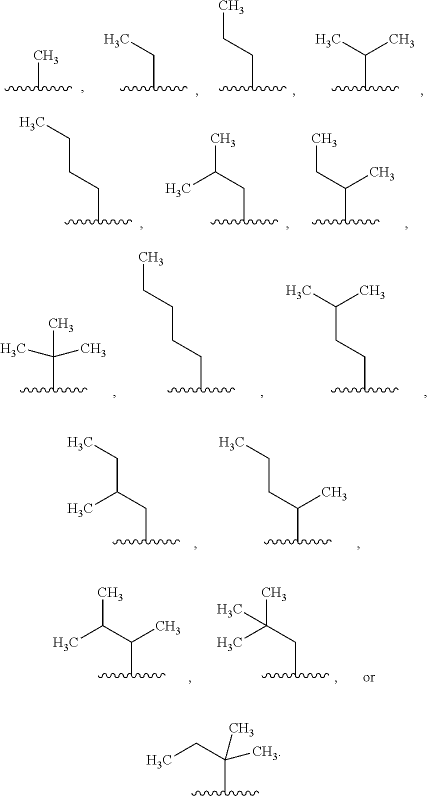

In some embodiments, R^{17a} is H or optionally substituted C₁-C₆ alkyl. In some embodiments, R^{17b} is H or optionally substituted C₁-C₆ alkyl.

In some embodiments, R^{17a} is H. In some embodiments, R^{17a} is optionally substituted C₁-C₆ alkyl.

In some embodiments, R^{17b} is H. In some embodiments, R^{17b} optionally substituted C₆-C₁₀ aryl.

In some embodiments, R^{17b} optionally substituted C₁-C₆ alkyl. In some embodiments, R^{17b} is OR^{17c}.

In some embodiments, R^{17c} is H,

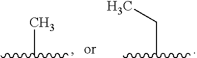

In some embodiments, R^{17c} is H. In some embodiments, R^{17c} is

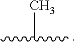

In some embodiments, R¹⁵ is

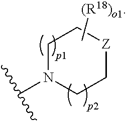

In some embodiments, each R¹⁸ is, independently,

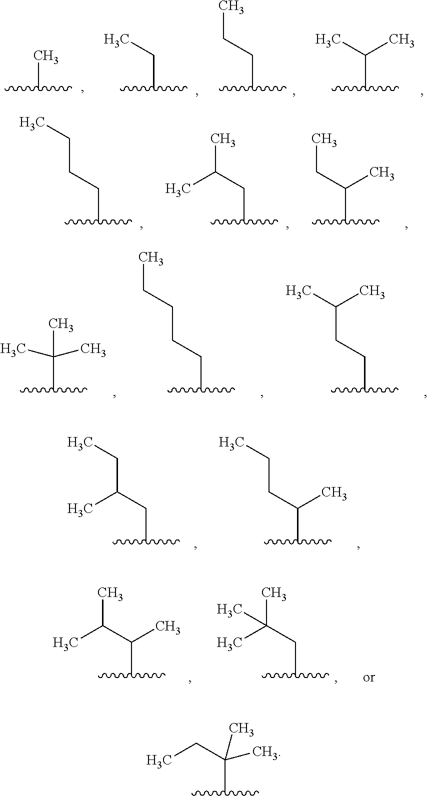

In some embodiments, Z is O or NR^{D}.

In some embodiments, Z is CH₂. In some embodiments, Z is O. In some embodiments, Z is NR^{D}.

In some embodiments, o1 is 0, 1, 2, 3, 4, 5, or 6.

In some embodiments, o1 is 0. In some embodiments, o1 is 1. In some embodiments, o1 is 2.

In some embodiments, o1 is 3. In some embodiments, o1 is 4. In some embodiments, o1 is 5. In some embodiments, o1 is 6.

In some embodiments, p1 is 0 or 1.

In some embodiments, p1 is 0. In some embodiments, p1 is 1.

In some embodiments, p2 is 0 or 1.

In some embodiments, p2 is 0. In some embodiments, p2 is 1.

In an aspect, the invention features a compound having the structure of Formula IV:

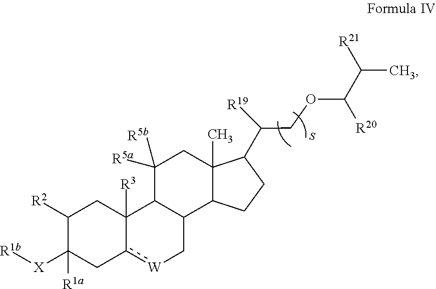

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

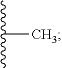

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

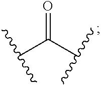 [SMILES: *C(*)=O]

s is 0 or 1;

R¹⁹ is H or C₁-C₆ alkyl;

R²⁰ is C₁-C₆ alkyl; and

R²¹ is H or C₁-C₆ alkyl,

or a pharmaceutically acceptable salt thereof.

In some embodiments, the compound has the structure of Formula IVa:

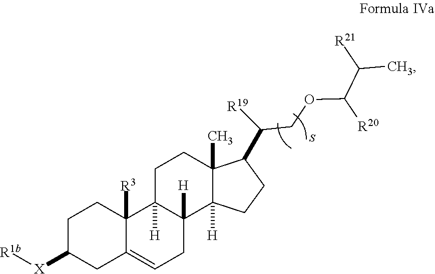

or a pharmaceutically acceptable salt thereof.

In some embodiments, the compound has the structure of Formula IVb:

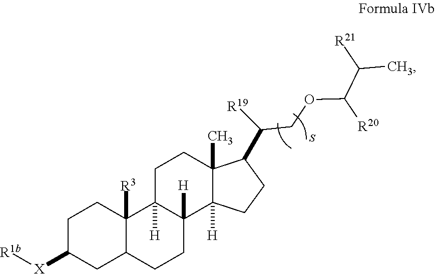

or a pharmaceutically acceptable salt thereof.

In some embodiments, R¹⁹ is

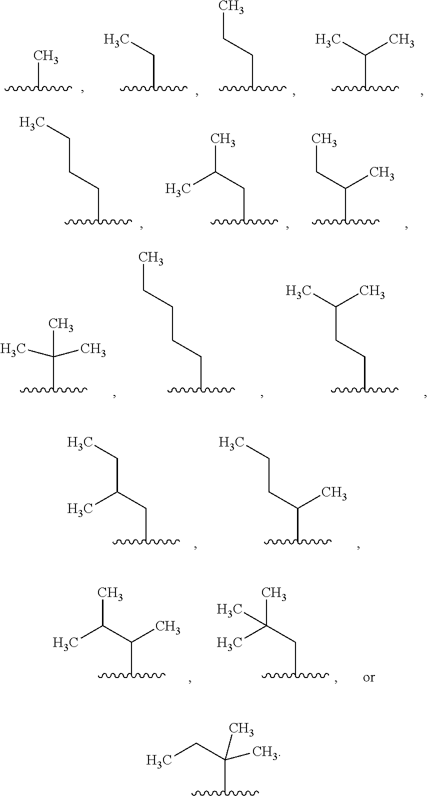

In some embodiments, R¹⁹ is

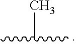

In some embodiments, R²⁰ is, independently,

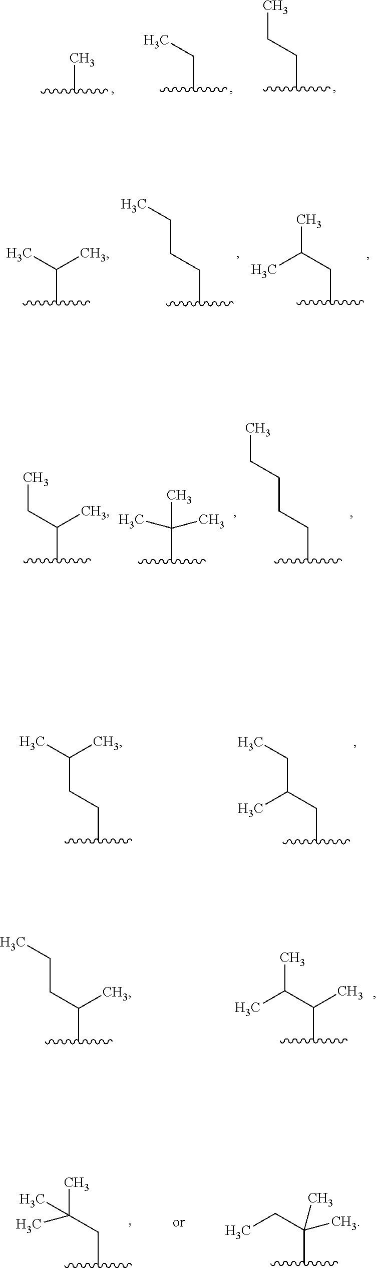

In some embodiments, R²¹ is

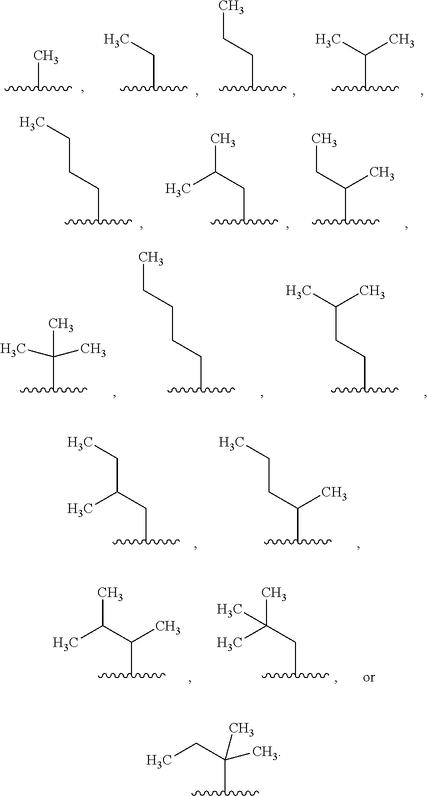

In some embodiments, each of R¹⁹, R²⁰, and R²¹ is, independently,

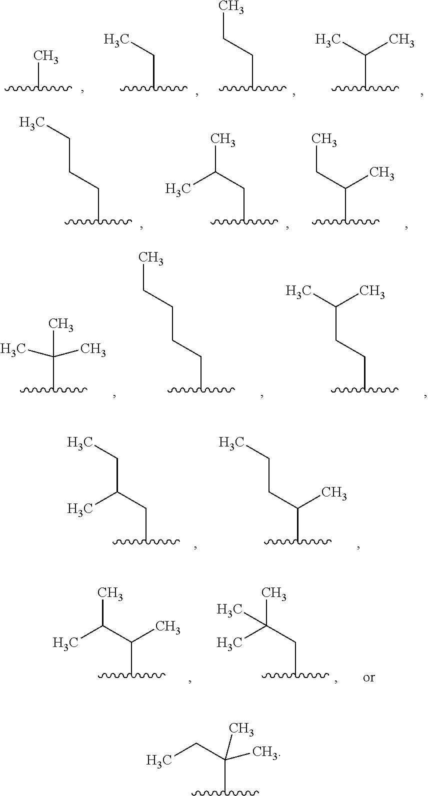

In an aspect, the invention features, a compound having the structure of Formula V:

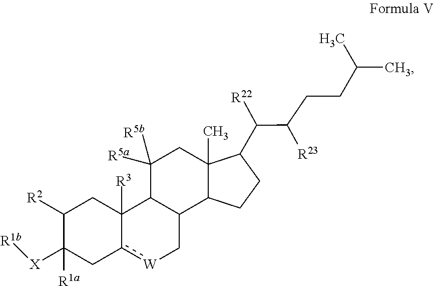

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

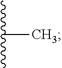

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

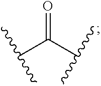 [SMILES: *C(*)=O]

R²² is H or C₁-C₆ alkyl; and

R²³ is halo, hydroxyl, optionally substituted C₁-C₆ alkyl, or optionally substituted C₁-C₆ heteroalkyl, or a pharmaceutically acceptable salt thereof.

In some embodiments, the compound has the structure of Formula Va:

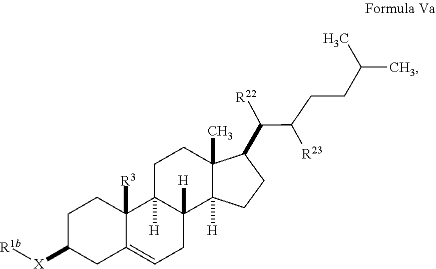

or a pharmaceutically acceptable salt thereof.

In some embodiments, the compound has the structure of Formula Vb:

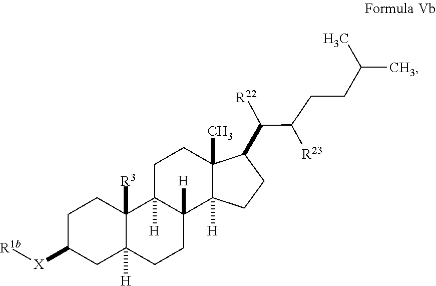

or a pharmaceutically acceptable salt thereof.

In some embodiments, R²² is

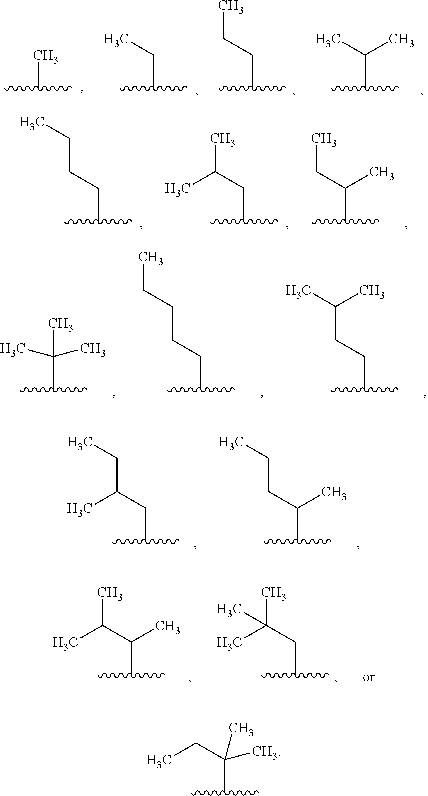

In some embodiments, R²² is

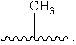

In some embodiments, R²³ is H or optionally substituted C₁-C₆ alkyl. In some embodiments, R²³ is halo. In some embodiments, R²³ is hydroxyl or optionally substituted C₁-C₆ heteroalkyl.

In some embodiments, R²³ is H. In some embodiments, R²³ is optionally substituted C₁-C₆ alkyl. In some embodiments, R²³ is halo. In some embodiments, R²³ is hydroxyl. In some embodiments, R²³ is optionally substituted C₁-C₆ heteroalkyl.

In some embodiments, R²³ is

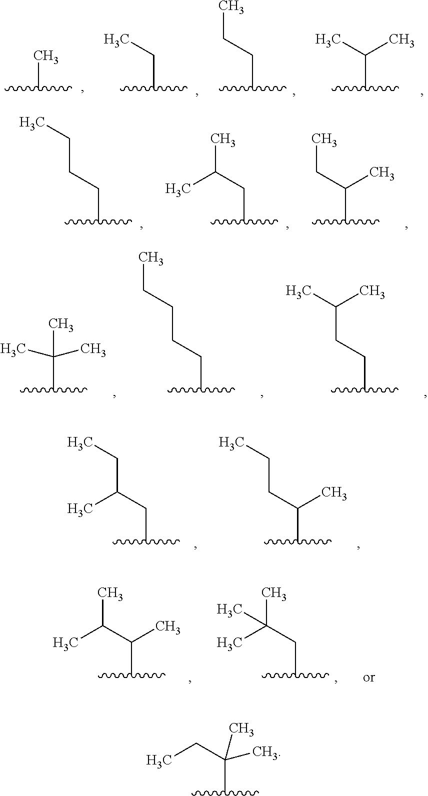

In some embodiments, each of R²² and R²³ is, independently,

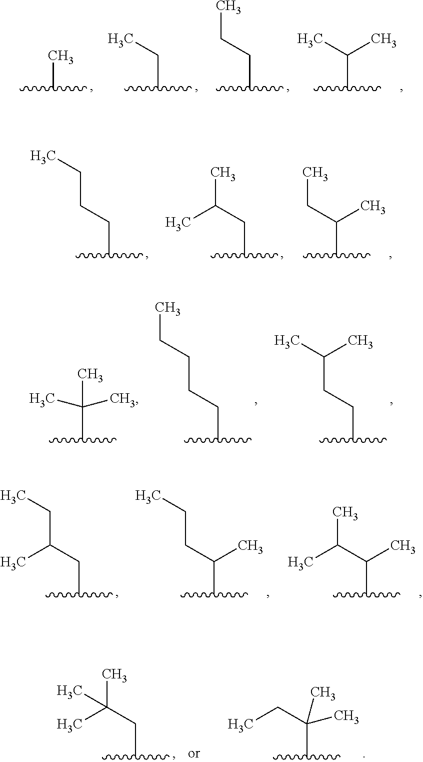

In an aspect, the invention features a compound having the structure of Formula VI:

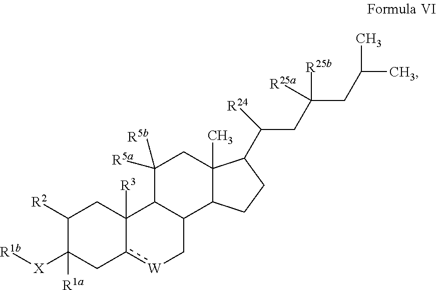

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

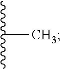

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

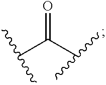 [SMILES: *C(*)=O]

R²⁴ is H or C₁-C₆ alkyl; and

each of R^{25a} and R^{25b} is C₁-C₆ alkyl,

or a pharmaceutically acceptable salt thereof.

In some embodiments, the compound has the structure of Formula VIa:

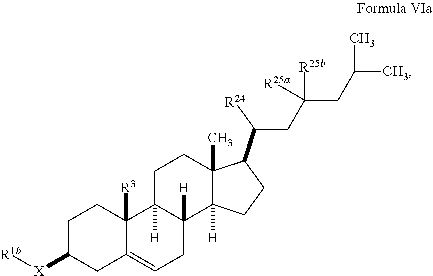

or a pharmaceutically acceptable salt thereof.

In some embodiments, the compound has the structure of Formula VIb:

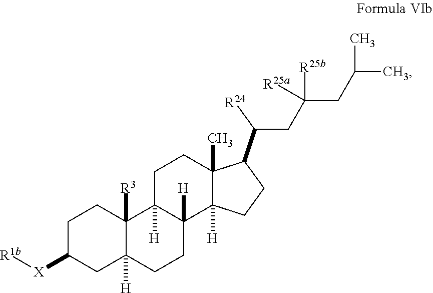

or a pharmaceutically acceptable salt thereof.

In some embodiments, R²⁴ is H,

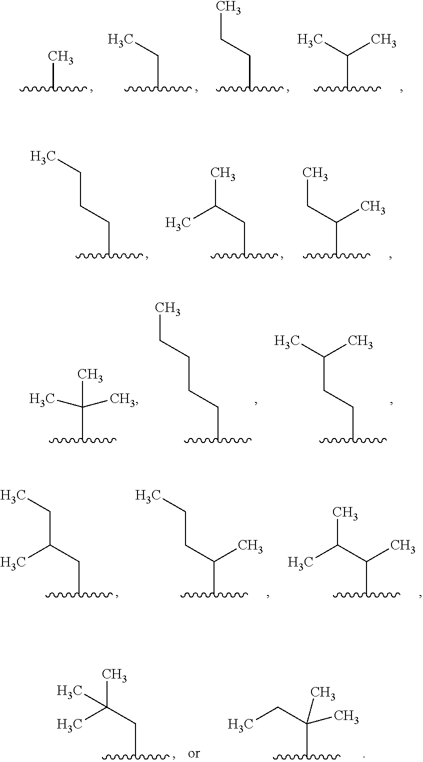

In some embodiments, R²⁴ is

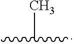

In some embodiments, each of R^{25a} and R^{25b} is, independently,

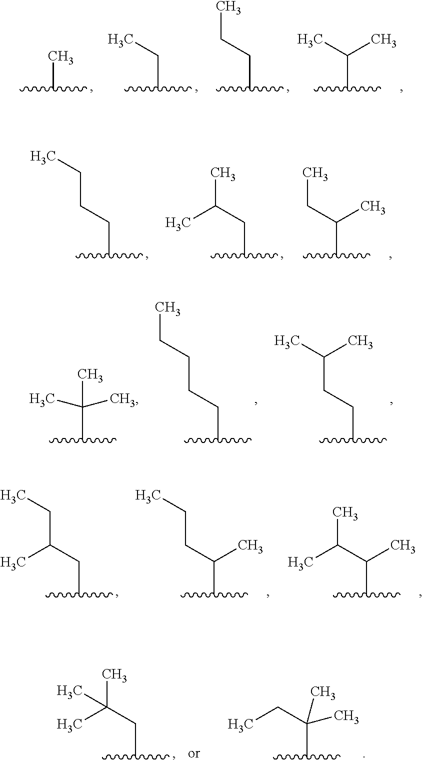

In some embodiments, each of R²⁴, R^{25a}, and R^{25b} is, independently,

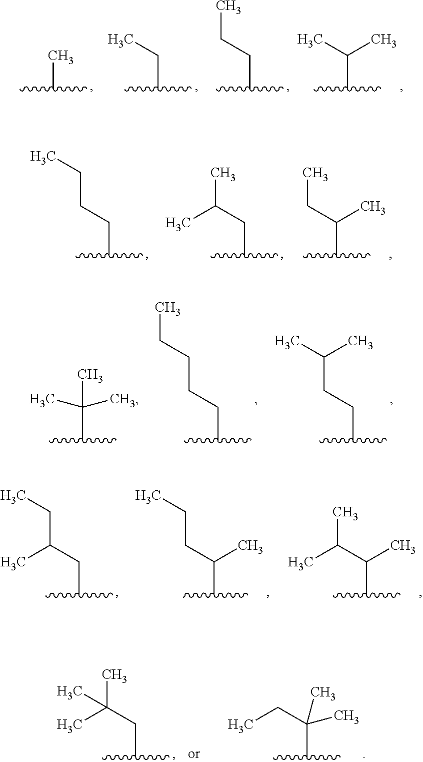

In an aspect, the invention features a compound having the structure of Formula VII:

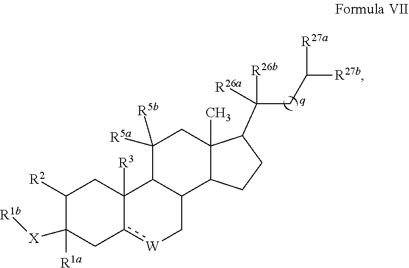

where

R^{1a} is H, optionally substituted C₁-C₆ alkyl, optionally substituted C₂-C₆ alkenyl, optionally substituted C₂-C₆ alkynyl, or

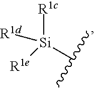

where each of R^{1c}, R^{1d}, and R^{1e} is, independently, optionally substituted C₁-C₆ alkyl or optionally substituted C₆-C₁₀ aryl;

X is O or S;

R^{1b} is H or optionally substituted C₁-C₆ alkyl;

R² is H or OR^{A}, where R^{A} is H or optionally substituted C₁-C₆ alkyl;

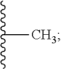

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

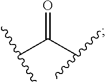 [SMILES: *C(*)=O]

q is 0 or 1;

each of R^{26a} and R^{26b} is, independently, H or optionally substituted C₁-C₆ alkyl, or R^{26a} and R^{26b}, together with the atom to which each is attached, combine to form

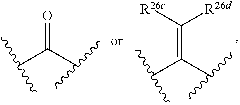

where each of R^{26c} and R²⁶ is, independently, H or optionally substituted C₁-C₆ alkyl; and

each of R^{27a} and R^{27b} is H, hydroxyl, or optionally substituted C₁-C₆ alkyl, or a pharmaceutically acceptable salt thereof.

In some embodiments, the compound has the structure of Formula VIIa:

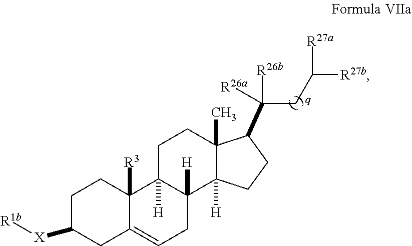

or a pharmaceutically acceptable salt thereof.

In some embodiments, the compound has the structure of Formula VIIb:

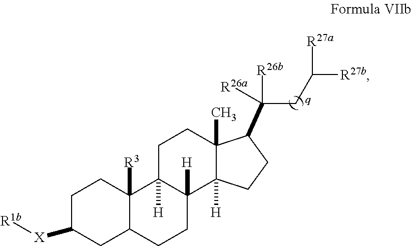

or a pharmaceutically acceptable salt thereof.

In some embodiments, each of R^{26a} and R^{26b} is, independently, H,

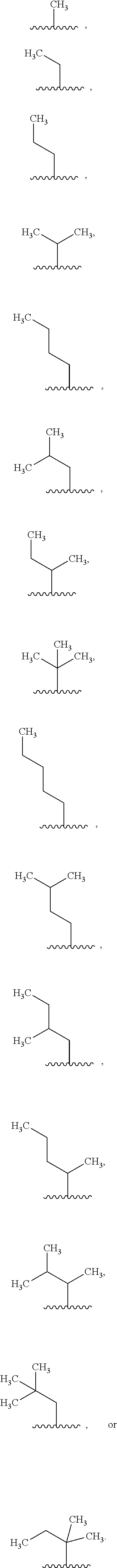

In some embodiments, R^{26a} and R^{26b}, together with the atom to which each is attached, combine to form

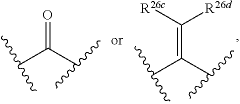

In some embodiments, R^{26a} and R^{26b}, together with the atom to which each is attached, combine to form

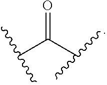 [SMILES: *C(*)=O]

In some embodiments, R^{26a} and R^{26b}, together with the atom to which each is attached, combine to form

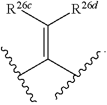

In some embodiments, where each of R^{26c} and R²⁶ is, independently, H,

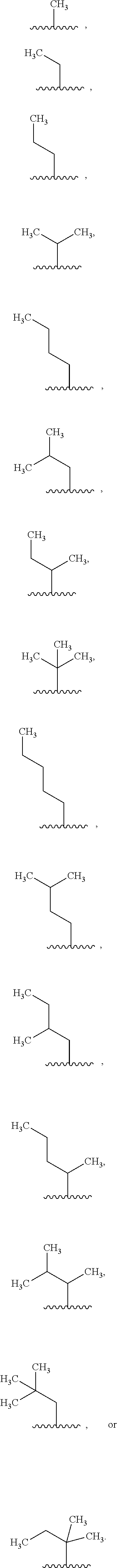

In some embodiments, each of R^{27a} and R^{27b} is H or optionally substituted C₁-C₃ alkyl.

In some embodiments, each of R^{27a} and R^{27b} is, independently, H, hydroxyl,

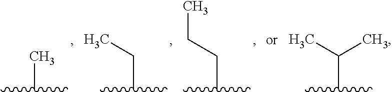

In some embodiments, each of R^{27a} and R^{27b} is, independently, H,

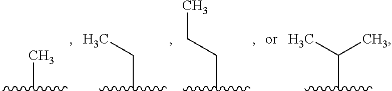

In some embodiments, each of R²⁶, R^{27a}, and R^{27b} is, independently,

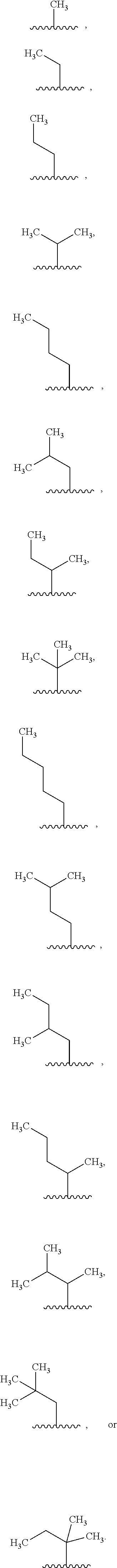

In an aspect, the invention features a compound having the structure of Formula VIII:

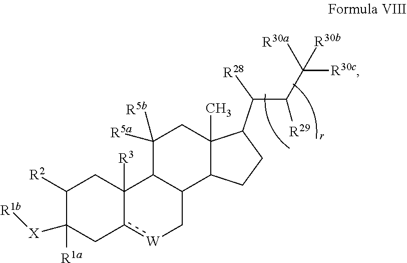

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

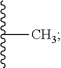

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

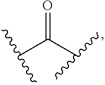 [SMILES: *C(*)=O]

R²⁸ is H or optionally substituted C₁-C₆ alkyl;

r is 1, 2, or 3;

each R²⁹ is, independently, H or optionally substituted C₁-C₆ alkyl; and

each of R^{30a}, R^{30b}, and R^{30c} is C₁-C₆ alkyl, or a pharmaceutically acceptable salt thereof.

In some embodiments, the compound has the structure of Formula VIIIa:

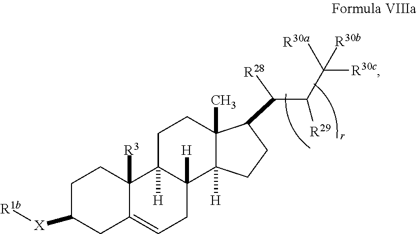

or a pharmaceutically acceptable salt thereof.

In some embodiments, the compound has the structure of Formula VIIIb:

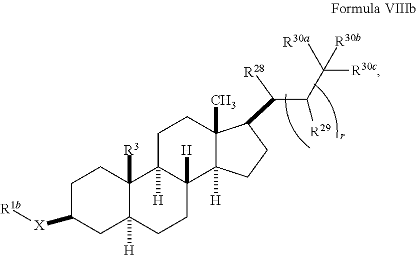

or a pharmaceutically acceptable salt thereof.

In some embodiments, R²⁸ is H,

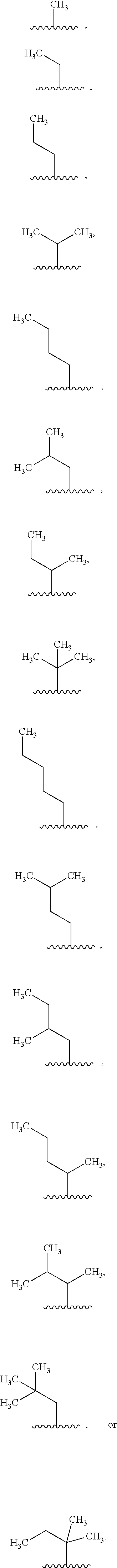

In some embodiments, R²⁸ is

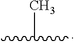

In some embodiments, each of R^{30a}, R^{30b}, and R^{30c} is, independently,

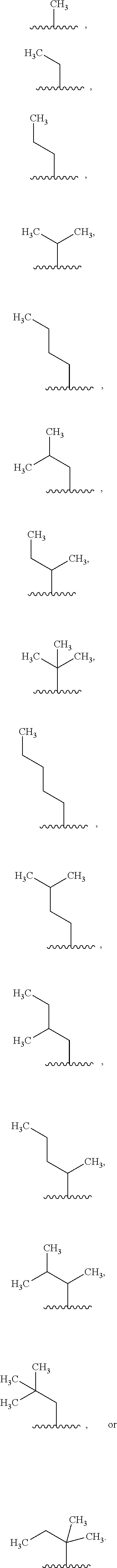

In some embodiments, each of each of R²⁸, R^{30a}, R^{30b}, and R^{30c} is, independently,

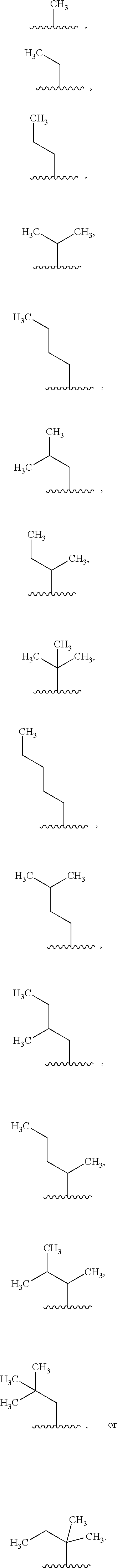

In some embodiments, r is 1. In some embodiments, r is 2. In some embodiments, r is 3.

In some embodiments, each R²⁹ is, independently, H,

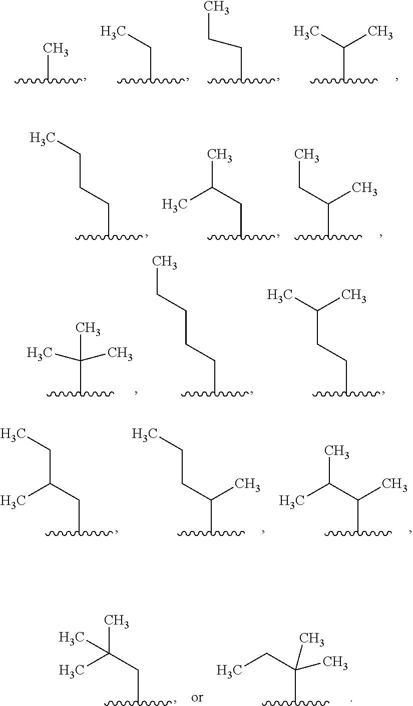

In some embodiments, each R²⁹ is, independently, H or

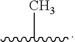

In an aspect, the invention features a compound having the structure of Formula IX:

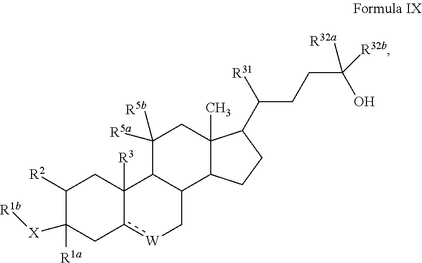

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

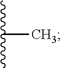

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

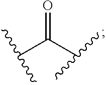 [SMILES: *C(*)=O]

R³¹ is H or C₁-C₆ alkyl; and

each of R^{32a} and R^{32b} is C₁-C₆ alkyl,

or a pharmaceutically acceptable salt thereof.

In some embodiments, the compound has the structure of Formula IXa:

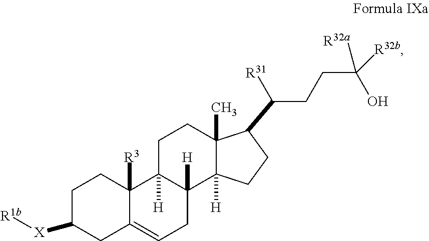

or a pharmaceutically acceptable salt thereof.

In some embodiments, the compound has the structure of Formula IXb:

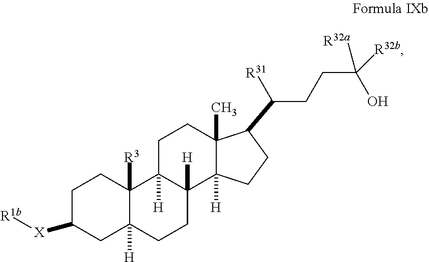

or a pharmaceutically acceptable salt thereof.

In some embodiments, R³¹ is H,

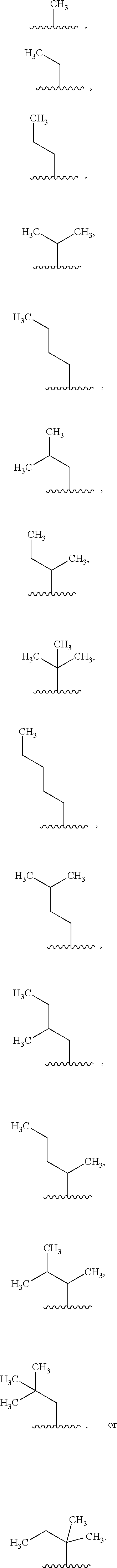

In some embodiments, R³¹ is

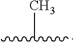

In some embodiments, each of R^{32a} and R^{32b} is, independently,

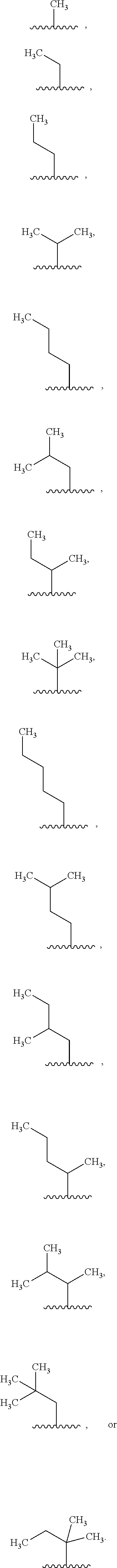

In some embodiments, each of R³¹, R^{32a}, and R^{32b} is, independently,

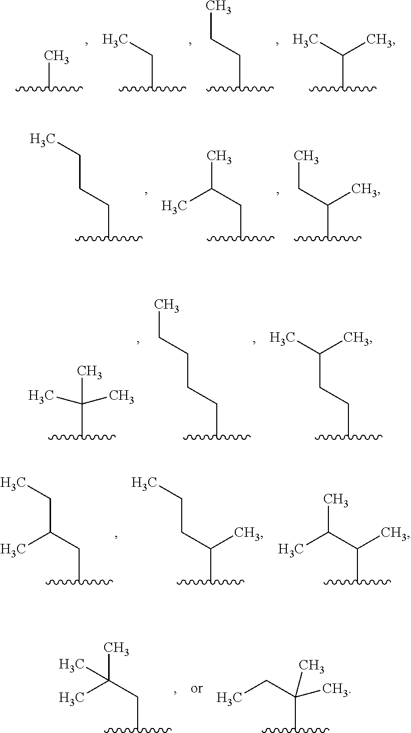

In an aspect, the invention features a compound having the structure of Formula X:

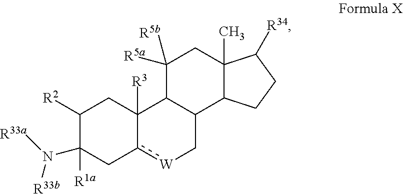

where

R^{1a} is H, optionally substituted C₁-C₆ alkyl, optionally substituted C₂-C₆ alkenyl, or optionally substituted C₂-C₆ alkynyl;

X is O or S;

R² is H or OR^{A}, where R^{A} is H or optionally substituted C₁-C₆ alkyl;

R³ is H or

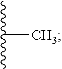

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

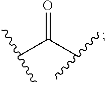 [SMILES: *C(*)=O]

R^{33a} is optionally substituted C₁-C₆ alkyl or

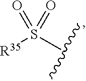

where R³⁵ is optionally substituted C₁-C₆ alkyl or optionally substituted C₆-C₁₀ aryl;

R^{33b} is H or optionally substituted C₁-C₆ alkyl; or

R³⁵ and R^{33b}, together with the atom to which each is attached, form an optionally substituted C₃-C₉ heterocyclyl; and

R³⁴ is optionally substituted C₁-C₆ alkyl or optionally substituted C₁-C₆ heteroalkyl, or a pharmaceutically acceptable salt thereof.

In some embodiments, the compound has the structure of Formula Xa:

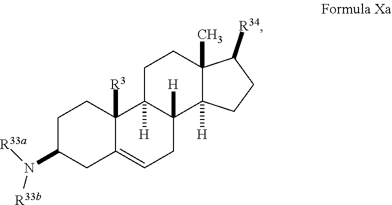

or a pharmaceutically acceptable salt thereof.

In some embodiments, the compound has the structure of Formula Xb:

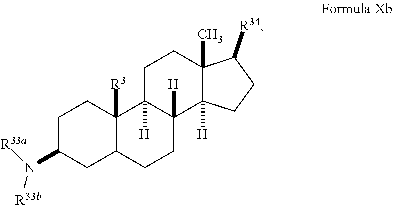

or a pharmaceutically acceptable salt thereof.

In some embodiments, R^{33a} is optionally substituted C₁-C₆ alkyl. In some embodiments, R^{33a} is

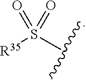

In some embodiments, R^{33b} is H. In some embodiments, R^{33b} is optionally substituted C₁-C₆ alkyl.

In some embodiments, R³⁵ is optionally substituted C₁-C₆ alkyl. In some embodiments, R³⁵ is optionally substituted C₆-C₁₀ aryl.

In some embodiments, R³⁵ is

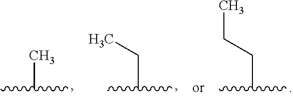

In some embodiments, R³⁵ is

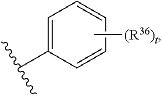

where

t is 0, 1, 2, 3, 4, or 5; and

each R³⁶ is, independently, halo, hydroxyl, optionally substituted C₁-C₆ alkyl, or optionally substituted C₁-C₆ heteroalkyl.

In some embodiments, R³⁵ and R^{33b}, together with the atom to which each is attached, form an optionally substituted C₃-C₉ heterocyclyl.

In some embodiments, R³⁴ is

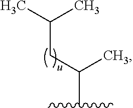

where u is 0, 1, 2, 3, or 4.

In some embodiments, u is 3 or 4.

In an aspect, the invention features a compound having the structure of Formula XI:

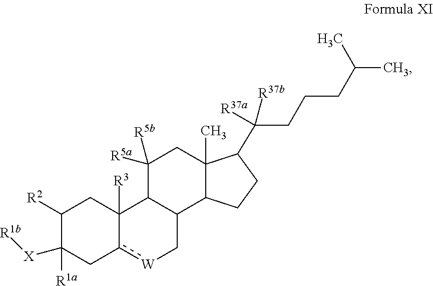

where

R^{1a} is H, optionally substituted C₁-C₆ alkyl, optionally substituted C₂-C₆ alkenyl, or optionally substituted C₂-C₆ alkynyl;

X is O or S;

R² is H or OR^{A}, where R^{A} is H or optionally substituted C₁-C₆ alkyl;

R³ is H or

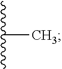

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

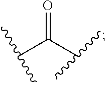 [SMILES: *C(*)=O]

and

each of R^{37a} and R^{37b} is, independently, optionally substituted C₁-C₆ alkyl, optionally substituted C₁-C₆ heteroalkyl, halo, or hydroxyl, or a pharmaceutically acceptable salt thereof.

In some embodiments, the compound has the structure of Formula XIa:

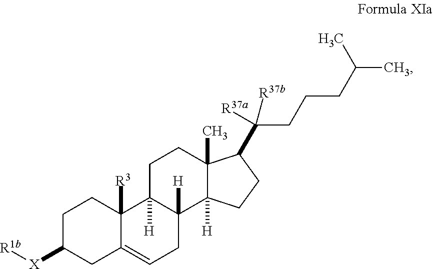

or a pharmaceutically acceptable salt thereof.

In some embodiments, the compound has the structure of Formula XIb:

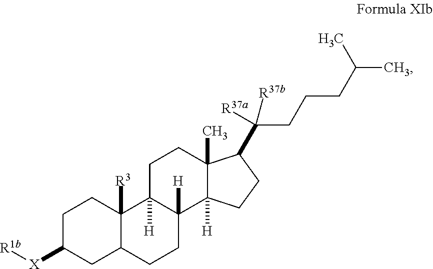

or a pharmaceutically acceptable salt thereof.

In some embodiments, R^{37a} is hydroxyl.

In some embodiments, R^{37b} is

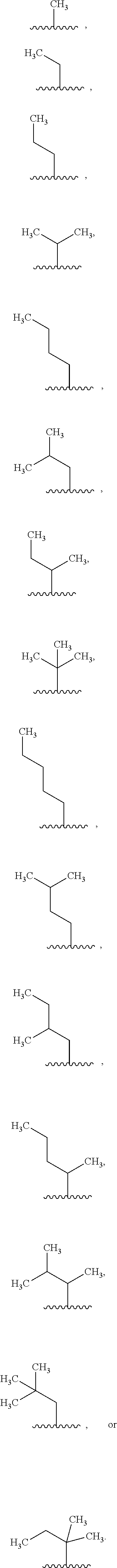

In an aspect, the invention features a compound having the structure of Formula XII:

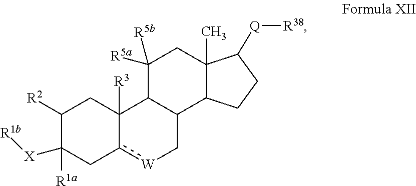

where

R^{1a} is H, optionally substituted C₁-C₆ alkyl, optionally substituted C₂-C₆ alkenyl, or optionally substituted C₂-C₆ alkynyl;

X is O or S;

R² is H or OR^{A}, where R^{A} is H or optionally substituted C₁-C₆ alkyl;

R³ is H or

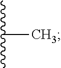

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

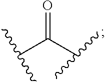 [SMILES: *C(*)=O]

and

Q is O, S, or NR^{E}, where R^{E} is H or optionally substituted C₁-C₆ alkyl; and

R³⁸ is optionally substituted C₁-C₆ alkyl,

or a pharmaceutically acceptable salt thereof.

In some embodiments, the compound has the structure of Formula XIIa:

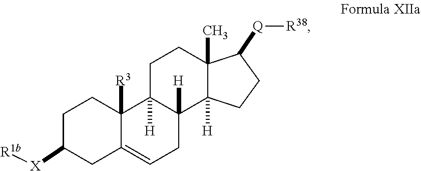

or a pharmaceutically acceptable salt thereof.

In some embodiments, the compound has the structure of Formula XIIb:

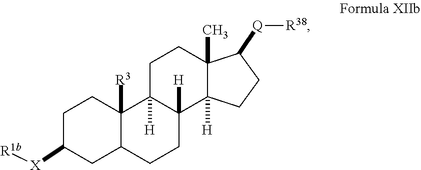

or a pharmaceutically acceptable salt thereof.

In some embodiments, Q is NR^{E}.

In some embodiments, R^{E} is H or

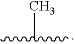

In some embodiments, R^{E} is H. In some embodiments, R^{E} is

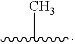

In some embodiments, R³⁸ is

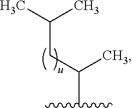

where u is 0, 1, 2, 3, or 4.

In an aspect, the invention features a compound having the structure of Formula XIII:

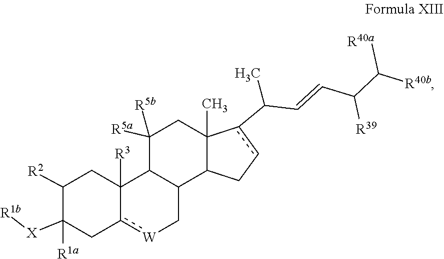

where

R^{1a} is H, optionally substituted C₁-C₆ alkyl, optionally substituted C₂-C₆ alkenyl, or optionally substituted C₂-C₆ alkynyl;

X is O or S;

R^{1b} is H, optionally substituted C₁-C₆ alkyl, or

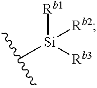

each of R^{b1}, R^{b2}, and R^{b3} is, independently, optionally substituted C₁-C₆ alkyl or optionally substituted C₆-C₁₀ aryl;

R² is H or OR^{A}, where R^{A} is H or optionally substituted C₁-C₆ alkyl;

R³ is H or

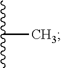

each [symbol] independently represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

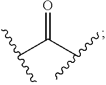 [SMILES: *C(*)=O]

R³⁹ is H or optionally substituted C₄-C₂₀ alkyl;

R^{40a} is C₃-C₂₀ alkyl; and

R^{40b} is C₃-C₂₀ alkyl,

or a pharmaceutically acceptable salt thereof.

In some embodiments, the compound has the structure of Formula XIIIa:

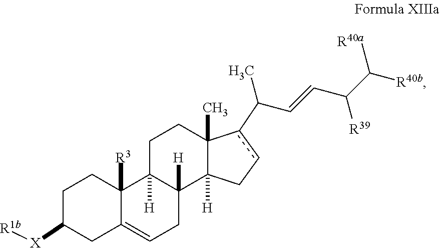

or a pharmaceutically acceptable salt thereof.

In some embodiments, the compound has the structure of Formula XIIIb:

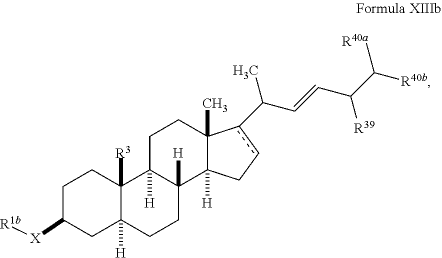

or a pharmaceutically acceptable salt thereof.

In some embodiments, the compound has the structure of Formula XIIIc:

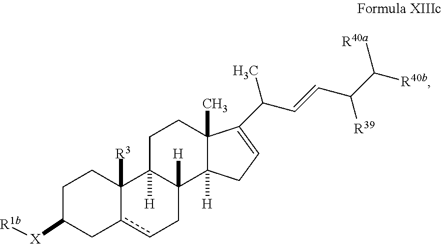

or a pharmaceutically acceptable salt thereof.

In some embodiments, the compound has the structure of Formula XIIId:

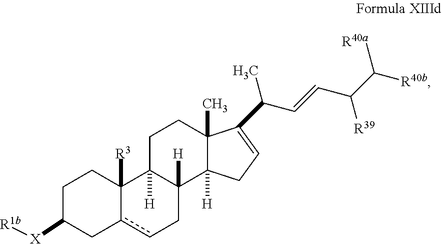

or a pharmaceutically acceptable salt thereof.

In some embodiments, R³⁹ is H. In some embodiments, R³⁹ is optionally substituted C₂-C₂₀ alkyl. In some embodiments, R³⁹ is optionally substituted C₂-C₁₂ alkyl. In some embodiments, R³⁹ is optionally substituted C₂-C₁₀ alkyl. In some embodiments, R³⁹ is optionally substituted C₃-C₂₀ alkyl. In some embodiments, R³⁹ is optionally substituted C₄-C₂₀ alkyl. In some embodiments, R³⁹ is optionally substituted C₅-C₂₀ alkyl. In some embodiments, R³⁹ is optionally substituted C₆-C₂₀ alkyl.

In some embodiments, R³⁹ is

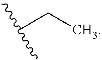 [SMILES: *CC]

In some embodiments, R^{40a} is optionally substituted C₃-C₁₂ alkyl. In some embodiments, R^{40a} is optionally substituted C₃-C₁₀ alkyl.

In some embodiments, R^{40a} is

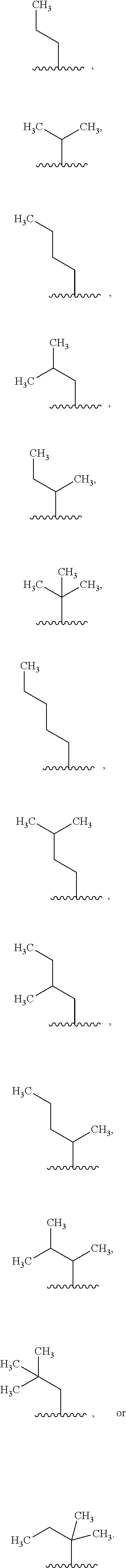

In some embodiments, R^{40a} is

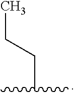 [SMILES: *CCC]

In some embodiments, R^{40a} is optionally substituted C₄-C₂₀ alkyl. In some embodiments, R^{40a} is optionally substituted C₅-C₂₀ alkyl. In some embodiments, R^{40a} is optionally substituted C₆-C₂₀ alkyl.

In some embodiments, R^{40a} is

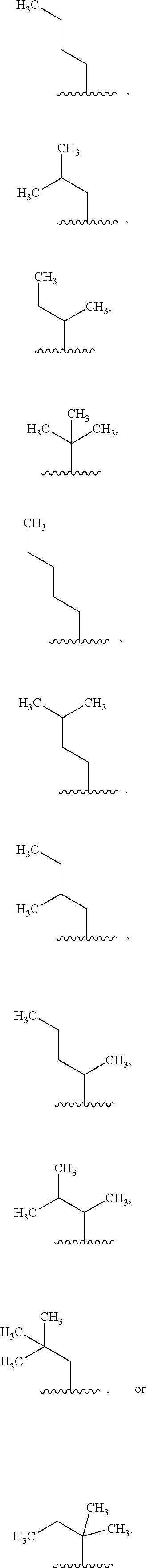

In some embodiments, R^{40a} is

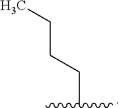 [SMILES: *CCCC]

In some embodiments, R^{40a} is

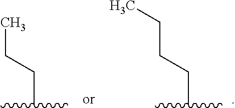

In some embodiments, R^{40b} is optionally substituted C₃-C₁₂ alkyl. In some embodiments, R^{40b} is optionally substituted C₃-C₁₀ alkyl.

In some embodiments, R^{40b} is

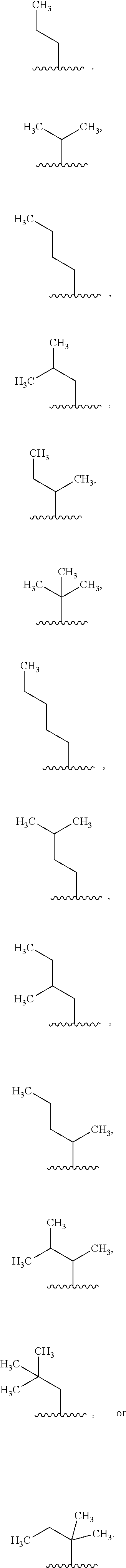

In some embodiments, R^{40b} is

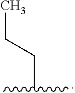 [SMILES: *CCC]

In some embodiments, R^{40b} is optionally substituted C₄-C₂₀ alkyl. In some embodiments, R^{40b} is optionally substituted C₅-C₂₀ alkyl. In some embodiments, R^{40b} is optionally substituted C₆-C₂₀ alkyl.

In some embodiments, R^{40b} is

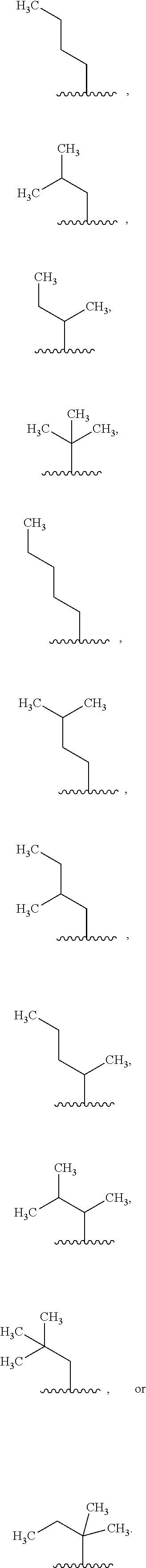

In some embodiments, R^{40b} is

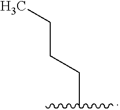 [SMILES: *CCCC]

In some embodiments, R^{40b} is

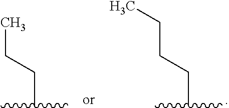

In some embodiments, X is O.

In some embodiments, R^{1a} is H or optionally substituted C₁-C₆ alkyl.

In some embodiments, R^{1a} is H.

In some embodiments, R^{1b} is H or optionally substituted C₁-C₆ alkyl.

In some embodiments, R^{1b} is H.

In some embodiments, R² is H.

In some embodiments, R^{4a} is H.

In some embodiments, R^{4b} is H.

In some embodiments, [symbol] represents a double bond. In some embodiments, [symbol] represents a single bond.

In some embodiments, R³ is H. In some embodiments, R³ is

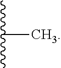

In some embodiments, R^{5a} is H.

In some embodiments, R^{5b} is H.

In some embodiments, the compound has the structure of any one of compounds 1-42, 150, 154, 162-165, 169-172, and 184 in Table 1, or any pharmaceutically acceptable salt thereof. In some embodiments, the compound has the structure of any one of compounds 1-42, 150, 154, 162-165, 169-172, and 184-209 in Table 1, or any pharmaceutically acceptable salt thereof. In some embodiments, the compound has the structure of any one of compounds 1-42, 150, 154, 162-165, 169-172, and 184-207 in Table 1, or any pharmaceutically acceptable salt thereof. In some embodiments, the compound has the structure of any one of compounds 1-42 in Table 1, or any pharmaceutically acceptable salt thereof. In some embodiments, the compound has the structure of any one of compounds 150, 154, 162-165, 169-172, and 184 in Table 1, or any pharmaceutically acceptable salt thereof. In some embodiments, the compound has the structure of any one of compounds 185-209 in Table 1, or any pharmaceutically acceptable salt thereof. In some embodiments, the compound has the structure of any one of compounds 185-207 in Table 1, or any pharmaceutically acceptable salt thereof.

In an aspect, the invention features a compound having the structure of any one of compounds 1-42, 150, 154, 162-165, 169-172, and 184 in Table 1, or any pharmaceutically acceptable salt thereof.

In an aspect, the invention features a compound having the structure of any one of compounds 1-42, 150, 154, 162-165, 169-172, and 184-209 in Table 1, or any pharmaceutically acceptable salt thereof.

In an aspect, the invention features a compound having the structure of any one of compounds 1-42, 150, 154, 162-165, 169-172, and 184-207 in Table 1, or any pharmaceutically acceptable salt thereof.

In an aspect, the invention features a compound having the structure of any one of compounds 1-42 in Table 1, or any pharmaceutically acceptable salt thereof.

In an aspect, the invention features a compound having the structure of any one of compounds 150, 154, 162-165, 169-172, and 184 in Table 1, or any pharmaceutically acceptable salt thereof.

In an aspect, the invention features a compound having the structure of any one of compounds 185-209 in Table 1, or any pharmaceutically acceptable salt thereof.

In an aspect, the invention features a compound having the structure of any one of compounds 185-207 in Table 1, or any pharmaceutically acceptable salt thereof.

As used herein, “CMPD” refers to “compound.”

TABLE 1
Compounds of Formula I

| CMPD | |
|---|---|
| No. | Structure |
| 1 | 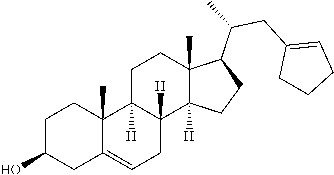 |
| 2 | 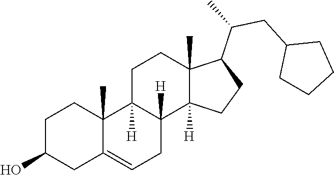 |
| 3 | 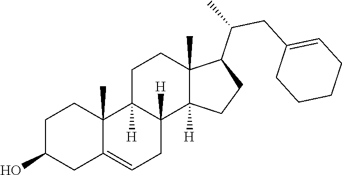 |
| 4 | 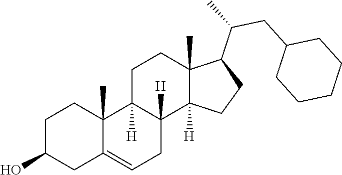 |
| 5 | 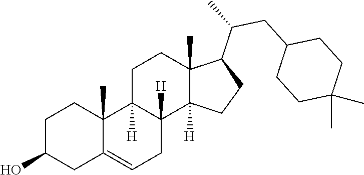 |
| 6 | 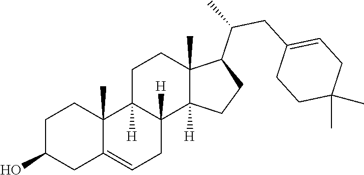 |
| 7 | 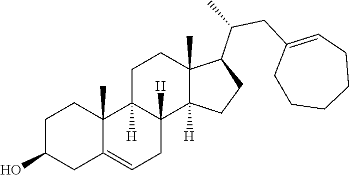 |
| 8 | 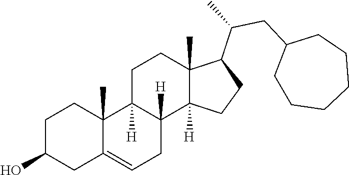 |
| 9 | 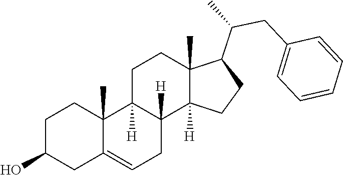 |
| 10 | 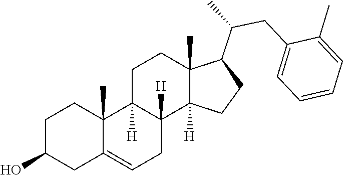 |
| 11 | 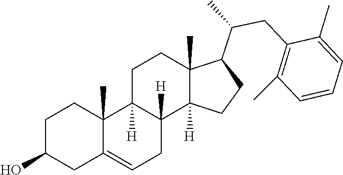 |
| 12 | 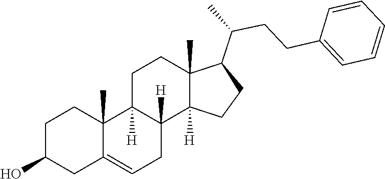 |
| 13 | 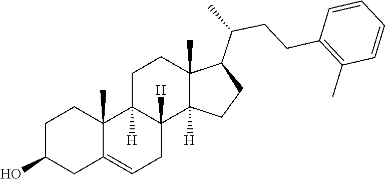 |
| 14 | 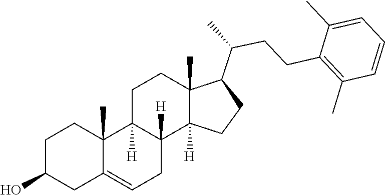 |
| 15 | 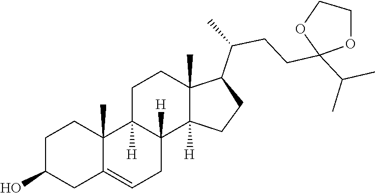 |
| 16 | 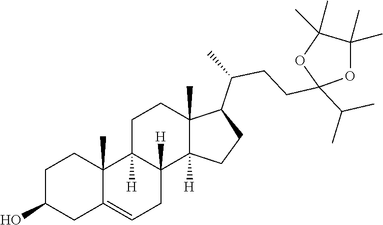 |
| 17 | 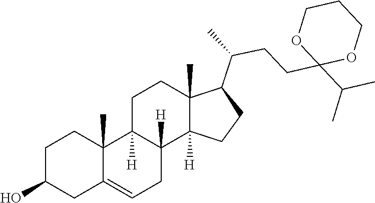 |
| 18 | 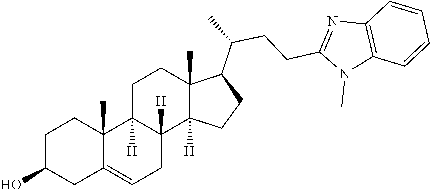 |
| 19 | 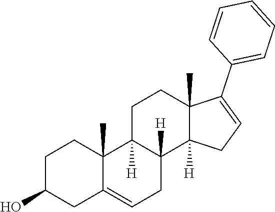 |
| 20 | 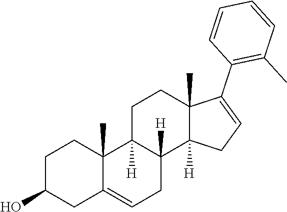 |
| 21 | 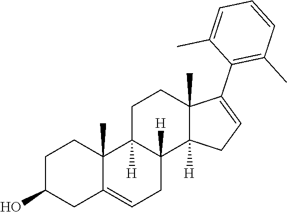 |
| 22 | 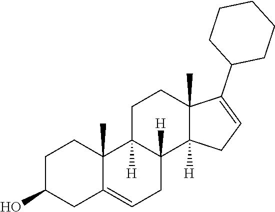 |
| 23 | 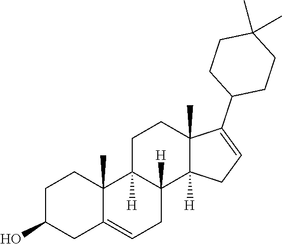 |
| 24 | 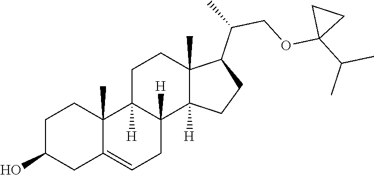 |
| 25 | 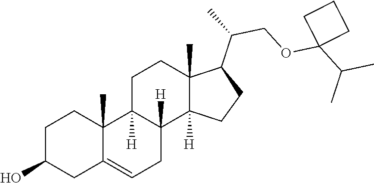 |
| 26 | 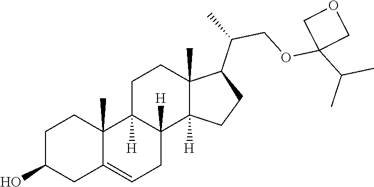 |
| 27 | 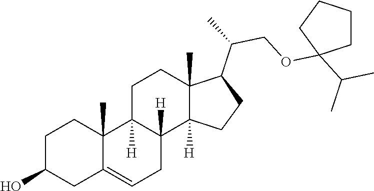 |
| 28 | 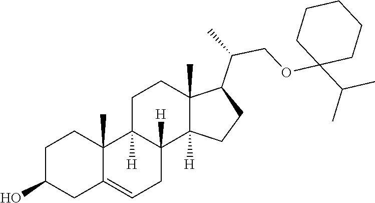 |
| 29 | 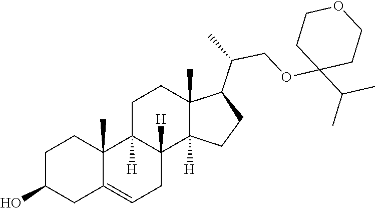 |
| 30 | 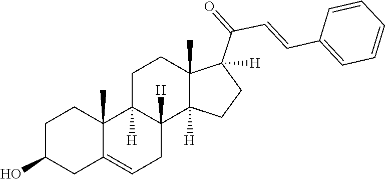 |
| 31 | 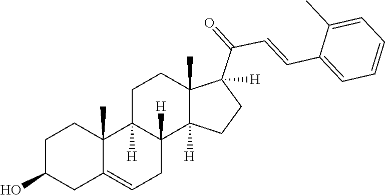 |
| 32 | 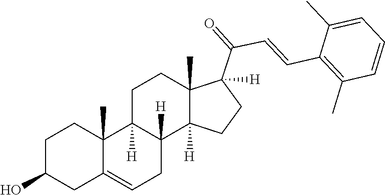 |
| 33 | 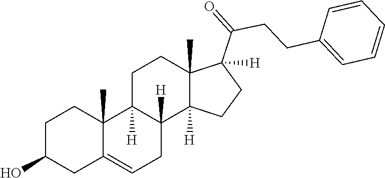 |
| 34 | 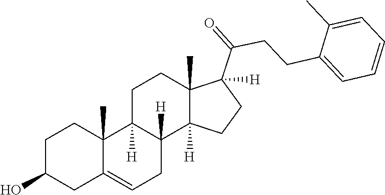 |
| 35 | 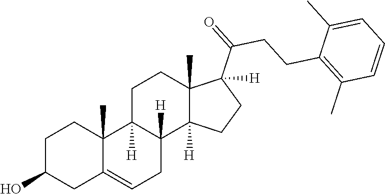 |
| 36 | 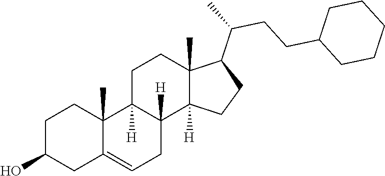 |
| 37 | 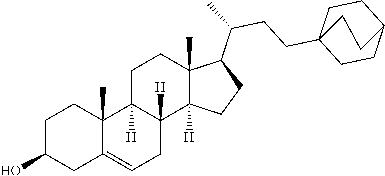 |
| 38 | 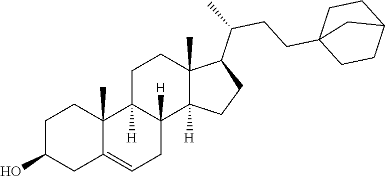 |
| 39 | 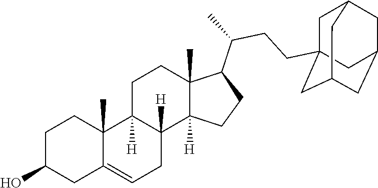 |
| 40 | 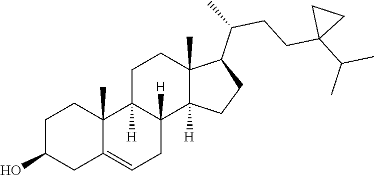 |
| 41 | 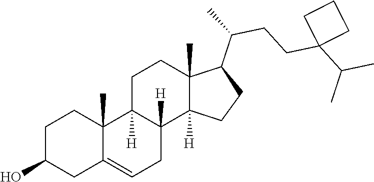 |
| 42 | 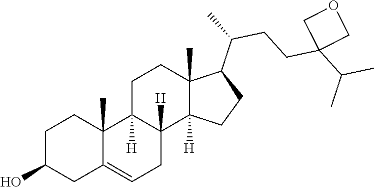 |
| 150 | 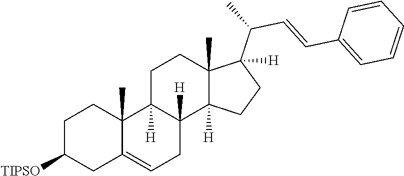 |
| 154 | 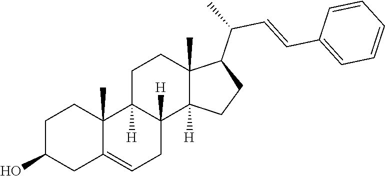 |
| 162 | 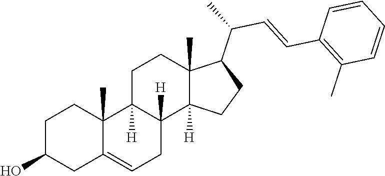 |
| 163 | 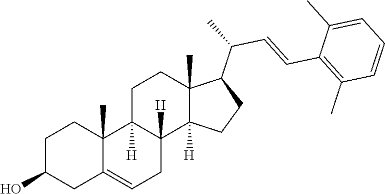 |
| 164 | 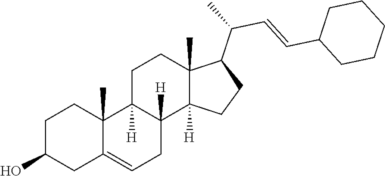 |
| 165 | 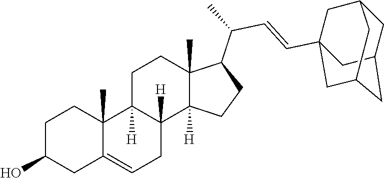 |
| 169 | 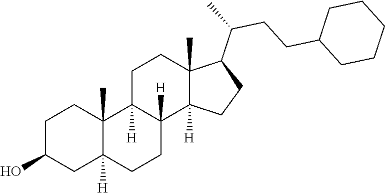 |
| 170 | 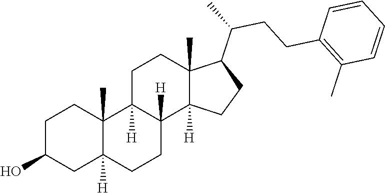 |
| 171 | 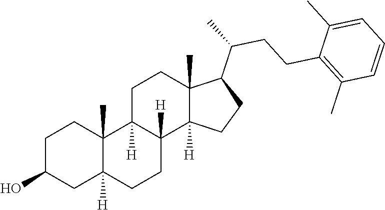 |
| 172 | 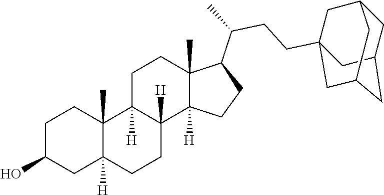 |
| 184 | 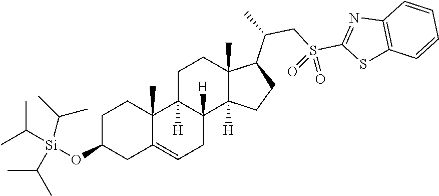 |
| 185 | 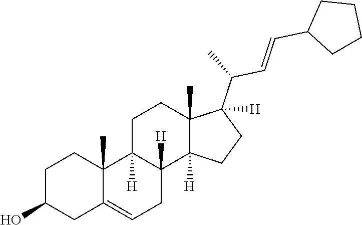 |
| 186 | 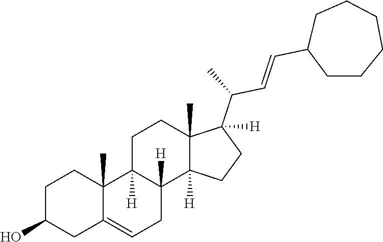 |
| 187 | 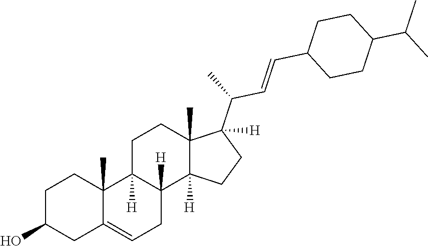 |
| 188 | 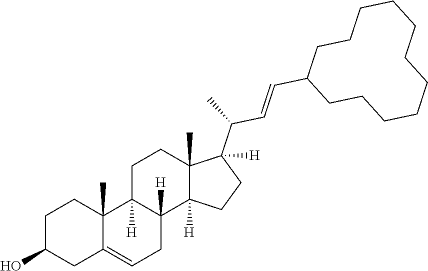 |
| 189 | 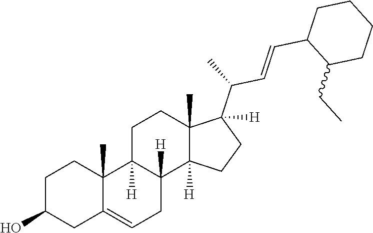 |
| 190 | 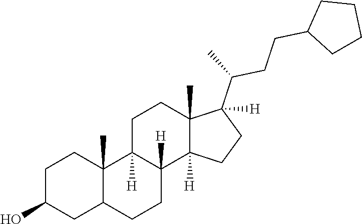 |
| 191 | 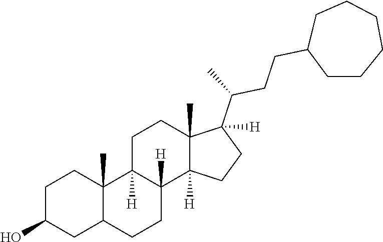 |
| 192 | 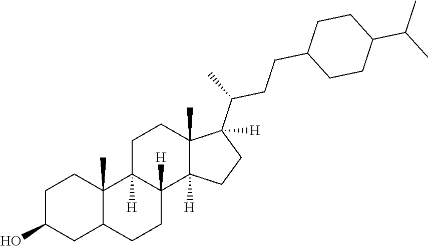 |
| 193 | 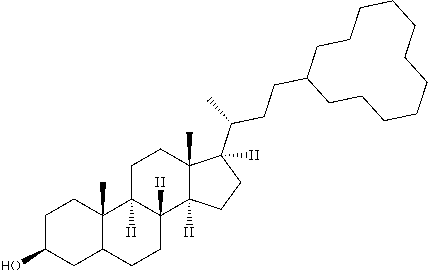 |
| 194 | 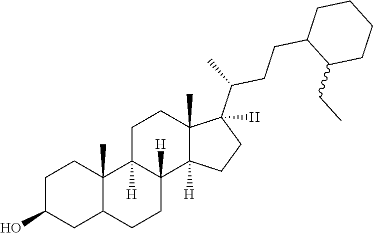 |
| 195 | 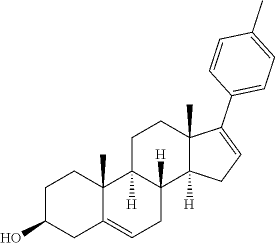 |
| 196 | 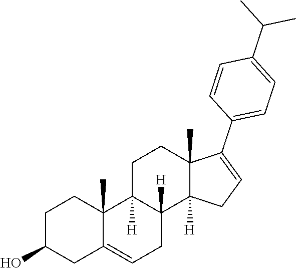 |
| 197 | 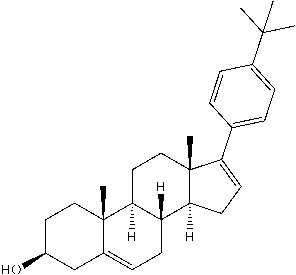 |
| 198 | 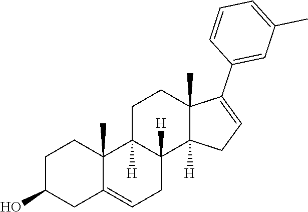 |
| 199 | 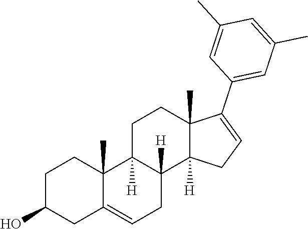 |
| 200 | 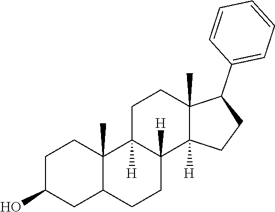 |
| 201 | 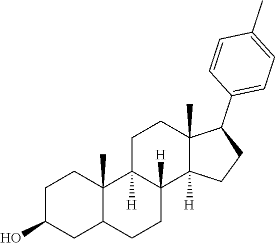 |
| 202 | 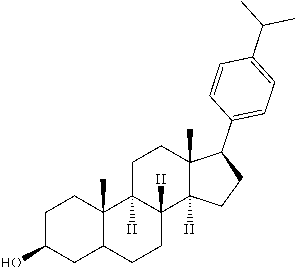 |
| 203 | 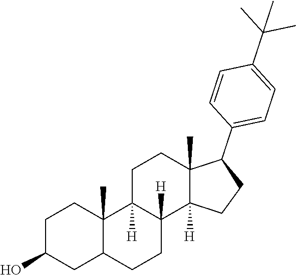 |
| 204 | 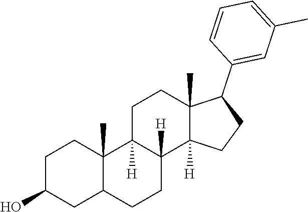 |
| 205 | 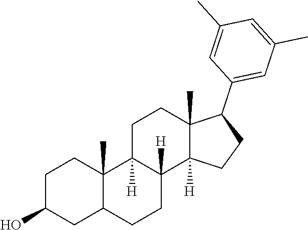 |
| 206 | 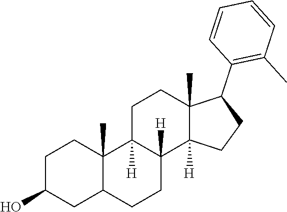 |
| 207 | 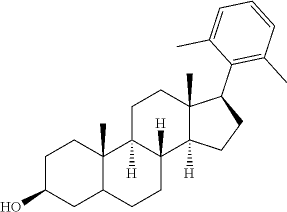 |
| 208 | 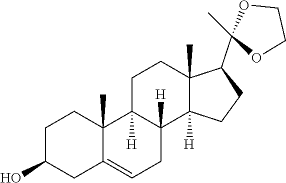 |
| 209 | 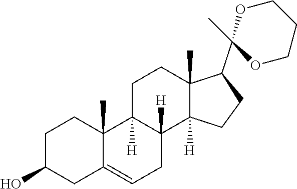 |

In some embodiments, the compound has the structure of any one of compounds 43-50 and 175-178 in Table 2, or any pharmaceutically acceptable salt thereof. In some embodiments, the compound has the structure of any one of compounds 43-50 in Table 2, or any pharmaceutically acceptable salt thereof. In some embodiments, the compound has the structure of any one of compounds 175-178 in Table 2, or any pharmaceutically acceptable salt thereof.

In an aspect, the invention features a compound having the structure of any one of compounds 43-50 and 175-178 in Table 2, or any pharmaceutically acceptable salt thereof.

In an aspect, the invention features a compound having the structure of any one of compounds 43-50 in Table 2, or any pharmaceutically acceptable salt thereof.

In an aspect, the invention features a compound having the structure of any one of compounds 175-178 in Table 2, or any pharmaceutically acceptable salt thereof.

TABLE 2
Compounds of Formula II

| CMPD | |
|---|---|
| No. | Structure |
| 43 | 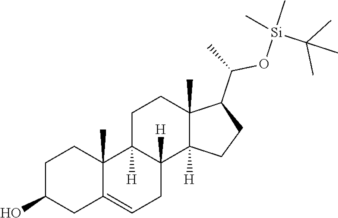 |
| 44 | 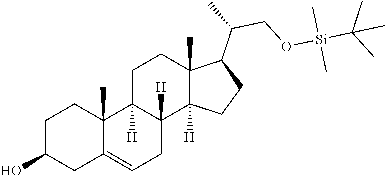 |
| 45 | 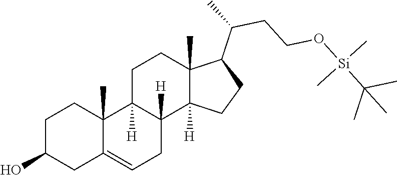 |
| 46 | 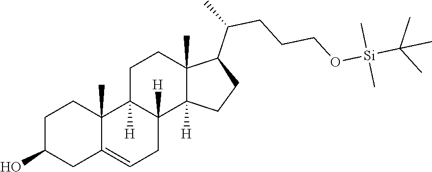 |
| 47 | 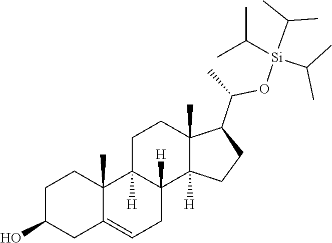 |
| 48 | 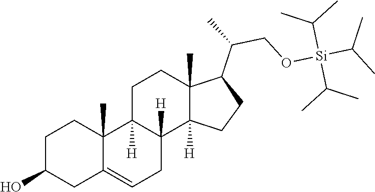 |
| 49 | 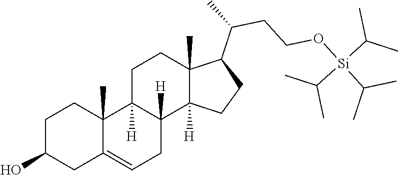 |
| 50 | 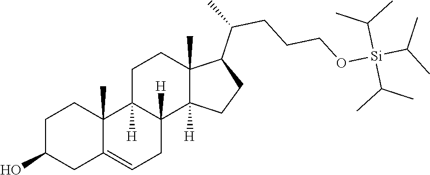 |
| 175 | 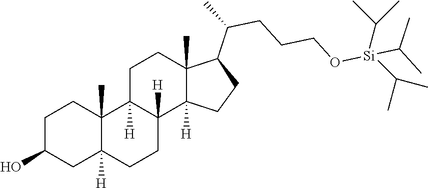 |
| 176 | 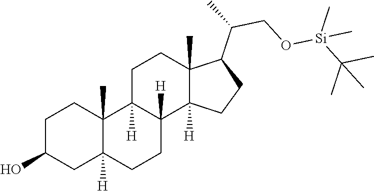 |
| 177 | 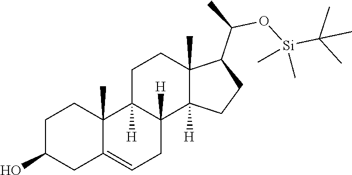 |
| 178 | 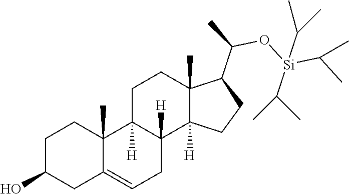 |

In some embodiments, the compound has the structure of any one of compounds 51-67, 149, and 153 in Table 3, or any pharmaceutically acceptable salt thereof. In some embodiments, the compound has the structure of any one of compounds 51-67 and 149 in Table 3, or any pharmaceutically acceptable salt thereof. In some embodiments, the compound has the structure of compound 153 in Table 3, or any pharmaceutically acceptable salt thereof.

In an aspect, the invention features a compound having the structure of any one of compounds 51-67, 149, and 153 in Table 3, or any pharmaceutically acceptable salt thereof.

In an aspect, the invention features a compound having the structure of any one of compounds 51-67 and 149 in Table 3, or any pharmaceutically acceptable salt thereof.

In an aspect, the invention features a compound having the structure of compound 153 in Table 3, or any pharmaceutically acceptable salt thereof.

TABLE 3
Compounds of Formula III

| CMPD | |
|---|---|
| No. | Structure |
| 51 | 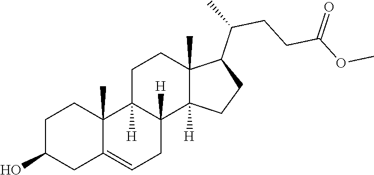 |
| 52 | 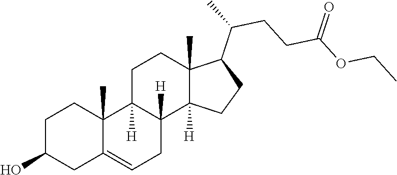 |
| 53 | 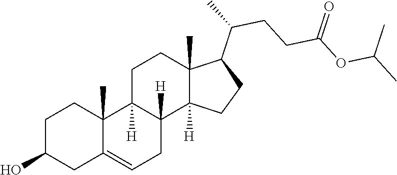 |
| 54 | 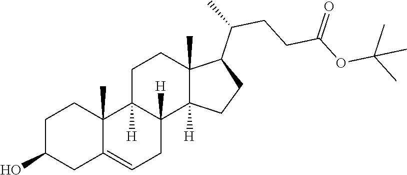 |
| 55 | 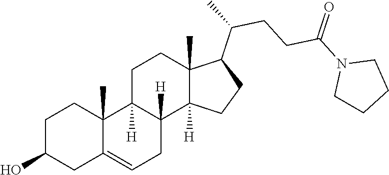 |
| 56 | 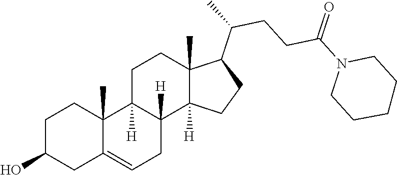 |
| 57 | 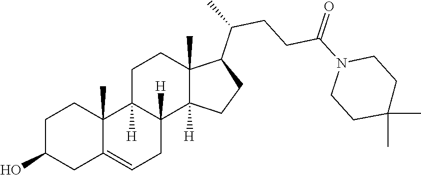 |
| 58 | 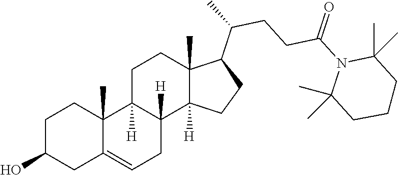 |
| 59 | 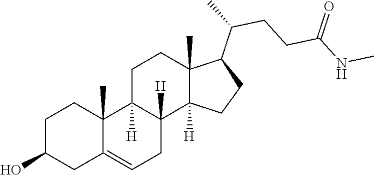 |
| 60 | 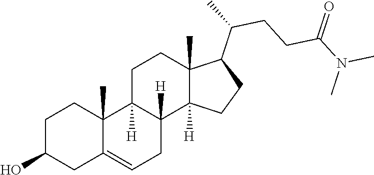 |
| 61 | 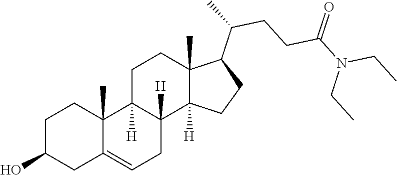 |
| 62 | 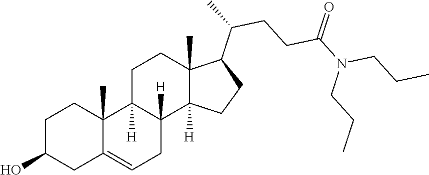 |
| 63 | 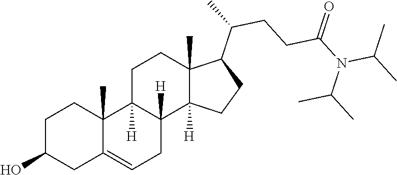 |
| 64 | 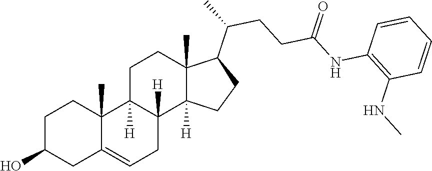 |
| 65 | 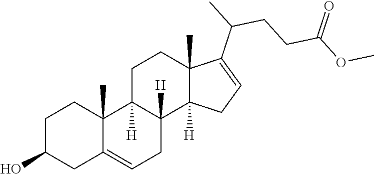 |
| 66 | 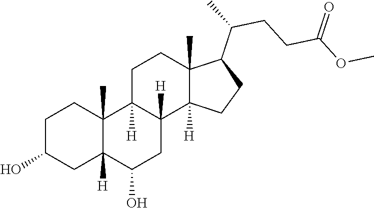 |
| 67 | 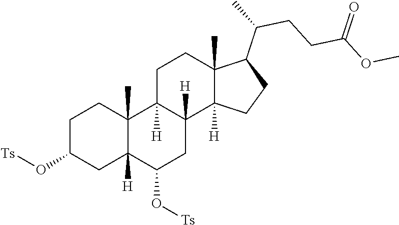 |
| 149 | 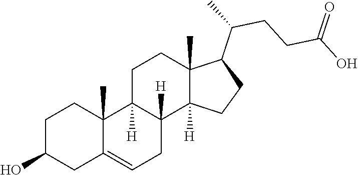 |
| 153 | 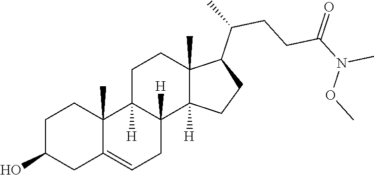 |

In some embodiments, the compound has the structure of any one of compounds 68-73 in Table 4, or any pharmaceutically acceptable salt thereof.

In an aspect, the invention features a compound having the structure of any one of compounds 68-73 in Table 4, or any pharmaceutically acceptable salt thereof.

TABLE 4
Compounds of Formula IV

| CMPD | |
|---|---|
| No. | Structure |
| 68 | 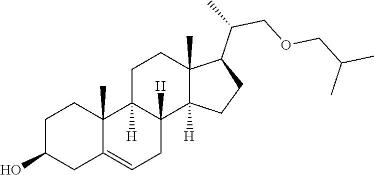 |
| 69 | 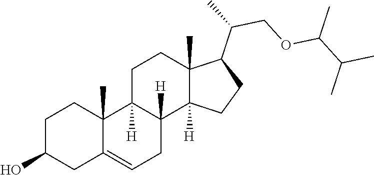 |
| 70 | 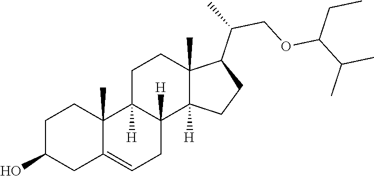 |
| 71 | 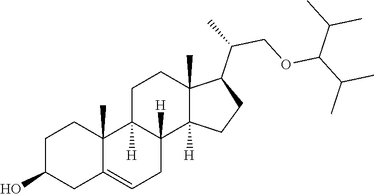 |
| 72 | 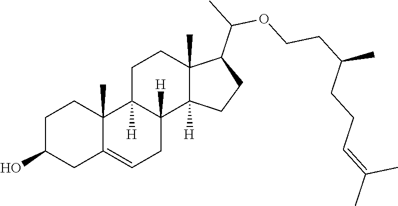 |
| 73 | 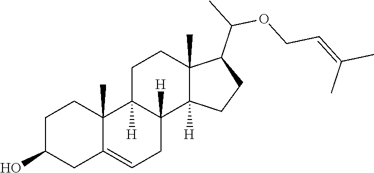 |

In some embodiments, the compound has the structure of any one of compounds 74-78 in Table 5, or any pharmaceutically acceptable salt thereof.

In an aspect, the invention features a compound having the structure of any one of compounds 74-78 in Table 5, or any pharmaceutically acceptable salt thereof.

TABLE 5
Compounds of Formula V

| CMPD | |
|---|---|
| No. | Structure |
| 74 | 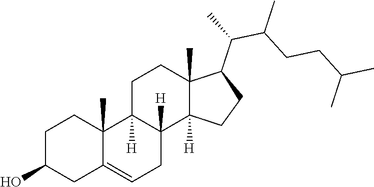 |
| 75 | 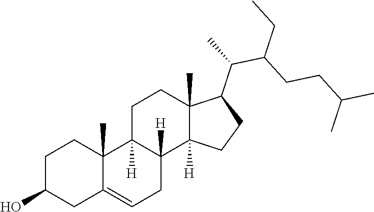 |
| 76 | 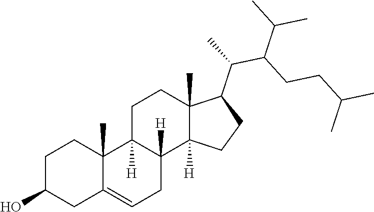 |
| 77 | 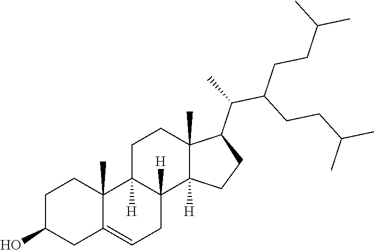 |
| 78 | 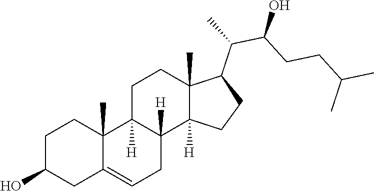 |

In some embodiments, the compound has the structure of any one of compounds 79 and 80 in Table 6, or any pharmaceutically acceptable salt thereof.

In an aspect, the invention features a compound having the structure of any one of compounds 79 and 80 in Table 6, or any pharmaceutically acceptable salt thereof.

TABLE 6
Compounds of Formula VI

| CMPD | |
|---|---|
| No. | Structure |
| 79 | 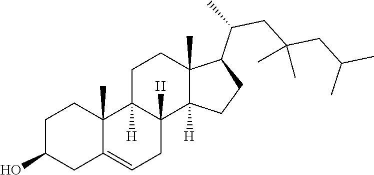 |
| 80 | 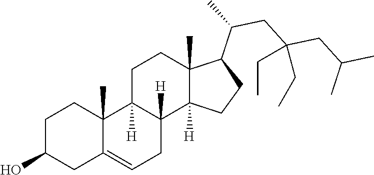 |

In an aspect, the invention features a compound having the structure of any one of compounds 81-87, 152, and 157 in Table 7, or any pharmaceutically acceptable salt thereof.

In some embodiments, the compound has the structure of any one of compounds 81-83, 85-87, 152, and 157 in Table 7, or any pharmaceutically acceptable salt thereof. In some embodiments, the compound has the structure of any one of compounds 81-83 and 85-87 in Table 7, or any pharmaceutically acceptable salt thereof. In some embodiments, the compound has the structure of any one of compounds 152 and 157 in Table 7, or any pharmaceutically acceptable salt thereof.

In an aspect, the invention features a compound having the structure of any one of compounds 81-83, 85-87, 152, and 157 in Table 7, or any pharmaceutically acceptable salt thereof.

In an aspect, the invention features a compound having the structure of any one of compounds 81-83 and 85-87 in Table 7, or any pharmaceutically acceptable salt thereof.

In an aspect, the invention features a compound having the structure of any one of compounds 152 and 157 in Table 7, or any pharmaceutically acceptable salt thereof.

TABLE 7
Compounds of Formula VII

| CMPD | |
|---|---|
| No. | Structure |
| 81 | 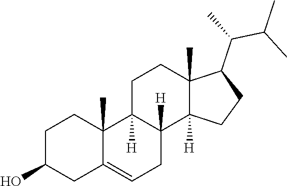 |
| 82 | 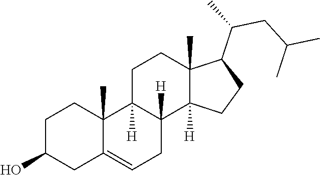 |
| 83 | 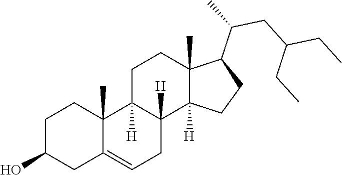 |
| 85 | 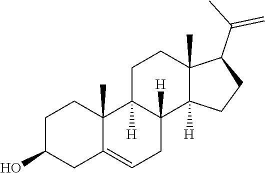 |
| 86 | 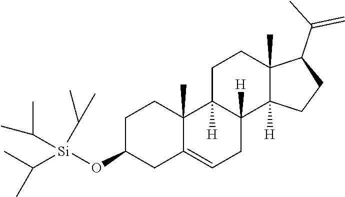 |
| 87 | 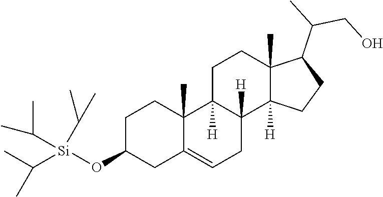 |
| 152 | 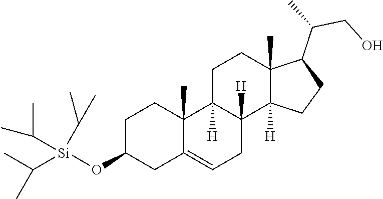 |
| 157 | 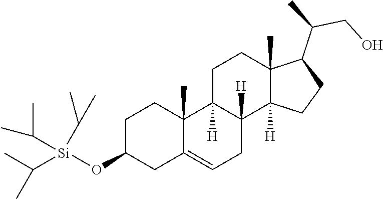 |

In some embodiments, the compound has the structure of any one of compounds 88-97 in Table 8, or any pharmaceutically acceptable salt thereof.

In an aspect, the invention features a compound having the structure of any one of compounds 88-97 in Table 8, or any pharmaceutically acceptable salt thereof.

TABLE 8
Compounds of Formula VIII

| CMPD | |
|---|---|
| No. | Structure |
| 88 | 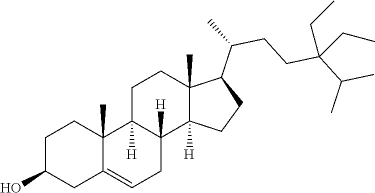 |
| 89 | 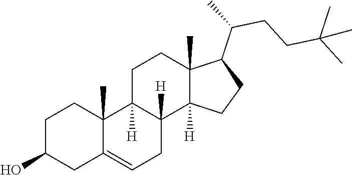 |
| 90 | 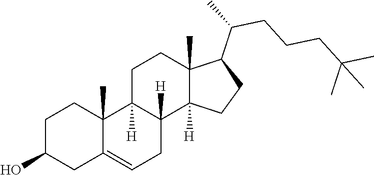 |
| 91 | 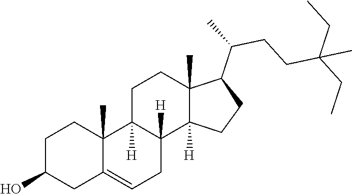 |
| 92 | 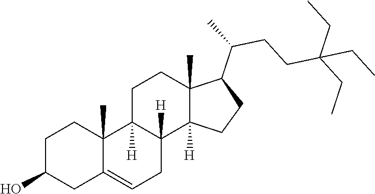 |
| 93 | 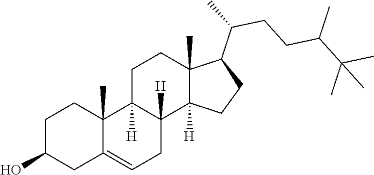 |
| 94 | 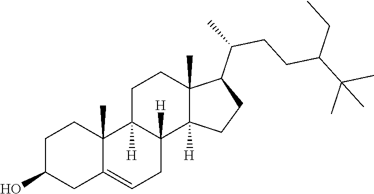 |
| 95 | 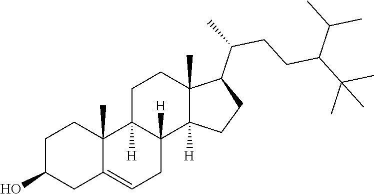 |
| 96 | 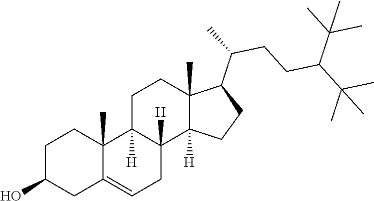 |
| 97 | 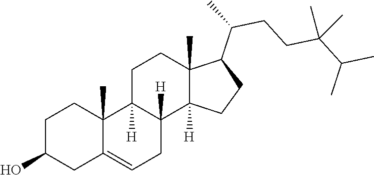 |

In some embodiments, the compound has the structure of any one of compounds 98-105 and 180-182 in Table 9, or any pharmaceutically acceptable salt thereof. In some embodiments, the compound has the structure of any one of compounds 98-105, 180-182, and 210-213 in Table 9, or any pharmaceutically acceptable salt thereof. In some embodiments, the compound has the structure of any one of compounds 98-105 in Table 9, or any pharmaceutically acceptable salt thereof. In some embodiments, the compound has the structure of any one of compounds 180-182 in Table 9, or any pharmaceutically acceptable salt thereof. In some embodiments, the compound has the structure of any one of compounds 210-213 in Table 9, or any pharmaceutically acceptable salt thereof.

In an aspect, the invention features a compound having the structure of any one of compounds 98-105 and 180-182 in Table 9, or any pharmaceutically acceptable salt thereof.

In an aspect, the invention features a compound having the structure of any one of compounds 98-105, 180-182, and 210-213 in Table 9, or any pharmaceutically acceptable salt thereof.

In an aspect, the invention features a compound having the structure of any one of compounds 98-105 in Table 9, or any pharmaceutically acceptable salt thereof.

In an aspect, the invention features a compound having the structure of any one of compounds 180-182 in Table 9, or any pharmaceutically acceptable salt thereof.

In an aspect, the invention features a compound having the structure of any one of compounds 210-213 in Table 9, or any pharmaceutically acceptable salt thereof.

TABLE 9
Compounds of Formula IX

| CMPD | |
|---|---|
| No. | Structure |
| 98 | 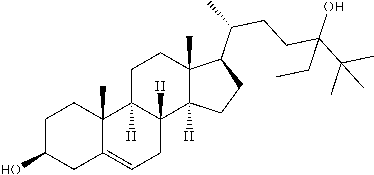 |
| 99 | 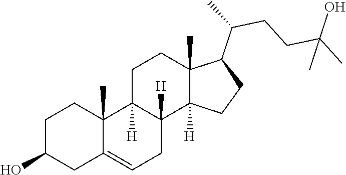 |
| 100 | 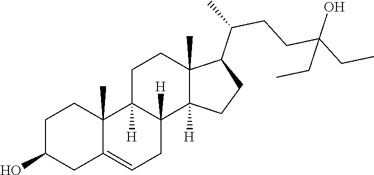 |
| 101 | 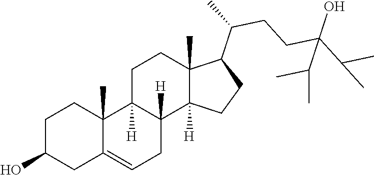 |
| 102 | 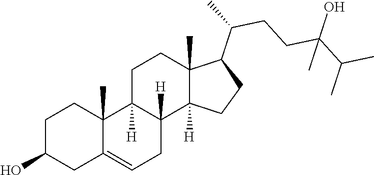 |
| 103 | 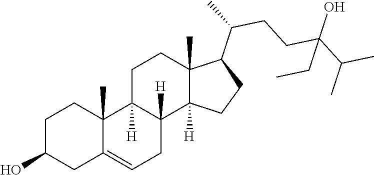 |
| 104 | 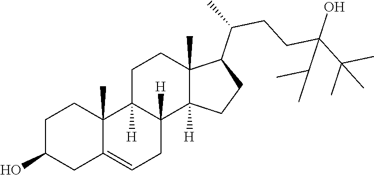 |
| 105 | 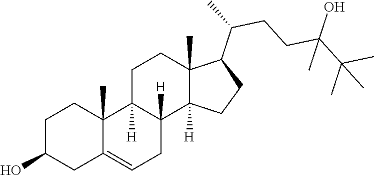 |
| 180 | 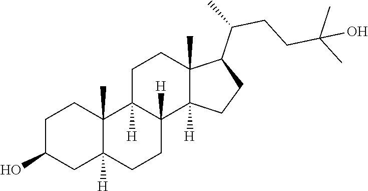 |
| 181 | 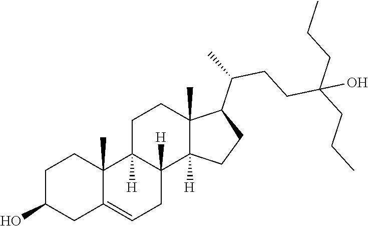 |
| 182 | 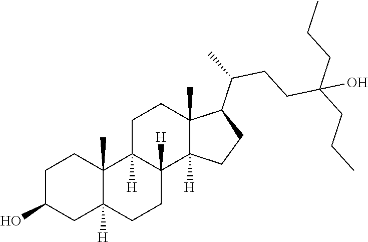 |
| 210 | 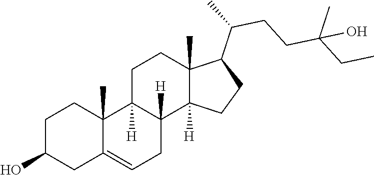 |
| 211 | 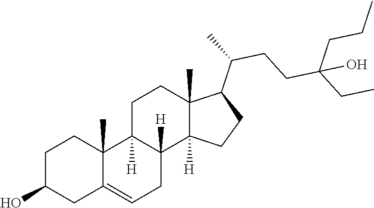 |
| 212 | 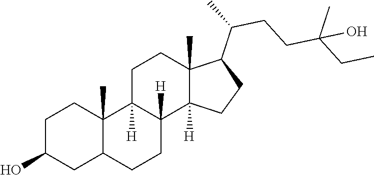 |
| 213 | 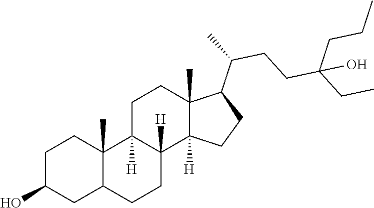 |

In some embodiments, the compound has the structure of compound 106 in Table 10, or any pharmaceutically acceptable salt thereof.

In an aspect, the invention features a compound having the structure of compound 106 in Table 10, or any pharmaceutically acceptable salt thereof.

TABLE 10
Compounds of Formula X

| CMPD | |
|---|---|
| No. | Structure |
| 106 | 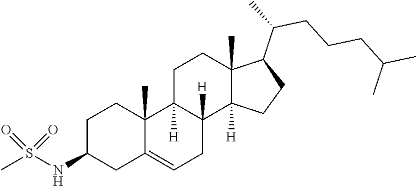 |

In some embodiments, the compound has the structure of compound 107 or 108 in Table 11, or any pharmaceutically acceptable salt thereof.

In an aspect, the invention features a compound having the structure of compound 107 or 108 in Table 11, or any pharmaceutically acceptable salt thereof.

TABLE 11
Compounds of Formula XI

| CMPD | |
|---|---|
| No. | Structure |
| 107 | 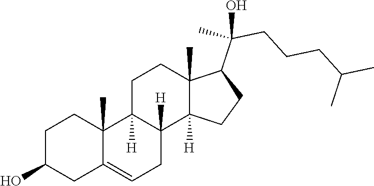 |
| 108 | 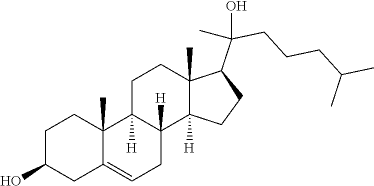 |

In some embodiments, the compound has the structure of compound 109 in Table 12, or any pharmaceutically acceptable salt thereof.

In an aspect, the invention features a compound having the structure of compound 109 in Table 12, or any pharmaceutically acceptable salt thereof.

TABLE 12
Compounds of Formula XII

| CMPD | |
|---|---|
| No. | Structure |
| 109 | 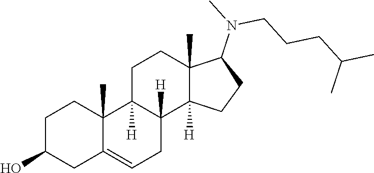 |

In some embodiments, the compound has the structure of any one of compounds 214-218 in Table 13, or any pharmaceutically acceptable salt thereof.

In an aspect, the invention features a compound having the structure of any one of compounds 214-218 in Table 13, or any pharmaceutically acceptable salt thereof.

TABLE 13
Compounds of Formula XIII

| CMPD | |
|---|---|
| No. | Structure |
| 214 | 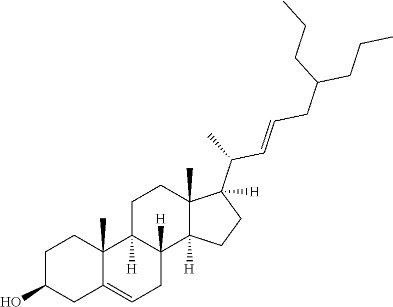 |
| 215 | 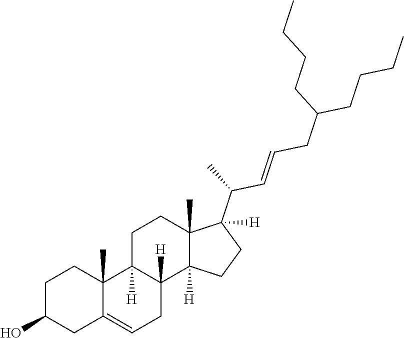 |
| 217 | 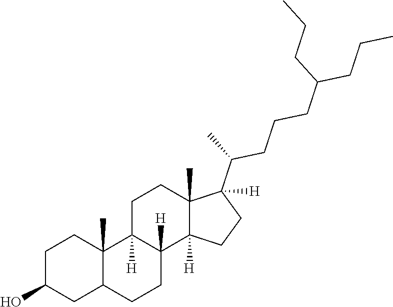 |
| 216 | 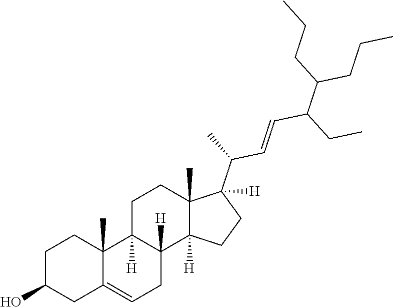 |
| 218 | 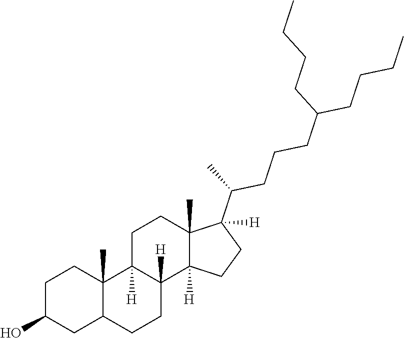 |

In some embodiments, the compound has the structure of any one of compounds 110-130, 155, 156, 158, 160, 161, 166-168, 173, 174, and 179 in Table 14, or any pharmaceutically acceptable salt thereof. In some embodiments, the compound has the structure of any one of compounds 110-130, 155, 156, 158, 160, 161, 166-168, 173, 174, 179, and 219-226 in Table 14, or any pharmaceutically acceptable salt thereof. In some embodiments, the compound has the structure of any one of compounds 219-226 in Table 14, or any pharmaceutically acceptable salt thereof.

In an aspect, the invention features a compound having the structure of any one of compounds 110-130, 155, 156, 158, 160, 161, 166-168, 173, 174, and 179 in Table 14, or any pharmaceutically acceptable salt thereof.

In an aspect, the invention features a compound having the structure of any one of compounds 219-226 in Table 14, or any pharmaceutically acceptable salt thereof.

In an aspect, the invention features a compound having the structure of any one of compounds 110-130, 155, 156, 158, 160, 161, 166-168, 173, 174, 179, and 219-226 in Table 14, or any pharmaceutically acceptable salt thereof.

TABLE 14
Compounds of the Invention

| CMPD | |
|---|---|
| No. | Structure |
| 110 | 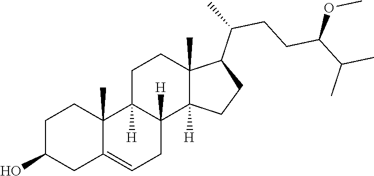 |
| 111 | 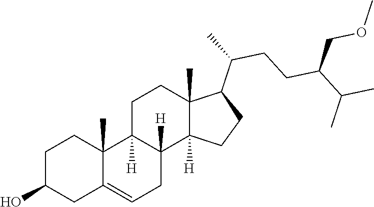 |
| 112 | 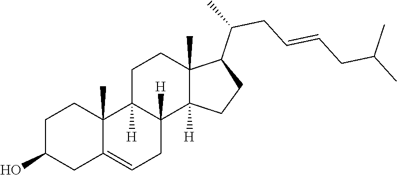 |
| 113 | 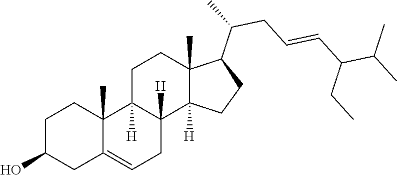 |
| 114 | 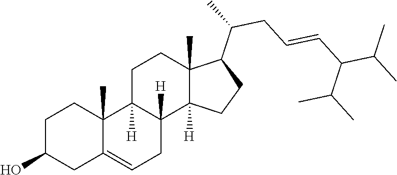 |
| 115 | 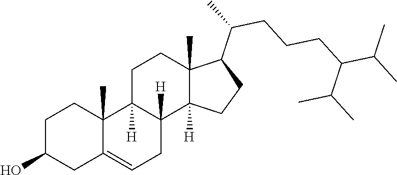 |
| 116 | 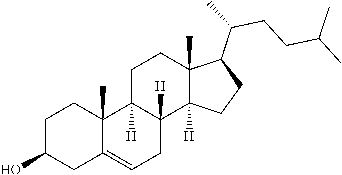 |
| 117 | 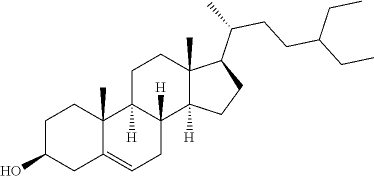 |
| 118 | 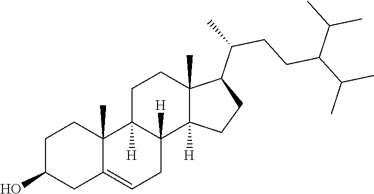 |
| 119 | 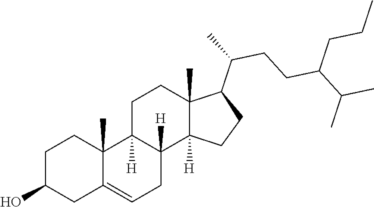 |
| 120 | 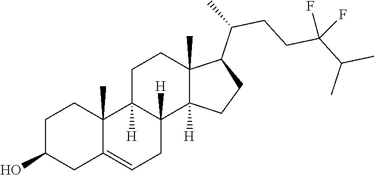 |
| 121 | 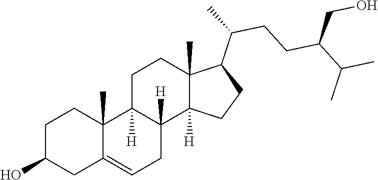 |
| 122 | 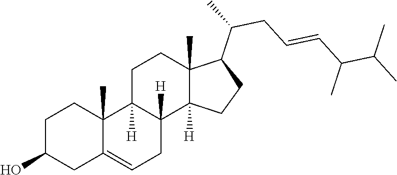 |
| 123 | 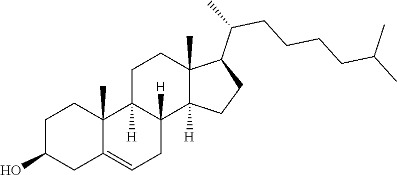 |
| 124 | 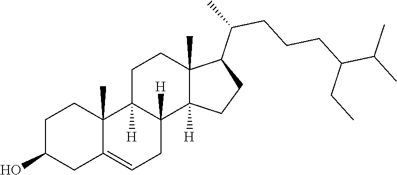 |
| 125 | 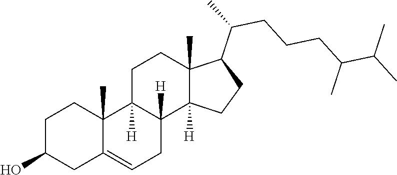 |
| 126 | 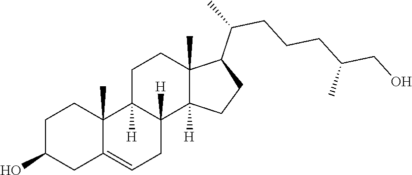 |
| 127 | 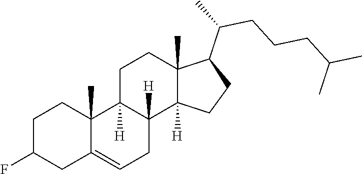 |
| 128 | 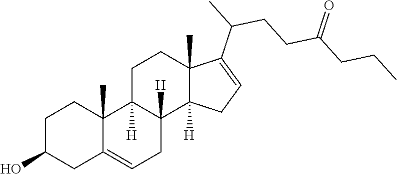 |
| 129 | 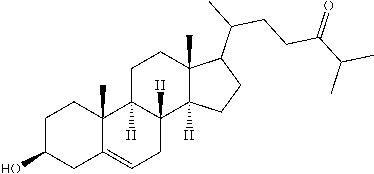 |
| 130 | 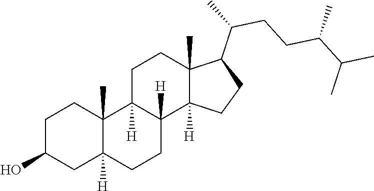 |
| 155 | 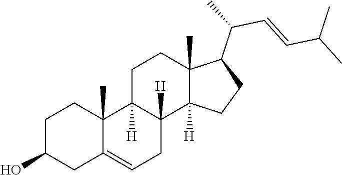 |
| 156 | 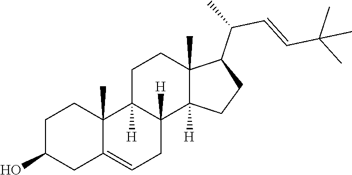 |
| 158 | 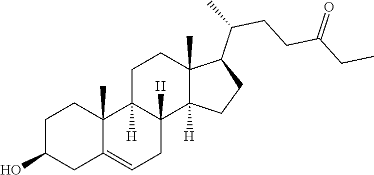 |
| 160 | 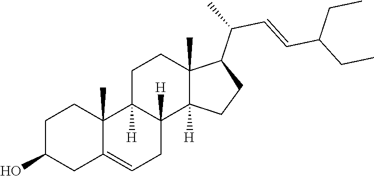 |
| 161 | 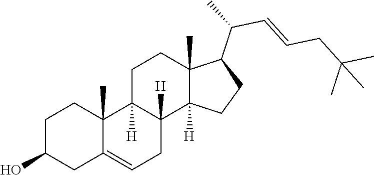 |
| 166 | 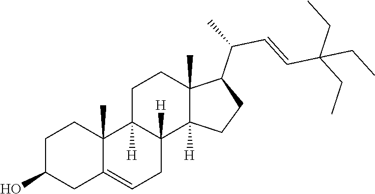 |
| 167 | 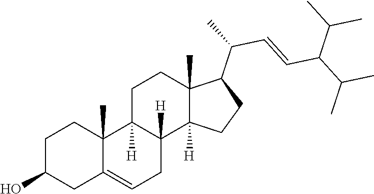 |
| 168 | 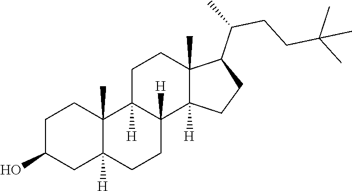 |
| 173 | 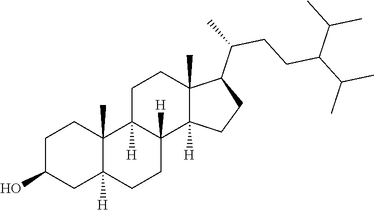 |
| 174 | 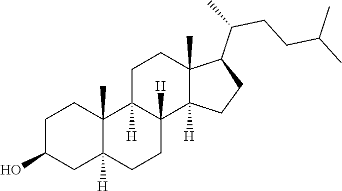 |
| 179 | 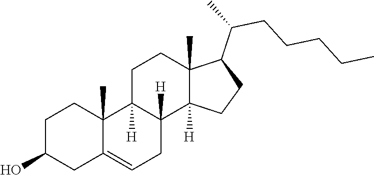 |
| 219 | 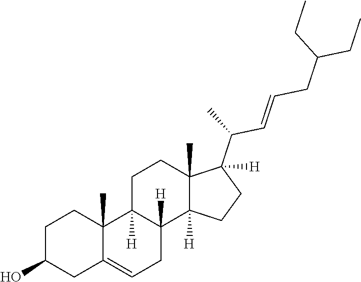 |
| 220 | 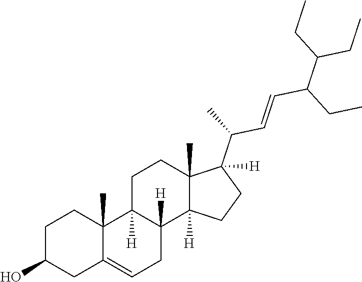 |
| 221 | 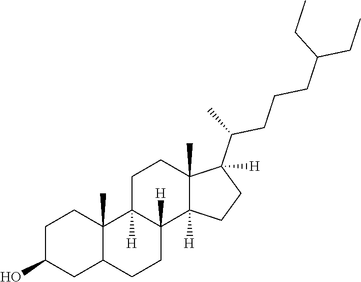 |
| 222 | 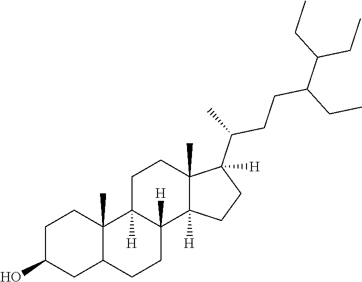 |
| 223 | 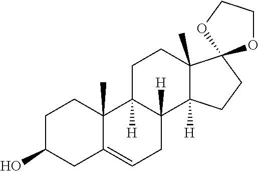 |
| 224 | 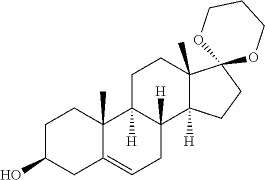 |
| 225 | 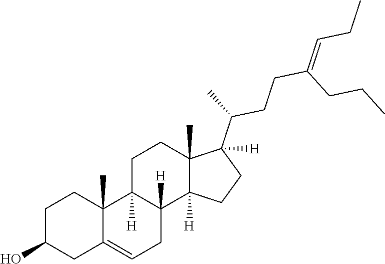 |
| 226 | 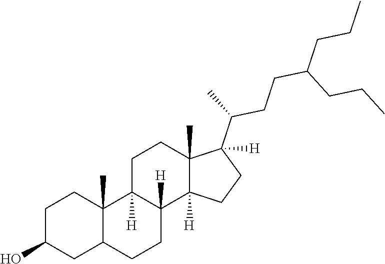 |

TABLE 15
Sterol Compounds for Structural Component

| CMPD | |
|---|---|
| No. | Structure |
| 131 | 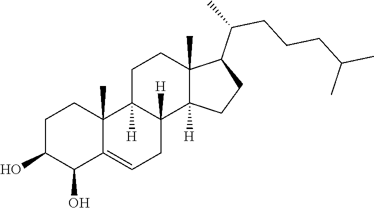 |
| 132 | 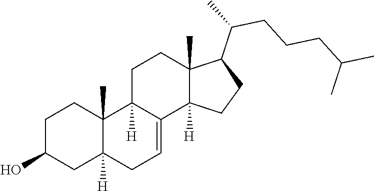 |
| 133 | 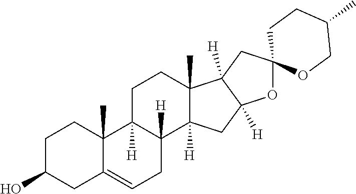 |

In an aspect, the invention features a lipid nanoparticle including:

(i) an ionizable lipid; and

(ii) a structural component,

where the structural component includes a compound having the structure of any of the foregoing compounds.

In some embodiments, the lipid nanoparticle further includes a nucleic acid molecule.

In an aspect, the invention features a lipid nanoparticle including:

(i) an ionizable lipid;

(ii) a structural component;

(iii) optionally, a non-cationic helper lipid;

(iv) optionally, a PEG-lipid; and

(v) a nucleic acid molecule,

where the structural component includes a compound having the structure of any of the foregoing compounds and optionally a structural lipid.

In some embodiments, the lipid nanoparticle includes the compound of any of the foregoing compounds in an amount that enhances delivery of the nucleic acid molecule to a cell relative to a lipid nanoparticle lacking said compound.

In some embodiments, the structural component further includes one or more structural lipids or salts thereof.

In some embodiments, the one or more structural lipids is a sterol.

In some embodiments, the one or more structural lipids is a phytosterol.

In some embodiments, the phytosterol is a sitosterol, a stigmasterol, a campesterol, a sitostanol, a campestanol, a brassicasterol, a fucosterol, beta-sitosterol, stigmastanol, beta-sitostanol, ergosterol, lupeol, cycloartenol, Δ5-avenaserol, Δ7-avenaserol or a Δ7-stigmasterol, including analogs, salts or esters thereof, alone or in combination. In some embodiments, the phytosterol component of a LNP of the disclosure is a single phytosterol. In some embodiments, the phytosterol component of a LNP of the disclosure is a mixture of different phytosterols (e.g. 2, 3, 4, 5 or 6 different phytosterols). In some embodiments, the phytosterol component of an LNP of the disclosure is a blend of one or more phytosterols and one or more zoosterols, such as a blend of a phytosterol (e.g., a sitosterol, such as beta-sitosterol) and cholesterol. In some embodiments, the phytosterol is β-sitosterol, campesterol, sigmastanol, or any combination thereof. In some embodiments, the phytosterol is β-sitosterol. In some embodiments, the one or more structural lipids comprises a mixture of β-sitosterol, campesterol, and stigmasterol.

In some embodiments, the one or more structural lipids comprises about 35% to about 85% of β-sitosterol, about 5% to about 35% stigmasterol, and about 5% to about 35% of campesterol. In some embodiments, the one or more structural lipids comprises about 40% to about 80% of β-sitosterol, about 10% to about 30% stigmasterol, and about 10% to about 30% of campesterol. In some embodiments, the one or more structural lipids comprises about 40% to about 70% of β-sitosterol, about 10% to about 25% stigmasterol, and about 10% to about 25% of campesterol. In some embodiments, the one or more structural lipids comprises about 40% to about 70% of β-sitosterol, about 15% to about 25% stigmasterol, and about 15% to about 25% of campesterol. In some embodiments, the one or more structural lipids comprises about 35% to about 45% of β-sitosterol, about 20% to about 30% stigmasterol, and about 20% to about 30% of campesterol. In some embodiments, the one or more structural lipids comprises about 40% to about 50% of β-sitosterol, about 25% to about 35% stigmasterol, and about 25% to about 35% of campesterol. In some embodiments, the one or more structural lipids comprises about 65% to about 75% of β-sitosterol, about 5% to about 15% stigmasterol, and about 5% to about 15% of campesterol.

In some embodiments, the one or more structural lipids comprises about 40% of β-sitosterol, about 25% stigmasterol, and about 25% of campesterol.

In some embodiments, the one or more structural lipids comprises about 70% of β-sitosterol, about 10% stigmasterol, and about 10% of campesterol.

In some embodiments, the one or more structural lipids comprises about 40% of β-sitosterol. In some embodiments, the one or more structural lipids comprises about 70% of β-sitosterol.

In some embodiments, the one or more structural lipids is a zoosterol. In some embodiments, the zoosterol is cholesterol.

In some embodiments, the mol % of the one or more structural lipids is between about 1% and 50% of the mol % of the compound having the structure of any of the foregoing compounds present in the lipid nanoparticle.

In some embodiments, the mol % of the one or more structural lipids is between about 10% and 40% of the mol % of the compound having the structure of any of the foregoing compounds present in the lipid nanoparticle.

In some embodiments, the mol % of the one or more structural lipids is between about 20% and 30% of the mol % of the compound having the structure of any of the foregoing compounds present in the lipid nanoparticle.

In some embodiments, the mol % of the one or more structural lipids is about 30% of the mol % of the compound having the structure of any of the foregoing compounds present in the lipid nanoparticle.

In some embodiments, the lipid nanoparticle includes one or more non-cationic helper lipids.

In some embodiments, the one or more non-cationic helper lipids is a phospholipid, fatty acid, or any combination thereof.

In some embodiments, the phospholipid is a phospholipid that includes a phosphocholine moiety, a phosphoethanolamine moiety, or a phosphor-1-glycerol moiety.

In some embodiments, the phospholipid is 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, or 1,2-didocosahexaenoyl-sn-glycero-3-phosphocholine.

In some embodiments, the phospholipid is DSPC.

In some embodiments, the phospholipid is 1,2-dioleoyl-sn-glycero-3-phosphoethanola mine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, or 1,2-dioleoyl-sn-glycero-3-phospho-rac-(1-glycerol) sodium salt (DOPG).

In some embodiments, the phospholipid is sphingomyelin.

In some embodiments, the fatty acid is a long-chain fatty acid. In some embodiments, the fatty acid is a very long-chain fatty acid. In some embodiments, the fatty acid is a medium-chain fatty acid.

In some embodiments, the fatty acid is palmitic acid, stearic acid, palmitoleic acid, or oleic acid.

In some embodiments, the fatty acid is oleic acid. In some embodiments, the fatty acid is stearic acid.

In some embodiments, the lipid nanoparticle includes one or more PEG-lipids.

In some embodiments, the one or more PEG-lipids is a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, or mixtures thereof.

In some embodiments, the one or more PEG-lipids is PEG-c-DOMG, PEG-DMG, PEG-DLPE, PEG-DMPE, PEG-DPPC, or PEG-DSPE lipid.

In some embodiments, the one or more PEG-lipids is PEG-DMG.

In some embodiments, the lipid nanoparticle includes about 30 mol % to about 60 mol % ionizable lipid or ionizable lipids, about 0 mol % to about 30 mol % to about 60 mol % one or more ionizable lipids, about 0 mol % to about 30 mol % one or more non-cationic helper lipids, about 18.5 mol % to about 48.5 mol % structural component, and about 0 mol % to about 10 mol % one or more PEG-lipids.

In some embodiments, the lipid nanoparticle includes about 35 mol % to about 55 mol % one or more ionizable lipids, about 5 mol % to about 25 mol % one or more non-cationic helper lipids, about 30 mol % to about 40 mol % structural component, and about 0 mol % to about 10 mol % one or more PEG-lipids.

In some embodiments, the lipid nanoparticle includes about 50 mol % one or more ionizable lipids, about 10 mol % one or more non-cationic helper lipids, about 38.5 mol % structural component, and about 1.5 mol % one or more PEG-lipids.

In some embodiments, the nucleic acid molecule is RNA or DNA.

In some embodiments, the nucleic acid is DNA.

In some embodiments, the nucleic acid molecule is ssDNA. In some embodiments, the nucleic acid is DNA including CRISPR.

In some embodiments, the nucleic acid is RNA.

In some embodiments, the nucleic acid molecule is a shortmer, an antagomir, an antisense, a ribozyme, a small interfering RNA (siRNA), an asymmetrical interfering RNA (aiRNA), a microRNA (miRNA), a Dicer-substrate RNA (dsRNA), a small hairpin RNA (shRNA), or a messenger RNA (mRNA).

In some embodiments, the nucleic acid molecule is an mRNA.

In some embodiments, the mRNA is a modified mRNA including one or more modified nucleobases.

In some embodiments, the mRNA includes one or more of a stem loop, a chain terminating nucleoside, a polyA sequence, a polyadenylation signal, and a 5′ cap structure.

In some embodiments, the structural component includes a compound of Formula I. In some embodiments, the structural component includes a compound of Formula III. In some embodiments, the structural component includes a compound of Formula III. In some embodiments, the structural component includes a compound of Formula IV. In some embodiments, the structural component includes a compound of Formula V. In some embodiments, the structural component includes a compound of Formula VI. In some embodiments, the structural component includes a compound of Formula VII. In some embodiments, the structural component includes a compound of Formula VIII. In some embodiments, the structural component includes a compound of Formula IX. In some embodiments, the structural component includes a compound of Formula X. In some embodiments, the structural component includes a compound of Formula XI. In some embodiments, the structural component includes a compound of Formula XII. In some embodiments, the structural component includes a compound of Formula XIII.

In some embodiments, the structural component includes a compound having the structure of any one of compounds 1-42, 150, 154, 162-165, 169-172, and 184-209 in Table 1. In some embodiments, the structural component includes a compound having the structure of any one of compounds 43-50 and 175-178 in Table 2. In some embodiments the structural component includes a compound having the structure of any one of compounds 51-67, 149, and 153 in Table 3. In some embodiments, the structural component includes a compound having the structure of any one of compounds 68-73 in Table 4. In some embodiments, the structural component includes a compound having the structure of any one of compounds 74-78 in Table 5. In some embodiments, the structural component includes a compound having the structure of any one of compounds 79-80 in Table 6. In some embodiments, the structural component includes a compound having the structure of any one of compounds 81-83, 85-87, 152, and 157 in Table 7. In some embodiments, the structural component includes a compound having the structure of any one of compounds 88-97 in Table 8. In some embodiments, the structural component includes a compound having the structure of any one of compounds 98-105, 180-182, and 210-213 in Table 9. In some embodiments, the structural component includes a compound having the structure of compound 106 in Table 10. In some embodiments, the structural component includes a compound having the structure of any one of compound 107 or 108 in Table 11. In some embodiments, the structural component includes a compound having the structure of compound 109 in Table 12. In some embodiments, the structural component includes a compound having the structure of any one of compounds 214-218 in Table 13. In some embodiments, the structural component includes a compound having the structure of any one of compounds 110-130, 155, 156, 160, 161, 166-168, 173, 174, 179, and 219-226 in Table 14.

In some embodiments, the lipid nanoparticle further includes an additional compound having the structure of any one of the foregoing compounds.

### Definitions

As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given component a lipid nanoparticle, “about” may mean +/−10% of the recited value. For instance, a lipid nanoparticle including a structural component having about 40% of a given compound may include 30-50% of the compound.

As used herein, the term “compound,” is meant to include all geometric isomers and isotopes of the structure depicted. “Isotopes” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

As used herein, the term “contacting” means establishing a physical connection between two or more entities. For example, contacting a mammalian cell with a composition means that the mammalian cell and a nanoparticle are made to share a physical connection. Methods of contacting cells with external entities both in vivo and ex vivo are well known in the biological arts. For example, contacting a composition and a mammalian cell disposed within a mammal may be performed by varied routes of administration (e.g., intravenous, intramuscular, intradermal, and subcutaneous) and may involve varied amounts of compositions. Moreover, more than one mammalian cell may be contacted by a composition.

As used herein, the term “delivering” means providing an entity to a destination. For example, delivering an mRNA to a subject may involve administering a composition including the mRNA to the subject (e.g., by an intravenous, intramuscular, intradermal, or subcutaneous route). Administration of a composition to a mammal or mammalian cell may involve contacting one or more cells with the composition.

As used herein, “encapsulation efficiency” refers to the amount of an mRNA that becomes part of a composition, relative to the initial total amount of mRNA used in the preparation of a composition. For example, if 97 mg of mRNA are encapsulated in a composition out of a total 100 mg of mRNA initially provided to the composition, the encapsulation efficiency may be given as 97%.

As used herein, “encapsulation” may refer to complete, substantial, or partial enclosure, confinement, surrounding, or encasement.

As used herein, “expression” of a nucleic acid sequence refers to translation of an mRNA into a polypeptide or protein and/or post-translational modification of a polypeptide or protein.

As used herein, “fatty acid” refers to a carboxylic acid with an aliphatic chain. As used herein, “short-chain fatty acids” or “SCFA” are fatty acids with aliphatic tails of fewer than six carbons (e.g., butyric acid). As used herein, “medium-chain fatty acids” or “MCFA” are fatty acids with aliphatic tails of 6-12 carbons (e.g., lauric acid) and can form medium-chain triglycerides. As used herein, “long-chain fatty acids” or “LCFA” are fatty acids with aliphatic tails of 13 to 21 carbons (e.g., arachidic acid or oleic acid). As used herein, “very long-chain fatty acids” or “VLCFA” are fatty acids with aliphatic tails of 22 or more carbons (e.g., cerotic acid).

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

As used herein, a “linker” is a moiety connecting two moieties, for example, the connection between two nucleosides of a cap species. A linker may include one or more groups including but not limited to phosphate groups (e.g., phosphates, boranophosphates, thiophosphates, selenophosphates, and phosphonates), alkyl groups, amidates, or glycerols. For example, two nucleosides of a cap analog may be linked at their 5′ positions by a triphosphate group or by a chain including two phosphate moieties and a boranophosphate moiety.

As used herein, “methods of administration” may include intravenous, intramuscular, intradermal, subcutaneous, or other methods of delivering a composition to a subject. A method of administration may be selected to target delivery to a specific region or system of a body.

As used herein, “modified” means non-natural. For example, an mRNA may be a modified mRNA. That is, an mRNA may include one or more nucleobases, nucleosides, nucleotides, or linkers that are non-naturally occurring. A “modified” species may also be referred to herein as an “altered” species. Species may be modified or altered chemically, structurally, or functionally. For example, a modified nucleobase species may include one or more substitutions that are not naturally occurring.

As used herein, “mRNA” refers to a messenger ribonucleic acid that may be naturally or non-naturally occurring. For example, an mRNA may include modified and/or nonnaturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide of interest. Translation of an mRNA, for example, in vivo translation of an mRNA inside a mammalian cell, may produce a polypeptide of interest.

As used herein, “non-cationic helper lipid” refers to a lipid including at least one fatty acid chain including at least 8 carbon atoms (e.g., 8, 9, 10, 11, 12, 13, 14, 15, 16, 17, 18, 19, or 20 carbon atoms) and at least one polar head group moiety. In some embodiments the non-cationic helper lipid is a phospholipid or a phospholipid substitute. In some embodiments, the non-cationic helper lipid is a DSPC analog, a DSPC substitute, oleic acid, or an oleic acid analog.

As used herein, “phytosterol” refers to plant sterol, including a salt or ester thereof.

As used herein, the “N:P ratio” is the molar ratio of ionizable (in the physiological pH range) nitrogen atoms in a lipid to phosphate groups in an RNA, e.g., in a composition including a lipid component (e.g., a lipid nanoparticle) and an RNA, such as an mRNA.

As used herein, “naturally occurring” means existing in nature without artificial aid.

As used herein, “patient” refers to a subject who may seek or be in need of treatment, requires treatment, is receiving treatment, will receive treatment, or a subject who is under care by a trained professional for a particular disease or condition.

As used herein, a “PEG-lipid” or “PEGylated lipid” refers to a lipid comprising a polyethylene glycol component.

As used herein, “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable excipient” refers to any ingredient other than the compounds described herein (for example, a vehicle capable of suspending, complexing, or dissolving the active compound) and having the properties of being substantially nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch, glycolate, sorbitol, starch (corn), stearic acid, sucrose, talc, titanium dioxide, vitamin A, vitamin E (alpha-tocopherol), vitamin C, xylitol, and other species disclosed herein.

As used herein, “pharmaceutically acceptable salts” refers to derivatives of the disclosed compounds wherein the parent compound is altered by converting an existing acid or base moiety to its salt form (e.g., by reacting the free base group with a suitable organic acid). Compositions of the invention may also include pharmaceutically acceptable salts of one or more compounds. Pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 30 1985, p. 1418, Pharmaceutical Salts: Properties, Selection, and Use, P.H. Stahl and C.G. Wermuth (eds.), Wiley-VCH, 2008, and Berge et al., Journal of Pharmaceutical Science, 66, 1-19 (1977), each of which is incorporated herein by reference in its entirety.

As used herein, the “polydispersity index” is a ratio that describes the homogeneity of the particle size distribution of a system. A small value, e.g., less than 0.3, indicates a narrow particle size distribution.

As used herein, “polypeptide” or “polypeptide of interest” refers to a polymer of amino acid residues typically joined by peptide bonds that can be produced naturally (e.g., isolated or purified) or synthetically.

As used herein, a “single unit dose” is a dose of any therapeutic administered in one dose/at one time/single route/single point of contact, i.e., single administration event.

As used herein, “size” or “mean size” in the context of compositions refers to the mean diameter of a composition.

As used herein, “sterol” refers to the subgroup of steroids also known as steroid alcohols, including a salt or ester thereof. Sterols are usually divided into two classes: 1) plant sterol (e.g., phytosterol); and 2) animal sterol (e.g., zoosterol). Zoosterols include, but are not limited to, cholesterol.

As used herein, “stanol” refers to the class of saturated sterols having no double bonds in the sterol ring structure.

As used herein, “structural lipid” refers to steroids and/or lipids containing steroidal moieties (e.g., sterols and/or lipids containing sterol moieties).

As used herein, a “split dose” is the division of single unit dose or total daily dose into two or more doses.

As used herein, “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

As used herein, a “total daily dose” is an amount given or prescribed in 24 hour period. It may be administered as a single unit dose.

As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

The term “therapeutic agent” refers to any agent that, when administered to a subject, has a therapeutic, diagnostic, and/or prophylactic effect and/or elicits a desired biological and/or pharmacological effect.

As used herein, the term “therapeutically effective amount” means an amount of an agent to be delivered (e.g., nucleic acid, drug, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

As used herein, “transfection” refers to the introduction of a species (e.g., an mRNA) into a cell. Transfection may occur, for example, in vitro, ex vivo, or in vivo.

As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the “zeta potential” is the electrokinetic potential of a lipid e.g., in a particle composition.

### Chemical Terms

Those skilled in the art will appreciate that certain compounds described herein can exist in one or more different isomeric (e.g., stereoisomers, geometric isomers, tautomers) and/or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion, e.g., the interconversion illustrated in the scheme below:

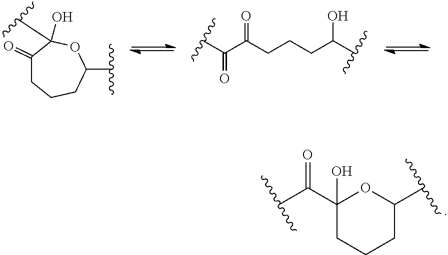 [SMILES: *C1CCCC(=O)C(*)(O)O1 ; *C(=O)C(=O)CCCC(*)O ; *C(=O)C1(O)CCCC(*)O1]

Those skilled in the art will appreciate that, in some embodiments, isotopes of compounds described herein may be prepared and/or utilized in accordance with the present invention. “Isotopes” refers to atoms having the same atomic number but different mass numbers resulting from a different number of neutrons in the nuclei. For example, isotopes of hydrogen include tritium and deuterium. In some embodiments, an isotopic substitution (e.g., substitution of hydrogen with deuterium) may alter the physicochemical properties of the molecules, such as metabolism and/or the rate of racemization of a chiral center.

As is known in the art, many chemical entities (in particular many organic molecules and/or many small molecules) can adopt a variety of different solid forms such as, for example, amorphous forms and/or crystalline forms (e.g., polymorphs, hydrates, solvates, etc). In some embodiments, such entities may be utilized in any form, including in any solid form. In some embodiments, such entities are utilized in a particular form, for example in a particular solid form.

In some embodiments, compounds described and/or depicted herein may be provided and/or utilized in salt form.

In certain embodiments, compounds described and/or depicted herein may be provided and/or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substitutes are disclosed in groups or in ranges, unless otherwise indicated, the present disclosure is intended to cover individual compounds and groups of compounds (e.g., genera and subgenera) containing each and every individual subcombination of members at each position.

Herein a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

It is to be understood that the terminology employed herein is for the purpose of describing particular embodiments and is not intended to be limiting.

The term “acyl,” as used herein, represents a hydrogen or an alkyl group, as defined herein that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms). An alkylene is a divalent alkyl group.

The term “alkenyl,” as used herein, alone or in combination with other groups, refers to a straight-chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight-chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “aryl,” as used herein, refers to an aromatic mono- or polycarbocyclic radical of 6 to 12 carbon atoms having at least one aromatic ring. Aryl groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₁₋₆ alkyl C₆₋₁₀ aryl, C₁₋₁₀ alkyl C₆₋₁₀ aryl, or C₁₋₂₀ alkyl C₆₋₁₀ aryl), such as, benzyl and phenethyl. In some embodiments, the akyl and the aryl each can be further substituted with 1, 2, 3, or 4 substituent groups as defined herein for the respective groups.

The terms “carbocyclyl,” as used herein, refer to a non-aromatic C₃-C₂₀ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, monovalent mono- or polycarbocyclic radical of three to twenty, preferably three to ten or three to six carbon atoms. This term is further exemplified by radicals such as cyclopropyl, cyclobutyl, cyclopentyl, cyclohexyl, cycloheptyl, and adamantyl.

The term “cycloalkenyl,” as used herein, refers to an unsaturated, non-aromatic, monovalent mono- or polycarbocyclic radical of three to twenty, preferably, three to ten or three to six carbon atoms.

This term is further exemplified by radicals such as cyclobutenyl, cyclopentenyl, cyclohexenyl, cycloheptenyl, and norbornyl.

The term “polycycloalkyl” mean a structure consisting of two or more cycloalkyl moieties that have two or more atoms in common. If the cycloalkyl moieties have exactly two atoms in common they are said to be “fused.” If the cycloalkyl moieties have more than two atoms in common they are said to be “bridged.”

The term “halo,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Heteroalkyl groups include, but are not excluded to, “alkoxy” which, as used herein, refers alkyl-O— (e.g., methoxy and ethoxy). A heteroalkylene is a divalent heteroalkyl group.

The term “heterocyclyl,” as used herein, denotes a mono- or polycyclic radical having 3 to 12 atoms having at least one ring containing one, two, three, or four ring heteroatoms selected from N, O or S, wherein no ring is aromatic. Heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁₋₆ alkyl C₂₋₉ heterocyclyl, C₁₋₁₀ alkyl C₂₋₉ heterocyclyl, or C₁₋₂₀ alkyl C₂₋₉ heterocyclyl). In some embodiments, the akyl and the heterocyclyl each can be further substituted with 1, 2, 3, or 4 substituent groups as defined herein for the respective groups.

The term “hydroxyl,” as used herein, represents an —OH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: aryl (e.g., substituted and unsubstituted phenyl), carbocyclyl (e.g., substituted and unsubstituted cycloalkyl), halogen (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbents or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99% or 99.9%) by weight relative to the other stereoisomers. When a single enantiomer is named or depicted by structure, the depicted or named enantiomer is at least 60%, 70%, 80%, 90%, 99% or 99.9% by weight optically pure. When a single diastereomer is named or depicted by structure, the depicted or named diastereomer is at least 60%, 70%, 80%, 90%, 99% or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers.

When the stereochemistry of a disclosed compound is named or depicted by structure, the named or depicted stereoisomer is at least 60%, 70%, 80%, 90%, 99% or 99.9% by mole fraction pure relative to the other stereoisomers. When a single enantiomer is named or depicted by structure, the depicted or named enantiomer is at least 60%, 70%, 80%, 90%, 99% or 99.9% by mole fraction pure. When a single diastereomer is named or depicted by structure, the depicted or named diastereomer is at least 60%, 70%, 80%, 90%, 99% or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

### DETAILED DESCRIPTION OF THE INVENTION

This invention features sterol compounds which, in one aspect, may be utilized in lipid-containing compositions for delivering mRNA into cells. Lipid-containing compositions have proven effective as transport vehicles into cells and/or intracellular compartments for a variety of RNAs. These compositions generally include one or more “cationic” and/or ionizable lipids, structural lipids (e.g., sterols or sterol analogs), and lipids containing polyethylene glycol (PEG-lipids). Cationic and/or ionizable lipids include, for example, amine-containing lipids that can be readily protonated.

The present disclosure relates to a lipid nanoparticle including a compound of the invention (e.g., a compound of Formula I, Formula II, Formula III, Formula IV, Formula V, Formula VI, Formula VII, Formula VIII, Formula IX, Formula X, Formula XI, Formula XII, or Formula XIII) and methods of using the same. For example, the invention provides a method of producing a polypeptide of interest in a cell that involves contacting a composition of the invention with a cell where the mRNA may be translated to produce the polypeptide of interest. The invention further includes a method of delivering an mRNA to a mammalian cell involving administration of a composition including mRNA to a subject, in which the administration involves contacting a cell with the composition where the mRNA is delivered to a cell.

A lipid nanoparticle of the invention includes an ionizable lipid and a compound of the invention.

Lipid Nanoparticle A lipid nanoparticle of the invention includes an ionizable lipid and a compound of the invention (e.g., a compound of Formula I, Formula II, Formula III, Formula IV, Formula V, Formula VI, Formula VII, Formula VIII, Formula IX, Formula X, Formula XI, Formula XII, or Formula XIII) or any one of compounds 131-133 in Table 15. The lipid nanoparticle of the invention optionally further includes a structural lipid, a non-cationic helper lipid, a PEG-lipid, and/or a nucleic acid molecule.

Ionizable Lipids

The lipid nanoparticle of the invention includes one or more ionizable lipids. For example, a lipid nanoparticle includes an ionizable lipid. The ionizable lipids described herein may be advantageously used in a lipid nanoparticle of the invention for the delivery of nucleic acid molecules to a cell (e.g., mammalian cell).

Ionizable lipids include, but are not limited to, 3-(didodecylamino)-N1,N1,4-tridodecyl-1-piperazineethanamine (KL10), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA),1,2-dioleyloxy-N,N-dimethylaminopropane (DODMA), 2-({8-[(3p)-cholest-5-en-3-yloxy]octyl)oxy)-N,N-dimethyl-3-[(9Z,12Z)-octadeca-9,12-dien-1-yloxy]propan-1-amine (Octyl-CLinDMA), (2R)-2-((8-[(3p)-cholest-5-en-3-yloxy]octyl}oxy)-N,N-dimethyl-3-[(9Z,12Z)-octadeca-9,12-dien-1-yloxy]pro pan-1-amine (Octyl-CLinDMA (2R)), and (2S)-2-({8-[(3)-cholest-5-en-3-yloxy]octyl)oxy)-N,N-dimethyl-3-[(9Z,12Z)-octadeca-9,12-dien-1-yloxy]propan-1-amine (Octyl-CLinDMA (2S)). In addition to these, an ionizable lipid may also be a lipid including a cyclic amine.

Ionizable lipids include, but are not limited to, the ionizable lipids disclosed in International Publication No. WO 2015/199952, WO 2017/075531, and/or WO 2017/049245.

Ionizable lipids can have a positive or partial positive charge at physiological pH. Such ionizable lipids can be referred to as cationic and/or ionizable lipids. Ionizable lipids can be zwitterionic.

In some embodiments, ionizable lipids have the following structure:

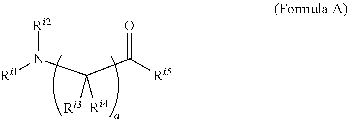

in which Rⁱ¹ is H or optionally substituted C₃-C₁₀ alkyl; each of Rⁱ² and Rⁱ⁵ is, independently, optionally substituted C₃-C₅₀ alkyl, optionally substituted C₃-C₅₀ heteroalkyl, or optionally substituted C₃-C₅₀ alkenyl; each of Rⁱ³ and Rⁱ⁴ is, independently, H or C₃-C₁₀ alkyl; and a is an integer between 5-20, or salts thereof. Examples of ionizable lipids having a structure according to Formula A include:

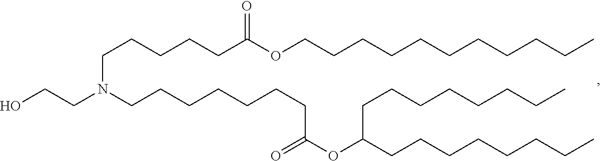 [SMILES: CCCCCCCCCCCOC(=O)CCCCCN(CCO)CCCCCCCC(=O)OC(CCCCCCCC)CCCCCCCC]

r a salt thereof.

In addition to the ionizable lipids disclosed herein, the lipid nanoparticle disclosed herein includes a compound of the invention (e.g., a compound of Formula I, Formula II, Formula III, Formula IV, Formula V, Formula VI, Formula VII, Formula VIII, Formula IX, Formula X, Formula XI, Formula XII, or Formula XIII) or any one of compounds 131-133 in Table 15. A lipid nanoparticle disclosed herein can optionally include a non-cationic helper lipid, a PEG-lipid, a structural lipid, and/or a nucleic acid molecule, or any combination thereof.

### Structural Component

A lipid nanoparticle of the invention includes a structural component. The structural component includes a compound of the invention (e.g., a compound of Formula I, Formula II, Formula III, Formula IV, Formula V, Formula VI, Formula VII, Formula VIII, Formula IX, Formula X, Formula XI, Formula XII, or Formula XIII; or any one of compounds 1-42, 150, 154, 162-165, 169-172, and 184-209 in Table 1, compounds 43-50 and 175-178 in Table 2, compounds 51-67, 149, and 153 in Table 3, compounds 68-73 in Table 4, compounds 74-78 in Table 5, compound 79 or 80 in Table 6, compounds 81-83, 85-87, 152, and 157 in Table 7, compounds 88-97 in Table 8, compounds 98-105, 180-182, and 210-213 in Table 9, compound 106 in Table 10, compound 107 or 108 in Table 11, compound 109 in Table 12, compounds 214-218 in Table 13, or compounds 110-130, 155, 156, 160, 161, 166-168, 173, 174, 179, and 219-226 in Table 14), or any one of compounds 131-133 in Table 15. The structural component can include a structural lipid. For example, the structural component includes a compound of the invention or any one of compounds 131-133 in Table 15 and a structural lipid.

The structural component can include an additional compound of the invention or any one of compounds 131-133 in Table 15.

For example, lipid nanoparticles can include a compound of the invention or one or more compounds of the invention (e.g., two or more compounds of the invention, three or more compounds of the invention, or four or more compounds of the invention). The compounds described herein may be advantageously used in lipid nanoparticles of the invention for the delivery of nucleic acid molecules to a cell (e.g., mammalian cell).

The structural component can include one or more structural lipids. For example, the structural component can include a compound of the invention, a mixture of one or more compounds of the invention, a mixture of a compound of the invention and a structural lipid, a mixture of a compound of the invention and one or more structural lipids, or a mixture of one or more compound of the invention and one or more structural lipids.

### Compounds of the Invention

Compound of the invention include compounds having a structure according to Formula I, Formula II, Formula III, Formula IV, Formula V, Formula VI, Formula VII, Formula VIII, Formula IX, Formula X, Formula XI, Formula XII, or Formula XIII:

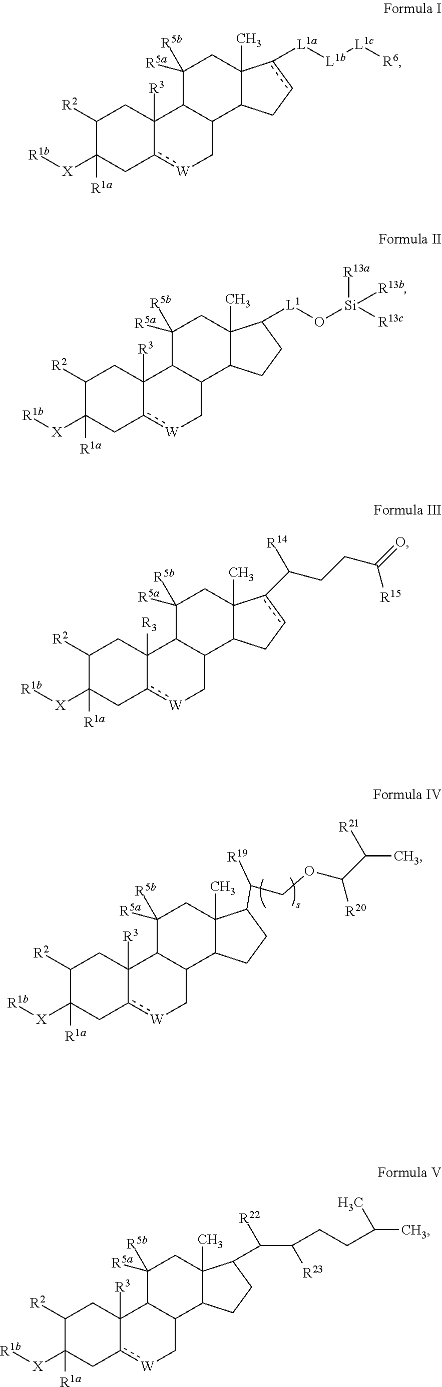

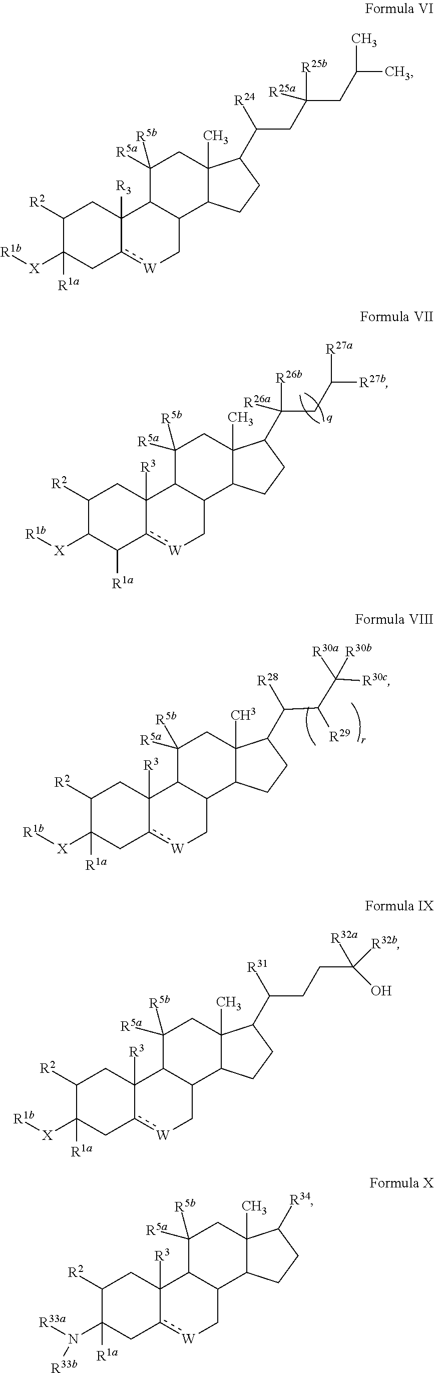

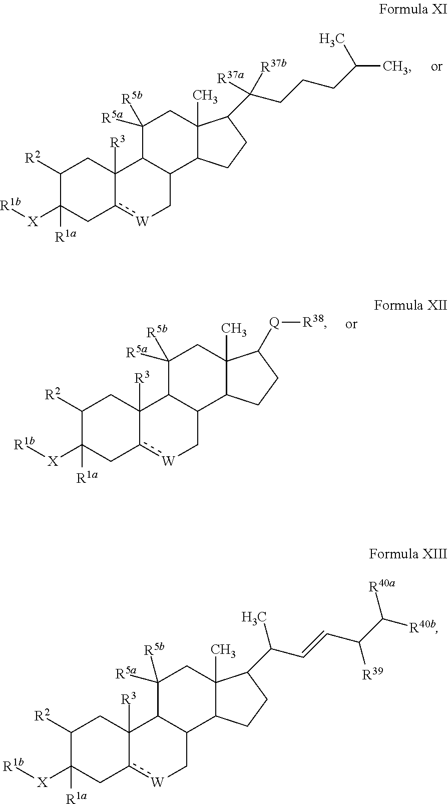

or a pharmaceutically acceptable salt thereof.

Compounds of the invention also include compounds having the structure of compounds 1-42, 150, 154, 162-165, 169-172, and 184-209 in Table 1, compounds 43-50 and 175-178 in Table 2, compounds 51-67, 149, and 153 in Table 3, compounds 68-73 in Table 4, compounds 74-78 in Table 5, compound 79 or 80 in Table 6, compounds 81-83, 85-87, 152, and 157 in Table 7, compounds 88-97 in Table 8, compounds 98-105, 180-182, and 210-213 in Table 9, compound 106 in Table 10, compound 107 or 108 in Table 11, compound 109 in Table 12, compounds 214-218 in Table 13, or compounds 110-130, 155, 156, 160, 161, 166-168, 173, 174, 179, and 219-226 in Table 14.

### Structural Lipids

The lipid nanoparticles of the invention can include one or more structural lipids. For example, lipid nanoparticles can include a structural lipid or one or more structural lipids (e.g., two or more structural lipids, three or more structural lipids, four or more structural lipids, or five or more structural lipids). The structural lipids described herein may be advantageously used in lipid nanoparticles of the invention for the delivery of nucleic acid molecules to a cell (e.g., mammalian cell).

Structural lipids can include, but are not limited to, sterols (e.g., phytosterols or zoosterols). For example, sterols can include, but are not limited to, cholesterol, 13-sitosterol, fecosterol, ergosterol, sitosterol, campesterol, stigmasterol, brassicasterol, ergosterol, tomatidine, tomatine, ursolic acid, alpha-tocopherol, or any one of compounds 84, 134-148, 151, and 159 in Table 16.

TABLE 16
Structural Lipids

| CMPD | |
|---|---|
| No. | Structure |
| 84 | 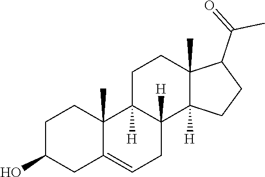 |
| 134 | 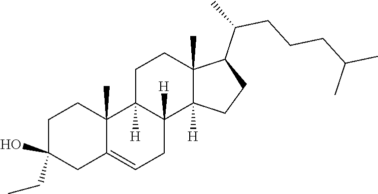 |
| 135 | 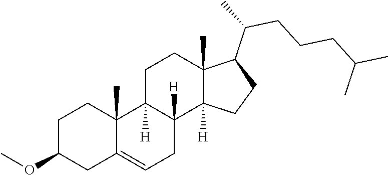 |
| 136 | 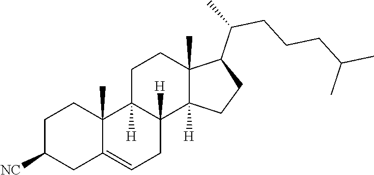 |
| 137 | 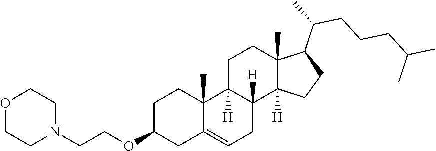 |
| 138 | 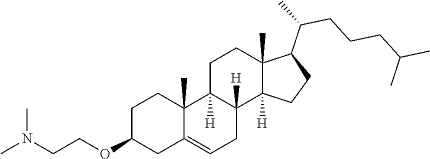 |
| 139 | 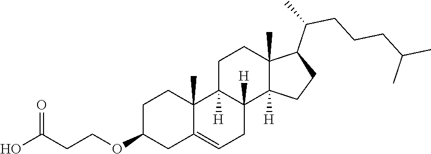 |
| 140 | 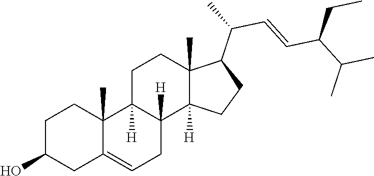 |
| 141 | 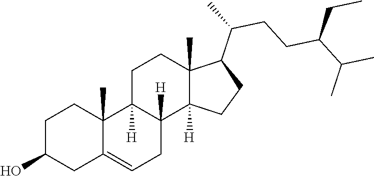 |
| 142 | 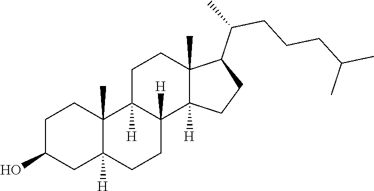 |
| 143 | 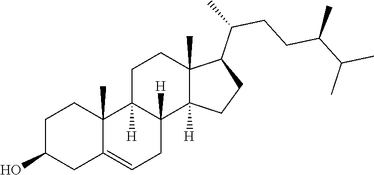 |
| 144 | 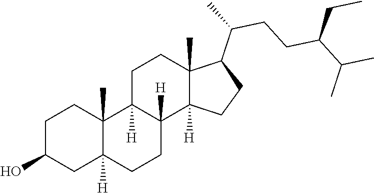 |
| 145 | 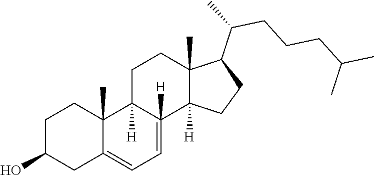 |
| 146 | 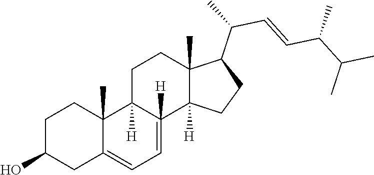 |
| 147 | 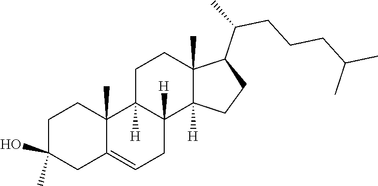 |
| 148 | 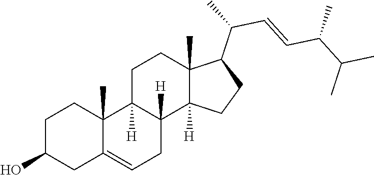 |
| 151 | 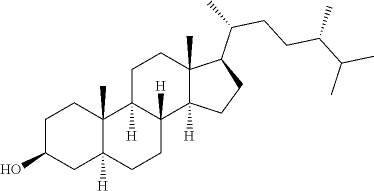 |
| 159 | 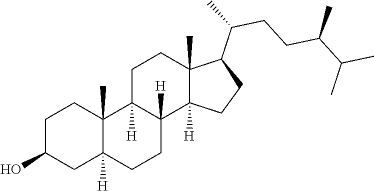 |

The one or more structural lipids of the lipid nanoparticles of the invention can be a composition of structural lipids (e.g., a mixture of two or more structural lipids, a mixture of three or more structural lipids, a mixture of four or more structural lipids, or a mixture of five or more structural lipids). A composition of structural lipids can include, but is not limited to, any combination of sterols (e.g., cholesterol, 13-sitosterol, fecosterol, ergosterol, sitosterol, campesterol, stigmasterol, brassicasterol, ergosterol, tomatidine, tomatine, ursolic acid, alpha-tocopherol, or any one of compounds 84, 134-148, 151, and 159 in Table 16). For example, the one or more structural lipids of the lipid nanoparticles of the invention can be composition 183 in Table 17.

TABLE 17
Structural Lipid Compositions

| Com- | |
|---|---|
| position | |
| No. | Structure |
| 183 | 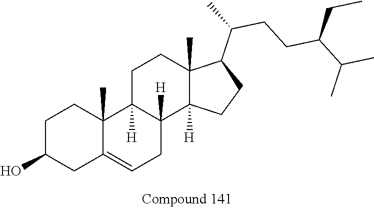 |
| | 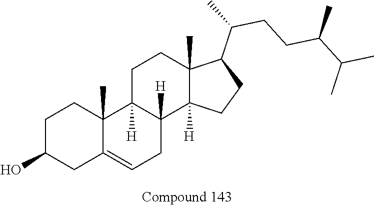 |
| | 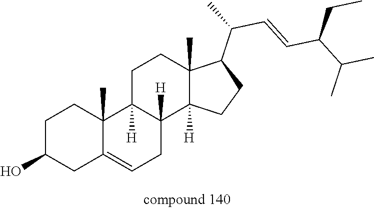 |
| | 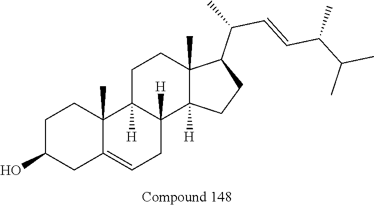 |

Composition 183 is a mixture of compounds 141, 140, 143, and 148. In some embodiments, composition 183 includes about 35% to about 45% of compound 141, about 20% to about 30% of compound 140, about 20% to about 30% compound 143, and about 5% to about 15% of compound 148. In some embodiments, composition 183 includes about 40% of compound 141, about 25% of compound 140, about 25% compound 143, and about 10% of compound 148.

Ratio of Compounds to Structural Lipids

A lipid nanoparticle of the invention includes a structural component. The structural component of the lipid nanoparticle can be a compound of the invention or any one of compounds 131-133 in Table 15, a mixture of one or more compounds of the invention and/or any one of compounds 131-133 in Table 15, a mixture of a compound of the invention or any one of compounds 131-133 in Table 15 and one or more structural lipids, or a mixture of one or more compound of the invention and one or more structural lipids.

For example, the structural component of the lipid nanoparticle can be a compound of the invention. The mol % of the structural lipid is 0% of the mol % of the compound present in the lipid nanoparticle.

In another example, the structural component of the lipid nanoparticle can be a mixture of a compound of the invention and a structural lipid. The mol % of the structural lipid present in the lipid nanoparticle can be 10 mol %. The mol % of the compound present in the lipid nanoparticle can be 20 mol %. In this example, the 10 mol % of the structural lipid is 50% of the 20 mol % of the compound.

In yet another example, the structural component of the lipid nanoparticle can be a mixture of a compound of the invention and two structural lipids: Lipid 1 and Lipid 2. The mol % of Lipid 1 present in the lipid nanoparticle can be 5 mol %. The mol % of Lipid 2 present in the lipid nanoparticle can be 10 mol %. The mol % of the compound present in the lipid nanoparticle can be 20 mol %. In this example, the 5 mol % plus 10 mol % of the two structural lipids is 75% of the 20 mol % of the compound.

In another example, the structural component of the lipid nanoparticle can be a mixture of one or more of any of the compounds of the invention and/or any one of compounds 131-133 in Table 15 with cholesterol. The mol % of the one or more of any of the compounds of the invention and/or any one of compounds 131-133 in Table 15 present in the lipid nanoparticle relative to cholesterol can be from 0-99 mol %. The mol % of the one or more of any of the compounds of the invention and/or any one of compounds 131-133 in Table 15 present in the lipid nanoparticle relative to cholesterol can be about 10 mol %, 20 mol %, 30 mol %, 40 mol %, 50 mol %, 60 mol %, 70 mol %, 80 mol %, or 90 mol %.

### Non-Cationic Helper Lipids

The lipid nanoparticle of the invention can include one or more non-cationic helper lipids (e.g., a phospholipid). For example, a lipid nanoparticle can include a non-cationic helper lipid or one or more non-cationic helper lipids (e.g., two or more non-cationic helper lipids, three or more non-cationic helper lipids, four or more non-cationic helper lipids, or five or more non-cationic helper lipids). The non-cationic helper lipids described herein may be advantageously used in a lipid nanoparticle of the invention for the delivery of nucleic acid molecules to a cell (e.g., mammalian cell).

Non-cationic helper lipids include, but are not limited to, phospholipids (e.g., polyunsaturated phospholipids) and fatty acids (e.g., oleic acid).

Phospholipids include a phospholipid moiety and one or more fatty acid moieties. For example, a phospholipid may be a lipid according to the formula:

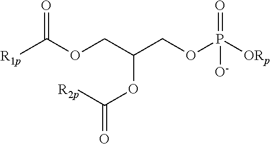

in which Rₚ represents a phospholipid moiety and R₁ₚ and R₂ₚ represent fatty acid moieties with or without saturation that may be the same or different. A phospholipid moiety may be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated.

Phospholipids include, but are not limited to, 1,2-distearoyl-sn-glycero-3-phosphocholine (DSPC), 1,2-dioleoyl-sn-glycero-3-phosphoethanolamine (DOPE), or both DSPC and DOPE. Phospholipids useful in the compositions and methods of the invention may be selected from the non-limiting group consisting of DSPC, DOPE, 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

Fatty acids include, but are not limited to, short-chain fatty acids (SCFA), medium-chain fatty acids (MCFA), long-chain fatty acids (LCFA), or very long-chain fatty acids (VLCFA).

Short-chain fatty acids include, but are not limited to, butyric acid, isobutyric acid, valeric acid, and isovaleric acid. Medium-chain fatty acids include, but are not limited to, caproic acid, caprylic acid, capric acid, and lauric acid. Long-chain fatty acids include, but are not limited to, pentadecylic acid, palmitic acid, margaric acid, stearic acid, nonadecylic acid, arachidic acid, heneicosylic acid, behenic acid, palmitoleic acid, oleic acid, elaidic acid, gondoic acid, erucic acid, sapienic acid, paullinic acid, myristic acid, myristoleic acid, vaccenic acid, eicosapentaenoic acid, erucic acid, linolelaidic acid, docsahexaenoic acid, myristic acid, or linoleic acid. Very long-chain fatty acids include, but are not limited to, tricosylic acid, lignoceric acid, cerotic acid, nervonic acid, pentacosylic acid, heptacosylic acid, montanic acid, nonacosylic acid, melissic acid, or henatriacontylic acid.

PEG-Lipids

A lipid nanoparticle of the invention can include one or more PEG-lipids. For example, a lipid nanoparticle can include a PEG-lipid or one or more PEG-lipids (e.g., two or more PEG-lipids, three or more PEG-lipids, four or more PEG-lipids, or five or more PEG-lipids). The PEG-lipids described herein may be advantageously used in a lipid nanoparticle of the invention for the delivery of nucleic acid molecules to a cell (e.g., mammalian cell).

PEG-lipids can be PEG-modified phosphatidylethanolamines, PEG-modified phosphatidic acids, PEG-modified ceramides, PEG-ceramide conjugates, PEG-modified dialkylamines, PEG-modified diacylglycerols, PEG-modified 1,2-diacyloxypropan-3-amines, and PEG-modified dialkylglycerols. PEG-lipids include, but are not limited to, 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), PEG-1,2-dimyristyloxlpropyl-3-amine (PEG-c-DMA), R-3-[(ω-methoxy poly(ethylene glycol)₂₀₀₀)carbamoyl)]-1,2-dimyristyloxlpropyl-3-amine (PEG-c-DOMG), PEG-1,2-dilauroyl-sn-glycero-3-phosphoethanolamine (PEG-DLPE), PEG-1,2-dimyristoyl-sn-glycero-3-phosphoethanolamine (PEG-DMPE), PEG-1,2-dipalmitoyl-sn-glycero-3-phosphocholine (PEG-DPPC), 1-O-(2′-(ω-methoxy-polyethylene-glycol)succinoyl)-2-N-myristoyl-sphingosine (PEG-CerC14), or 1-O-(2′-(ω-methoxy-polyethylene-glycol)succinoyl)-2-N-arachidoyl-sphingosine (PEG-CerC20).

The aliphatic chains of the PEG-lipids can each have 14 to 22 carbons (e.g., 14 to 16, 16 to 18, 14 to 20, or 14 to 18 carbons). In some embodiments, a PEG moiety, for example an mPEG-NH₂, has a size of about 1000, 2000, 5000, 10,000, 15,000 or 20,000 daltons. In some embodiments, the PEG-lipid is PEG₂ₖ-DMG.

A lipid nanoparticle described herein can include a PEG-lipid which is a non-diffusible PEG. Non-limiting examples of non-diffusible PEGs include PEG-DSG and PEG-DSPE.

PEG-lipids can include those described in U.S. Pat. No. 8,158,601 and International Publication No. WO 2015/130584 and WO 2012/099755. The PEG-lipids described herein can be synthesized as described in International Patent Application No. PCT/US2016/000129.

In some embodiments, the PEG-lipid is a modified form of PEG-DMG. PEG-DMG has the following structure:

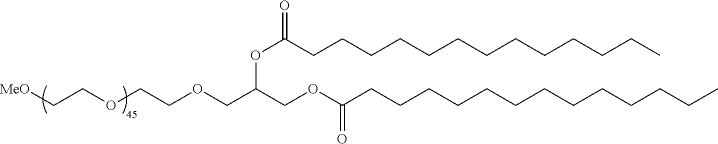

In certain embodiments, a PEG lipid useful in the present invention is a PEGylated fatty acid.

In one embodiment, the amount of PEG-lipid in the lipid composition of a pharmaceutical composition disclosed herein ranges from about 0.1 mol % to about 5 mol %, from about 0.5 mol % to about 5 mol %, from about 1 mol % to about 5 mol %, from about 1.5 mol % to about 5 mol %, from about 2 mol % to about 5 mol %, from about 0.1 mol % to about 4 mol %, from about 0.5 mol % to about 4 mol %, from about 1 mol % to about 4 mol %, from about 1.5 mol % to about 4 mol %, from about 2 mol % to about 4 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 0.1 mol % to about 2 mol %, from about 0.5 mol % to about 2 mol %, from about 1 mol % to about 2 mol %, from about 1.5 mol % to about 2 mol %, from about 0.1 mol % to about 1.5 mol %, from about 0.5 mol % to about 1.5 mol %, or from about 1 mol % to about 1.5 mol %.

In one embodiment, the amount of PEG-lipid in the lipid composition disclosed herein is about 2 mol %. In one embodiment, the amount of PEG-lipid in the lipid composition disclosed herein is about 1.5 mol %.

In one embodiment, the amount of PEG-lipid in the lipid composition disclosed herein is at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 mol %.

In some aspects, the lipid composition of the pharmaceutical compositions disclosed herein does not comprise a PEG-lipid.

### Other Components

A composition of the invention may include one or more components in addition to those described in the preceding sections. For example, a composition may include one or more small hydrophobic molecules such as a vitamin (e.g., vitamin A or vitamin E) or a sterol.

Compositions may also include one or more permeability enhancer molecules, carbohydrates, polymers, therapeutic agents, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a composition. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, polyoxamines, poly(ortho)esters, poly(butyric acid), poly(valeric acid), poly(lactide-co-caprolactone), and trimethylene carbonate, polyvinylpyrrolidone.

Therapeutic agents may include, but are not limited to, cytotoxic, chemotherapeutic, and other therapeutic agents. Cytotoxic agents may include, for example, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, rachelmycin, and analogs thereof. Radioactive ions may also be used as therapeutic agents and may include, for example, radioactive iodine, strontium, phosphorous, palladium, cesium, iridium, cobalt, yttrium, samarium, and praseodymium. Other therapeutic agents may include, for example, antimetabolites (e.g., methotrexate, 6-mercaptopurine, 6-thioguanine, cytarabine, and 5-fluorouracil, and decarbazine), alkylating agents (e.g., mechlorethamine, thiotepa, chlorambucil, rachelmycin, melphalan, carmustine, lomustine, cyclophosphamide, busulfan, dibromomannitol, streptozotocin, mitomycin C, and cis-dichlorodiamine platinum (II) (DDP), and cisplatin), anthracyclines (e.g., daunorubicin and doxorubicin), antibiotics (e.g., dactinomycin, bleomycin, mithramycin, and anthramycin), and anti-mitotic agents (e.g., vincristine, vinblastine, taxol, and maytansinoids).

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a composition (e.g., by coating, adsorption, covalent linkage, or other process).

In addition to these components, compositions of the invention may include any substance useful in pharmaceutical compositions. For example, the composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21^{st} Edition, A. R. Gennaro; Lippincott, Williams & Wilkins, Baltimore, Md., 2006).

Diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite (aluminum silicate) and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

A binding agent may be starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Preservatives include, but are not limited to, antioxidants, chelating agents, antimicrobial preservatives, antifungal preservatives, alcohol preservatives, acidic preservatives, and/or other preservatives. Antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Alcohol preservatives include, but are not limited to, ethanol, polyethylene glycol, phenol, benzyl alcohol, phenolic compounds, bisphenol, chlorobutanol, hydroxybenzoate, and/or phenylethyl alcohol. Acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g. HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

### RNA

An RNA may be a messenger RNA (mRNA). An mRNA may be a naturally or non-naturally occurring mRNA. An mRNA may include one or more modified nucleobases, nucleosides, or nucleotides. A nucleobase of an mRNA is an organic base such as a purine or pyrimidine or a derivative thereof. A nucleobase may be a canonical base (e.g., adenine, guanine, uracil, and cytosine) or a non-canonical or modified base including one or more substitutions or modifications including but not limited to alkyl, aryl, halo, oxo, hydroxyl, alkyloxy, and/or thio substitutions; one or more fused or open rings; oxidation; and/or reduction. Thus, a nucleobase may be selected from the non-limiting group consisting of adenine, guanine, uracil, cytosine, 7-methylguanine, 5-methylcytosine, 5-hydroxymethylcytosine, thymine, pseudouracil, dihydrouracil, hypoxanthine, and xanthine.

A nucleoside of an mRNA is a compound including a sugar molecule (e.g., a 5-carbon or 6-carbon sugar, such as pentose, ribose, arabinose, xylose, glucose, galactose, or a deoxy derivative thereof) in combination with a nucleobase. A nucleoside may be a canonical nucleoside (e.g., adenosine, guanosine, cytidine, uridine, 5-methyluridine, deoxyadenosine, deoxyguanosine, deoxycytidine, deoxyuridine, and thymidine) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase and/or sugar component.

A nucleotide of an mRNA is a compound containing a nucleoside and a phosphate group or alternative group (e.g., boranophosphate, thiophosphate, selenophosphate, phosphonate, alkyl group, amidate, and glycerol). A nucleotide may be a canonical nucleotide (e.g., adenosine, guanosine, cytidine, uridine, 5-methyluridine, deoxyadenosine, deoxyguanosine, deoxycytidine, deoxyuridine, and thymidine monophosphates) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase, sugar, and/or phosphate or alternative component. A nucleotide may include one or more phosphate or alternative groups. For example, a nucleotide may include a nucleoside and a triphosphate group. A “nucleoside triphosphate” (e.g., guanosine triphosphate, adenosine triphosphate, cytidine triphosphate, and uridine triphosphate) may refer to the canonical nucleoside triphosphate or an analog or derivative thereof and may include one or more substitutions or modifications as described herein. For example, “guanosine triphosphate” should be understood to include the canonical guanosine triphosphate, 7-methylguanosine triphosphate, or any other definition encompassed herein.

An mRNA may include a 5′ untranslated region, a 3′ untranslated region, and/or a coding or translating sequence. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine.

In some embodiments, an mRNA may include a 5′ cap structure, a chain terminating nucleotide, a stem loop, a polyA sequence, and/or a polyadenylation signal.

A cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m⁷G(5′)ppp(5′)G, commonly written as m⁷GpppG. A cap species may also be an anti-reverse cap analog. Cap species include m⁷GpppG, m⁷Gpppm⁷G, m⁷3′dGpppG, m₂^{7,O3}′GpppG, m₂^{7,O3}′GppppG, m₂^{7,O2}′GppppG, m⁷Gpppm⁷G, m⁷3′dGpppG, m₂^{7,O3}′GpppG, m₂^{7,O3}′GppppG, and m₂^{7,O2}′GppppG.

An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine.

An mRNA may instead or additionally include a stem loop, such as a histone stem loop. A stem loop may include 1, 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. An mRNA may encode any polypeptide of interest, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell.

### Compositions

A lipid nanoparticle of the invention includes an ionizable lipid and a structural component, where the structural component includes a compound of the invention (e.g., a compound of Formula I, Formula II, Formula III, Formula IV, Formula V, Formula VI, Formula VII, Formula VIII, Formula IX, Formula X, Formula XI, Formula XII, or Formula XIII, or a compound shown in Tables 1-14) or any one of compounds 131-133 in Table 15. The lipid nanoparticle can further include one or more structural lipids, one or more non-cationic helper lipids, one or more PEG-lipids, or any combination thereof. For example, a lipid nanoparticle can include 40 mol % of ionizable lipid, about 15 mol % non-cationic helper lipid, about 43.5 mol % structural component, and about 1.5% PEG-lipid. The lipid nanoparticle can further include a nucleic acid molecule (e.g., mRNA).

Exemplary compounds of the invention include compounds of Formula I, Formula II, Formula III, Formula IV, Formula V, Formula VI, Formula VII, Formula VIII, Formula IX, Formula X, Formula XI, Formula XII, and Formula XIII. Further exemplary compounds of the invention include compounds shown in Tables 1-14.

A composition of the invention may be designed for one or more specific applications or targets. For example, a composition may be designed to deliver mRNA to a particular cell, tissue, organ, or system or group thereof in a mammal's body, such as the renal system. Physiochemical properties of compositions may be altered in order to increase selectivity for particular bodily targets. For instance, particle sizes may be adjusted based on the fenestration sizes of different organs. The mRNA included in a composition may also depend on the desired delivery target or targets. For example, an mRNA may be selected for a particular indication, condition, disease, or disorder and/or for delivery to a particular cell, tissue, organ, or system or group thereof (e.g., localized or specific delivery). A composition may include one or more mRNA molecules encoding one or more polypeptides of interest.

The amount of mRNA in a composition may depend on the size, sequence, and other characteristics of the mRNA. The amount of mRNA in a composition may also depend on the size, composition, desired target, and other characteristics of the composition. The relative amounts of mRNA and other elements (e.g., lipids) may also vary. In some embodiments, the wt/wt ratio of one or more ionizable lipids, structural component, one or more non-cationic helper lipids, one or more PEG-lipids, or any combination thereof to an mRNA in a composition may be from about 5:1 to about 50:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, and 50:1. For example, the wt/wt ratio of one or more ionizable lipids, structural component, one or more non-cationic helper lipids, one or more PEG-lipids, or any combination thereof to an mRNA may be from about 10:1 to about 40:1. The amount of mRNA in a composition may, for example, be measured using absorption spectroscopy (e.g., ultraviolet-visible spectroscopy).

In some embodiments, mRNA, lipid nanoparticles, and amounts thereof may be selected to provide a specific N:P ratio. The N:P ratio of the composition refers to the molar ratio of nitrogen atoms in one or more lipids to the number of phosphate groups in an mRNA. In general, a lower N:P ratio is preferred. The mRNA, lipid nanoparticles, and amounts thereof may be selected to provide an N:P ratio from about 2:1 to about 8:1, such as 2:1, 3:1, 4:1, 5:1, 6:1, 7:1, and 8:1. In certain embodiments, the N:P ratio may be from about 2:1 to about 5:1.

### Physical Properties

The characteristics of a composition may depend on the components thereof. Similarly, the characteristics of a composition may depend on the absolute or relative amounts of its components. For instance, a composition including a higher molar fraction of a cationic lipid may have different characteristics than a composition including a lower molar fraction of a cationic lipid. Characteristics may also vary depending on the method and conditions of preparation of the composition.

Compositions may be characterized by a variety of methods. For example, microscopy (e.g., transmission electron microscopy or scanning electron microscopy) may be used to examine the morphology and size distribution of a composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a composition, such as particle size, polydispersity index, and zeta potential.

The mean size of a composition of the invention may be between 10 s of nm and 100 s of nm. For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a composition may be from about 80 nm to about 120 nm. In a particular embodiment, the mean size may be about 90 nm.

A composition of the invention may be relatively homogenous. A polydispersity index may be used to indicate the homogeneity of a composition, e.g., the particle size distribution of the compositions. A small (e.g., less than 0.3) polydispersity index generally indicates a narrow particle size distribution. A composition of the invention may have a polydispersity index from about 0 to about 0.18, such as 0.01, 0.02, 0.03, 0.04, 0.05, 0.06, 0.07, 0.08, 0.09, 0.10, 0.11, 0.12, 0.13, 0.14, 0.15, 0.16, 0.17, or 0.18. In some embodiments, the polydispersity index of a composition may be from about 0.13 to about 0.17.

The zeta potential of a composition may be used to indicate the electrokinetic potential of the composition. For example, the zeta potential may describe the surface charge of a composition. Compositions with relatively low charges, positive or negative, are generally desirable, as more highly charged species may interact undesirably with cells, tissues, and other elements in the body. In some embodiments, the zeta potential of a composition of the invention may be from about −10 mV to about +20 mV.

The efficiency of encapsulation of an mRNA describes the amount of mRNA that is encapsulated or otherwise associated with a composition after preparation, relative to the initial amount provided. The encapsulation efficiency is desirably high (e.g., close to 100%). The encapsulation efficiency may be measured, for example, by comparing the amount of mRNA in a solution containing the composition before and after breaking up the composition with one or more organic solvents or detergents. Fluorescence may be used to measure the amount of free mRNA in a solution. For the compositions of the invention, the encapsulation efficiency of an mRNA may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A composition of the invention may optionally comprise one or more coatings. For example, a composition may be formulated in a capsule, film, or tablet having a coating. A capsule, film, or tablet including a composition of the invention may have any useful size, tensile strength, hardness, or density.

### Pharmaceutical Compositions

Compositions of the invention may be formulated in whole or in part as pharmaceutical compositions. Pharmaceutical compositions of the invention may include one or more compositions. For example, a pharmaceutical composition may include one or more compositions including one or more different mRNAs. Pharmaceutical compositions of the invention may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, Md., 2006. Conventional excipients and accessory ingredients may be used in any pharmaceutical composition of the invention, except insofar as any conventional excipient or accessory ingredient may be incompatible with one or more components of a composition of the invention. An excipient or accessory ingredient may be incompatible with a component of a composition if its combination with the component may result in any undesirable biological effect or otherwise deleterious effect.

In some embodiments, one or more excipients or accessory ingredients may make up greater than 50% of the total mass or volume of a pharmaceutical composition including a composition of the invention. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more compositions, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more compositions.

Compositions and/or pharmaceutical compositions including one or more compositions may be administered to any patient or subject, including those patients or subjects that may benefit from a therapeutic effect provided by the delivery of an mRNA to one or more particular cells, tissues, organs, or systems or groups thereof, such as the renal system. Although the descriptions provided herein of compositions and pharmaceutical compositions including compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats.

A pharmaceutical composition including one or more compositions may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., composition). The amount of the active ingredient is generally equal to the dosage of the active ingredient which would be administered to a subject and/or a convenient fraction of such a dosage such as, for example, one-half or one-third of such a dosage.

Pharmaceutical compositions of the invention may be prepared in a variety of forms suitable for a variety of routes and methods of administration. For example, pharmaceutical compositions of the invention may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, films, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay, silicates), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Embedding compositions which can be used include, but are not limited to, polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a composition may include ointments, pastes, creams, lotions, gels, powders, solutions, sprays, inhalants, and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (wt/wt) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are conveniently in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (wt/wt) of the composition, and active ingredient may constitute 0.1% to 20% (wt/wt) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (wt/wt) and as much as 100% (wt/wt) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (wt/wt) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (wt/wt) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this present disclosure.

### Methods of Producing Polypeptides in Cells

The present disclosure provides methods of producing a polypeptide of interest in a mammalian cell. Methods of producing polypeptides involve contacting a cell with a composition including an mRNA encoding the polypeptide of interest. Upon contacting the cell with the composition, the mRNA may be taken up and translated in the cell to produce the polypeptide of interest.

In general, the step of contacting a mammalian cell with a composition including an mRNA encoding a polypeptide of interest may be performed in vivo, ex vivo, in culture, or in vitro. The amount of composition contacted with a cell, and/or the amount of mRNA therein, may depend on the type of cell or tissue being contacted, the means of administration, the physiochemical characteristics of the composition and the mRNA (e.g., size, charge, and chemical composition) therein, and other factors. In general, an effective amount of the composition will allow for efficient polypeptide production in the cell. Metrics for efficiency may include polypeptide translation (indicated by polypeptide expression), level of mRNA degradation, and immune response indicators.

The step of contacting a composition including an mRNA with a cell may involve or cause transfection. A phospholipid including in the non-cationic helper lipid of a composition may facilitate transfection and/or increase transfection efficiency, for example, by interacting and/or fusing with a cellular or intracellular membrane. Transfection may allow for the translation of the mRNA within the cell.

In some embodiments, the compositions described herein may be used as therapeutic agents. For example, an mRNA included in a composition may encode a therapeutic polypeptide (e.g., in a translatable region) and produce the therapeutic polypeptide upon contacting and/or entry (e.g., transfection) into a cell. In other embodiments, an mRNA included in a composition of the invention may encode a polypeptide that may improve or increase the immunity of a subject. For example, an mRNA may encode a granulocyte-colony stimulating factor or trastuzumab.

In certain embodiments, an mRNA included in a composition of the invention may encode a recombinant polypeptide that may replace one or more polypeptides that may be substantially absent in a cell contacted with the composition. The one or more substantially absent polypeptides may be lacking due to a genetic mutation of the encoding gene or a regulatory pathway thereof. Alternatively, a recombinant polypeptide produced by translation of the mRNA may antagonize the activity of an endogenous protein present in, on the surface of, or secreted from the cell. An antagonistic recombinant polypeptide may be desirable to combat deleterious effects caused by activities of the endogenous protein, such as altered activities or localization caused by mutation. In another alternative, a recombinant polypeptide produced by translation of the mRNA may indirectly or directly antagonize the activity of a biological moiety present in, on the surface of, or secreted from the cell. Antagonized biological moieties may include, but are not limited to, lipids (e.g., cholesterol), lipoproteins (e.g., low density lipoprotein), nucleic acids, carbohydrates, and small molecule toxins. Recombinant polypeptides produced by translation of the mRNA may be engineered for localization within the cell, such as within a specific compartment such as the nucleus, or may be engineered for secretion from the cell or for translocation to the plasma membrane of the cell.

In some embodiments, contacting a cell with a composition including an mRNA may reduce the innate immune response of a cell to an exogenous nucleic acid. A cell may be contacted with a first composition including a first amount of a first exogenous mRNA including a translatable region and the level of the innate immune response of the cell to the first exogenous mRNA may be determined. Subsequently, the cell may be contacted with a second composition including a second amount of the first exogenous mRNA, the second amount being a lesser amount of the first exogenous mRNA compared to the first amount. Alternatively, the second composition may include a first amount of a second exogenous mRNA that is different from the first exogenous mRNA. The steps of contacting the cell with the first and second compositions may be repeated one or more times. Additionally, efficiency of polypeptide production (e.g., translation) in the cell may be optionally determined, and the cell may be re-contacted with the first and/or second composition repeatedly until a target protein production efficiency is achieved.

Methods of Delivering mRNA to Cells

The present disclosure provides methods of delivering an mRNA to a mammalian cell. Delivery of an mRNA to a cell involves administering a composition including the mRNA to a subject, where administration of the composition involves contacting the cell with the composition. Upon contacting the cell with the composition, a translatable mRNA may be translated in the cell to produce a polypeptide of interest. However, mRNAs that are substantially not translatable may also be delivered to cells. Substantially non-translatable mRNAs may be useful as vaccines and/or may sequester translational components of a cell to reduce expression of other species in the cell.

In some embodiments, a composition of the invention may target a particular type or class of cells. For example, an mRNA that encodes a protein-binding partner (e.g., an antibody or functional fragment thereof, a scaffold protein, or a peptide) or a receptor on a cell surface may be included in a composition. An mRNA may additionally or instead be used to direct the synthesis and extracellular localization of lipids, carbohydrates, or other biological moieties. Alternatively, other elements (e.g., lipids or ligands) of a composition may be selected based on their affinity for particular receptors (e.g., low density lipoprotein receptors) such that a composition may more readily interact with a target cell population including the receptors. For example, ligands may include, but are not limited to, members of a specific binding pair, antibodies, monoclonal antibodies, Fv fragments, single chain Fv (scFv) fragments, Fab′ fragments, F(ab′)₂ fragments, single domain antibodies, camelized antibodies and fragments thereof, humanized antibodies and fragments thereof, and multivalent versions thereof; multivalent binding reagents including mono- or bi-specific antibodies such as disulfide stabilized Fv fragments, scFv tandems, diabodies, tridobdies, or tetrabodies; and aptamers, receptors, and fusion proteins.

In some embodiments, a ligand may be a surface-bound antibody, which can permit tuning of cell targeting specificity. This is especially useful since highly specific antibodies can be raised against an epitope of interest for the desired targeting site. In one embodiment, multiple antibodies are expressed on the surface of a cell, and each antibody can have a different specificity for a desired target. Such approaches can increase the avidity and specificity of targeting interactions.

A ligand can be selected, e.g., by a person skilled in the biological arts, based on the desired localization or function of the cell. For example an estrogen receptor ligand, such as tamoxifen, can target cells to estrogen-dependent breast cancer cells that have an increased number of estrogen receptors on the cell surface. Ligand/receptor interactions include, but are not limited to, CCR1 (e.g., for treatment of inflamed joint tissues or brain in rheumatoid arthritis, and/or multiple sclerosis), CCR7, CCR8 (e.g., targeting to lymph node tissue), CCR6, CCR9,CCR10 (e.g., to target to intestinal tissue), CCR4, CCR10 (e.g., for targeting to skin), CXCR4 (e.g., for general enhanced transmigration), HCELL (e.g., for treatment of inflammation and inflammatory disorders, bone marrow), Alpha4beta7 (e.g., for intestinal mucosa targeting), and VLA-4NCAM-1 (e.g., targeting to endothelium). In general, any receptor involved in targeting (e.g., cancer metastasis) can be harnessed for use in the methods and compositions described herein.

Targeted cells may include, but are not limited to, hepatocytes, epithelial cells, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells.

In particular embodiments, a composition of the invention may target hepatocytes. Apolipoprotiens such as apolipoprotein E (apoE) have been shown to associate with neutral or near neutral lipid-containing compositions in the body, and are known to associate with receptors such as low-density lipoprotein receptors (LDLRs) found on the surface of hepatocytes. Thus, a composition including a lipid nanoparticle with a neutral or near neutral charge that is administered to a subject may acquire apoE in a subject's body and may subsequently deliver mRNA to hepatocytes including LDLRs in a targeted manner.

Compositions of the invention may be useful for treating a disease, disorder, or condition characterized by missing or aberrant protein or polypeptide activity. Upon delivery of an mRNA encoding the missing or aberrant polypeptide to a cell, translation of the mRNA may produce the polypeptide, thereby reducing or eliminating an issue caused by the absence of or aberrant activity caused by the polypeptide. Because translation may occur rapidly, the methods and compositions of the invention may be useful in the treatment of acute diseases, disorders, or conditions such as sepsis, stroke, and myocardial infarction. An mRNA included in a composition of the invention may also be capable of altering the rate of transcription of a given species, thereby affecting gene expression.

Diseases, disorders, and/or conditions characterized by dysfunctional or aberrant protein or polypeptide activity for which a composition of the invention may be administered include, but are not limited to, cancer and proliferative diseases, genetic diseases (e.g., cystic fibrosis), autoimmune diseases, diabetes, neurodegenerative diseases, cardio- and reno-vascular diseases, and metabolic diseases. Multiple diseases, disorders, and/or conditions may be characterized by missing (or substantially diminished such that proper protein function does not occur) protein activity. Such proteins may not be present, or they may be essentially non-functional. A specific example of a dysfunctional protein is the missense mutation variants of the cystic fibrosis transmembrane conductance regulator (CFTR) gene, which produce a dysfunctional protein variant of CFTR protein, which causes cystic fibrosis. The present disclosure provides a method for treating such diseases, disorders, and/or conditions in a subject by administering a composition including an mRNA and a ionizable lipid including KL22, a non-cationic helper lipid (e.g., phospholipid that is optionally unsaturated), a PEG-lipid, and a structural lipid, wherein the mRNA encodes a polypeptide that antagonizes or otherwise overcomes an aberrant protein activity present in the cell of the subject.

The invention provides methods involving administering compositions including mRNA or pharmaceutical compositions including the same. Compositions of the invention, or imaging, diagnostic, or prophylactic compositions thereof, may be administered to a subject using any reasonable amount and any route of administration effective for preventing, treating, diagnosing, or imaging a disease, disorder, and/or condition and/or any other purpose. The specific amount administered to a given subject may vary depending on the species, age, and general condition of the subject; the purpose of the administration; the particular composition; the mode of administration; and the like. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment. The specific therapeutically effective, prophylactically effective, or otherwise appropriate dose level (e.g., for imaging) for any particular patient will depend upon a variety of factors including the severity and identify of a disorder being treated, if any; the one or more mRNAs employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

A composition including one or more mRNAs may be administered by any route. In some embodiments, compositions of the invention, including prophylactic, diagnostic, or imaging compositions including one or more compositions of the invention, are administered by one or more of a variety of routes, including oral, intravenous, intramuscular, intra-arterial, intramedullary, intrathecal, subcutaneous, intraventricular, trans- or intra-dermal, interdermal, rectal, intravaginal, intraperitoneal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, or subcutaneously. However, the present disclosure encompasses the delivery of compositions of the invention by any appropriate route taking into consideration likely advances in the sciences of drug delivery. In general, the most appropriate route of administration will depend upon a variety of factors including the nature of the composition including one or more mRNAs (e.g., its stability in various bodily environments such as the bloodstream and gastrointestinal tract), the condition of the patient (e.g., whether the patient is able to tolerate particular routes of administration), etc.

In certain embodiments, compositions in accordance with the present disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.1 mg/kg to about 10 mg/kg, from about 1 mg/kg to about 5 mg/kg, from about 2 mg/kg to about 5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, or from about 0.1 mg/kg to about 1 mg/kg in a given dose, where a dose of 1 mg/kg provides 1 mg of a composition per 1 kg of subject body weight. In particular embodiments, a dose of about 0.005 mg/kg to about 5 mg/kg of a composition of the invention may be administrated. A dose may be administered one or more times per day, in the same or a different amount, to obtain a desired level of mRNA expression and/or therapeutic, diagnostic, prophylactic, or imaging effect. The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In some embodiments, a single dose may be administered, for example, prior to or after a surgical procedure or in the instance of an acute disease, disorder, or condition.

Compositions including one or more mRNAs may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. For example, one or more compositions including one or more different mRNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the invention, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

### EXAMPLES

### Example 1. Synthesis of Methyl (R)-4-((3R,5R,6S,8S,9S,10R,13R,14S,17R)-3,6-dihydroxy-10,13-dimethylhexadecahydro-1H-cyclopenta[a]phenanthren-17-yl)pentanoate (66)

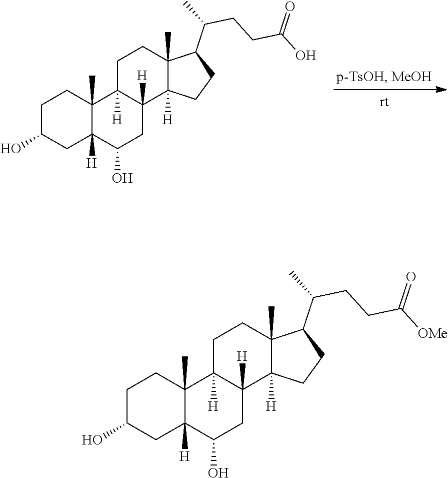

A mixture of hyodeoxycholic acid (10.0 g, 25.5 mmol) and p-toluenesulfonic acid monohydrate (1.21 g, 6.37 mmol) was dissolved in MeOH (100 mL), and allowed to stir at room temperature for 24 h.

The solvent was removed in vacuo. EtOAc and water were added, the EtOAc phase was separated, and the aqueous phase was extracted with EtOAc (3×). The organic extracts were combined, washed with brine (2×), dried (MgSO₄), filtered, and concentrated in vacuo to afford the desired product (10.4 g, quantitative) as a white amorphous solid, which was used in the next step without further purification. ¹H NMR: (300 MHz, CDCl₃) δ 4.05 (ddd, J=9.0, 6.0, 6.0 Hz, 1H), 3.66 (s, 3H), 3.64-3.54 (m, 1H), 2.41-2.29 (m, 1H), 2.27-2.14 (m, 1H), 2.00-1.52 (m, 12H), 1.51-0.98 (m, 16H), 0.91 (d, J=6.0 Hz, 3H), 0.90 (s, 3H), 0.63 (s, 3H).

### Example 2. Synthesis of Methyl (R)-4-((3R,5R,6S,8S,9S,10R,13R,14S,17R)-10,13-dimethyl-3,6-bis(tosyloxy)hexadecahydro-1H-cyclopenta[a]phenanthren-17-yl)pentanoate (67)

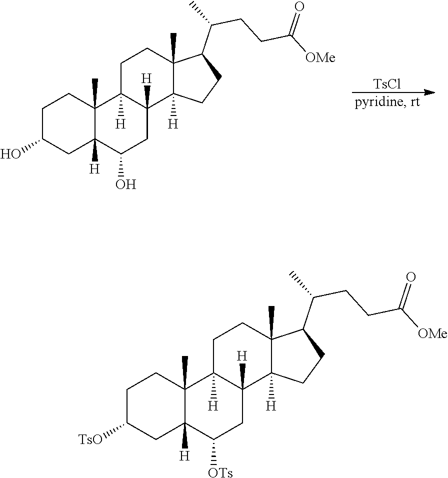

Pyridine (9 mL) was added to a mixture of compound 66 (1.00 g, 2.46 mmol) and p-toluenesulfonyl chloride (1.88 g, 9.84 mmol). The resulting solution was allowed to stir at room temperature for 18 h. Ice chips and water were added to the reaction mixture, followed by dilution with CH₂Cl₂. The layers were separated and the organic layer was washed with 1 M HCl, water, and brine. The organic layer was then dried over MgSO₄, filtered, and concentrated in vacuo to afford the desired product (1.76 g, quantitative) as a white amorphous solid, which was used in the next step without further purification. ¹H NMR: (300 MHz, CDCl₃) δ 7.78 (d, J=6.0 Hz, 2H), 7.72 (d, J=9.0 Hz, 2H), 7.35 (d, J=6.0 Hz, 2H), 7.32 (d, J=6.0 Hz, 2H), 4.78 (ddd, J=12.0, 6.0, 6.0 Hz, 1H), 4.36-4.23 (m, 1H), 3.65 (s, 3H), 2.46 (s, 6H), 2.39-2.27 (m, 1H), 2.26-2.14 (m, 1H), 2.00-0.92 (m, 26H), 0.88 (d, J=6.0 Hz, 3H), 0.80 (s, 3H), 0.58 (s, 3H).

### Example 3. Synthesis of Methyl (R)-4-((3S,8S,9S,10R,13R,14S,17R)-3-hydroxy-10,13-dimethyl-2,3,4,7,8,9,10,11,12,13,14,15,16,17-tetradecahydro-1H-cyclopenta[a]phenanthren-17-yl)pentanoate (51)

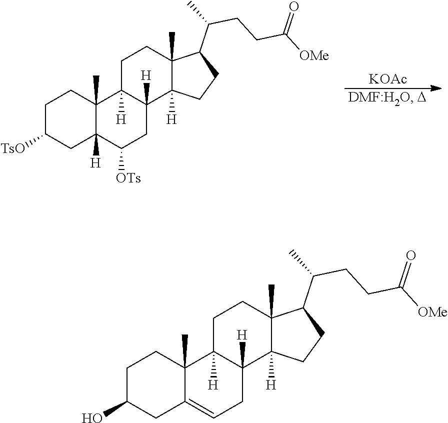

A solution of ditosylate 67 (67.0 g, 93.7 mmol) and potassium acetate (18.4 g, 187 mmol) dissolved in water (62 mL) and DMF (402 mL) was refluxed for 24 h. Upon cooling to room temperature, the reaction mixture was diluted with EtOAc and water. Layers were separated and the aqueous phase was extracted with EtOAc (3×). The organic extracts/layers were combined, washed with brine (2×), dried over MgSO₄, filtered and concentrated in vacuo. The crude material was purified by silica gel chromatography (0-10-30-50-80% EtOAc:hexanes) to afford the desired product (12.3 g, 34%) as a white solid. ¹H NMR: (300 MHz, CDCl₃) δ 5.35 (br d, J=3.0 Hz. 1H), 3.66 (s, 3H), 3.58-3.46 (m, 1H), 2.41-2.15 (m, 4H), 2.05-1.73 (m, 6H), 1.65-0.87 (m, 18H), 1.00 (s, 3H), 0.92 (d, J=6.0 Hz, 3H), 0.68 (s, 3H).

### Example 4. Synthesis of (R)-4-((3S,8S,9S,10R,13R,14S,17R)-3-Hydroxy-10,13-dimethyl-2,3,4,7,8,9,10,11,12,13,14,15,16,17-tetradecahydro-1H-cyclopenta[a]phenanthren-17-yl)pentanoic acid (149)

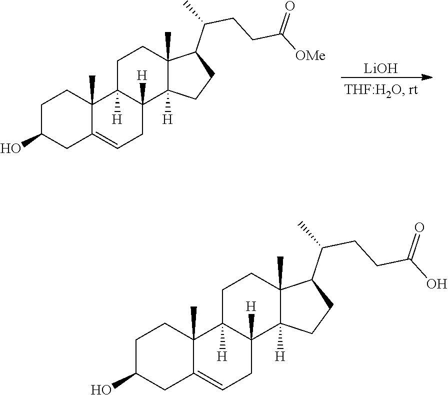

To a solution of cholenic acid methyl ester (4.00 g, 10.3 mmol) in water (55.3 mL) and THE (55.3 mL) was added lithium hydroxide (1.38 g, 57.6 mmol). The resulting mixture was stirred at room temperature for 18 h. The crude reaction mixture was rotavaped to remove the organic layer and the aqueous residue was acidified to pH 3-4 with 1 M HCl. Methanol was added to the aqueous solution to promote solubility and the aqueous layer was extracted with EtOAc (3×). The organic extracts were combined, washed with brine, dried (MgSO₄), filtered, and concentrated in vacuo to yield a the product (3.76 g, 97%) as a white solid, which was used without further purification. ¹H NMR: (300 MHz, MeOD) δ 5.35 (br d, J=3.0 Hz, 1H), 3.46-3.31 (m, 1H), 2.40-2.14 (m, 4H), 2.09-1.74 (m, 6H), 1.70-0.88 (m, 18H), 1.03 (s, 3H), 0.96 (d, J=6.0 Hz, 3H), 0.73 (s, 3H).

### Example 5. Synthesis of Ethyl (R)-4-((3S,8S,9S,10R,13R,14S,17R)-3-hydroxy-10,13-dimethyl-2,3,4,7,8,9,10,11,12,13,14,15,16,17-tetradecahydro-1H-cyclopenta[a]phenanthren-17-yl)pentanoate (52)

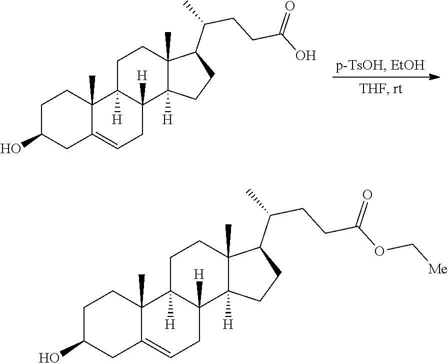

A mixture of cholenic acid 149 (175 mg, 0.467 mmol) and p-toluenesulfonic acid monohydrate (22 mg, 0.117 mmol) was dissolved in EtOH (6 mL) and THE (5 mL). The resulting mixture was allowed to stir at room temperature for 24 h. The solvent was removed in vacuo. EtOAc and water were added, the EtOAc phase was separated, and the aqueous phase was extracted with EtOAc (3×). The organic extracts were combined, washed with brine (2×), dried (MgSO₄), and concentrated in vacuo. The crude material was purified by silica gel chromatography (0-10-30-50 EtOAc:hexanes) to afford the desired product (122 mg, 65%) as a white solid. ¹H NMR: (300 MHz, CDCl₃) δ 5.34 (br d, J=6.0 Hz, 1H), 4.11 (q, J=6.0 Hz, 2H), 3.59-3.45 (m, 1H), 2.40-2.14 (m, 4H), 2.05-1.73 (m, 6H), 1.64-0.87 (m, 17H), 1.25 (t, J=6.0 Hz, 3H), 1.00 (s, 3H), 0.92 (d, J=6.0 Hz, 3H), 0.68 (s, 3H).

### Example 6. Synthesis of Isopropyl (R)-4-((3S,8S,9S,10R,13R,14S,17R)-3-hydroxy-10,13-dimethyl-2,3,4,7,8,9,10,11,12,13,14,15,16,17-tetradecahydro-1H-cyclopenta[a]phenanthren-17-yl)pentanoate (53)

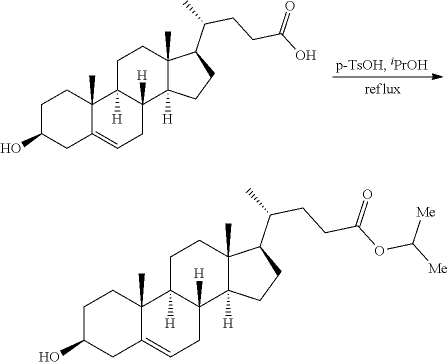

To a round-bottom flask equipped with a stir bar was added cholenic acid 149 (175 mg, 0.467 mmol), isopropyl alcohol (10 mL), and p-toluenesulfonic acid monohydrate (22 mg, 0.117 mmol). The resulting mixture was heated to reflux and stirred for 16 h. The solvent was removed under reduced pressure and EtOAc and water were added. The EtOAc phase was separated, and the aqueous phase was extracted with EtOAc (3×). The organic extracts we combined, washed with brine (2×), dried (MgSO₄), and concentrated in vacuo. The crude material was purified by silica gel chromatography (0-10-30-50-80% EtOAc:hexanes) to afford the desired product (86 mg, 44%) as a white solid. ¹H NMR: (300 MHz, CDCl₃) δ 5.34 (br d, J=3.0 Hz, 1H), 4.99 (septet, J=6.0 Hz, 1H), 3.58-3.44 (m, 1H), 2.36-2.11 (m, 4H), 2.03-1.68 (m, 7H), 1.63-0.78 (m, 19H), 1.21 (d, J=6.0 Hz, 6H), 0.99 (s, 3H), 0.92 (d, J=6.0 Hz, 3H), 0.67 (s, 3H).

### Example 7. General Procedure for the Synthesis of Sterol Amides

To a solution of cholenic acid (1 equiv.) and triethylamine (1.44 equiv.) in THE (0.013 M) was added isobutyl chloroformate (1.54 equiv.) at 0° C. The mixture was stirred at 0° C. for 10 min prior to the addition of the amine (20 equiv.) at 0° C. The resulting solution was allowed to stir at room temperature for 16 h. The reaction was diluted with EtOAc, and the organic layer was washed with saturated aqueous NH₄Cl and brine. Organic layer was dried (MgSO₄), filtered, and concentrated in vacuo. The crude material was purified as indicated below.

### (R)-4-((3S,8S,9S,10R,13R,14S,17R)-3-Hydroxy-10,13-dimethyl-2,3,4,7,8,9,10,11,12,13,14,15,16,17-tetradecahydro-1H-cyclopenta[a]phenanthren-17-yl)-1-(pyrrolidin-1-yl)pentan-1-one (55)

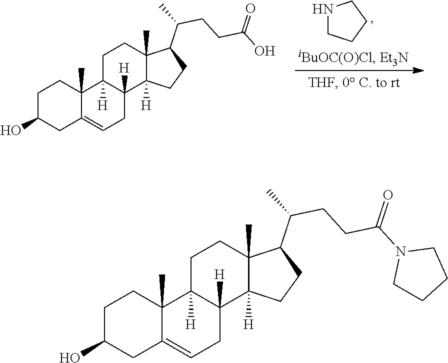

Synthesized according to the general procedure. Cholenic acid (175 mg, 0.467 mmol), triethylamine (94.0 μL, 0.673 mmol), isobutyl chloroformate (94.0 μL, 0.720 mmol), pyrrolidine (767 μL, 9.34 mmol), and THF (37 mL). The crude material was purified by silica gel chromatography (50-75-100% EtOAc:hexanes) to afford the desired product (151 mg, 76%) as a white solid. ¹H NMR: (300 MHz, CDCl₃) δ 5.35 (br d, J=6.0 Hz, 1H), 3.60-3.38 (m, 5H), 2.40-2.12 (m, 4H), 2.06-1.74 (m, 13H), 1.65-0.85 (m, 16H), 1.01 (s, 3H), 0.95 (d, J=6.0 Hz, 3H), 0.68 (s, 3H).

### (R)-4-((3S,8S,9S,10R,13R,14S,17R)-3-Hydroxy-10,13-dimethyl-2,3,4,7,8,9,10,11,12,13,14,15,16,17-tetradecahydro-1H-cyclopenta[a]phenanthren-17-yl)-1-(piperidin-1-yl)pentan-1-one (56)

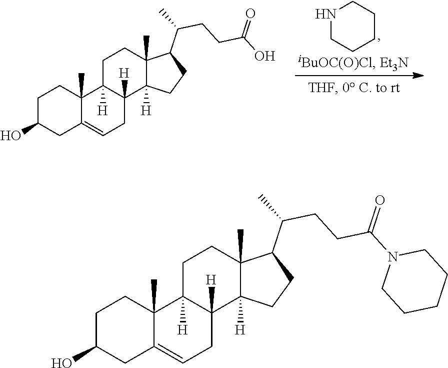

Synthesized according to the general procedure. Cholenic acid (175 mg, 0.467 mmol), triethylamine (94.0 μL, 0.673 mmol), isobutyl chloroformate (94.0 μL, 0.720 mmol), piperidine (923 μL, 9.34 mmol), and THF (37 mL). The crude material was purified by silica gel chromatography (30-50-70-100% EtOAc:hexanes) to afford the desired product (119 mg, 58%) as a white solid. ¹H NMR: (300 MHz, CDCl₃) δ 5.34 (br d, J=3.0 Hz, 1H), 3.64-3.28 (br m, 5H), 2.43-2.14 (m, 4H), 2.05-0.86 (m, 31H), 1.00 (s, 3H), 0.95 (d, J=6.0 Hz, 3H), 0.68 (s, 3H).

### (R)-1-(4,4-Dimethylpiperidin-1-yl)-4-((3S,8S,9S,10R,13R,14S,17R)-3-hydroxy-10,13-dimethyl-2,3,4,7,8,9,10,11,12,13,14,15,16,17-tetradecahydro-1H-cyclopenta[a]phenanthren-17-yl)pentan-1-one (57)

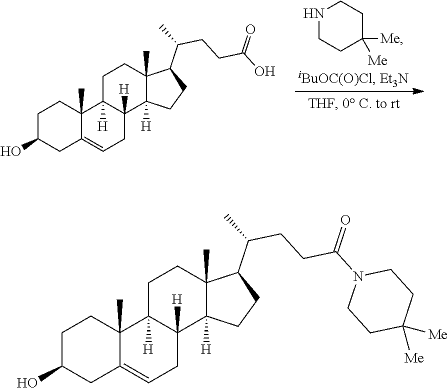

Synthesized according to the general procedure. Cholenic acid (175 mg, 0.467 mmol), triethylamine (94.0 μL, 0.673 mmol), isobutyl chloroformate (94.0 μL, 0.720 mmol), 4,4-dimethylpiperidine (1.40 mL, 9.34 mmol), and THF (37 mL). The crude material was purified by silica gel chromatography (0-10-30-50-80% EtOAc:hexanes) to afford the desired product (156 mg, 71%) as a clear oil. ¹H NMR: (300 MHz, CDCl₃) δ 5.35 (br d, J=6.0 Hz, 1H), 3.60-3.37 (br m, 5H), 2.46-2.17 (m, 4H), 2.06-0.80 (m, 32H), 1.01 (s, 3H), 0.98 (s, 6H), 0.96 (d, J=9.0 Hz, 3H), 0.68 (s, 3H).

### (R)-4-((3S,8S,9S,10R,13R,14S,17R)-3-Hydroxy-10,13-dimethyl-2,3,4,7,8,9,10,11,12,13,14,15,16,17-tetradecahydro-1H-cyclopenta[a]phenanthren-17-yl)-N-methylpentanamide (59)

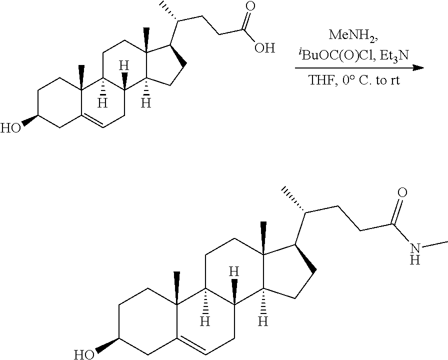

Synthesized according to the general procedure. Cholenic acid (200 mg, 0.534 mmol), triethylamine (107 μL, 0.769 mmol), isobutyl chloroformate (107 μL, 0.822 mmol), methylamine (2 M in THF, 5.34 mL, 10.7 mmol), and THF (43 mL). The crude material was purified by silica gel chromatography (50-75-100% EtOAc:hexanes) to afford the desired product (135 mg, 65%) as a white solid. ¹H NMR: (300 MHz, MeOD) δ 5.34 (br d, J=6.0 Hz, 1H), 3.46-3.28 (m, 1H), 2.70 (s, 3H), 2.30-0.89 (m, 27H), 1.02 (s, 3H), 0.97 (d, J=6.0 Hz, 3H), 0.72 (s, 3H).

### (R)-4-((3S,8S,9S,10R,13R,14S,17R)-3-Hydroxy-10,13-dimethyl-2,3,4,7,8,9,10,11,12,13,14,15,16,17-tetradecahydro-1H-cyclopenta[a]phenanthren-17-yl)-N,N-dimethylpentanamide (60)

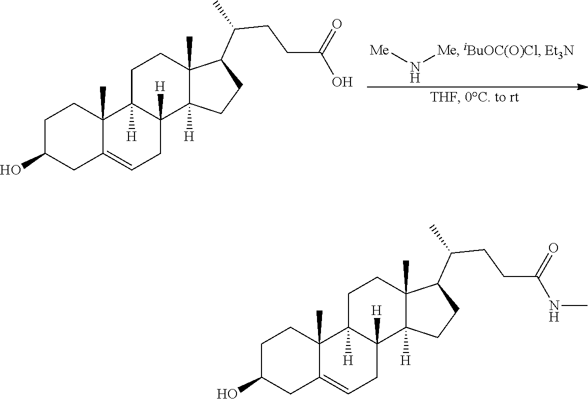

Synthesized according to the general procedure. Cholenic acid (200 mg, 0.534 mmol), triethylamine (107 μL, 0.769 mmol), isobutyl chloroformate (107 μL, 0.822 mmol), dimethylamine (2 M in THF, 5.34 mL, 10.7 mmol), and THF (43 mL). The crude material was purified by silica gel chromatography (25-50-75-100% EtOAc:hexanes) to afford the desired product (142 mg, 66%) as a white solid. ¹H NMR: (300 MHz, CDCl₃) δ 5.34 (br d, J=6.0 Hz, 1H), 3.59-3.45 (m, 1H), 2.97 (br s, 6H), 2.42-2.14 (m, 4H), 2.05-0.86 (m, 23H), 1.00 (s, 3H), 0.94 (d, J=6.0 Hz, 3H), 0.68 (s, 3H).

### (R)-N,N-Diethyl-4-((3S,8S,9S,10R,13R,14S,17R)-3-hydroxy-10,13-dimethyl-2,3,4,7,8,9,10,11,12,13,14,15,16,17-tetradecahydro-1H-cyclopenta[a]phenanthren-17-yl)pentanamide (61)

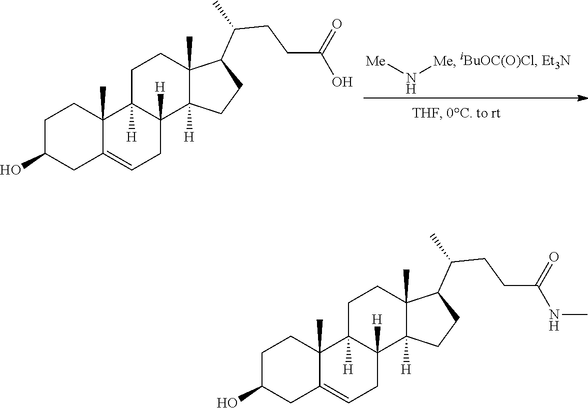

Synthesized according to the general procedure. Cholenic acid (200 mg, 0.534 mmol), triethylamine (107 μL, 0.769 mmol), isobutyl chloroformate (107 μL, 0.822 mmol), diethylamine (1.10 mL, 10.7 mmol), and THE (43 mL). The crude material was purified by silica gel chromatography (0-20-40-70-100% EtOAc:hexanes) to afford the desired product (148 mg, 65%) as a white solid. ¹H NMR: (300 MHz, MeOD) δ 5.34 (br d, J=1H), 3.45-3.31 (m, 5H), 2.45-2.14 (m, 4H), 2.10-0.89 (m, 23H), 1.21 (t, J=9.0 Hz, 3H), 1.10 (t, J=9.0 Hz, 3H), 1.03 (s, 3H), 0.99 (d, J=6.0 Hz, 3H), 0.73 (s, 3H).

### (R)-4-((3S,8S,9S,10R,13R,14S,17R)-3-Hydroxy-10,13-dimethyl-2,3,4,7,8,9,10,11,12,13,14,15,16,17-tetradecahydro-1H-cyclopenta[a]phenanthren-17-yl)-N,N-dipropylpentanamide (62)

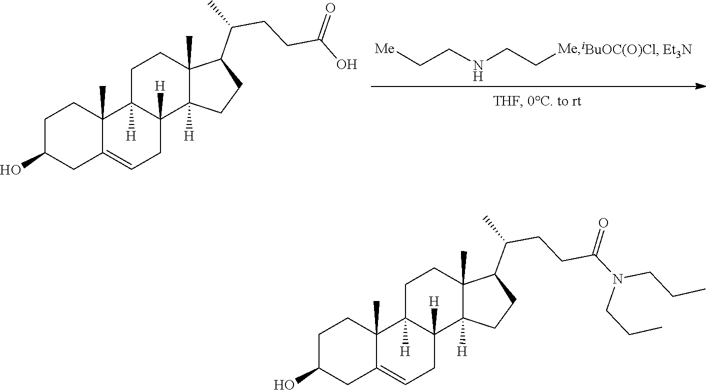

Synthesized according to the general procedure. Cholenic acid (200 mg, 0.534 mmol), triethylamine (107 μL, 0.769 mmol), isobutyl chloroformate (107 μL, 0.822 mmol), dipropylamine (1.46 mL, 10.7 mmol), and THE (43 mL). The crude material was purified by silica gel chromatography (0-10-20-50-80% EtOAc:hexanes) to afford the desired product (173 mg, 71%) as a white solid. ¹H NMR: (300 MHz, CDCl₃) δ 5.31 (br d, 1H), 3.56-3.42 (m, 1H), 3.32-3.10 (m, 4H), 2.38-2.09 (m, 4H), 2.03-1.70 (m, 6H), 1.62-0.80 (m, 29H), 0.97 (s, 3H), 0.65 (s, 3H).

### (R)-4-((3S,8S,9S,10R,13R,14S,17R)-3-Hydroxy-10,13-dimethyl-2,3,4,7,8,9,10,11,12,13,14,15,16,17-tetradecahydro-1H-cyclopenta[a]phenanthren-17-yl)-N,N-diisopropylpentanamide (63)

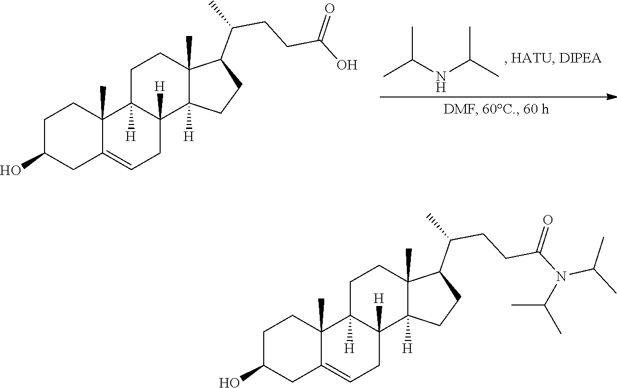

A round bottom flask equipped with a stir bar was charged with cholenic acid 149 (200 mg, 0.534 mmol), DMF (3 mL), and THE (1 mL). HATU (305 mg, 0.801 mmol) was added and dissolved prior to the addition of N,N-diisopropylethylamine (465 μL, 2.67 mmol). Diisopropylamine (150 μL, 1.07 mmol) was added and the resulting mixture stirred at 60° C. for 60 h. The reaction was diluted with water and EtOAc, layers were separated, and the aqueous layer was extracted with EtOAc (2×). Organics were combined, dried (MgSO₄), filtered, and concentrated in vacuo. The crude material was purified by silica gel chromatography (0-20-40-60-80% EtOAc:hexanes) afforded the desired product (213 mg, 87%) as an off-white solid. ¹H NMR: (300 MHz, CDCl₃) δ 5.33 (br d, J=6.0 Hz, 1H), 4.03-3.86 (m, 1H), 3.62-3.35 (m, 2H), 2.38-0.80 (m, 31H), 1.35 (d, J=6.0 Hz, 6H), 1.19 (d, J=6.0 Hz, 6H), 0.99 (s, 3H), 0.94 (d, J=6.0 Hz, 3H), 0.67 (s, 3H).

### (R)-4-((3S,8S,9S,10R,13R,14S,17R)-3-Hydroxy-10,13-dimethyl-2,3,4,7,8,9,10,11,12,13,14,15,16,17-tetradecahydro-1H-cyclopenta[a]phenanthren-17-yl)-N-(2-(methylamino)phenyl)pentanamide (64)

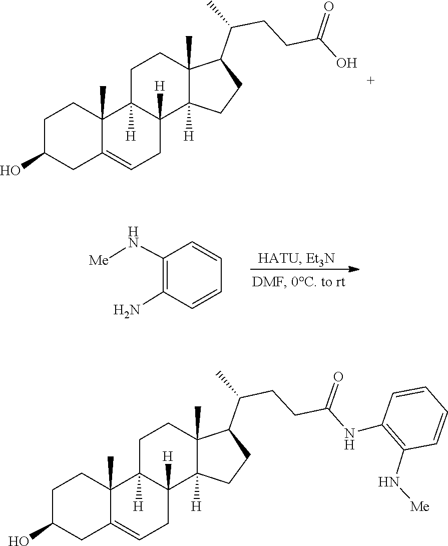

A 100 mL round-bottom flask containing N-methyl-1,2-phenylenediamine (91.0 μL, 0.801 mmol) was charged with cholenic acid 149 (300 mg, 0.801 mmol), anhydrous DMF (3.6 mL), and anhydrous THE (1 mL). The solution was treated with triethylamine (117 μL, 0.841 mmol) and was cooled to 0° C. before HATU (320 mg, 0.841 mmol) was added. The resulting solution was allowed to stir at 0° C. with slow warming to room temperature overnight. The reaction mixture was diluted with water and EtOAc. Layers were separated and the aqueous layer was extracted EtOAc (2×). Organics were combined, washed with brine, dried (MgSO₄), filtered, and concentrated in vacuo. The crude material was purified by silica gel chromatography (50-75-100% EtOAc:hexanes) to afford the desired product (206 mg, 54%) as a white powder. ¹H NMR: (300 MHz, CDCl₃) δ 7.30-6.99 (m, 2H), 6.81-6.54 (m, 2H), 5.35 (br d, J=3.0 Hz, 1H), 3.59-3.44 (m, 1H), 2.83 (br s, 3H), 2.52-2.39 (m, 1H), 2.36-2.13 (m, 3H), 2.05-0.86 (m, 23H), 1.00 (s, 3H), 0.99 (d, J=6.0 Hz, 3H), 0.75 (br d, J=6.0 Hz, 1H), 0.70 (s, 3H).

### Example 8. Synthesis of (3S,8S,9S,10R,13S,14S,17R)-10,13-Dimethyl-17-(prop-1-en-2-yl)-2,3,4,7,8,9,10,11,12,13,14,15,16,17-tetradecahydro-1H-cyclopenta[a]phenanthren-3-ol (85)

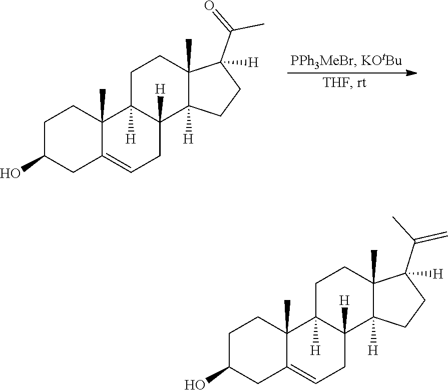

To a suspension of methyltriphenylphosphonium bromide (22.5 g, 63.0 mmol) in anhydrous THE (100 mL) under N₂ atmosphere was added potassium tert-butoxide (7.07 g, 63.0 mmol). The resulting solution was stirred at 60° C. for 30 min prior to the addition of pregnenolone (6.65 g, 21 mmol). The resulting solution was stirred at 60° C. for 16 h. The reaction mixture was then poured into ice water (˜100-150 mL) and extracted with EtOAc (2×). The combined organic layers were dried (MgSO₄), filtered, and concentrated in vacuo. The crude material was purified by silica gel chromatography (0-25-50% EtOAc:hexanes) to afford the desired product (5.99 g, 91%) as a white solid. ¹H NMR: (300 MHz, CDCl₃) δ 5.36 (br d, J=6.0 Hz, 1H), 4.85 (s, 1H), 4.71 (s, 1H), 3.59-3.46 (m, 1H), 2.35-2.15 (m, 2H), 2.07-1.94 (m, 2H), 1.92-1.63 (m, 6H), 1.76 (s, 3H), 1.63-1.37 (m, 6H), 1.28-0.90 (m, 5H), 1.01 (s, 3H), 0.59 (s, 3H).

### Example 9. Synthesis of (((3S,8S,9S,10R,13S,14S,17R)-10,13-Dimethyl-17-(prop-1-en-2-yl)-2,3,4,7,8,9,10,11,12,13,14,15,16,17-tetradecahydro-1H-cyclopenta[a]phenanthren-3-yl)oxy)triisopropylsilane (86)

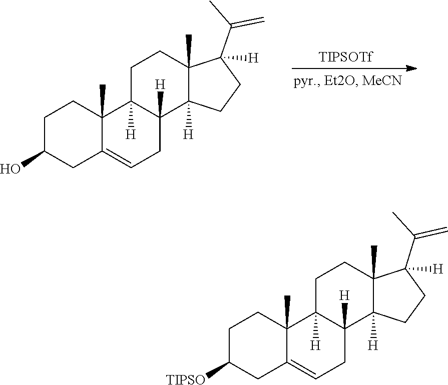

A flask equipped with a stir bar was charged with Et₂O (22 mL) and MeCN (15 mL) and chilled to −20° C. Triisopropylsilyl trifluoromethanesulfonate (6.82 g, 5.98 mL, 22.3 mmol) and pyridine (1.2 mL) were added at −20° C. The flask was further chilled to −40° C., charged with alkene 85 (3.5 g, 11.1 mmol) in Et₂O (25 mL) and allowed to stir at −40° C. for 2 h. The solution was then poured over saturated aqueous NaHCO₃, extracted with hexanes, washed with water, dried (MgSO₄) and concentrated. The crude material was purified by silica gel chromatography (0-15-30% EtOAc:hexanes) to afford the desired product (4.95 g, 95%) as a white solid. ¹H NMR: (300 MHz, CDCl₃) δ 5.32 (br d, J=6.0 Hz, 1H), 4.85 (s, 1H), 4.71 (s, 1H), 3.62-3.49 (m, 1H), 2.36-2.20 (m, 2H), 2.08-1.36 (m, 14H), 1.76 (s, 3H), 1.29-0.78 (m, 9H), 1.06 (s, 18H), 1.01 (s, 3H), 0.58 (s, 3H).

### Example 10. Synthesis of (S)-2-((3S,8S,9S,10R,13S,14S,17R)-10,13-Dimethyl-3-((triisopropylsilyl)oxy)-2,3,4,7,8,9,10,11,12,13,14,15,16,17-tetradecahydro-1H-cyclopenta[a]phenanthrene-17-yl)propan-1-ol (152)

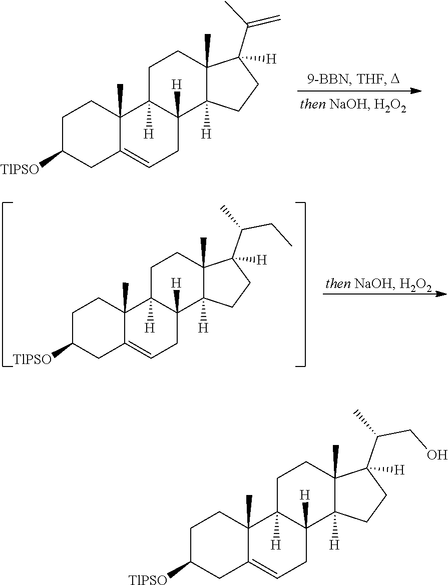

To a solution of alkene 86 (1.50 g, 3.19 mmol) dissolved in anhydrous THF (30 mL) was added 9-BBN (0.5 M in THF, 22.5 mL, 11.2 mmol) at 0° C. over 15 min under argon atmosphere. The reaction was stirred at room temperature for 1 hour, then warmed to reflux and stirred for an additional 16 hours. The reaction was cooled to 0° C., and 2 N NaOH (30 mL) and 30% H₂O₂ (30 mL) were added. The resulting mixture was warmed to room temperature and allowed to stir for an additional 18 h. After the aqueous layer was extracted with Et₂O, the organic layer was washed with brine, dried (MgSO₄), and concentrated in vacuo. The crude material was purified by silica gel chromatography (0-15-30% EtOAc:hexanes) to afford the desired product (1.16 g, 74%) as a white amorphous solid. ¹H NMR: (300 MHz, CDCl₃, for major diastereomer only) δ 5.31 (br d, J=6.0 Hz, 1H), 3.64 (dd, J=9.0, 3.0 Hz, 1H), 3.62-3.49 (m, 1H), 3.37 (dd, J=12.0, 6.0 Hz, 1H), 2.35-2.19 (m 2H), 2.05-1.91 (m, 2H), 1.89-1.74 (m, 3H), 1.67-0.78 (m, 25H), 1.06 (s, 18H), 1.05 (d, J=6.0 Hz, 3H), 0.70 (s, 3H).

### Example 11. Synthesis of (((3S,8S,9S,10R,13R,14S,17R)-10,13-Dimethyl-17-((R,E)-4-phenylbut-3-en-2-yl)-2,3,4,7,8,9,10,11,12,13,14,15,16,17-tetradecahydro-1H-cyclopenta[a]phenanthren-3-yl)oxy)triisopropylsilane (150)

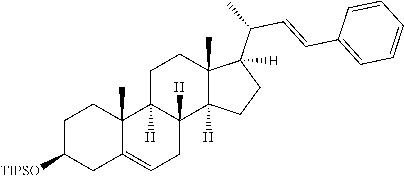

### Step a. (S)-2-((3S,8S,9S,10R,13S,14S,17R)-10,13-Dimethyl-3-((triisopropylsilyl)oxy)-2,3,4,7,8,9,10,11,12,13,14,15,16,17-tetradecahydro-1H-cyclopenta[a]phenanthren-17-yl)propyl 4-methylbenzenesulfonate (i-11a)

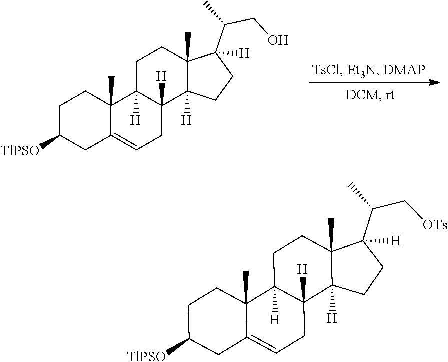

To a stirred solution of alcohol 87 (1.58 g, 3.23 mmol) in CH₂Cl₂ (27 mL) was added triethylamine (1.35 mL, 9.70 mmol) and 4-dimethylaminopyridine (4.00 mg, 32.0 μmol) under N₂ atmosphere at room temperature. P-Toluenesulfonyl chloride (740 mg, 3.88 mmol) was added and the solution was stirred for 16 h at room temperature. The solution was partitioned between EtOAc and 0.5 M HCl. Aqueous layer was extracted with EtOAc (2×), and the combined organic layers were washed with 5% NaOH (w/v), brine, and dried over MgSO₄. The crude material was purified by silica gel chromatography (0-5-10-20% EtOAc:hexanes) to afford the product (1.85 g, 89%) as a clear oil. ¹H NMR: (300 MHz, CDCl₃) δ 7.79 (d, J=9.0 Hz, 2H), 7.34 (d, J=6.0 Hz, 2H), 5.30 (br d, J=6.0 Hz, 1H), 3.97 (dd, J=9.0, 3.0 Hz, 1H), 3.79 (dd, J=12.0, 9.0 Hz, 1H), 3.62-3.49 (m, 1H), 2.45 (s, 3H), 2.34-2.18 (m, 2H), 2.01-0.82 (m, 26H), 1.05 (s, 18H), 0.99 (s, 3H), 0.98 (d, J=6.0 Hz, 3H), 0.64 (s, 3H).

### Step b. 2-(((S)-2-((3S,8S,9S,10R,13S,14S,17R)-10,13-dimethyl-3-((triisopropylsilyl)oxy)-2,3,4,7,8,9,10,11,12,13,14,15,16,17-tetradecahydro-1H-cyclopenta[a]phenanthren-17-yl)propyl)thio)benzo[d]thiazole (i-11b)

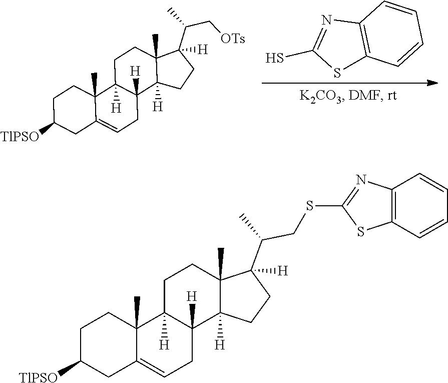

A solution of tosylated alcohol i-11a (535 mg, 0.832 mmol) in DMF (12 mL) was treated with 2-mercaptobenzothiazole (445 mg, 2.66 mmol) and potassium carbonate (805 mg, 5.82 mg). The resulting suspension was allowed to stir at room temperature for 18 h. The mixture was then partitioned between EtOAc and water, and the layers were separated. The organic phase was washed with brine, dried (MgSO₄), filtered, and concentrated in vacuo. The crude material was purified by silica gel chromatography (0-2-5-10% EtOAc:hexanes) to afford the product (465 mg, 88%) as a white amorphous solid. ¹H NMR: (300 MHz, CDCl₃) δ 7.85 (d, J=9.0 Hz, 1H), 7.74 (d, J=9.0 Hz, 1H), 7.40 (ddd, J=9.0, 9.0, 3.0 Hz, 1H), 7.28 (ddd, J=9.0, 3.0, 3.0 Hz, 1H), 5.32 (br d, J=6.0 Hz, 1H), 3.66 (dd, J=12.0, 3.0 Hz, 1H), 3.63-3.50 (m, 1H), 3.06 (dd, J=12.0, 6.0 Hz, 1H), 2.36-2.20 (m, 2H), 2.07-1.75 (m, 6H), 1.73-0.79 (m, 20H), 1.15 (d, J=6.0 Hz, 3H), 1.06 (s, 18H), 1.01 (s, 3H), 0.71 (s, 3H).

### Step c. 2-(((S)-2-((3S,8S,9S,10R,13S,14S,17R)-10,13-dimethyl-3-((triisopropylsilyl)oxy)-2,3,4,7,8,9,10,11,12,13,14,15,16,17-tetradecahydro-1H-cyclopenta[a]phenanthren-17-yl)propyl)sulfonyl)benzo[d]thiazole (i-11c)

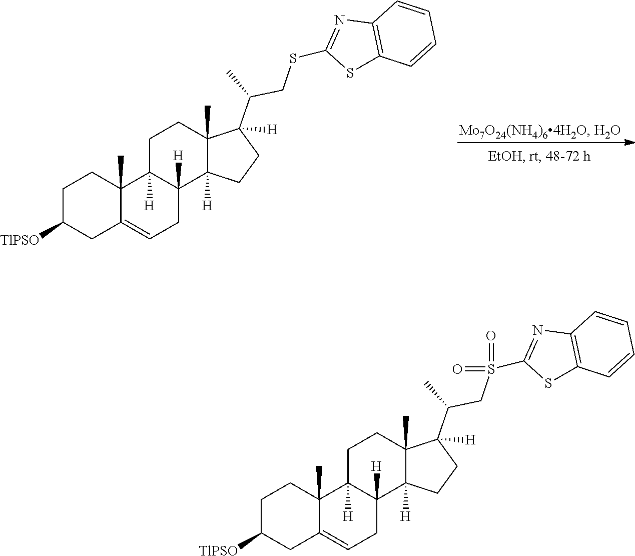

To a solution of sulfide i-11b (461 mg, 0.722 mmol) in 95% EtOH (13.4 mL) was added hexanes (5 mL) to ensure solubility. The resulting solution was cooled to 0° C., followed by the dropwise addition of ammonium heptamolybdate tetrahydrate (87.4 mg, 72.0 μmol) as a solution in 30% H₂O₂ (246 mg, 802 μL, 7.23 mmol). The reaction mixture was warmed to room temperature and allowed to stir for 60 h. More ammonium heptamolybdate tetrahydrate (545 mg, 0.441 mmol) as a solution in 30% H₂O₂ (1.54 g, 5.00 mL, 7.23 mmol) was added at room temperature, and the resulting mixture stirred for an additional 16 h. The reaction mixture was partitioned between water and and Et₂O and the layers were separated. The aqueous layer was extracted with Et₂O, and the organic layers were combined, washed with 5% sodium thiosulfate solution, saturated aqueous NaHCO₃, and brine. The organic layer was dried (MgSO₄), filtered, and solvent was removed in vacuo. The crude material was redissolved in DCM (5-10 mL) and more ammonium heptamolybdate tetrahydrate (545 mg, 0.441 mmol) as a solution in 30% H₂O₂ (1.54 g, 5.00 mL, 7.23 mmol) was added at room temperature. The resulting mixture was allowed to stir for an additional 16 h. The reaction mixture was partitioned between water and and Et₂O and the layers were separated. The aqueous layer was extracted with Et₂O, and the organic layers were combined, washed with 5% sodium thiosulfate solution, saturated aqueous NaHCO₃, and brine. The organic layer was dried (MgSO₄), filtered, and solvent was removed in vacuo. The crude material was purified by silica gel chromatography (0-1-2-5-10% EtOAc:hexanes) to afford the product (305 mg, 63%) as a white solid. ¹H NMR: (300 MHz, CDCl₃) δ 8.21 (dd, J=9.0, 3.0 Hz, 1H), 8.01 (dd, J=6.0, 3.0 Hz, 1H), 7.67-7.55 (m, 2H), 5.29 (br d, J=6.0 Hz, 1H), 3.64 (dd, J=15.0, 3.0 Hz, 1H), 3.61-3.48 (m, 1H), 3.27 (dd, J=12.0, 9.0 Hz, 1H), 2.40-2.18 (m, 3H), 2.03-1.73 (m, 5H), 1.65-0.81 (m, 21H), 1.27 (d, J=9.0 Hz, 3H), 1.05 (s, 18H), 0.99 (s, 3H), 0.70 (s, 3H).

### Step d. (((3S,8S,9S,10R,13R,14S,17R)-10,13-Dimethyl-17-((R,E)-4-phenylbut-3-en-2-yl)-2,3,4,7,8,9,10,11,12,13,14,15,16,17-tetradecahydro-1H-cyclopenta[a]phenanthren-3-yl)oxy)triisopropylsilane (150)

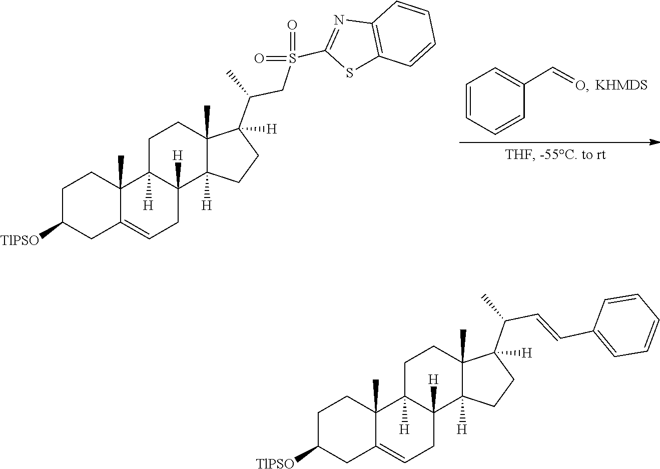

To a solution of KHMDS (1.0 M in THF, 246 μL, 246 μmol) in anhydrous THF (314 μL) under a N₂ atmosphere was added a solution of sulfone i-11c (150 mg, 224 μmol) in THF (1.2 mL) at −55° C. The resulting mixture was stirred at −55° C. for 30 min before a solution of benzaldehyde (25.0 μL, 246 μmol) in THF (560 μL) was added dropwise. The reaction was allowed to stir at −55° C. for 1 h and then slowly warmed to room temperature overnight. The reaction mixture was quenched with sat'd aqueous NH₄Cl and diluted with Et₂O. Layers were separated and the aqueous layer was extracted with Et₂O (3×). Organic extracts were combined, washed with brine, dried over MgSO₄, filtered, and concentrated. The crude material was purified by silica gel chromatography (0-5-10% EtOAc:hexanes) to afford the product (64 mg, 51%) as a clear oil. ¹H NMR: (300 MHz, CDCl₃) δ 7.35-7.23 (m, 4H), 7.20-7.13 (m, 1H), 6.30 (d, J=15.0 Hz, 1H), 6.06 (dd, J=15.0, 6.0 Hz, 1H), 5.31 (br d, J=6.0 Hz, 1H), 3.62-3.49 (m, 1H), 2.35-2.18 (m, 3H), 2.07-1.90 (m, 2H), 1.87-1.66 (m, 3H), 1.66-0.80 (m, 24H), 1.12 (d, J=6.0 Hz, 3H), 1.06 (s, 18H), 1.02 (s, 3H), 0.74 (s, 3H).

### Example 12. Synthesis of (3S,8S,9S,10R,13R,14S,17R)-10,13-Dimethyl-17-((R)-4-(1-methyl-1H-benzo[d]imidazol-2-yl)butan-2-yl)-2,3,4,7,8,9,10,11,12,13,14,15,16,17-tetradecahydro-1H-cyclopenta[a]phenanthren-3-ol (18)

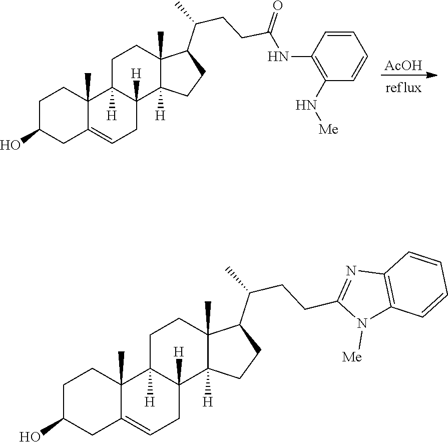

A round-bottom flask containing amide 64 (217 mg, 0.453 mmol) was charged with glacial AcOH (5 mL). The resulting mixture was heated to 65° C., and allowed to stir for 2 h. The AcOH was removed under reduced pressure and the resulting oil was purified by silica gel chromatography (40-70-100% EtOAc:hexanes) to provide the desired product (60 mg, 29%) as a white solid. ¹H NMR: (300 MHz, CDCl₃) δ 7.71 (br t, J=6.0 Hz, 1H), 7.32-7.19 (m, 3H), 5.35 (br d, J=6.0 Hz, 1H), 3.73 (s, 3H), 3.60-3.46 (m, 1H), 2.96 (ddd, J=15.0, 12.0, 3.0 Hz, 1H), 2.77 (ddd, J=15.0, 9.0, 3.0 Hz, 1H), 2.35-2.17 (m, 2H), 2.09-1.78 (m, 6H), 1.69-0.87 (m, 17H), 1.09 (d, J=6.0 Hz, 3H), 1.01 (s, 3H), 0.70 (s, 3H).

### Example 13. Synthesis of (E)-1-((3S,8S,9S,10R,13S,14S)-3-hydroxy-10,13-dimethyl-2,3,4,7,8,9,10,11,12,13,14,15,16,17-tetradecahydro-1H-cyclopenta[a]phenanthren-17-yl)-3-phenylprop-2-en-1-one (30)

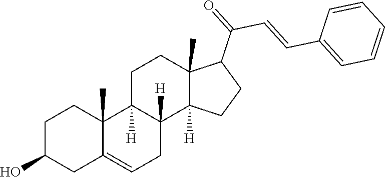

To a solution of pregnenolone (2.0 g, 6.32 mmol) in THE (60 mL) and EtOH (120 mL) was added KOH (0.71 g, 12.64 mmol) and benzaldehyde (0.77 mL, 7.58 mmol). The reaction mixture was allowed to stir at room temperature for 48 hours. The reaction mixture was quenched with 1 N HCl until pH was about 5-6 by pH paper. The reaction mixture was concentrated in vacuo to remove EtOH, brought up in water and was extracted with EtOAc (3 times). The organic layers were combined, washed with brine (3 times), dried over anhydrous Na₂SO₄, filtered and concentrated in vacuo. The residue was purified by silica gel chromatography (10-40% EtOAc in hexanes) to afford (E)-1-((3S,8S,9S,10R,13S,14S)-3-hydroxy-10,13-dimethyl-2,3,4,7,8,9,10,11,12,13,14,15,16,17-tetradecahydro-1H-cyclopenta[a]phenanthren-17-yl)-3-phenylprop-2-en-1-one as a 4:1 mixture of diastereomers (0.081 g, 0.2 mmol, 3%). UPLC/ELSD: RT=2.26 min. MS (ES): m/z (MH⁺) 405.6 for C₂₈H₃₆O₂. ¹H NMR (300 MHz, CDCl₃) δ: ppm 7.58-7.53 (br. m, 3H); 7.40-7.38 (m, 3H); 6.81-6.73 (br. m, 1H); 5.37 (m, 1H); 3.59-3.47 (br. m, 1H); 3.15 (dd, 0.25H); 2.86 (t, 0.75H); 2.41-2.19 (br. m, 3H); 2.06-1.23 (br. m, 17H); 1.00 (s, 3H); 0.65 (s, 3H).

### Example 14. Synthesis of (E)-1-((3S,8S,9S,10R,13S,14S)-3-hydroxy-10,13-dimethyl-2,3,4,7,8,9,10,11,12,13,14,15,16,17-tetradecahydro-1H-cyclopenta[a]phenanthren-17-yl)-3-(o-tolyl)prop-2-en-1-one (31)

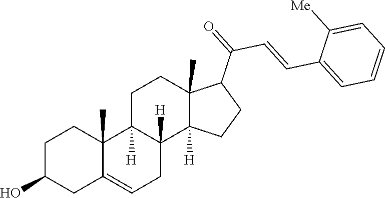

To a solution of pregnenolone (2.0 g, 6.32 mmol) in THE (60 mL) and EtOH (120 mL) was added KOH (0.71 g, 12.64 mmol) and 2-methylbenzaldehyde (0.87 mL, 7.58 mmol). The reaction mixture was allowed to stir at room temperature for 48 hours. The reaction mixture was quenched with 1 N HCl until pH was about 5-6 by pH paper. The reaction mixture was concentrated in vacuo to remove EtOH, brought up in water and was extracted with EtOAc (3 times). The organic layers were combined, washed with brine (3 times), dried over anhydrous Na₂SO₄, filtered and concentrated in vacuo. The residue was purified by silica gel chromatography (10-40% EtOAc in hexanes) to afford (E)-1-((3S,8S,9S,10R,13S,14S)-3-hydroxy-10,13-dimethyl-2,3,4,7,8,9,10,11,12,13,14,15,16,17-tetradecahydro-1H-cyclopenta[a]phenanthren-17-yl)-3-(o-tolyl)prop-2-en-1-one as a 3.5:1 mixture of diastereomers (0.36 g, 0.86 mmol, 13.6%). UPLC/ELSD: RT=244 min. MS (ES): m/z (MH⁺) 419.7 for C₂₉H₃₈O₂. ¹H NMR (300 MHz, CDCl₃) δ: ppm 7.90-7.79 (br. m, 1H); 7.60 (d, 1H); 7.33-7.21 (br. m, 3H); 6.75-6.67 (br. m, 1H); 5.38 (m, 1H); 3.60-3.47 (br. m, 1H); 3.15 (dd, 0.3H); 2.88 (t, 0.7H); 2.47 (s, 3H); 2.41-1.07 (br. m, 19H); 1.03 (s, 3H); 0.98-0.85 (br. m, 1H); 0.67 (s, 3H).

### Example 15. Synthesis of (E)-3-(2,6-dimethylphenyl)-1-((3S,8S,9S,10R,13S,14S)-3-hydroxy-10,13-dimethyl-2,3,4,7,8,9,10,11,12,13,14,15,16,17-tetradecahydro-1H-cyclopenta[a]phenanthren-17-yl)prop-2-en-1-one (32)

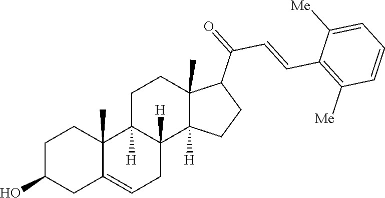

To a solution of pregnenolone (2.0 g, 6.32 mmol) in THE (60 mL) and EtOH (120 mL) was added KOH (0.71 g, 12.64 mmol) and 2,6-dimethylbenzaldehyde (0.98 mL, 7.58 mmol). The reaction mixture was allowed to stir at room temperature for 48 hours. The reaction mixture was quenched with 1 N HCl until pH was about 5-6 by pH paper. The reaction mixture was concentrated in vacuo to remove EtOH, brought up in water and was extracted with EtOAc (3 times). The organic layers were combined, washed with brine (3 times), dried over anhydrous Na₂SO₄, filtered and concentrated in vacuo. The residue was purified by silica gel chromatography (10-40% EtOAc in hexanes) to afford (E)-3-(2,6-dimethylphenyl)-1-((3S,8S,9S,10R,13S,14S)-3-hydroxy-10,13-dimethyl-2,3,4,7,8,9,10,11,12,13,14,15,16,17-tetradecahydro-1H-cyclopenta[a]phenanthren-17-yl)prop-2-en-1-one as a 1.5:1 mixture of diastereomers (0.80 g, 1.85 mmol, 29%). UPLC/ELSD: RT=2.61 min. MS (ES): m/z (MH⁺) 433.6 for C₃₀H₄₀O₂. ¹H NMR (300 MHz, CDCl₃) δ: ppm 7.73-7.62 (br. m, 1H); 7.16-7.06 (d, 3H); 6.45-6.35 (br. m, 1H); 5.36 (m, 1H); 3.58-3.48 (br. m, 1H); 3.11 (dd, 0.35H); 2.83 (t, 0.65H); 2.35 (s, 6H); 2.30-1.05 (br. m, 19H); 1.01 (s, 3H); 0.96-0.86 (br. m, 1H); 0.69 (s, 3H).

### Example 16. General Procedure for Modified Julia Olefination

To a solution of KHMDS (1.0 M in THF, 1.1 equiv.) in anhydrous THE (0.78 M) under a N₂ atmosphere was added a solution of sulfone (1 equiv.) in THE (0.19 M) at −55° C. The resulting mixture was stirred at −55° C. for 30 min before a solution of aldehyde (1.1 equiv.) in THE (0.44 M) was added dropwise. The reaction was allowed to stir at −55° C. for 1 hour and then slowly warmed to room temperature. The solution continued to stir at room temperature for the allotted time indicated below. The reaction mixture was then quenched with saturated aqueous NH₄Cl and diluted with Et₂O. Layers were separated and the aqueous layer was extracted with Et₂O (3×). Organic extracts were combined, washed with brine, dried over MgSO₄, filtered, and concentrated. The crude material was purified as indicated below.

### (((3S,8S,9S,10R,13R,14S,17R)-10,13-Dimethyl-17-((R,E)-4-phenylbut-3-en-2-yl)-2,3,4,7,8,9,10,11,12,13,14,15,16,17-tetradecahydro-1H-cyclopenta[a]phenanthren-3-yl)oxy)triisopropylsilane (150)

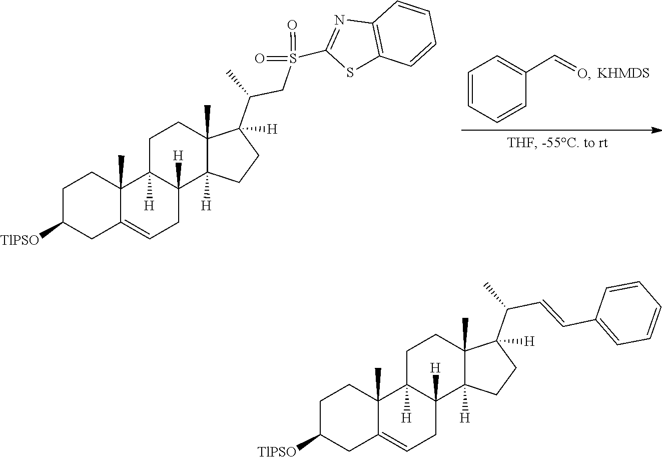

Synthesized according to the general procedure for modified Julia olefination described above. Sulfone (150 mg, 224 μmol), benzaldehyde (25.0 μL, 246 μmol), KHMDS (246 μL, 246 μmol), and THF (2.2 mL). The reaction stirred at room temperature overnight. The crude material was purified by silica gel chromatography (0-5-10% EtOAc:hexanes) to afford the product (64 mg, 51%) as a clear oil (complete E selectivity). ¹H NMR: (300 MHz, CDCl₃) δ 7.35-7.23 (m, 4H), 7.20-7.13 (m, 1H), 6.30 (d, J=15.0 Hz, 1H), 6.06 (dd, J=15.0, 6.0 Hz, 1H), 5.31 (br d, J=6.0 Hz, 1H), 3.62-3.49 (m, 1H), 2.35-2.18 (m, 3H), 2.07-1.90 (m, 2H), 1.87-1.66 (m, 3H), 1.66-0.80 (m, 24H), 1.12 (d, J=6.0 Hz, 3H), 1.06 (s, 18H), 1.02 (s, 3H), 0.74 (s, 3H).

### (((3S,8S,9S,10R,13R,14S,17R)-10,13-Dimethyl-17-((R,E)-5-methylhex-3-en-2-yl)-2,3,4,7,8,9,10,11,12,13,14,15,16,17-tetradecahydro-1H-cyclopenta[a]phenanthren-3-yl)oxy)triisopropylsilane (i-16a)

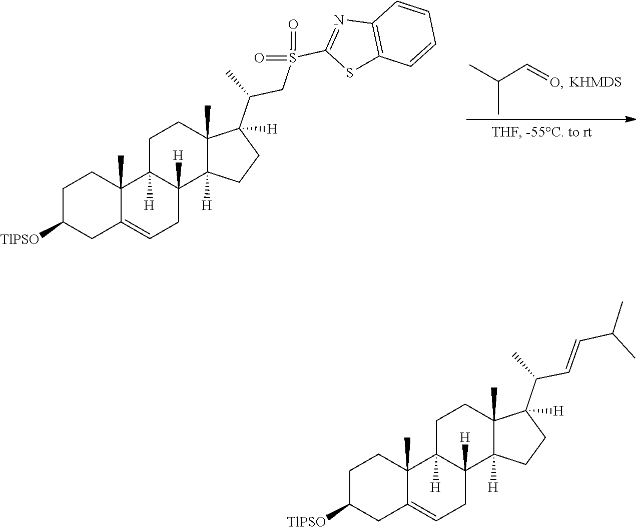

Synthesized according to the genera procedure or modified Julia olefination described above.

Sulfone (200 mg, 298 μmol), isobutyraldehyde (29.9 μL, 328 μmol), KHMDS (328 μL, 328 μmol), and THF (3.0 mL). The reaction stirred at room temperature for 3 h. The crude material was purified by silica gel chromatography (0-5-10% EtOAc:hexanes) to afford the product (86 mg, 55%) as a clear oil, and as a mixture of geometric isomers (approximately 2:1 E:Z selectivity). ¹H NMR: (300 MHz, CDCl₃, reported as seen in spectrum) δ 5.34-5.28 (br m, 1.80H), 5.25 (d, J=6.0 Hz, 0.81H), 5.19 (d, J=6.0 Hz, 0.80H), 5.14 (d, J=6.0 Hz, 0.26H), 5.05-4.94 (m, 0.75H), 3.62-3.49 (m, 1.51H), 2.69-2.54 (m, 0.51H), 2.51-2.38 (m, 0.53H), 2.35-2.13 (m, 4.42H), 2.08-1.90 (m, 4.33H), 1.87-1.38 (m, 15.7H), 1.33-0.79 (m, 66.9H), 0.72 (s, 1.37H), 0.69 (s, 3.00H).

### (((3S,8S,9S,10R,13R,14S,17R)-17-((R,E)-5,5-Dimethylhex-3-en-2-yl)-10,13-dimethyl-2,3,4,7,8,9,10,11,12,13,14,15,16,17-tetradecahydro-1H-cyclopenta[a]phenanthren-3-yl)oxy)triisopropylsilane (i-16b)

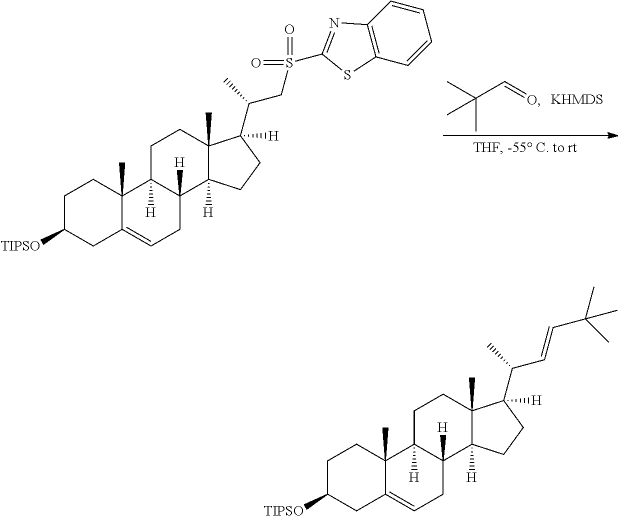

Synthesized according to the general procedure for modified Julia olefination described above. Sulfone (200 mg, 298 μmol), pivaldehyde (35.6 μL, 328 μmol), KHMDS (328 μL, 328 μmol), and THF (3.0 mL). The reaction stirred at room temperature for 3 h. The crude material was purified by silica gel chromatography (0-5-10% EtOAc:hexanes) to afford the product (105 mg, 65%) as a clear oil, and as a mixture of geometric isomers (approximately 4.5:1 E:Z selectivity). ¹H NMR: (300 MHz, CDCl₃, reported as seen in spectrum) δ 5.37-5.29 (m, 2.34H), 5.14 (d, J=9.0 Hz, 0.74H), 5.09 (d, J=9.0 Hz, 0.50H), 4.96 (dd, J=18.0, 9.0 Hz, 0.19H), 3.63-3.49 (m, 1.23H), 2.77-2.62 (m, 0.19H), 2.36-2.19 (m, 2.64H), 2.09-1.88 (m, 3.85H), 1.88-1.74 (m, 2.72H), 1.74-1.38 (m, 10.6H), 1.36-0.80 (m, 58.2H), 0.73 (s, 0.65H), 0.69 (s, 3.00H).

### (((3S,8S,9S,10R,13R,14S,17R)-17-((R,E)-5-Ethylhept-3-en-2-yl)-10,13-dimethyl-2,3,4,7,8,9,10,11,12,13,14,15,16,17-tetradecahydro-1H-cyclopenta[a]phenanthren-3-yl)oxy)triisopropylsilane (i-16c)

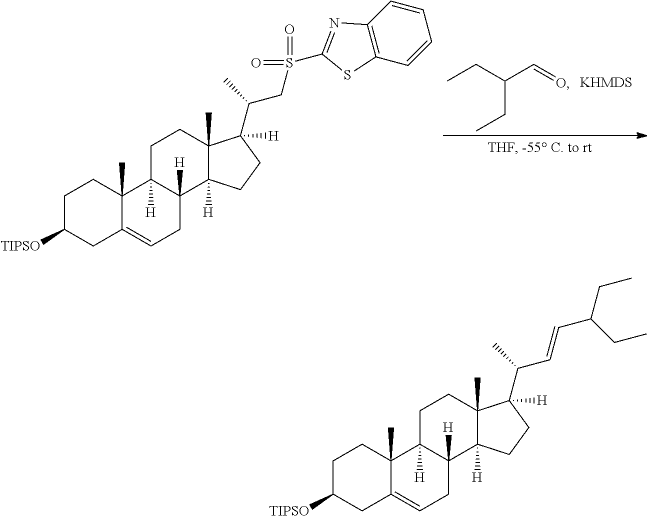

Synthesized according to the general procedure for modified Julia olefination described above. Sulfone (500 mg, 746 μmol), 2-ethylbutyraldehyde (101 μL, 821 μmol), KHMDS (821 μL, 821 μmol), and THF (7.5 mL). The reaction stirred at room temperature overnight. The crude material was purified by silica gel chromatography (0-5-10% EtOAc:hexanes) to afford the product (78 mg, 19%) as a clear oil, and as a mixture of geometric isomers (approximately 1:1 E:Z selectivity). ¹H NMR: (300 MHz, CDCl₃, reported as seen in spectrum) δ 5.31 (br d, J=6.0 Hz, 1.93H), 5.23-5.12 (m, 1.83H), 5.03-4.82 (m, 1.94H), 3.64-3.49 (m, 1.94H), 2.50-1.88 (m, 11.9H), 1.88-0.78 (m, 117H), 0.72 (s, 3.00H), 0.70 (s, 2.76H).

### (((3S,8S,9S,10R,13R,14S,17R)-17-((R,E)-6,6-Dimethylhept-3-en-2-yl)-10,13-dimethyl-2,3,4,7,8,9,10,11,12,13,14,15,16,17-tetradecahydro-1H-cyclopenta[a]phenanthren-3-yl)oxy)triisopropylsilane (i-16d)

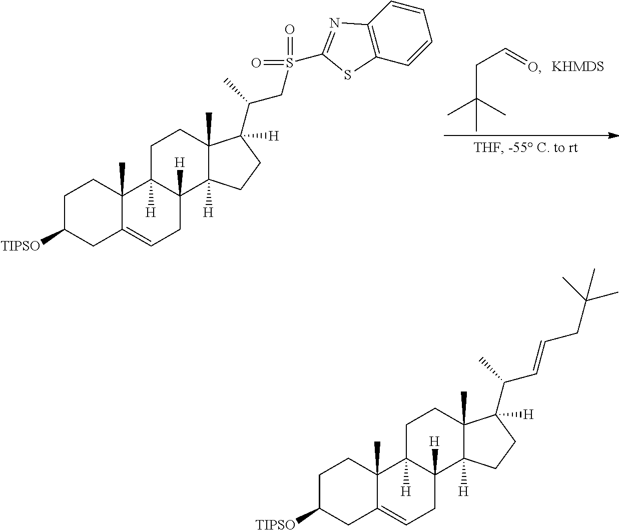

Synthesized according to the general procedure for modified Julia olefination described above. Sulfone (500 mg, 746 μmol), 3,3-dimethylbutanal (103 μL, 821 μmol), KHMDS (821 μL, 821 μmol), and THF (7.5 mL). The reaction stirred at room temperature overnight. The crude material was purified by silica gel chromatography (0-5-10% EtOAc:hexanes) to afford the product (62 mg, 15%) as a clear oil, and as a mixture of geometric isomers (approximately 2:1 E:Z selectivity). ¹H NMR: (300 MHz, CDCl₃, reported as seen in spectrum) δ 5.40-5.15 (m, 4.63H), 3.64-3.49 (m, 1.56H), 2.53-2.36 (m, 1.20H), 2.36-2.18 (m, 3.42H), 2.11-1.38 (m, 23.6H), 1.33-0.93 (m, 54.9H), 0.90 (s, 9.14H), 0.86 (s, 3.95H), 0.72 (s, 3.00H), 0.70 (s, 1.35H).

### (((3S,8S,9S,10R,13R,14S,17R)-17-((R,E)-4-Cyclohexylbut-3-en-2-yl)-10,13-dimethyl-2,3,4,7,8,9,10,11,12,13,14,15,16,17-tetradecahydro-1H-cyclopenta[a]phenanthren-3-yl)oxy)triisopropylsilane (i-16e)

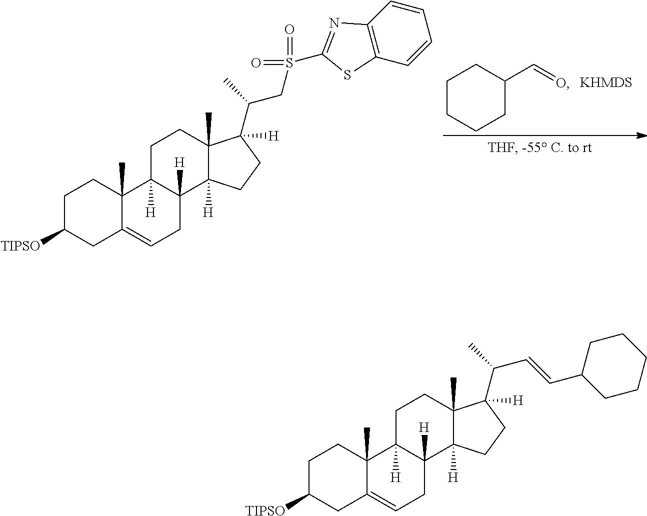

Synthesized according to the general procedure for modified Julia olefination described above. Sulfone (500 mg, 746 μmol), cyclohexanecarboxaldehyde (100 μL, 821 μmol), KHMDS (821 μL, 821 μmol), and THF (7.5 mL). The reaction stirred at room temperature for 2 h. The crude material was purified by silica gel chromatography (0-5-10% EtOAc:hexanes) to afford the product (351 mg, 83%) as a clear oil, and as a mixture of geometric isomers (approximately 1:1 E:Z selectivity). ¹H NMR: (300 MHz, CDCl₃, reported as seen in spectrum) δ 5.31 (br d, J=3.0 Hz, 1.96H), 5.28 (d, J=6.0 Hz, 0.20H), 5.21 (dd, J=9.0, 6.0 Hz, 1.64H), 5.15 (d, J=9.0 Hz, 0.20H), 5.08-4.95 (m, 1.74H), 3.63-3.49 (m, 1.87H), 2.52-2.36 (m, 0.88H), 2.36-2.17 (m, 4.89H), 2.14-1.37 (m, 38.2H), 1.37-0.79 (m, 84.5H), 0.73 (s, 2.64H), 0.69 (s, 3.00H).

### (((3S,8S,9S,10R,13R,14S,17R)-10,13-Dimethyl-17-((R,E)-4-(o-tolyl)but-3-en-2-yl)-2,3,4,7,8,9,10,11,12,13,14,15,16,17-tetradecahydro-1H-cyclopenta[a]phenanthren-3-yl)oxy)triisopropylsilane (i-16f)

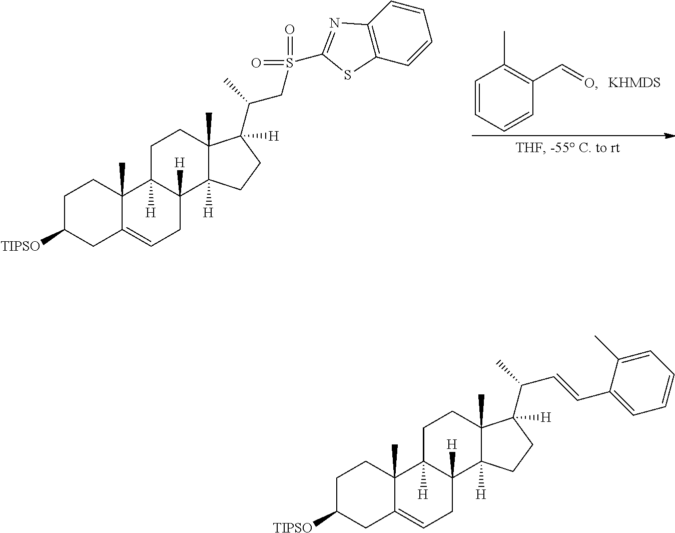

Synthesized according to the general procedure for modified Julia olefination described above. Sulfone (500 mg, 746 μmol), o-tolualdehyde (94.9 μL, 821 μmol), KHMDS (821 μL, 821 μmol), and THF (7.5 mL). The reaction stirred at room temperature overnight. The crude material was purified by silica gel chromatography (0-5-10% EtOAc:hexanes) to afford the product (230 mg, 54%) as a clear oil (complete E selectivity). ¹H NMR: (300 MHz, CDCl₃) δ 7.40 (br d, J=6.0 Hz, 1H), 7.22-7.11 (m, 3H), 6.52 (d, J=15.0 Hz, 1H), 5.94 (dd, J=15.0, 9.0 Hz, 1H), 5.35 (br d, J=1H), 3.67-3.54 (m, 1H), 2.36 (s, 3H), 2.41-2.25 (m, 3H), 2.12-1.95 (m, 2H), 1.92-1.44 (m, 11H), 1.42-0.86 (m, 39H), 0.79 (s, 3H).

### (((3S,8S,9S,10R,13R,14S,17R)-17-((R,E)-4-(2,6-Dimethylphenyl)but-3-en-2-yl)-10,13-dimethyl-2,3,4,7,8,9,10,11,12,13,14,15,16,17-tetradecahydro-1H-cyclopenta[a]phenanthren-3-yl)oxy)triisopropylsilane (i-16g)

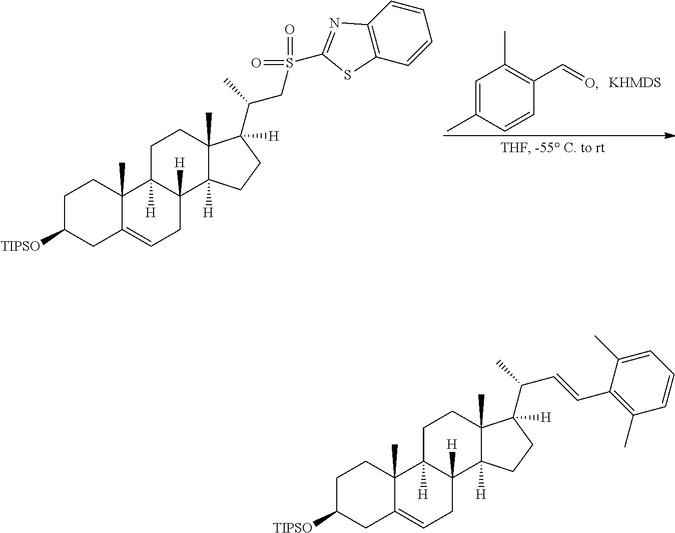

Synthesized according to the general procedure for modified Julia olefination described above. Sulfone (500 mg, 746 μmol), 2,6-dimethylbenzaldehyde (110 mg, 821 μmol), KHMDS (821 μL, 821 μmol), and THF (7.5 mL). The reaction stirred at room temperature overnight. The crude material was purified by silica gel chromatography (0-5-10% EtOAc:hexanes) to afford the product (344 mg, 78%) as a clear oil, and as a mixture of geometric isomers (approximately 3:1 E:Z selectivity). ¹H NMR: (300 MHz, CDCl₃, reported as seen in spectrum) δ 7.12-6.99 (m, 4H), 6.26 (d, J=15.0 Hz, 1H), 6.15 (d, J=12.0 Hz, 0.30H), 5.55 (dd, J=12.0, 3.0 Hz, 0.34H), 5.51 (dd, J=15.0, 9.0 Hz, 1H), 5.35 (br d, J=6.0 Hz, 1H), 5.31 (br s, 0.33H), 3.67-3.51 (m, 1.34H), 2.41-2.23 (m, 4H), 2.31 (s, 6H), 2.27 (s, 2H), 2.12-1.71 (m, 8.37H), 1.68-0.86 (m, 65.5H), 0.79 (s, 3H), 0.52 (s, 1H).

### (((3S,8S,9S,10R,13R,14S,17R)-17-((R,E)-4-((3R,5R,7R)-Adamantan-1-yl)but-3-en-2-yl)-10,13-dimethyl-2,3,4,7,8,9,10,11,12,13,14,15,16,17-tetradecahydro-1H-cyclopenta[a]phenanthren-3-yl)oxy)triisopropylsilane (i-16h)

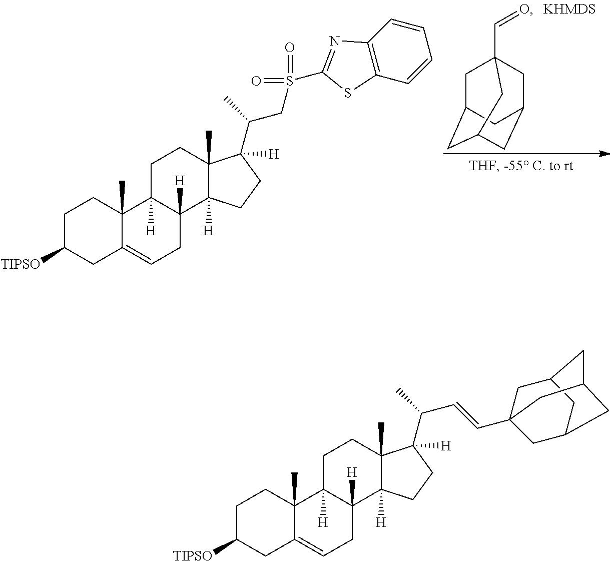

Synthesized according to the general procedure for modified Julia olefination described above. Sulfone (500 mg, 746 μmol), 1-adamantanecarboxaldehyde (135 mg, 821 μmol), KHMDS (821 μL, 821 μmol), and THF (7.5 mL). The reaction stirred at room temperature overnight. The crude material was purified by silica gel chromatography (0-5-10% EtOAc:hexanes) to afford the product (151 mg, 33%) as a clear oil, and as a mixture of geometric isomers (approximately 3:1 E:Z selectivity). ¹H NMR: (300 MHz, CDCl₃, reported as seen in spectrum) δ 5.31 (br d, J=3.0 Hz, 1.37H), 5.19 (d, J=15.0 Hz, 1H), 5.06 (dd, J=15.0, 9.0 Hz, 1H), 5.01-4.83 (m, 0.69H), 3.64-3.49 (m, 1.34H), 2.79-2.63 (m, 0.30H), 2.37-2.19 (m, 2.82H), 2.08-1.37 (41.6H), 1.35-0.80 (m, 54.9H), 0.73 (s, 1.14H), 0.69 (s, 3H).

### Triisopropyl(((3S,8S,9S,10R,13R,14S,17R)-17-((R,E)-5-isopropyl-6-methylhept-3-en-2-yl)-10,13-dimethyl-2,3,4,7,8,9,10,11,12,13,14,15,16,17-tetradecahydro-1H-cyclopenta[a]phenanthren-3-yl)oxy)silane (i-16i)

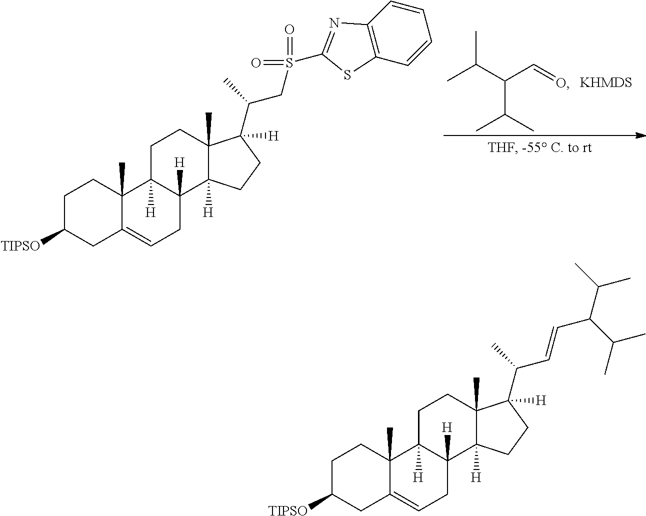

Synthesized according to the general procedure for modified Julia olefination described above. Sulfone (500 mg, 746 μmol), 2-isopropyl-3-methylbutanal (105 mg, 821 μmol), KHMDS (821 μL, 821 μmol), and THF (7.5 mL). The reaction stirred at room temperature for 3 h. The crude material was purified by silica gel chromatography (0-5-10% EtOAc:hexanes) to afford the product (213 mg, 49%) as a clear oil, and as a mixture of geometric isomers (approximately 1.5:1 E:Z selectivity). ¹H NMR: (300 MHz, CDCl₃, reported as seen in spectrum) δ 5.31 (br d, J=6.0 Hz, 2H), 5.25 (d, J=12.0 Hz, 0.77H), 5.18-4.91 (m, 2.19H), 3.64-3.49 (m, 1.68H), 2.63-2.48 (m, 0.31H), 2.46-2.13 (m, 5H), 2.13-0.66 (m, 114H), 0.71 (s, 3H), 0.70 (s, 1.74H).

### (((3S,8S,9S,10R,13R,14S,17R)-17-((R,E)-5,5-Diethylhept-3-en-2-yl)-10,13-dimethyl-2,3,4,7,8,9,10,11,12,13,14,15,16,17-tetradecahydro-1H-cyclopenta[a]phenanthren-3-yl)oxy)triisopropylsilane (i-16j)

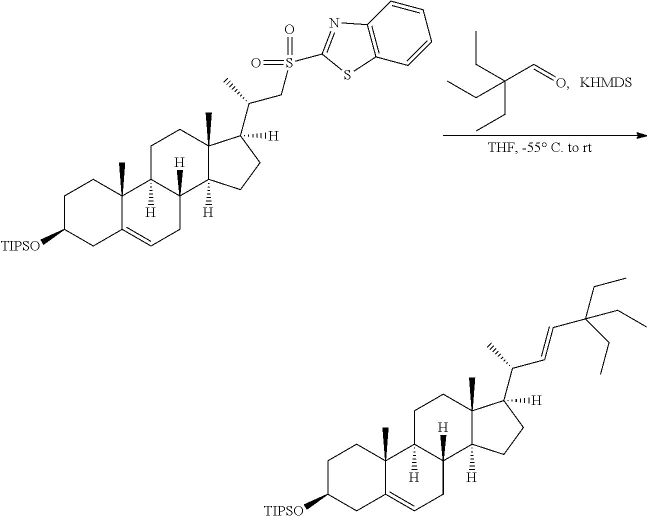

Synthesized according to the general procedure for modified Julia olefination described above. Sulfone (500 mg, 746 μmol), 2,2-diethylbutanal (105 mg, 821 μmol), KHMDS (821 μL, 821 μmol), and THF (7.5 mL). The reaction stirred at room temperature overnight. The crude material was purified by silica gel chromatography (0-5-10% EtOAc:hexanes) to afford the product (117 mg, 27%) as a clear oil, and as a mixture of geometric isomers (approximately 5:1 E:Z selectivity). ¹H NMR: (300 MHz, CDCl₃, reported as seen in spectrum) δ 5.31 (br d, J=6.0 Hz, 1.17H), 5.14-4.98 (m, 1.18H), 4.92 (dd, J=9.0, 3.0 Hz, 0.21H), 4.76 (d, J=12.0 Hz, 0.08H), 3.65-3.47 (m, 1H), 2.67-2.52 (m, 0.14H), 2.47-2.15 (m, 2.53H), 2.12-1.35 (m, 15.8H), 1.34-0.63 (m, 51.5H).

### (((3S,8S,9S,10R,13R,14S,17R)-17-((R,E)-4-Cyclopentylbut-3-en-2-yl)-10,13-dimethyl-2,3,4,7,8,9,10,11,12,13,14,15,16,17-tetradecahydro-1H-cyclopenta[a]phenanthren-3-yl)oxy)triisopropylsilane (i-16k)

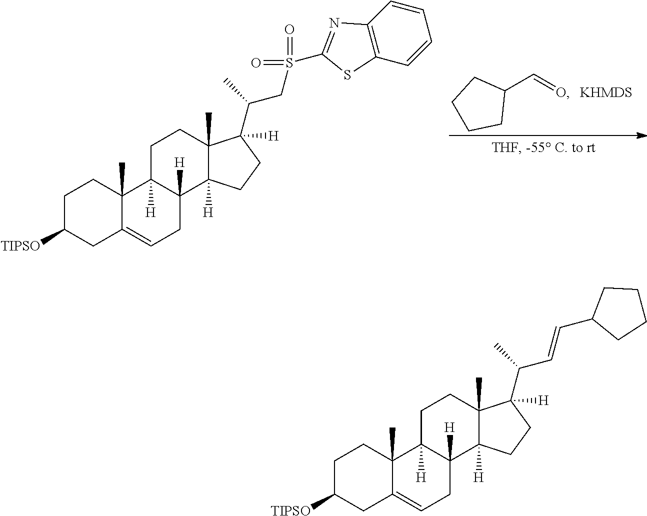

Synthesized according to general procedure for modified Julia olefination described above. Sulfone (500 mg, 746 μmol), cyclopentanecarboxaldehyde (88.0 μL, 821 μmol), KHMDS (821 μL, 821 μmol), and THF (7.5 mL). The reaction stirred at room temperature for 2 hours. The crude material was purified by silica gel chromatography (0-5-10% EtOAc:hexanes) to afford the product (156 mg, 38%) as a clear oil, and as a mixture of geometric isomers (approximately 3:2 E:Z selectivity). ¹H NMR: (300 MHz, CDCl₃, reported as seen in spectrum) δ 5.35-5.00 (m, 5H), 3.63-3.49 (m, 1.64H), 2.77-2.61 (m, 0.61H), 2.53-2.19 (m, 5.17H), 2.09-1.88 (m, 4.58H), 1.88-1.36 (m, 27H), 1.36-0.79 (m, 64H), 0.73 (s, 1.89H), 0.69 (s, 3H).

### (((3S,8S,9S,10R,13R,14S,17R)-17-((R,E)-4-Cycloheptylbut-3-en-2-yl)-10,13-dimethyl-2,3,4,7,8,9,10,11,12,13,14,15,16,17-tetradecahydro-1H-cyclopenta[a]phenanthren-3-yl)oxy)triisopropylsilane (i-161)

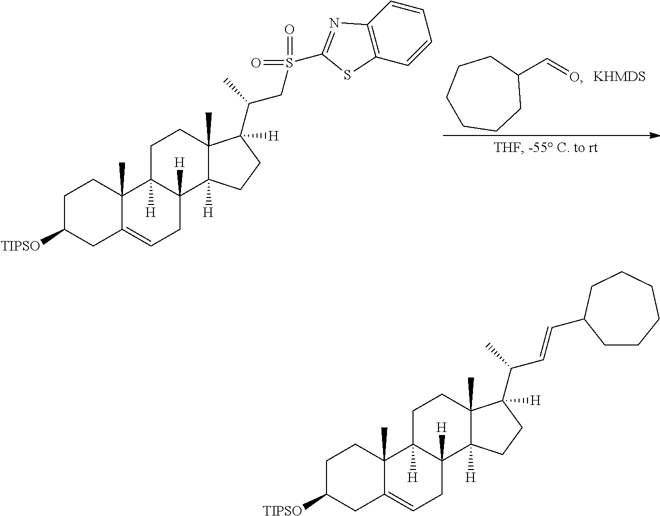

Synthesized according to general procedure for modified Julia olefination described above. Sulfone (500 mg, 746 μmol), cycloheptanecarbaldehyde (113 μL, 821 μmol), KHMDS (821 μL, 821 μmol), and THF (7.5 mL). The reaction stirred at room temperature for 2 hours. The crude material was purified by silica gel chromatography (0-5-10% EtOAc:hexanes) to afford the product (170 mg, 39%) as a clear oil, and as a mixture of geometric isomers (approximately 1:1 E:Z selectivity). ¹H NMR: (300 MHz, CDCl₃, reported as seen in spectrum) δ 5.36-5.26 (m, 3H), 5.20-5.08 (m, 1.91H), 4.97 (dd, J=12.0, 12.0 Hz, 1H), 3.64-3.49 (m, 2H), 2.56-2.37 (m, 2.36H), 2.36-2.16 (m, 4.85H), 2.16-1.88 (m, 7.25H), 1.88-1.37 (m, 46.2H), 1.36-0.82 (m, 78.2H), 0.73 (s, 3H), 0.69 (s, 3H).

### Triisopropyl(((3S,8S,9S,10R,13R,14S,17R)-17-((R,E)-4-(4-isopropylcyclohexyl)but-3-en-2-yl)-10,13-dimethyl-2,3,4,7,8,9,10,11,12,13,14,15,16,17-tetradecahydro-1H-cyclopenta[a]phenanthren-3-_yl)oxy)silane (i-16m)

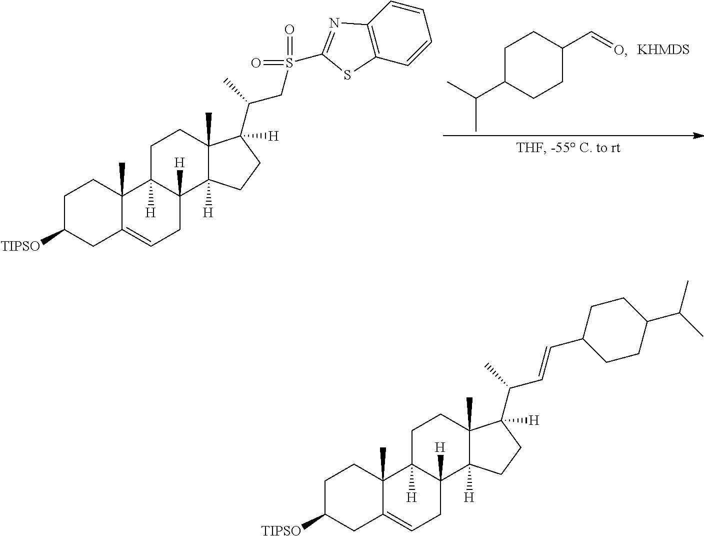

Synthesized according to general procedure for modified Julia olefination described above. Sulfone (500 mg, 746 μmol), 4-isopropylcyclohexane-1-carbaldehyde (127 mg, 821 μmol), KHMDS (821 μL, 821 μmol), and THF (7.5 mL). The reaction stirred at room temperature for 2 hours. The crude material was purified by silica gel chromatography (0-10% EtOAc:hexanes) to afford the product (230 mg, 51%) as a clear oil, and as a mixture of geometric isomers (approximately 1:1 E:Z selectivity). ¹H NMR: (300 MHz, CDCl₃, reported as seen in spectrum) δ 5.44-4.93 (m, 5.83H), 3.64-3.49 (m, 1.89H), 2.65-2.53 (m, 0.42H), 2.52-2.36 (m, 1.08H), 2.36-2.10 (m, 5.3H), 2.10-1.88 (m, 5.92H), 1.88-1.34 (m, 33.9H), 1.34-0.78 (m, 91.9H), 0.72 (d, J=3.0 Hz, 3H), 0.69 (d, J=3.0 Hz, 3H).

### (((3S,8S,9S,10R,13R,14S,17R)-17-((R,E)-4-Cyclododecylbut-3-en-2-yl)-10,13-dimethyl-2,3,4,7,8,9,10,11,12,13,14,15,16,17-tetradecahydro-1H-cyclopenta[a]phenanthren-3-yl)oxy)triisopropylsilane (i-16n)

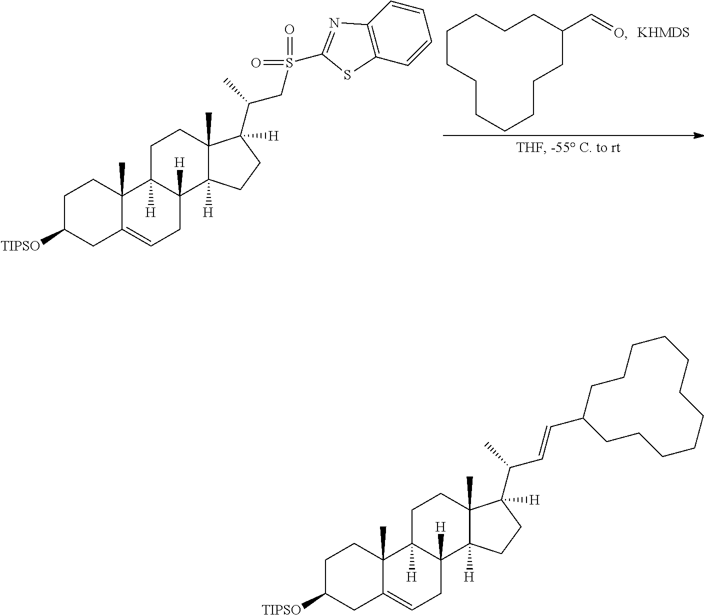

Synthesized according to general procedure for modified Julia olefination described above. Sulfone (500 mg, 746 μmol), cyclododecanecarbaldehyde (161 mg, 821 μmol), KHMDS (821 μL, 821 μmol), and THF (7.5 mL). The reaction stirred at room temperature for 2 hours. The crude material was purified by silica gel chromatography (0-10% EtOAc:hexanes) to afford the product (305 mg, 63%) as a clear oil, and as a mixture of geometric isomers (approximately 2:1 E:Z selectivity). ¹H NMR: (300 MHz, CDCl₃, reported as seen in spectrum) δ 5.32 (br d, J=6.0 Hz, 1.63H), 5.24-5.04 (m, 2.61H), 4.99 (dd, J=9.0, 9.0 Hz, 0.47H), 3.65-3.49 (m, 1.58H), 2.62-2.39 (m, 1.23H), 2.39-2.18 (m, 3.39H), 2.13-1.89 (m, 5.89H), 1.89-0.81 (m, 125H), 0.74 (s, 1.59H), 0.70 (s, 3H).

### (((3S,8S,9S,10R,13R,14S,17R)-17-((2R,E)-4-(2-Ethylcyclohexyl)but-3-en-2-yl)-10,13-dimethyl-2,3,4,7,8,9,10,11,12,13,14,15,16,17-tetradecahydro-1H-cyclopenta[a]phenanthren-3-yl)oxy)triisopropylsilane (i-16o)

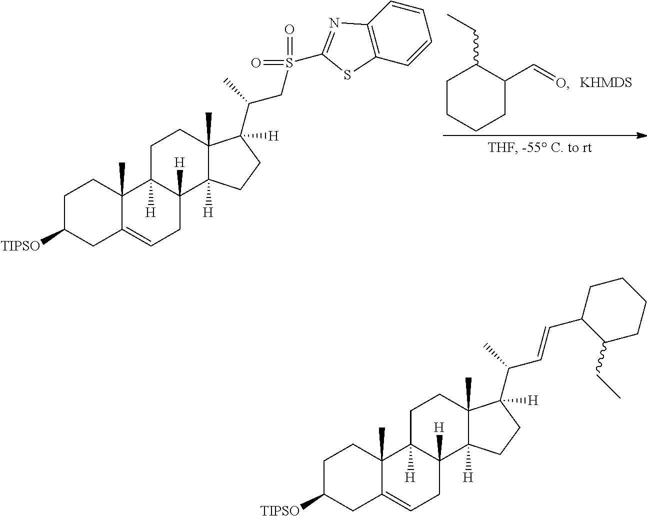

Synthesized according to general procedure for modified Julia olefination described above. Sulfone (500 mg, 746 μmol), 2-ethylcyclohexane-1-carbaldehyde (115 mg, 821 μmol), KHMDS (821 μL, 821 μmol), and THF (7.5 mL). The reaction stirred at room temperature for 2 hours. The crude material was purified by silica gel chromatography (0-10% EtOAc:hexanes) to afford the product (203 mg, 46%) as a clear oil, and as a mixture of geometric isomers (approximately 1:1 E:Z selectivity). ¹H NMR: (300 MHz, CDCl₃, reported as seen in spectrum) δ 5.53-4.85 (m, 5.51H), 3.63-3.49 (m, 1.83H), 2.73-2.59 (br m, 0.59H), 2.53-2.18 (m, 5.61H), 2.13-1.37 (m, 40.2H), 1.37-0.77 (m, 93.8H), 0.72 (s, 3H), 0.69 (d, J=3.0 Hz, 2.57H).

### (((3S,8S,9S,10R,13R,14S,17R)-10,13-Dimethyl-17-((R,E)-6-propyinon-3-en-2-yl)-2,3,4,7,8,9,10,11,12,13,14,15,16,17-tetradecahydro-1H-cyclopenta[a]phenanthren-3-yl)oxy)triisopropylsilane (i-16p)

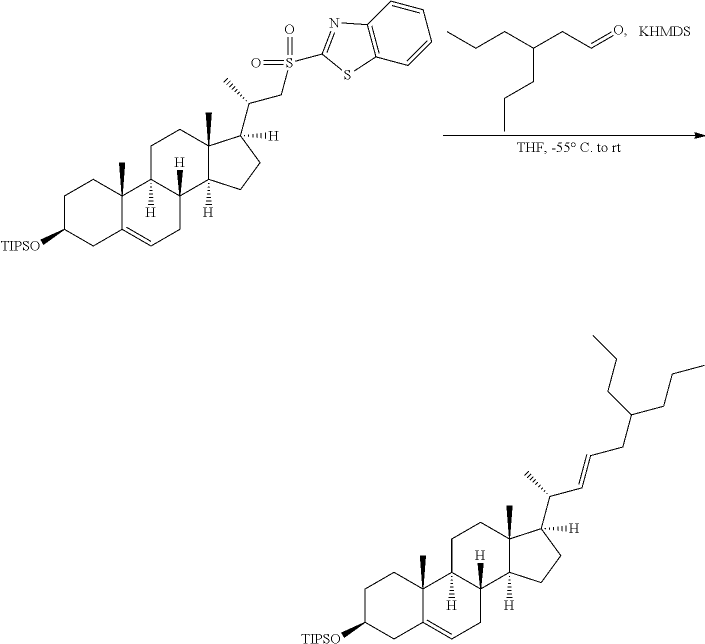

Synthesized according to general procedure for modified Julia olefination described above. Sulfone (500 mg, 746 μmol), 3-propylhexanal (117 mg, 821 μmol), KHMDS (821 μL, 821 μmol), and THF (7.5 mL). The reaction stirred at room temperature for 4 hours. The crude material was purified by silica gel chromatography (10-20-40% EtOAc:hexanes) to afford the product (245 mg, 55%) as a clear oil, and as a mixture of geometric isomers (approximately 4:3 E:Z selectivity). ¹H NMR: (300 MHz, CDCl₃, reported as seen in spectrum) δ 5.31 (br d, J=3.0 Hz, 2H), 5.27-5.12 (m, 3.57H), 3.63-3.50 (m, 1.86H), 2.52-2.18 (m, 5.21H), 2.12-1.88 (m, 8.70H), 1.88-1.74 (m, 4.13H), 1.74-1.42 (m, 14.3H), 1.41-0.79 (m, 97H), 0.72 (s, 3H), 0.70 (s, 3H).

### (((3S,8S,9S,10R,13R,14S,17R)-17-((R,E)-6-Butyldec-3-en-2-yl)-10,13-dimethyl-2,3,4,7,8,9,10,11,12,13,14,15,16,17-tetradecahydro-1H-cyclopenta[a]phenanthren-3-yl)oxy)triisopropylsilane (i-16q)

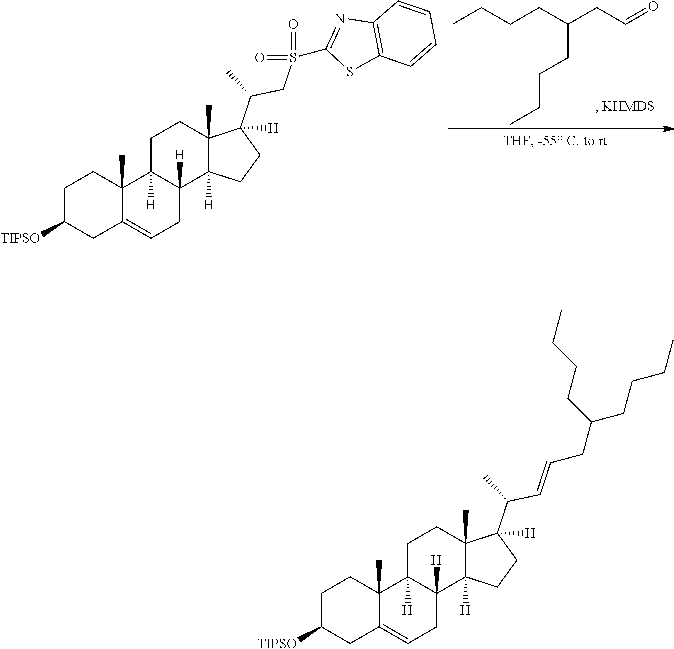

Synthesized according to general procedure for modified Julia olefination described above. Sulfone (500 mg, 746 μmol), 3-butylheptanal (140 mg, 821 μmol), KHMDS (821 μL, 821 μmol), and THF (7.5 mL). The reaction stirred at room temperature for 4 hours. The crude material was purified by silica gel chromatography (0-5-10-20% EtOAc:hexanes) to afford the product (152 mg, 33%) as a clear oil, and as a mixture of geometric isomers (approximately 3:2 E:Z selectivity). ¹H NMR: (300 MHz, CDCl₃, reported as seen in spectrum) δ 5.32 (br d, J=3.0 Hz, 1.90H), 5.27-5.12 (m, 3.29H), 3.63-3.49 (m, 1.81H), 2.54-2.36 (m, 1.19H), 2.36-2.19 (m, 3.84H), 2.10-1.88 (m, 8.22H), 1.88-1.75 (m, 3.96H), 1.75-1.39 (m, 14.7H), 1.38-0.81 (m, 100H), 0.73 (s, 3H), 0.70 (s, 2.17H).

### (((3S,8S,9S,10R,13R,14S,17R)-17-((R,E)-6-Ethyloct-3-en-2-yl)-10,13-dimethyl-2,3,4,7,8,9,10,11,12,13,14,15,16,17-tetradecahydro-1H-cyclopenta[a]phenanthren-3-yl)oxy)triisopropylsilane (i-16r)

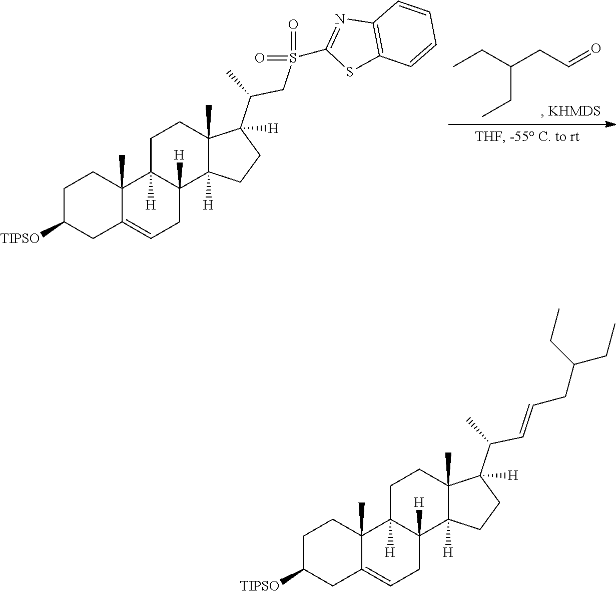

Synthesized according to general procedure for modified Julia olefination described above. Sulfone (500 mg, 746 μmol), 3-ethylpentanal (94.0 mg, 821 μmol), KHMDS (821 μL, 821 μmol), and THF (7.5 mL). The reaction stirred at room temperature for 2 h. The crude material was purified by silica gel chromatography (0-5-10-20% EtOAc:hexanes) to afford the product (273 mg, 64%) as a clear oil, and as a mixture of geometric isomers (approximately 2:1 E:Z selectivity). ¹H NMR: (300 MHz, CDCl₃, reported as seen in spectrum) δ 5.32 (br d, J=6.0 Hz, 1.69H), 5.28-5.12 (m, 3.08H), 3.64-3.49 (m, 1.60H), 2.53-2.37 (m, 1H), 2.37-2.19 (m, 3.35H), 2.10-1.88 (m, 7.31H), 1.88-1.38 (m, 17.8H), 1.38-0.78 (m, 83.3H), 0.73 (s, 3H), 0.70 (s, 1.85H).

### (((3S,8S,9S,10R,13R,14S,17R)-17-((2R,E)-5,6-Diethyloct-3-en-2-yl)-10,13-dimethyl-2,3,4,7,8,9,10,11,12,13,14,15,16,17-tetradecahydro-1H-cyclopenta[a]phenanthren-3-yl)oxy)triisopropylsilane (i-16s)

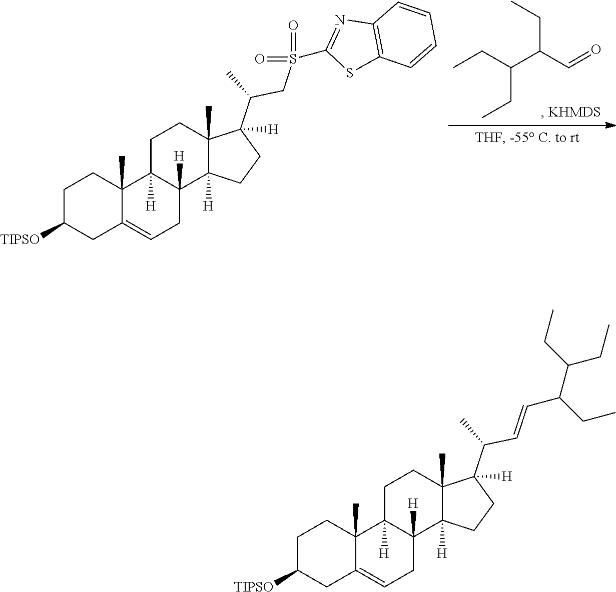

Synthesized according to general procedure for modified Julia olefination described above. Sulfone (500 mg, 746 μmol), 2,3-diethylpentanal (117 mg, 821 μmol), KHMDS (821 μL, 821 μmol), and THF (7.5 mL). The reaction stirred at room temperature for 2 hours. The crude material was purified by silica gel chromatography (0-5-10% EtOAc:hexanes) to afford the product (188 mg, 42%) as a clear oil, and as a mixture of geometric isomers (approximately 3:2 E:Z selectivity). ¹H NMR: (300 MHz, CDCl₃, reported as seen in spectrum) δ 5.32 (br d, J=6.0 Hz, 1.77H), 5.25-5.11 (m, 1.75H), 5.10-4.95 (m, 1.80H), 3.65-3.48 (m, 1.76H), 2.49-2.20 (m, 5.84H), 2.10-1.88 (m, 4.62H), 1.88-1.75 (m, 4.78H), 1.75-0.94 (m, 92.6H), 0.94-0.77 (m, 19.4H), 0.73 (s, 3H), 0.70 (s, 2.14H).

### (((3S,8S,9S,10R,13R,14S,17R)-17-((2R,E)-5-ethyl-6-propyinon-3-en-2-yl)-10,13-dimethyl-2,3,4,7,8,9,10,11,12,13,14,15,16,17-tetradecahydro-1H-cyclopenta[a]phenanthren-3-yl)oxy)triisopropylsilane (i-16t)

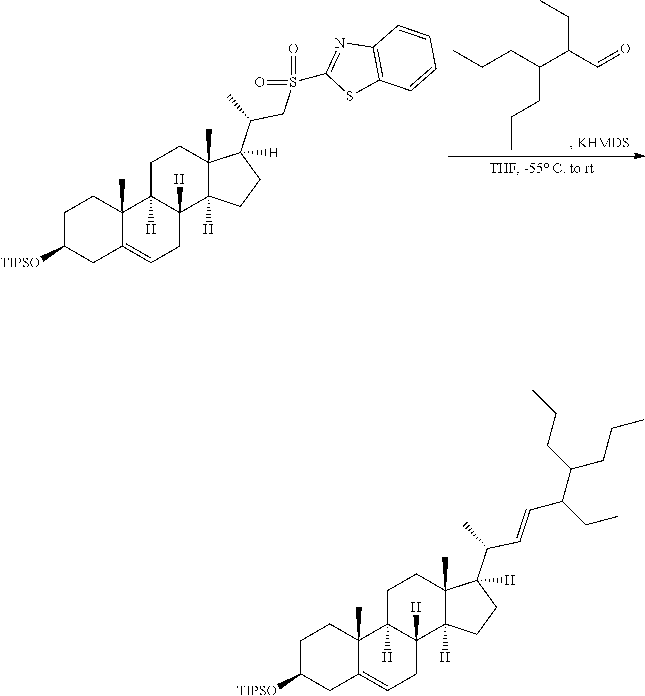

Synthesized according to general procedure for modified Julia olefination described above. Sulfone (500 mg, 746 μmol), 2-ethyl-3-propylhexanal (140 mg, 821 μmol), KHMDS (821 μL, 821 μmol), and THF (7.5 mL). The reaction stirred at room temperature for 18 hours. The crude material was purified by silica gel chromatography (0-10% EtOAc:hexanes) to afford the product (29 mg, 6%) as a clear oil, and as a mixture of geometric isomers (approximately 1:1 E:Z selectivity). ¹H NMR: (300 MHz, CDCl₃, reported as seen in spectrum) δ 5.31 (br d, J=6.0 Hz, 2.06H), 5.24-4.88 (m, 2.73H), 3.65-3.47 (m, 2H), 2.66-2.49 (m, 0.35H), 2.46-2.18 (m, 6.31H), 2.09-1.88 (m, 5.43H), 1.87-1.72 (m, 4.92H), 1.72-0.76 (m, 113H), 0.71 (s, 3H), 0.68 (s, 3H).

### Example 17. General Procedure for Silyl Group Deprotection

To a vial equipped with a stir bar was added the sterol (1 equiv.) dissolved in THF (0.1 M). Tetrabutylammonium fluoride (1.0 M in THF, 5 equiv.) was added and the resulting mixture was allowed to stir at room temperature for 3 hours, prior to TLC analysis. Reaction was quenched with saturated aqueous NaHCO₃ and extracted with EtOAc (2×). Organic extracts were combined, dried (MgSO₄), filtered, and concentrated. The crude material was purified as indicated below.

### (3S,8S,9S,10R,13R,14S,17R)-10,13-Dimethyl-17-((R,E)-4-phenylbut-3-en-2-yl)-2,3,4,7,8,9,10,11,12,13,14,15,16,17-tetradecahydro-1H-cyclopenta[a]phenanthren-3-ol (154)

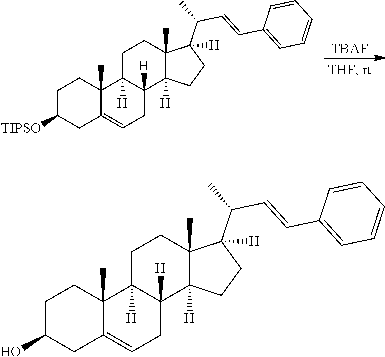

Synthesized according to the general procedure for silyl group deprotection described above. Sterol 150 (60 mg, 107 μmol), TBAF (535 μL, 535 μmol), and THF (1.1 mL). The crude material was purified by silica gel chromatography (10-20-40-60% EtOAc:hexanes) to afford the product (34 mg, 79%) as a white solid (complete E selectivity). ¹H NMR: (300 MHz, CDCl₃) δ 7.36-7.24 (m, 4H), 7.21-7.14 (m, 1H), 6.30 (d, J=15.0 Hz, 1H), 6.07 (dd, J=15.0, 9.0 Hz, 1H), 5.35 (br d, J=3.0 Hz, 1H), 3.60-3.46 (m, 1H), 2.35-2.17 (m, 3H), 2.10-1.66 (m, 5H), 1.63-0.89 (m, 16H), 1.13 (d, J=6.0 Hz, 3H), 1.02 (s, 3H), 0.75 (s, 3H).

### (3S,8S,9S,10R,13R,14S,17R)-10,13-Dimethyl-17-((R,E)-5-methylhex-3-en-2-yl)-2,3,4,7,8,9,10,11,12,13,14,15,16,17-tetradecahydro-1H-cyclopenta[a]phenanthren-3-ol (155)

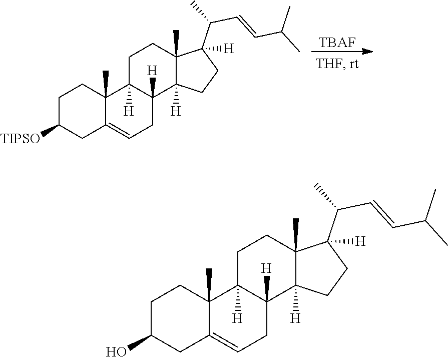

Synthesized according to the general procedure for silyl group deprotection described above. Sterol i-16a (86 mg, 163 μmol), TBAF (816 μL, 816 μmol), and THF (1.6 mL). The crude material was purified by silica gel chromatography (10-20-40-60% EtOAc:hexanes) to afford the product (54 mg, 88%) as a white solid, and as a mixture of geometric isomers (approximately 2:1 E:Z selectivity). ¹H NMR: (300 MHz, CDCl₃, reported as seen in spectrum) δ 5.35 (m, 1.52H), 5.27 (dd, J=15.0, 6.0 Hz, 1.15H), 5.16 (dd, J=15.0, 9.0 Hz, 1.08H), 5.05-4.94 (m, 0.81H), 3.59-3.46 (m, 1.52H), 2.69-2.52 (m, 0.45H), 2.51-2.36 (m, 0.50H), 2.35-2.12 (m, 4.33H), 2.09-1.77 (m, 7.72H), 1.77-1.36 (m, 13.5H), 1.34-0.84 (m, 25H), 1.01 (s, 3H), 0.94 (d, J=6.0 Hz, 3H), 0.72 (s, 1.33H), 0.69 (m, 3H).

### (3S,8S,9S,10R,13R,14S,17R)-17-((R,E)-5,5-Dimethylhex-3-en-2-yl)-10,13-dimethyl-2,3,4,7,8,9,10,11,12,13,14,15,16,17-tetradecahydro-1H-cyclopenta[a]phenanthren-3-ol (156)

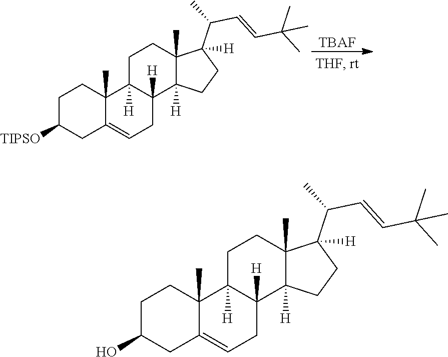

Synthesized according to the general procedure for silyl group deprotection described above. Sterol i-16b (105 mg, 194 μmol), TBAF (970 μL, 970 μmol), and THF (1.9 mL). The crude material was purified by silica gel chromatography (10-20-40-60% EtOAc:hexanes) to afford the product (62 mg, 83%) as a white solid, and as a mixture of geometric isomers (approximately 5:1 E:Z selectivity). ¹H NMR: (300 MHz, CDCl₃, reported as seen in spectrum) δ 5.37-5.29 (m, 2.31H), 5.11 (dd, J=15.0, 9.0 Hz, 1.22H), 4.93 (dd, J=12.0, 9.0 Hz, 0.15H), 3.59-3.45 (m, 1.24H), 2.76-2.61 (m, 0.18H), 2.34-2.16 (m, 2.58H), 2.08-1.90 (m, 3.75H), 1.90-1.77 (m, 2.75H), 1.73-1.36 (m, 11H), 1.34-0.86 (m, 16.7H), 1.01 (s, 3H), 0.96 (s, 9H), 0.72 (s, 0.60H), 0.69 (s, 3H).

### (3S,8S,9S,10R,13R,14S,17R)-17-((R,E)-5-Ethylhept-3-en-2-yl)-10,13-dimethyl-2,3,4,7,8,9,10,11,12,13,14,15,16,17-tetradecahydro-1H-cyclopenta[a]phenanthren-3-ol (160)

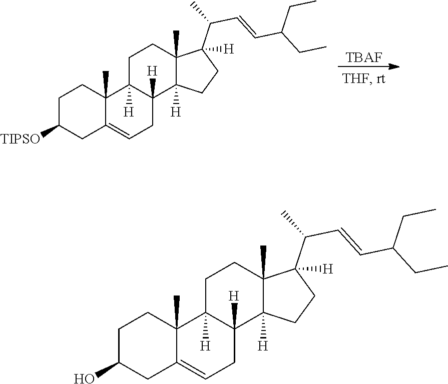

Synthesized according to the general procedure for silyl group deprotection described above. Sterol i-16c (78 mg, 141 μmol), TBAF (703 μL, 703 μmol), and THF (1.4 mL). The crude material was purified by silica gel chromatography (10-20-40-60% EtOAc:hexanes) to afford the product (39 mg, 70%) as a white solid, and as a mixture of geometric isomers (approximately 1:1 E:Z selectivity). ¹H NMR: (300 MHz, CDCl₃, reported as seen in spectrum) δ 5.35 (br d, J=6.0 Hz, 1.93H), 5.22-5.12 (m, 1.93H), 4.97 (dd, J=15.0, 9.0 Hz, 0.91H), 4.87 (dd, J=12.0, 9.0 Hz, 1H), 3.60-3.45 (m, 1.95H), 2.50-1.78 (m, 18.4H), 1.77-0.77 (m, 67.3H), 1.01 (s, 3H), 0.71 (s, 3H), 0.69 (s, 2.56H).

### (3S,8S,9S,10R,13R,14S,17R)-17-((R,E)-6,6-Dimethylhept-3-en-2-yl)-10,13-dimethyl-2,3,4,7,8,9,10,11,12,13,14,15,16,17-tetradecahydro-1H-cyclopenta[a]phenanthren-3-ol (161)

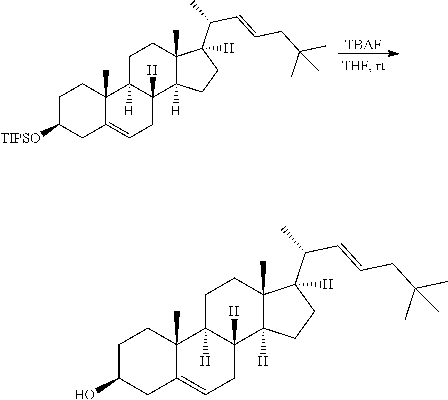

Synthesized according to the general procedure for silyl group deprotection described above. Sterol i-16d (62 mg, 112 μmol), TBAF (559 μL, 559 μmol), and THF (1.1 mL). The crude material was purified by silica gel chromatography (10-20-40-60% EtOAc:hexanes) to afford the product (45 mg, 100%) as a white solid, and as a mixture of geometric isomers (approximately 2:1 E:Z selectivity). ¹H NMR: (300 MHz, CDCl₃, reported as seen in spectrum) δ 5.39-5.14 (m, 4.42H), 3.59-3.45 (m, 1.49H), 2.50-2.16 (m, 5H), 2.11-1.77 (m, 9.85H), 1.76-1.38 (m, 11.3H), 1.37-0.84 (m, 26.3H), 1.01 (s, 3H), 0.95 (d, J=6.0 Hz, 3H), 0.89 (s, 9H), 0.72 (s, 3H), 0.70 (s, 1.33H).

### (3S,8S,9S,10R,13R,14S,17R)-17-((R,E)-4-Cyclohexylbut-3-en-2-yl)-10,13-dimethyl-2,3,4,7,8,9,10,11,12,13,14,15,16,17-tetradecahydro-1H-cyclopenta[a]phenanthren-3-ol (164)

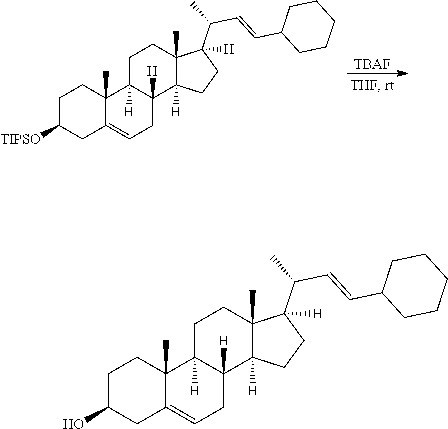

Synthesized according to the general procedure for silyl group deprotection described above. Sterol i-16e (351 mg, 619 μmol), TBAF (3.10 mL, 3.10 mmol), and THF (6.2 mL). The crude material was purified by silica gel chromatography (10-20-40-60% EtOAc:hexanes) to afford the product (189 mg, 74%) as a white solid, and as a mixture of geometric isomers (approximately 1:1 E:Z selectivity). ¹H NMR: (300 MHz, CDCl₃) δ 5.34 (br d, J=3.0 Hz, 2H), 5.29-5.12 (m, 2H), 5.06-4.95 (m, 2H), 3.59-3.45 (m, 2H), 2.50-2.35 (m, 1H), 2.35-2.16 (m, 5H), 2.03-1.91 (m, 5H), 1.90-1.78 (m, 5H), 1.76-1.37 (m, 26H), 1.34-0.87 (m, 33H), 1.00 (s, 3H), 0.72 (s, 3H), 0.68 (s, 3H).

### (3S,8S,9S,10R,13R,14S,17R)-10,13-Dimethyl-17-((R,E)-4-(o-tolyl)but-3-en-2-yl)-2,3,4,7,8,9,10,11,12,13,14,15,16,17-tetradecahydro-1H-cyclopenta[a]phenanthren-3-ol (162)

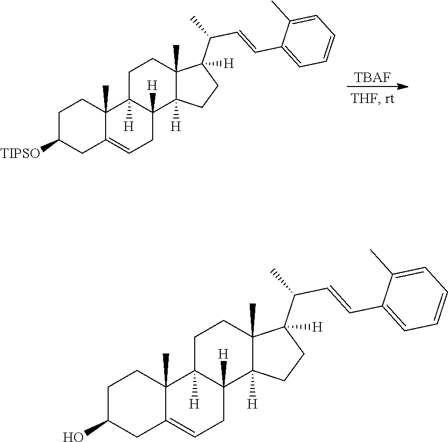

Synthesized according to the general procedure for silyl group deprotection described above. Sterol i-16f (230 mg, 400 μmol), TBAF (2.00 mL, 2.00 mmol), and THF (4.0 mL). The crude material was purified by silica gel chromatography (10-20-40-60% EtOAc:hexanes) to afford the product (157 mg, 94%) as a white solid (complete E selectivity). ¹H NMR: (300 MHz, CDCl₃) δ 7.38 (br d, J=6.0 Hz, 1H), 7.18-7.07 (m, 3H), 6.49 (d, J=15.0 Hz, 1H), 5.92 (dd, J=15.0, 9.0 Hz, 1H), 5.36 (br d, J=6.0 Hz, 1H), 3.60-3.46 (m, 1H), 2.37-2.18 (m, 3H), 2.33 (s, 3H), 2.09-1.93 (m, 2H), 1.91-1.66 (m, 4H), 1.65-0.85 (m, 14H), 1.15 (d, J=6.0 Hz, 3H), 1.03 (s, 3H), 0.77 (s, 3H).

### (3S,8S,9S,10R,13R,14S,17R)-17-((R,E)-4-(2,6-Dimethylphenyl)but-3-en-2-yl)-10,13-dimethyl-2,3,4,7,8,9,10,11,12,13,14,15,16,17-tetradecahydro-1H-cyclopenta[a]phenanthren-3-ol (163)

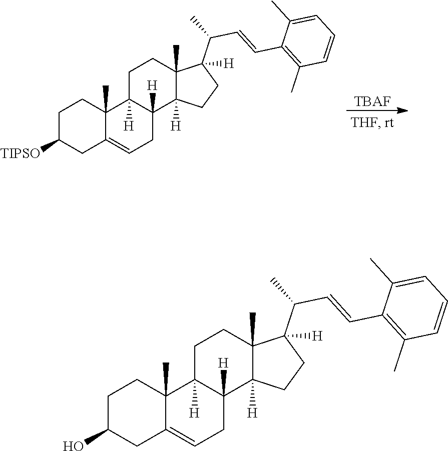

Synthesized according to the general procedure for silyl group deprotection described above. Sterol i-16g (344 mg, 584 μmol), TBAF (2.92 mL, 2.92 mmol), and THF (5.8 mL). The crude material was purified by silica gel chromatography (10-20-40-60% EtOAc:hexanes) to afford the product (239 mg, 95%) as a white solid, and as a mixture of geometric isomers (approximately 3:1 E:Z selectivity). ¹H NMR: (300 MHz, CDCl₃, reported as seen in spectrum) δ 7.11-6.98 (m, 4H), 6.25 (d, J=15.0 Hz, 1H), 6.13 (d, J=12.0 Hz, 0.31H), 5.52 (dd, J=12.0, 9.0 Hz, 0.34H), 5.50 (dd, J=15.0, 6.0 Hz, 1H), 5.40-5.31 (br m, 1.30H), 3.59-3.44 (m, 1.31H), 2.37-2.21 (m, 3H), 2.30 (s, 6H), 2.26 (s, 2H), 2.11-1.73 (m, 9H), 1.67-1.35 (m, 9H), 1.33-0.88 (m, 12H), 1.18 (d, J=9.0 Hz, 3H), 1.04 (s, 3H), 0.78 (s, 3H), 0.50 (s, 1H).

### (3S,8S,9S,10R,13R,14S,17R)-17-((2R,E)-4-((1 S,3S)-Adamantan-1-yl)but-3-en-2-yl)-10,13-dimethyl-2,3,4,7,8,9,10,11,12,13,14,15,16,17-tetradecahydro-1H-cyclopenta[a]phenanthren-3-ol (165)

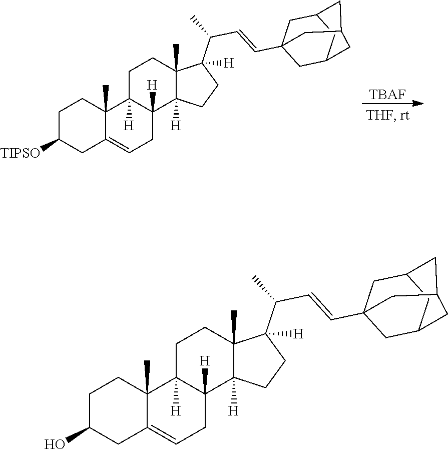

Synthesized according to the general procedure for silyl group deprotection described above. Sterol i-16h (151 mg, 244 μmol), TBAF (1.22 mL, 1.22 mmol), and THF (2.4 mL). The crude material was purified by silica gel chromatography (10-20-40-60% EtOAc:hexanes) to afford the product (103 mg, 91%) as a white solid, and as a mixture of geometric isomers (approximately 3:1 E:Z selectivity). ¹H NMR: (300 MHz, CDCl₃, reported as seen in spectrum) δ 5.34 (br dd, J=6.0, 3.0 Hz, 1.39H), 5.18 (d, J=15.0 Hz, 1H), 5.05 (dd, J=15.0, 9.0 Hz, 1H), 4.97-4.82 (m, 0.73H), 3.59-3.44 (m, 1.44H), 2.78-2.62 (m, 0.33H), 2.37-2.14 (m, 2.85H), 2.07-1.37 (m, 42H), 1.31-0.88 (m, 16H), 1.00 (s, 3H), 0.72 (s, 1.20H), 0.68 (s, 3H).

### (3S,8S,9S,10R,13R,14S,17R)-17-((R,E)-5-Isopropyl-6-methylhept-3-en-2-yl)-10,13-dimethyl-2,3,4,7,8,9,10,11,12,13,14,15,16,17-tetradecahydro-1H-cyclopenta[a]phenanthren-3-ol (167)

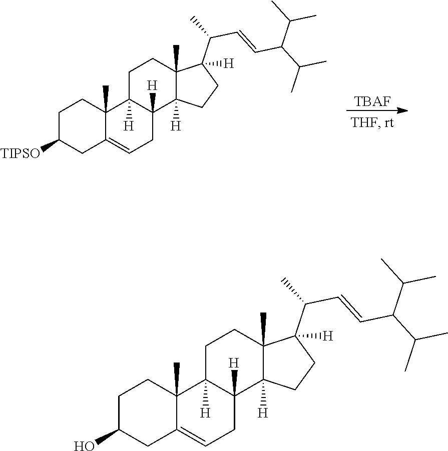

Synthesized according to the general procedure for silyl group deprotection described above. Sterol i-16i (213 mg, 365 μmol), TBAF (1.83 mL, 1.83 mmol), and THF (3.7 mL). The crude material was purified by silica gel chromatography (10-20-40-60% EtOAc:hexanes) to afford the product (132 mg, 85%) as a white solid, and as a mixture of geometric isomers (approximately 2:1 E:Z selectivity). ¹H NMR: (300 MHz, CDCl₃, reported as seen in spectrum) δ 5.35 (br d, J=3.0 Hz, 1.66H), 5.26 (dd, J=12.0, 12.0 Hz, 1H), 5.12 (dd, J=15.0, 6.0 Hz, 0.62H), 5.01 (dd, J=15.0, 9.0 Hz, 0.64H), 4.95 (dd, J=12.0, 12.0 Hz, 1H), 3.59-3.46 (m, 1.65), 2.45-2.16 (m, 4.52H), 2.12-1.38 (m, 26H), 1.37-0.74 (m, 41H), 1.01 (s, 3H), 0.71 (s, 3H), 0.70 (s, 1.75H).

### (3S,8S,9S,10R,13R,14S,17R)-17-((R,E)-5,5-Diethylhept-3-en-2-yl)-10,13-dimethyl-2,3,4,7,8,9,10,11,12,13,14,15,16,17-tetradecahydro-1H-cyclopenta[a]phenanthren-3-ol (166)

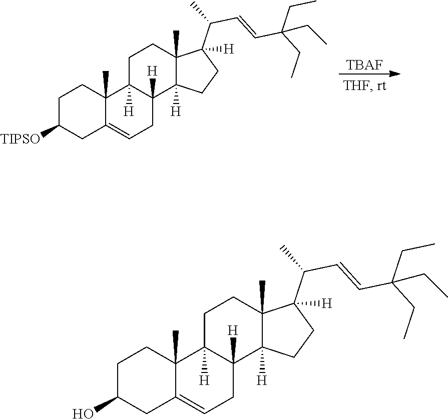

Synthesized according to the general procedure for silyl group deprotection described above. Sterol i-16j (117 mg, 201 μmol), TBAF (1.00 mL, 1.00 mmol), and THF (2.0 mL). The crude material was purified by silica gel chromatography (10-20-40-60% EtOAc:hexanes) to afford the product (40 mg, 47%) as a white solid, and as a mixture of geometric isomers (approximately 5:1 E:Z selectivity). ¹H NMR: (300 MHz, CDCl₃, reported as seen in spectrum) δ 5.35 (br d, J=6.0 Hz, 1.20H), 5.11-4.98 (m, 2.16H), 4.76 (d, J=12.0 Hz, 0.20H), 3.59-3.46 (m, 1.21H), 2.67-2.52 (m, 0.23H), 2.35-2.16 (m, 2.55H), 2.11-1.91 (m, 3H), 1.90-1.77 (m, 2.53H), 1.76-1.36 (m. 12H), 1.35-0.86 (m, 20H), 1.01 (s, 3H), 0.81-0.65 (m, 14.7H).

### (3S,8S,9S,10R,13R,14S,17R)-17-((R,E)-4-Cyclopentylbut-3-en-2-yl)-10,13-dimethyl-2,3,4,7,8,9,10,11,12,13,14,15,16,17-tetradecahydro-1H-cyclopenta[a]phenanthren-3-ol (185)

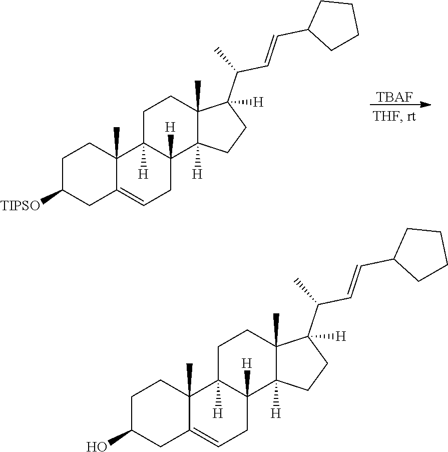

Synthesized according to the general procedure for silyl group deprotection described above. Sterol i-16k (156 mg, 282 μmol), TBAF (1.41 mL, 1.41 mmol), and THE (2.8 mL). The crude material was purified by silica gel chromatography (10-20-40-60% EtOAc:hexanes) to afford the product (104 mg, 93%) as a white solid, and as a mixture of geometric isomers (approximately 3:2 E:Z selectivity). ¹H NMR: (300 MHz, CDCl₃, reported as seen in spectrum) δ 5.39-4.99 (m, 5H), 3.59-3.45 (m, 1.66H), 2.76-2.59 (m, 0.61H), 2.50-2.14 (m, 5.1 OH), 2.08-1.90 (m, 4.53H), 1.90-1.37 (m, 28.1H), 1.33-0.86 (m, 26.1H), 0.72 (s, 1.93H), 0.68 (s, 3H).

### (3S,8S,9S,10R,13R,14S,17R)-17-((R,E)-4-Cycloheptylbut-3-en-2-yl)-10,13-dimethyl-2,3,4,7,8,9,10,11,12,13,14,15,16,17-tetradecahydro-1H-cyclopenta[a]phenanthren-3-ol (186)

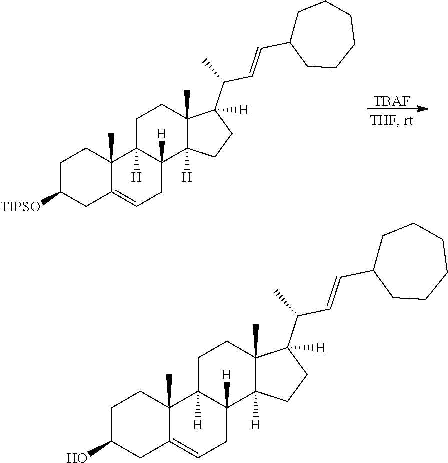

Synthesized according to the general procedure for silyl group deprotection described above. Sterol i-161 (170 mg, 293 μmol), TBAF (1.46 mL, 1.46 mmol), and THE (2.9 mL). The crude material was purified by silica gel chromatography (10-20-40-60% EtOAc:hexanes) to afford the product (104 mg, 84%) as a white solid, and as a mixture of geometric isomers (approximately 1:1 E:Z selectivity). ¹H NMR: (300 MHz, CDCl₃, reported as seen in spectrum) δ 5.38-5.24 (m, 3H), 5.19-5.06 (m, 2H), 4.95 (dd, J=9.0, 9.0 Hz, 1H), 3.59-3.45 (m, 2H), 2.53-2.35 (m, 2H), 2.35-2.15 (m, 4H), 2.10-1.90 (m, 6H), 1.89-1.78 (m, 4H), 1.76-1.36 (m, 37H), 1.35-0.87 (m, 31H), 0.72 (s, 3H), 0.68 (s, 3H).

### (3S,8S,9S,10R,13R,14S,17R)-17-((R,E)-4-(4-Isopropylcyclohexyl)but-3-en-2-yl)-10,13-dimethyl-2,3,4,7,8,9,10,11,12,13,14,15,16,17-tetradecahydro-1H-cyclopenta[a]phenanthren-3-ol (187)

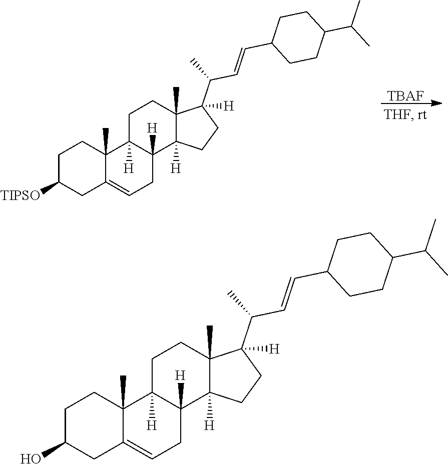

Synthesized according to the general procedure for silyl group deprotection described above. Sterol i-16m (230 mg, 378 μmol), TBAF (1.89 mL, 1.89 mmol), and THF (3.8 mL). The crude material was purified by silica gel chromatography (0-60% EtOAc:hexanes) to afford the product (138 mg, 81%) as a white solid, and as a mixture of geometric isomers (approximately 1:1 E:Z selectivity). ¹H NMR: (300 MHz, CDCl₃, reported as seen in spectrum) δ 5.43-4.91 (m, 5.77H), 3.59-3.44 (m, 1.92H), 2.63-2.51 (m, 0.43H), 2.50-2.35 (m, 1.13H), 2.35-2.10 (m, 5.23H), 2.10-1.91 (m, 5.31H), 1.91-1.77 (m, 4.30H), 1.77-0.78 (m, 75.2H), 0.72 (d, J=3.0 Hz, 3H), 0.69 (d, J=3.0 Hz, 2.57H).

### (3S,8S,9S,10R,13R,14S,17R)-17-((R,E)-4-Cyclododecylbut-3-en-2-yl)-10,13-dimethyl-2,3,4,7,8,9,10,11,12,13,14,15,16,17-tetradecahydro-1H-cyclopenta[a]phenanthren-3-ol (188)

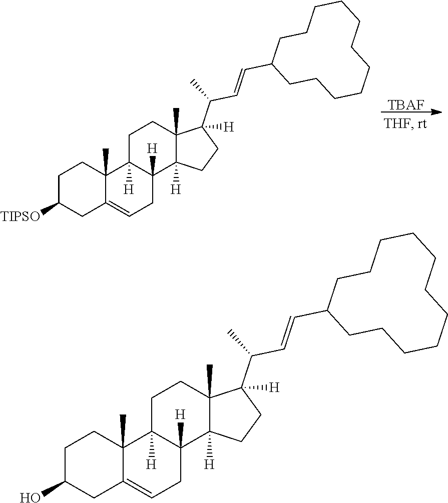

Synthesized according to the general procedure for silyl group deprotection described above. Sterol i-16n (305 mg, 468 μmol), TBAF (2.34 mL, 2.34 mmol), and THF (4.7 mL). The crude material was purified by silica gel chromatography (0-60% EtOAc:hexanes) to afford the product (221 mg, 95%) as a white solid, and as a mixture of geometric isomers (approximately 2:1 E:Z selectivity). ¹H NMR: (300 MHz, CDCl₃, reported as seen in spectrum) δ 5.34 (br d, J=6.0 Hz, 1.58H), 5.23-4.91 (m, 3.14H), 3.59-3.44 (m, 1.54H), 2.61-2.38 (m, 1H), 2.36-2.15 (m, 3.14H), 2.11-1.90 (m, 5.38H), 1.90-1.76 (m, 3.19H), 1.75-0.86 (m, 70H), 0.73 (s, 1.57H), 0.68 (s, 3H).

### (3S,8S,9S,10R,13R,14S,17R)-17-((2R,E)-4-(2-Ethylcyclohexyl)but-3-en-2-yl)-10,13-dimethyl-2,3,47,89,10,11,12,13,14,15,16,17-tetradecahydro-1H-cyclopenta[a]phenanthren-3-ol (189)

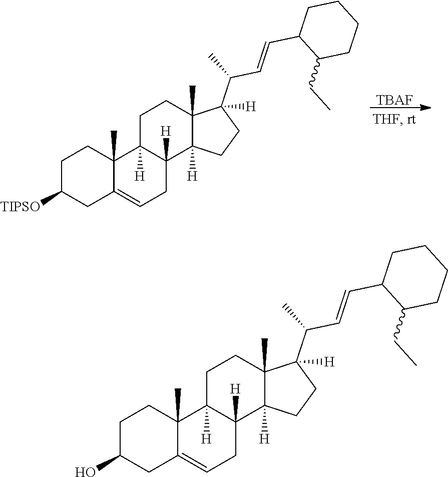

Synthesized according to the general procedure for silyl group deprotection described above. Sterol i-16o (203 mg, 341 μmol), TBAF (1.71 mL, 1.71 mmol), and THF (3.4 mL). The crude material was purified by silica gel chromatography (0-60% EtOAc:hexanes) to afford the product (122 mg, 82%) as a white solid, and as a mixture of geometric isomers (approximately 1:1 E:Z selectivity). ¹H NMR: (300 MHz, CDCl₃, reported as seen in spectrum) δ 5.52-5.29 (m, 3H), 5.27-4.83 (m, 2.65H), 3.59-3.44 (m, 1.89H), 2.72-2.57 (m, 0.65H), 2.51-2.16 (m, 5.59H), 2.09-1.90 (m, 5.38H), 1.90-1.77 (m, 4.62H), 1.77-1.36 (m, 26.8H), 1.35-0.76 (m, 42.5H), 0.71 (s, 3H), 0.69 (d, J=3.0 Hz, 2.44H).

### (3S,8S,9S,10R,13R,14S,17R)-10,13-Dimethyl-17-((R,E)-6-propylnon-3-en-2-yl)-2,3,4,7,8,9,10,11,12,13,14,15,16,17-tetradecahydro-1H-cyclopenta[a]phenanthren-3-ol (214)

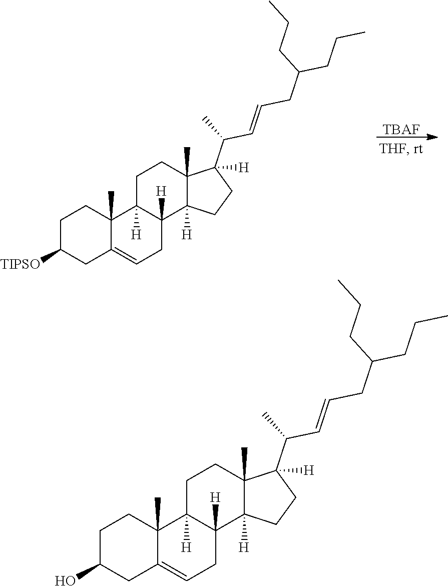

Synthesized according to the general procedure for silyl group deprotection described above. Sterol i-16p (245 mg, 410 μmol), TBAF (2.05 mL, 2.05 mmol), and THF (4.1 mL). The crude material was purified by silica gel chromatography (0-5-10-20-40% EtOAc:hexanes) to afford the product (168 mg, 93%) as a clear viscous oil, and as a mixture of geometric isomers (approximately 4:3 E:Z selectivity). ¹H NMR: (300 MHz, CDCl₃, reported as seen in spectrum) δ 5.39-5.10 (m, 5.40H), 3.59-3.44 (m, 1.78H), 2.52-2.35 (m, 1.15H), 2.34-2.15 (m, 3.80H), 2.10-1.77 (m, 12.3H), 1.76-1.60 (m, 4.15H), 1.60-1.09 (m, 35.8H), 1.09-0.80 (m, 29.1H), 0.71 (s, 3H), 0.69 (s, 2.16H).

### (3S,8S,9S,10R,13R,14S,17R)-17-((R,E)-6-Butyldec-3-en-2-yl)-10,13-dimethyl-2,3,4,7,8,9,10,11,12,13,14,15,16,17-tetradecahydro-1H-cyclopenta[a]phenanthren-3-ol (215)

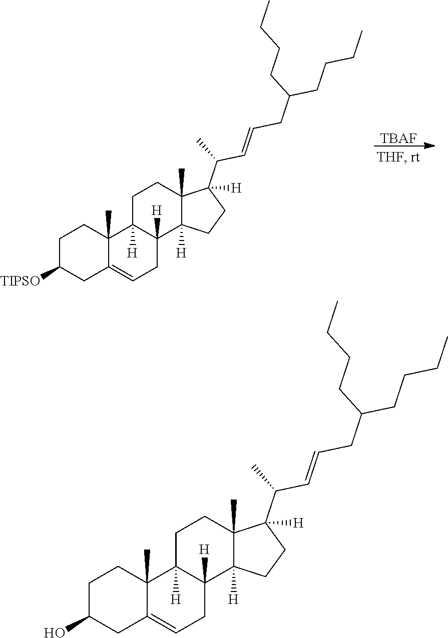

Synthesized according to the general procedure for silyl group deprotection described above. Sterol i-16q (152 mg, 243 μmol), TBAF (1.22 mL, 1.22 mmol), and THF (2.4 mL). The crude material was purified by silica gel chromatography (0-10-20-40% EtOAc:hexanes) to afford the product (107 mg, 94%) as a clear viscous oil, and as a mixture of geometric isomers (approximately 4:3 E:Z selectivity). ¹H NMR: (300 MHz, CDCl₃, reported as seen in spectrum) δ 5.35 (br d, J=3.0 Hz, 1.80H), 5.31-5.11 (m, 3.64H), 3.59-3.45 (m, 1.82H), 2.51-2.35 (m, 1.16H), 2.35-2.15 (m, 3.88H), 2.10-1.77 (m, 12.5H), 1.77-1.38 (m, 16.3H), 1.37-0.81 (m, 62H), 0.72 (m, 3H), 0.69 (s, 2.27H).

### (3S,8S,9S,10R,13R,14S,17R)-17-((R,E)-6-Ethyloct-3-en-2-yl)-10,13-dimethyl-2,3,4,7,8,9,10,11,12,13,14,15,16,17-tetradecahydro-1H-cyclopenta[a]phenanthren-3-ol (219)

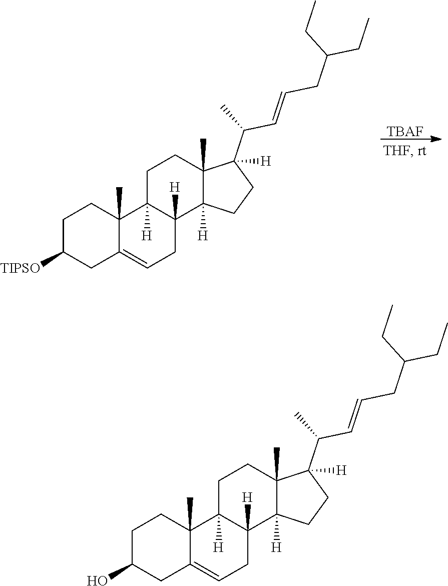

Synthesized according to the general procedure for silyl group deprotection described above. Sterol i-16r (214 mg, 376 μmol), TBAF (1.88 mL, 1.88 mmol), and THF (3.8 mL). The crude material was purified by silica gel chromatography (0-10-20-40-60% EtOAc:hexanes) to afford the product (155 mg, 81%) as a clear viscous oil, and as a mixture of geometric isomers (approximately 3:2 E:Z selectivity). ¹H NMR: (300 MHz, CDCl₃, reported as seen in spectrum) δ 5.34 (br d, J=6.0 Hz, 1.68H), 5.31-5.11 (m, 3.43H), 3.59-3.44 (m, 1.72H), 2.54-2.36 (m, 1.20H), 2.36-2.14 (m, 3.61H), 2.10-1.76 (m, 11.4H), 1.76-1.37 (m, 14.6H), 1.36-0.78 (m, 42.7H), 0.72 (s, 3H), 0.69 (s, 1.88H).

### (3S,8S,9S,10R,13R,14S,17R)-17-((2R,E)-5,6-Diethyloct-3-en-2-yl)-10,13-dimethyl-2,3,4,7,8,9,10,11,12,13,14,15,16,17-tetradecahydro-1H-cyclopenta[a]phenanthren-3-ol (220)

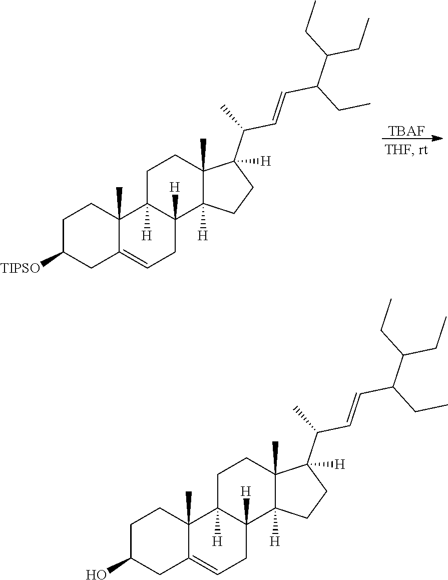

Synthesized according to the general procedure for silyl group deprotection described above. Sterol i-16s (188 mg, 315 μmol), TBAF (1.57 mL, 1.57 mmol), and THF (3.2 mL). The crude material was purified by silica gel chromatography (0-10-20-40% EtOAc:hexanes) to afford the product (123 mg, 89%) as a white solid, and as a mixture of geometric isomers (approximately 3:2 E:Z selectivity). ¹H NMR: (300 MHz, CDCl₃, reported as seen in spectrum) δ 5.34 (br d, J=3.0 Hz, 1.83H), 5.24-5.09 (m, 1.73H), 5.08-4.94 (m, 1.71H), 3.60-3.43 (m, 1.74H), 2.48-2.15 (m, 5.75H), 2.12-1.90 (m, 4.42H), 1.90-1.74 (m, 4.54H), 1.73-0.76 (m, 67.7H), 0.71 (m, 3H), 0.69 (m, 2.17H).

### (3S,8S,9S,10R,13R,14S,17R)-17-((2R,E)-5-Ethyl-6-propyinon-3-en-2-yl)-10,13-dimethyl-2,3,4,7,8,9,10,11,12,13,14,15,16,17-tetradecahydro-1H-cyclopenta[a]phenanthren-3-ol (216)

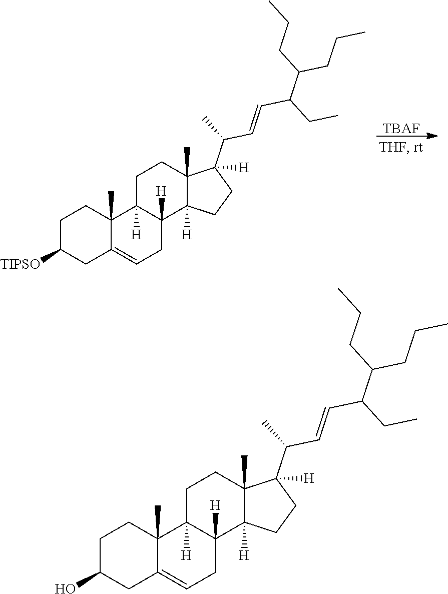

Synthesized according to the general procedure for silyl group deprotection described above. Sterol i-16t (29 mg, 46.0 μmol), TBAF (232 μL, 232 μmol), and THF (464 μL). The crude material was purified by silica gel chromatography (0-10-20-40% EtOAc:hexanes) to afford the product (9.4 mg, 43%) as a clear oil, and as a mixture of geometric isomers (approximately 3:2 E:Z selectivity). ¹H NMR: (300 MHz, CDCl₃, reported as seen in spectrum) δ 5.35 (br d, J=6.0 Hz, 1.77H), 5.21-5.09 (m, 1.68H), 5.09-4.95 (m, 1.69H), 3.60-3.45 (m, 1.80H), 2.46-2.15 (m, 5.92H), 2.09-1.91 (m, 4.48H), 1.90-1.77 (m, 4.17H), 1.73-1.61 (m, 2.08H), 1.61-1.42 (m, 13.6H), 1.42-1.11 (m, 25.8H), 1.10-0.94 (m, 18.6H), 0.94-0.77 (m, 17.3H), 0.72 (s, 3H), 0.69 (s, 2H).

### Example 18. General Procedure A for Reduction

The sterol (1 equiv.) was added to a steel parr reactor and dissolved in THF (0.1 M). Ethanol (0.03 M) and palladium hydroxide on carbon (1 equiv.) were subsequently added to the reactor. The parr reactor was sealed, evacuated, and refilled with H₂ gas, and the pressure was set to 200 psi. The reaction vessel was heated to 80° C. and stirred at 500 rpm for 18 hours. The vessel was then cooled to room temperature, evacuated, refilled with N₂ gas, and opened. The crude reaction mixture was filtered through a syringe filter into a 100 mL round bottom flask and concentrated in vacuo. The crude material was purified as indicated below.

### (3S,8R,9S,10S,13R,14S,17R)-10,13-Dimethyl-17-((R)-5-methylhexan-2-yl)hexadecahydro-1H-cyclopenta[a]phenanthren-3-ol (174)

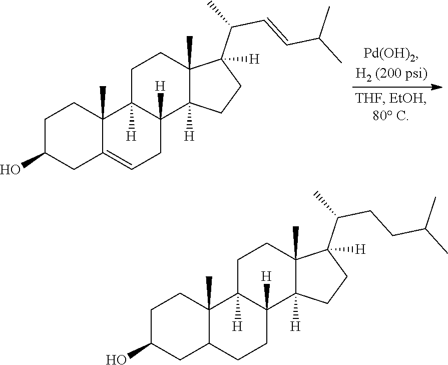

Synthesized according to general procedure A for reduction described above. Sterol 155 (34.0 mg, 92.0 μmol), Pd(OH)₂/C (12.9 mg, 92.0 μmol), THF (1.0 mL), and EtOH (3.1 mL). The crude material was purified by silica gel chromatography (0-10-20-40-70% EtOAc:hexanes) to afford the product (25 mg, 71%) as a white solid. ¹H NMR: (300 MHz, CDCl₃) δ 3.65-3.52 (m, 1H), 1.96 (ddd, J=12.0, 3.0, 3.0 Hz, 1H), 1.88-0.82 (m, 38H), 0.80 (s, 3H), 0.64 (s, 3H), 0.67-0.56 (m, 1H).

### (3S,8R,9S,10S,13R,14S,17R)-17-((R)-5,5-Dimethylhexan-2-yl)-10,13-dimethylhexadecahydro-1H-cyclopenta[a]phenanthren-3-ol (168)

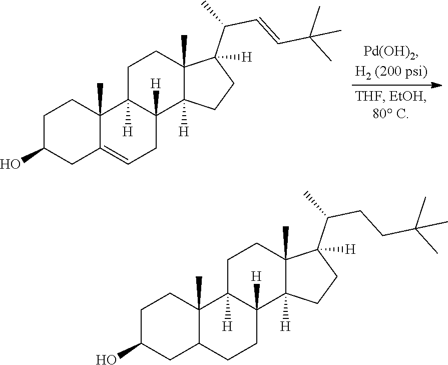

Synthesized according to general procedure A for reduction described above. Sterol 156 (41.0 mg, 107 μmol), Pd(OH)₂/C (15.0 mg, 107 μmol), THF (1.1 mL), and EtOH (3.6 mL). The crude material was purified by silica gel chromatography (0-10-20-40-70% EtOAc:hexanes) to afford the product (31 mg, 75%) as a white solid. ¹H NMR: (300 MHz, CDCl₃) δ 3.65-3.52 (m, 1H), 1.95 (ddd, J=12.0, 3.0, 3.0 Hz, 1H), 1.89-0.77 (m, 29H), 0.89 (d, J=6.0 Hz, 3H), 0.84 (s, 9H), 0.80 (s, 3H), 0.64 (s, 3H), 0.68-0.56 (m, 1H).

### (3S,8R,9S,10S,13R,14S,17R)-10,13-Dimethyl-17-((R)-4-(o-tolyl)butan-2-yl)hexadecahydro-1H-cyclopenta[a]phenanthren-3-ol (170)

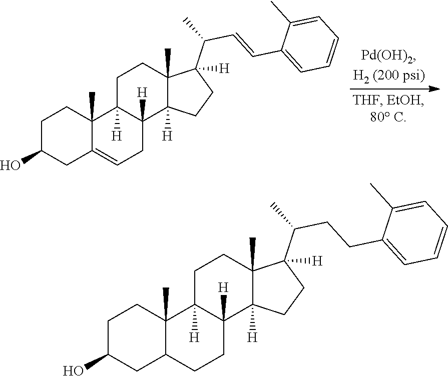

Synthesized according to general procedure A for reduction described above. Sterol 162 (75.0 mg, 179 μmol), Pd(OH)₂/C (25.2 mg, 179 μmol), THF (1.8 mL), and EtOH (6.0 mL). The crude material was purified by silica gel chromatography (0-10-20-40-70% EtOAc:hexanes) to afford the product (37 mg, 49%) as a white solid. ¹H NMR: (300 MHz, CDCl₃) δ 7.16-7.04 (m, 4H), 3.66-3.52 (m, 1H), 2.66 (ddd, J=12.0, 12.0, 6.0 Hz, 1H), 2.44 (ddd, J=15.0, 9.0, 6.0 Hz, 1H), 2.30 (s, 3H), 1.99 (ddd, J=12.0, 3.0, 3.0 Hz, 1H), 1.93-0.78 (m, 27H), 1.05 (d, J=6.0 Hz, 3H), 0.81 (s, 3H), 0.67 (s, 3H), 0.71-0.57 (m, 1H).

### (3S,8R,9S,10S,13R,14S,17R)-17-((R)-4-(2,6-Dimethylphenyl)butan-2-yl)-10,13-dimethylhexadecahydro-1H-cyclopenta[a]phenanthren-3-ol (171)

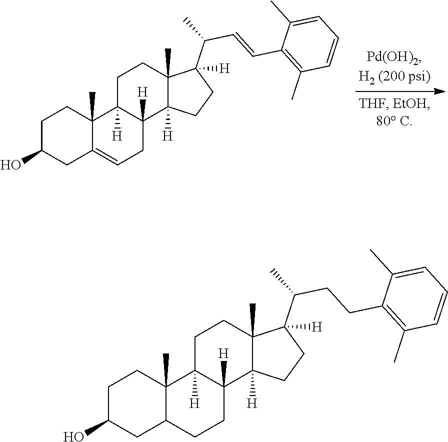

Synthesized according to general procedure A for reduction described above. Sterol 163 (75.0 mg, 173 μmol), Pd(OH)₂/C (24.3 mg, 173 μmol), THF (1.7 mL), and EtOH (5.8 mL). The crude material was purified by silica gel chromatography (0-10-20-40-70% EtOAc:hexanes) to afford the product (42 mg, 55%) as a white solid. ¹H NMR: (300 MHz, CDCl₃) δ 6.98 (br s, 3H), 3.65-3.53 (m, 1H), 2.70 (ddd, J=12.0, 12.0, 6.0 Hz, 1H), 2.43 (ddd, J=12.0, 12.0, 6.0 Hz, 1H), 2.31 (s, 6H), 2.00 (ddd, J=12.0, 3.0, 3.0 Hz, 1H), 1.93-0.78 (m, 27H), 1.08 (d, J=9.0 Hz, 3H), 0.81 (s, 3H), 0.69 (s, 3H), 0.69-0.57 (m, 1H).

### (3S,8R,9S,10S,13R,14S,17R)-17-((R)-4-Cyclohexylbutan-2-yl)-10,13-dimethylhexadecahydro-1H-cyclopenta[a]phenanthren-3-ol (169)

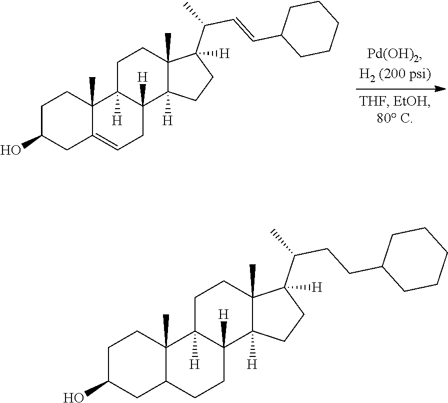

Synthesized according to general procedure A for reduction described above. Sterol 164 (100 mg, 243 μmol), Pd(OH)₂/C (34.2 mg, 243 μmol), THF (2.4 mL), and EtOH (8.1 mL). The crude material was purified by silica gel chromatography (0-10-20-40-70% EtOAc:hexanes) to afford the product (83 mg, 82%) as a white solid. ¹H NMR: (300 MHz, CDCl₃) δ 3.64-3.51 (m, 1H), 1.95 (ddd, J=12.0, 3.0, 3.0 Hz, 1H), 1.87-0.75 (m, 40H), 0.88 (d, J=6.0 Hz, 3H), 0.79 (s, 3H), 0.64 (s, 3H), 0.67-0.55 (m, 1H).

### (3S,8R,9S,10S,13R,14S,17R)-17-((R)-4-((3R,5R,7R)-Adamantan-1-yl)butan-2-yl)-10,13-dimethylhexadecahydro-1H-cyclopenta[a]phenanthren-3-ol (172)

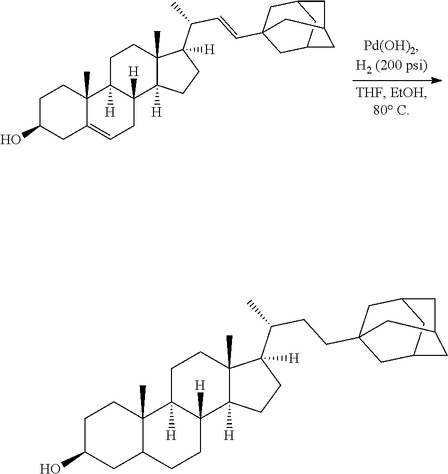

Synthesized according to general procedure A for reduction described above. Sterol 165 (60 mg, 130 μmol), Pd(OH)₂/C (18.2 mg, 130 μmol), THF (1.3 mL), and EtOH (4.3 mL). The crude material was purified by silica gel chromatography (0-10-20-40-70% EtOAc:hexanes) to afford the product (38 mg, 63%) as a white solid. ¹H NMR: (300 MHz, CDCl₃) δ 3.65-3.52 (m, 1H), 1.99-1.88 (m, 4H), 1.88-0.77 (m, 43H), 0.88 (d, J=6.0 Hz, 3H), 0.80 (s, 3H), 0.64 (s, 3H), 0.68-0.56 (m, 1H).

### (3S,8R,9S,10S,13R,14S,17R)-17-((R)-5-Isopropyl-6-methylheptan-2-yl)-10,13-dimethylhexadecahydro-1H-cyclopenta[a]phenanthren-3-ol (173)

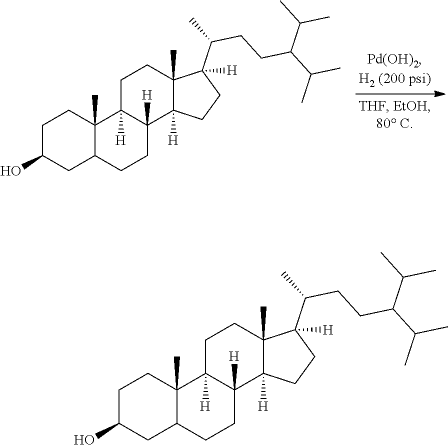

Synthesized according to general procedure A for reduction described above. Sterol 167 (80 mg, 187 μmol), Pd(OH)₂/C (26.3 mg, 187 μmol), THF (1.9 mL), and EtOH (6.2 mL). The crude material was purified by silica gel chromatography (0-10-20-40-70% EtOAc:hexanes) to afford the product (66 mg, 82%) as a white solid. ¹H NMR: (300 MHz, CDCl₃) δ 3.64-3.52 (m, 1H), 1.96 (ddd, J=12.0, 3.0, 3.0 Hz, 1H), 1.90-0.71 (m, 47H), 0.92 (d, J=6.0 Hz, 3H), 0.64 (s, 3H), 0.68-0.56 (m, 1H).

### (3S,8R,9S,10S,13R,14S,17R)-17-((R)-5-Hydroxy-5-methylhexan-2-yl)-10,13-dimethylhexadecahydro-1H-cyclopenta[a]phenanthren-3-ol (180)

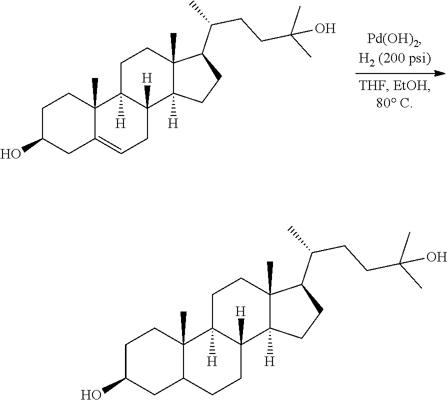

Synthesized according to general procedure A for reduction described above. Sterol 99 (90.0 mg, 232 μmol), Pd(OH)₂/C (32.5 mg, 232 μmol), THF (2.3 mL), and EtOH (7.7 mL). The crude material was purified by silica gel chromatography (0-10-20-40-70% EtOAc:hexanes) to afford the product (66 mg, 73%) as a white solid. ¹H NMR: (300 MHz, CDCl₃) δ 3.65-3.52 (m, 1H), 1.95 (ddd, J=12.0, 3.0, 3.0 Hz, 1H), 1.90-1.17 (m, 22H), 1.19 (s, 6H), 1.16-0.78 (m, 8H), 0.91 (d, J=6.0 Hz, 3H), 0.80 (s, 3H), 0.65 (s, 3H), 0.62 (ddd, J=15.0, 12.0, 6.0 Hz, 1H).

### Example 19. Synthesis of (R)-4-((3S,8S,9S,10R,13R,14S,17R)-3-Hydroxy-10,13-dimethyl-2,3,4,7,8,9,10,11,12,13,14,15,16,17-tetradecahydro-1H-cyclopenta[a]phenanthren-17-yl)-N-methoxy-N-methylpentanamide (153)

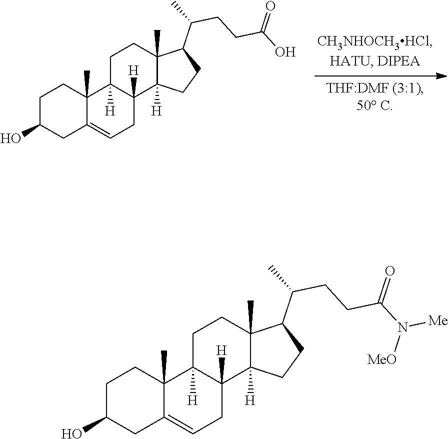

To a solution of cholenic acid (1.00 g, 2.67 mmol) dissolved in THE (30 mL) and DMF (10 mL) at room temperature was added HATU (1.22 g, 3.20 mmol) and N,N-diisopropylethylamine (1.63 mL, 9.34 mmol). The resulting mixture was stirred at 50° C. for 1 hour prior to the addition of N,O-dimethylhydroxylamine hydrochloride (521 mg, 5.34 mmol). The resulting mixture stirred at 50° C. overnight. The reaction mixture was partitioned between EtOAc and water, the layers were separated, and the aqueous layer was extracted with EtOAc. The organic extracts were combined, washed with water (3×), brine, dried over MgSO₄, filtered and concentrated. The crude material was purified by silica gel chromatography (25-50-75% EtOAc:hexanes) to afford the product (888 mg, 80%) as an off-white solid. ¹H NMR: (300 MHz, CDCl₃) δ 5.32 (br d, J=6.0 Hz, 1H), 3.67 (s, 3H), 3.56-3.42 (m, 1H), 3.15 (s, 3H), 2.49-2.14 (m, 4H), 2.03-1.71 (m, 7H), 1.62-0.85 (m, 16H), 0.98 (s, 3H), 0.93 (d, J=6.0 Hz, 3H), 0.67 (s, 3H).

### Example 20. Synthesis of (R)-6-((3S,8S,9S,10R,13R,14S,17R)-3-Hydroxy-10,13-dimethyl-2,3,4,7,8,9,10,11,12,13,14,15,16,17-tetradecahydro-1H-cyclopenta[a]phenanthren-17-yl)heptan-3-one (158)

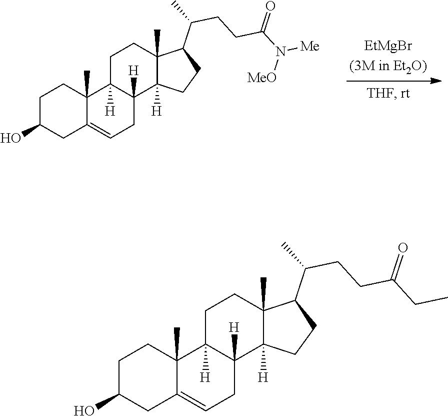

To a solution of the Weinreb amide 153 (200 mg, 479 μmol) dissolved in THE (4.1 mL) was added dropwise a solution of ethylmagnesium bromide (3 M in Et₂O, 798 μL, 2.39 mmol) at room temperature, over 30 minutes, under an argon atmosphere. The resulting mixture was stirred at room temperature for 16 hours. Reaction quenched with sat'd aqueous NH₄Cl and extracted with EtOAc (2×). Organics were combined, dried (MgSO₄), filtered, and concentrated. The crude material was purified by silica gel chromatography (0-10-20-40-60% EtOAc:hexanes) to afford the product (152 mg, 82%) as a white solid. ¹H NMR: (300 MHz, CDCl₃) δ 5.32 (br d, J=3.0 Hz, 1H), 3.57-3.43 (m, 1H), 2.50-2.15 (m, 4H), 2.40 (q, J=9.0 Hz, 2H), 2.02-1.65 (m, 7H), 1.62-0.86 (m, 17H), 1.03 (t, J=9.0 Hz, 3H), 0.98 (s, 3H), 0.89 (d, J=6.0 Hz, 3H), 0.65 (s, 3H).

### Example 21. Synthesis of (3S,8S,9S,10R,13R,14S,17R)-17-((R)-Heptan-2-yl)-10,13-dimethyl-2,3,4,7,8,9,10,11,12,13,14,15,16,17-tetradecahydro-1H-cyclopenta[a]phenanthren-3-ol (179)

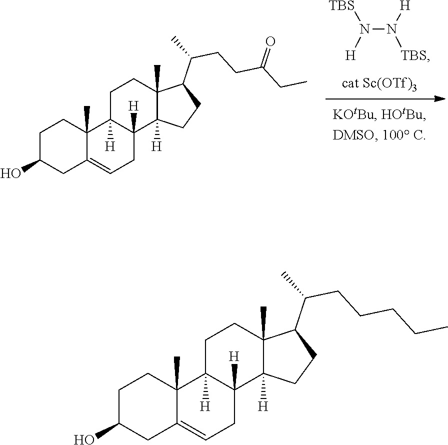

A 25-mL round bottom flask equipped with a stir bar was charged with scandium(III) triflate (14.3 mg, 29.0 μmol) and the ketone 158 (112 mg, 290 μmol). A septum cap was affixed, and a needle connected to an argon balloon was inserted through the septum cap. 1,2-Bis(tert-butyldimethylsilyl)hydrazine (166 mg, 637 μmol) and dry chloroform (750 μL) were then introduced sequentially via syringe. The reaction flask was heated to 55° C. and stirred overnight. Additional chloroform (1 mL) was added to re-achieve stirring, and the resulting mixture was allowed to stir at 55° C. for an additional 2 hours. The septum cap was removed, and the reaction mixture was filtered through a kimwipe plug into another 25 mL round bottom flask. The filtration was quantified with additional hexanes. The solvents were removed in vacuo, and the flask was charged with a stir bar. The flask was attached to a vacuum/nitrogen manifold, and the flask was carefully evacuated with stirring. After stirring under vacuum for 1 hour at rt, the flask was heated to 35° C. and stirred under vacuum for an additional 4 hours. The flask was cooled back to room temperature, flushed with dry nitrogen, a septum cap was affixed, and a needle connected to an argon balloon was inserted through the septum cap. A separate 25-mL round-bottom flask with a stir bar was charged with potassium tert-butoxide (325 mg, 2.90 mmol) and a needle affixed to a nitrogen balloon was inserted through the septum cap. Dry DMSO (2.25 mL) was added via syringe and the mixture was stirred at rt until all particles had dissolved (approximately 5 min). tert-Butanol (275 μL, 2.90 mmol) was then added via syringe and the resulting solution was transferred by syringe to the flask containing the white solid TBSH derivative. The reaction flask was heated to 100° C. and stirred for 16 hours. After the 16 hour period, the reaction was checked by TLC. The reaction was cooled to room temperature, and the reaction was diluted with DCM and brine. The resulting mixture was extracted with DCM (4×). The organic extracts were combined, dried (MgSO₄), filtered, and concentrated in vacuo. The crude residue was purified by silica gel chromatography (0-10-20-40-80% EtOAc:hexanes) to afford the product (50 mg, 46%) as a light-brown solid. ¹H NMR: (300 MHz, CDCl₃) δ 5.34 (br d, J=3.0 Hz, 1H), 3.59-3.45 (m, 1H), 2.34-2.16 (m, 2H), 2.05-1.91 (m, 2H), 1.90-1.76 (m, 3H), 1.62-0.84 (m, 31H), 1.00 (s, 3H), 0.67 (s, 3H). Additionally, ¹³C NMR indicates disappearance of a ketone peak that was present in the starting material.

### Example 21. Synthesis of (3S,8S,9S,10R,13R,14S,17R)-17-((R)-5-Hydroxy-5-methylhexan-2-yl)-10,13-dimethyl-2,3,4,7,8,9,10,11,12,13,14,15,16,17-tetradecahydro-1H-cyclopenta[a]phenanthren-3-ol (99)

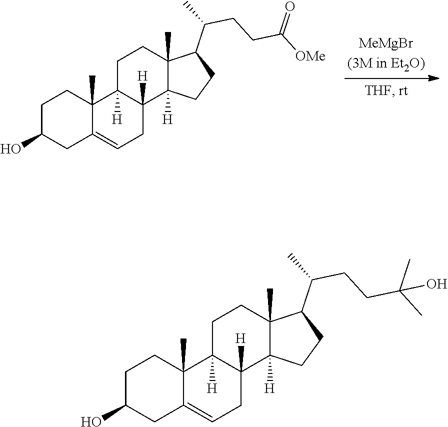

Cholenic acid methyl ester (200 mg, 515 μmol) was added to a round-bottom flask equipped with a stir bar and dissolved in anhydrous THF (12 mL). Methylmagnesium bromide (3 M in Et₂O, 4.29 mL, 12.9 mmol) was added dropwise and the reaction was allowed to stir at room temperature for 2 hours. The reaction mixture was then quenched with saturated aqueous NH₄Cl (exothermic), and the aqueous layer was extracted with EtOAc (2×). Organic extracts were washed with water and brine, dried over MgSO₄, filtered, and concentrated in vacuo. The crude residue was purified by silica gel chromatography (0-10-20-40-60-80% EtOAc:hexanes) to afford the product (171 mg, 85%) as a white solid. ¹H NMR: (300 MHz, CDCl₃) δ 5.35 (br d, J=6.0 Hz, 1H), 3.59-3.46 (m, 1H), 2.34-2.16 (m, 2H), 2.06-1.92 (m, 2H), 1.92-1.76 (m, 3H), 1.64-0.87 (m, 25H), 1.20 (s, 6H), 1.01 (s, 3H), 0.93 (d, J=6.0 Hz, 3H), 0.68 (s, 3H).

### Example 22. Synthesis of (3S,8S,9S,10R,13R,14S,17R)-17-((R)-5-Hydroxy-5-propyloctan-2-yl)-10,13-dimethyl-2,3,4,7,8,9,10,11,12,13,14,15,16,17-tetradecahydro-1H-cyclopenta[a]phenanthren-3-ol (181)

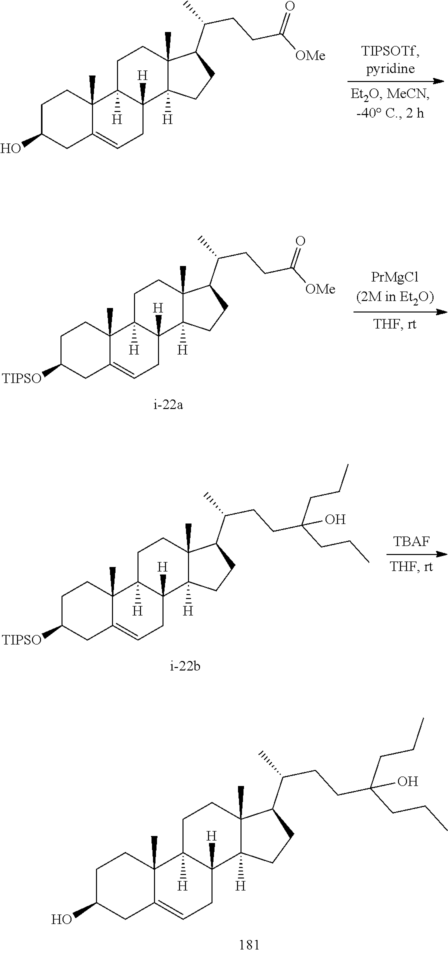

### Step 1: Methyl-(R)-4-((3S,8S,9S,10R,13R,14S,17R)-10,13-dimethyl-3-((triisopropylsilyl)oxy)-2,3,4,7,8,9,10,11,12,13,14,15,16,17-tetradecahydro-1H-cyclopenta[a]phenanthren-17-yl)pentanoate (i-22a)

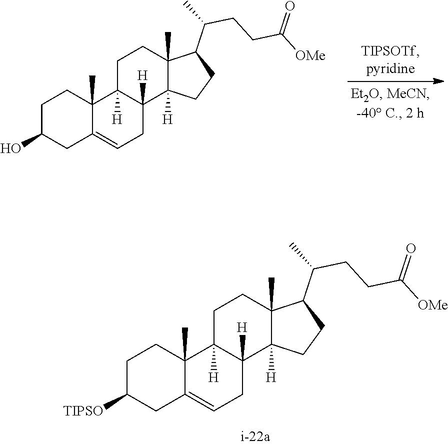

A flask equipped with a stir bar was charged with Et₂O (5.5 mL) and MeCN (3.4 mL) and chilled to −20° C. Triisopropylsilyl trifluoromethanesulfonate (1.58 g, 1.38 mL, 5.15 mmol) and pyridine (275 μL) were added at −20° C. The flask was further chilled to −40° C., charged with cholenic acid methyl ester (1.00 g, 2.57 mmol) in Et₂O (5.5 mL) and allowed to stir at −40° C. for 2 hours. The solution was then poured over saturated aqueous NaHCO₃, extracted with hexanes, washed with water, dried (MgSO₄) and concentrated. The crude material was purified by silica gel chromatography (0-15-30% EtOAc:hexanes) to afford the desired product (1.40 g, 94%) as a clear oil. ¹H NMR: (300 MHz, CDCl₃) δ 5.31 (br d, J=6.0 Hz, 1H), 3.66 (s, 3H), 3.62-3.48 (m, 1H), 2.42-2.15 (m, 4H), 2.03-1.65 (m, 7H), 1.65-1.21 (m, 16H), 1.21-0.82 (m, 36H), 1.00 (s, 3H), 0.67 (s, 3H).

### Step 2: (R)-7-((3S,8S,9S,10R,13R,14S,17R)-10,13-Dimethyl-3-((triisopropylsilyl)oxy)-2,3,4,7,8,9,10,11,12,13,14,15,16,17-tetradecahydro-1H-cyclopenta[a]phenanthren-17-yl)-4-propyloctan-4-ol (i-22b)

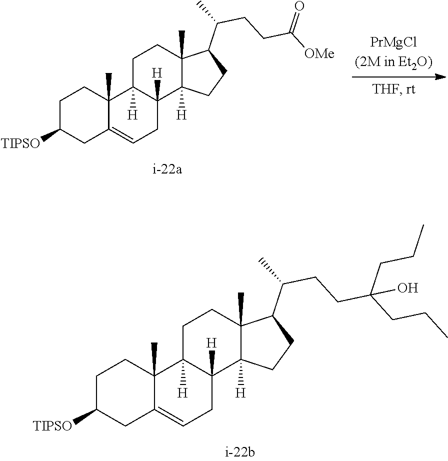

The methyl ester (400 mg, 734 μmol) was added to a round bottom flask equipped with a stir bar and dissolved in anhydrous THF (17 mL). Propylmagnesium chloride (2 M in Et₂O, 9.18 mL, 18.4 mmol) was added dropwise and the reaction was allowed to stir at room temperature overnight. The reaction mixture was then quenched with saturated aqueous NH₄Cl (exothermic), and the aqueous layer was extracted with EtOAc (2×). Organic extracts were washed with water and brine, dried over MgSO₄, filtered, and concentrated in vacuo. The crude residue was purified by silica gel chromatography (0-5-10% EtOAc:hexanes) to afford the product (316 mg, 72%) as a clear oil. ¹H NMR: (300 MHz, CDCl₃) δ 5.30 (br d, J=6.0 Hz, 1H), 3.62-3.48 (m, 1H), 2.34-2.20 (m, 2H), 2.02-1.90 (m, 2H), 1.88-1.74 (m, 3H), 1.64-1.19 (m, 22H), 1.18-0.82 (m, 40H), 1.00 (s, 3H), 0.67 (s, 3H).

### Step 3: (3S,8S,9S,10R, 13R, 14S, 17R)-17-((R)-5-Hydroxy-5-propyloctan-2-yl)-10,13-dimethyl-2,3,4,7,8,9,10,11,12,13,14,15,16,17-tetradecahydro-1H-cyclopenta[a]phenanthren-3-ol (181)

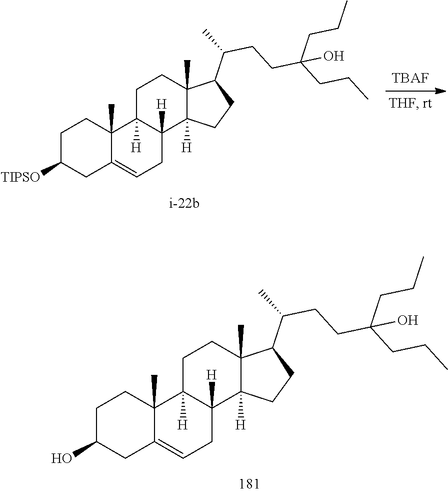

Synthesized according to the general procedure in Example 17. Sterol i-22b (130 mg, 216 μmol), TBAF (1.08 mL, 1.08 mmol), and THF (2.2 mL). The crude material was purified by silica gel chromatography (10-20-40-60% EtOAc:hexanes) to afford the product (88 mg, 91%) as a white solid. ¹H NMR: (300 MHz, CDCl₃) δ 5.34 (br d, J=6.0 Hz, 1H), 3.58-3.44 (m, 1H), 2.35-2.16 (m, 2H), 2.04-1.77 (m, 5H), 1.64-0.86 (m, 37H), 1.00 (s, 3H), 0.92 (d, J=6.0 Hz, 3H), 0.67 (s, 3H).

### Example 23. Synthesis of (3S,8R,9S,10S,13R,14S,17R)-17-((R)-5-Hydroxy-5-propyloctan-2-yl)-10,13-dimethylhexadecahydro-1H-cyclopenta[a]phenanthren-3-ol (182)

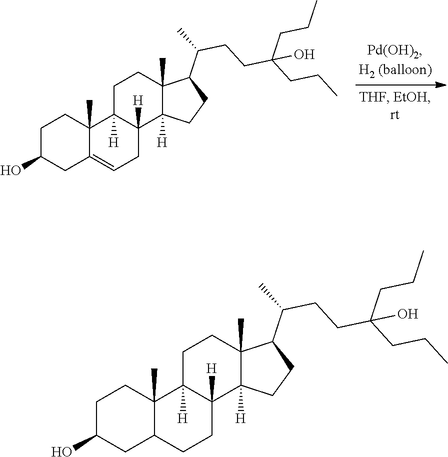

To a flask equipped with a stir bar was added was added palladium hydroxide on carbon (12.0 mg, 85.4 μmol). The sterol 181 (38.0 mg, 85.4 μmol) dissolved in THF (1.3 mL) was added to the flask followed by the addition of EtOH (3.1 mL). The flask was sealed with a septum, evacuated, and subsequently refilled with N₂ gas. The evacuation/backfill process was repeated (2×) followed by a final evacuation. A balloon filled with H₂ was put through the septum (via syringe needle), and the resulting reaction was allowed to stir at room temperature for 2 h. The crude reaction mixture was filtered through a syringe filter into a 100 mL round bottom flask and concentrated in vacuo. The crude material was purified by silica gel chromatography (0-10-20-40-60-80% EtOAc:hexanes) to afford the desired product (24 mg, 63%) as a white solid. ¹H NMR: (300 MHz, CDCl₃) δ 3.65-3.51 (m, 1H), 2.01-1.90 (br d, J=12.0 Hz, 1H), 1.89-1.17 (m, 30H), 1.16-0.83 (m, 17H), 0.80 (s, 3H), 0.64 (s, 3H), 0.68-0.54 (m, 1H).

### Example 24. Synthesis of (R)-2-((3S,8S,9S,10R,13S,14S,17R)-10,13-Dimethyl-3-((triisopropylsilyl)oxy)-2,3,4,7,8,9,10,11,12,13,14,15,16,17-tetradecahydro-1H-cyclopenta[a]phenanthren-17-yl)propan-1-ol (157)

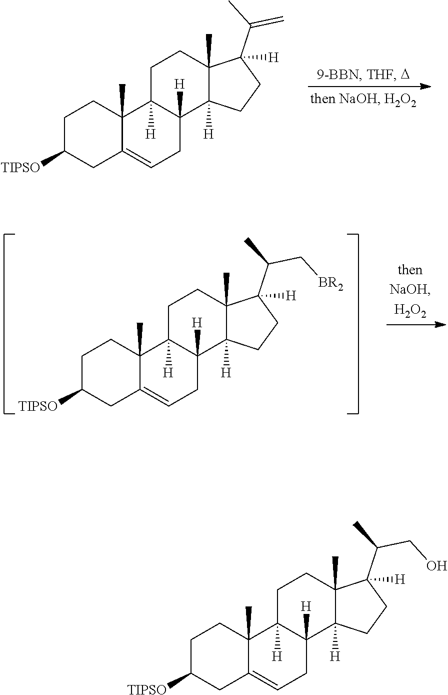

To a solution of alkene 86 (13.3 g, 28.3 mmol) dissolved in anhydrous THF (267 mL) was added 9-BBN (0.5 M in THF, 200 mL, 99.8 mmol) at 0° C. over 15 minutes under argon atmosphere. The reaction was stirred at room temperature for 1 hour, and then warmed to reflux and stirred for an additional 16 hours. The reaction was cooled to 0° C., and 2 N NaOH (267 mL) and 30% H₂O₂ (267 mL) were added. The resulting mixture was warmed to room temperature and allowed to stir for an additional 18 h. After the aqueous layer was extracted with Et₂O, the organic layer was washed with brine, dried (MgSO₄), and concentrated in vacuo. The crude material was purified by silica gel chromatography (0-15-30% EtOAc:hexanes) to afford the minor diastereomer of the desired product (500 mg, 4%) as a white amorphous solid. ¹H NMR: (300 MHz, CDCl₃, for minor diastereomer only) δ 5.29 (br d, J=6.0 Hz, 1H), 3.72 (dd, J=9.0, 3.0 Hz, 1H), 3.60-3.47 (m, 1H), 3.43 (dd, J=9.0, 6.0 Hz, 1H), 2.39 (ddd, J=6.0, 6.0, 3.0 Hz, 1H), 2.32-2.17 (m, 2H), 2.01-1.72 (m, 6H), 1.69-0.79 (m, 44H), 0.99 (s, 3H), 0.93 (d, J=9.0 Hz, 3H), 0.68 (s, 3H).

### Example 25. General Procedure B for Reduction

The sterol (1 equiv.) was added to a steel parr reactor equipped with a stir bar and dissolved in THF (0.07 M). Ethanol (0.05 M) and palladium hydroxide on carbon (1 equiv.) were subsequently added to the reactor. The parr reactor was sealed, evacuated, and refilled with H₂ gas (3×), and the pressure was set to 200 psi. The reaction was stirred at 500 rpm at rt for 18 h. The vessel was then evacuated, refilled with N₂ gas, and opened. The crude reaction mixture was filtered through a Celite pad. The Celite pad was washed with MeOH and the crude material was concentrated and purified as indicated below.

### (3S,8R,9S,10S,13R,14S,17R)-17-((R)-4-Cyclopentylbutan-2-yl)-10,13-dimethylhexadecahydro-1H-cyclopenta[a]phenanthren-3-ol (190)

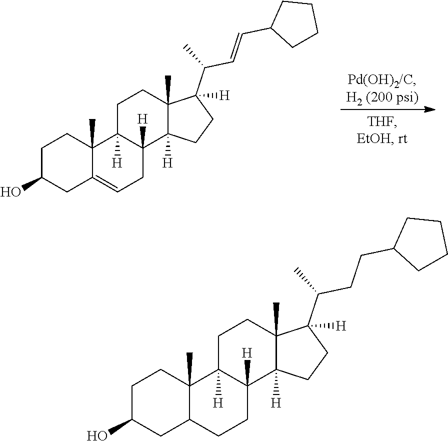

Synthesized according to general procedure B for reduction described above. Sterol 185 (50.0 mg, 126 μmol), Pd(OH)₂/C (17.7 mg, 126 μmol), THF (1.8 mL), and EtOH (2.5 mL). The crude material was purified by silica gel chromatography (0-10-20-40-70% EtOAc:hexanes) to afford the product (25 mg, 49%) as a white solid. ¹H NMR: (300 MHz, CDCl₃) δ 3.65-3.51 (m, 1H), 1.95 (ddd, J=12.0, 3.0, 3.0 Hz, 1H), 1.88-1.16 (m, 27H), 1.16-0.93 (m, 9H), 0.89 (d, J=6.0 Hz, 4H), 0.80 (s, 3H), 0.64 (m, 3H), 0.67-0.56 (m, 1H).

### (3S,8R,9S,10S,13R,14S,17R)-17-((R)-4-Cycloheptylbutan-2-yl)-10,13-dimethylhexadecahydro-1H-cyclopenta[a]phenanthren-3-ol (191)

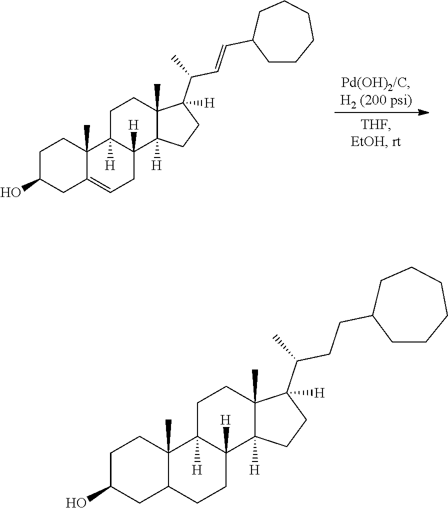

Synthesized according to general procedure B for reduction described above. Sterol 186 (45.0 mg, 106 μmol), Pd(OH)₂/C (14.9 mg, 106 μmol), THF (1.5 mL), and EtOH (2.1 mL). The crude material was purified by silica gel chromatography (0-10-20-40-70% EtOAc:hexanes) to afford the product (21 mg, 46%) as a white solid. ¹H NMR: (300 MHz, CDCl₃) δ 3.65-3.51 (m, 1H), 1.95 (ddd, J=12.0, 3.0, 3.0 Hz, 1H), 1.88-1.19 (m, 30H), 1.19-0.94 (m, 10H), 0.89 (d, J=6.0 Hz, 4H), 0.80 (s, 3H), 0.64 (s, 3H), 0.69-0.55 (m, 1H).

### (3S,8R,9S,10S,13R,14S,17R)-17-((R)-4-(4-Isopropylcyclohexyl)butan-2-yl)-10,13-dimethylhexadecahydro-1H-cyclopenta[a]phenanthren-3-ol (192)

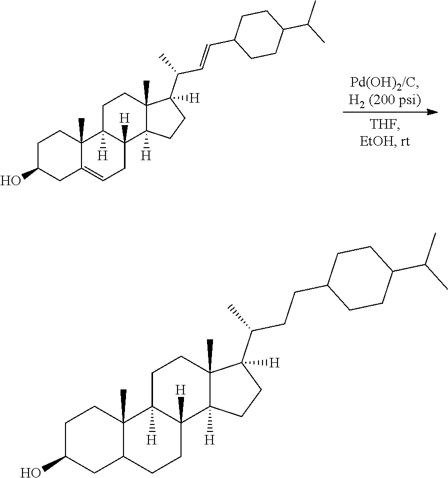

Synthesized according to general procedure B for reduction described above. Sterol 187 (70.0 mg, 155 μmol), Pd(OH)₂/C (22.0 mg, 155 μmol), THF (2.2 mL), and EtOH (3.1 mL). The crude material was purified by silica gel chromatography (0-80% EtOAc:hexanes) to afford the product (64 mg, 91%) as a white solid. ¹H NMR: (300 MHz, CDCl₃) δ 3.65-3.51 (m, 1H), 1.95 (br d, J=12.0 Hz, 1H), 1.87-1.60 (m, 6H), 1.60-0.77 (m, 41H), 0.80 (s, 3H), 0.64 (s, 3H), 0.68-0.55 (m, 1H).

### (3S,8R,9S,10S,13R,14S,17R)-17-((R)-4-Cyclododecylbutan-2-yl)-10,13-dimethylhexadecahydro-1H-cyclopenta[a]phenanthren-3-ol (193)

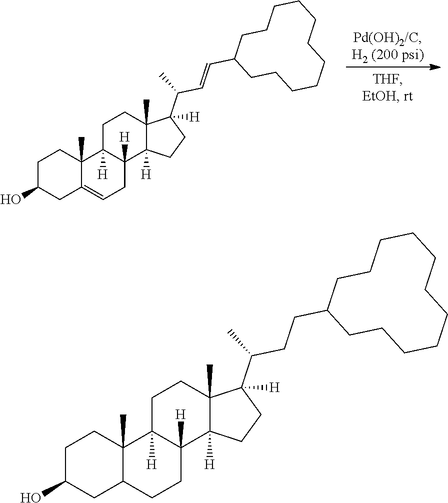

Synthesized according to general procedure B for reduction described above. Sterol 188 (100 mg, 202 μmol), Pd(OH)₂/C (28.0 mg, 202 μmol), THF (2.9 mL), and EtOH (4.0 mL). The crude material was purified by silica gel chromatography (0-20-40-60-80% EtOAc:hexanes) to afford the product (74 mg, 73%) as a white solid. ¹H NMR: (300 MHz, CDCl₃) δ 3.65-3.51 (m, 1H), 1.96 (ddd, J=12.0, 3.0, 3.0 Hz, 1H), 1.90-0.94 (m, 50H), 0.89 (d, J=6.0 Hz, 4H), 0.80 (s, 3H), 0.64 (s, 3H), 0.68-0.56 (m, 1H).

### (3S,8R,9S,10S,13R,14S,17R)-17-((2R)-4-(2-Ethylcyclohexyl)butan-2-yl)-10,13-dimethylhexadecahydro-1H-cyclopenta[a]phenanthren-3-ol (194)

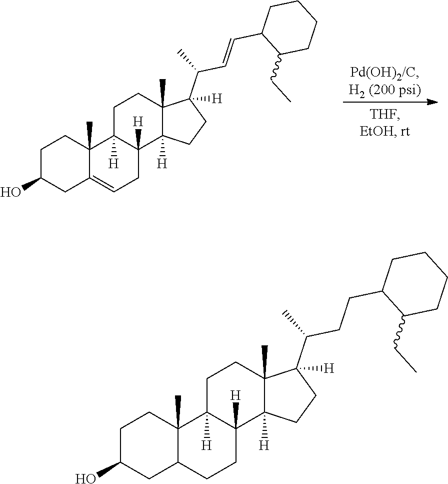

Synthesized according to general procedure B for reduction described above. Sterol 189 (60.0 mg, 137 μmol), Pd(OH)₂/C (19.2 mg, 137 μmol), THF (2.0 mL), and EtOH (2.7 mL). The crude material was purified by silica gel chromatography (0-80% EtOAc:hexanes) to afford the product (50 mg, 83%) as a white solid. ¹H NMR: (300 MHz, CDCl₃) δ 3.65-3.51 (m, 1H), 1.96 (ddd, J=12.0, 3.0, 3.0 Hz, 1H), 1.88-1.61 (m, 6H), 1.60-0.77 (m, 40H), 0.80 (s, 3H), 0.64 (s, 3H), 0.68-0.56 (m, 1H).

### (3S,8R,9S,10S,13R,14S,17R)-10,13-Dimethyl-17-((R)-6-propylnonan-2-yl)hexadecahydro-1H-cyclopenta[a]phenanthren-3-ol (217)

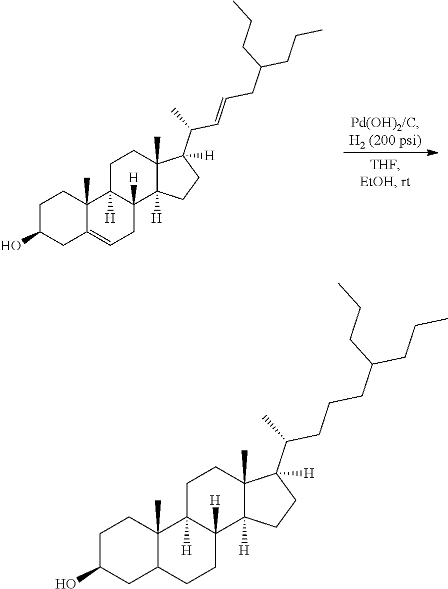

Synthesized according to general procedure B for reduction described above. Sterol 214 (75.0 mg, 170 μmol), Pd(OH)₂/C (23.9 mg, 170 μmol), THF (2.4 mL), and EtOH (3.4 mL). The crude material was purified by silica gel chromatography (0-10-20-40-60% EtOAc:hexanes) to afford the product (69 mg, 91%) as a white solid. ¹H NMR: (300 MHz, CDCl₃) δ 3.65-3.50 (m, 1H), 1.96 (ddd, J=12.0, 3.0, 3.0 Hz, 1H), 1.88-0.83 (m, 47H), 0.80 (s, 3H), 0.64 (s, 3H), 0.62 (ddd, J=15.0, 9.0, 3.0 Hz, 1H).

### (3S,8R,9S,10S,13R,14S,17R)-17-((R)-6-Butyldecan-2-yl)-10,13-dimethylhexadecahydro-1H-cyclopenta[a]phenanthren-3-ol (218)

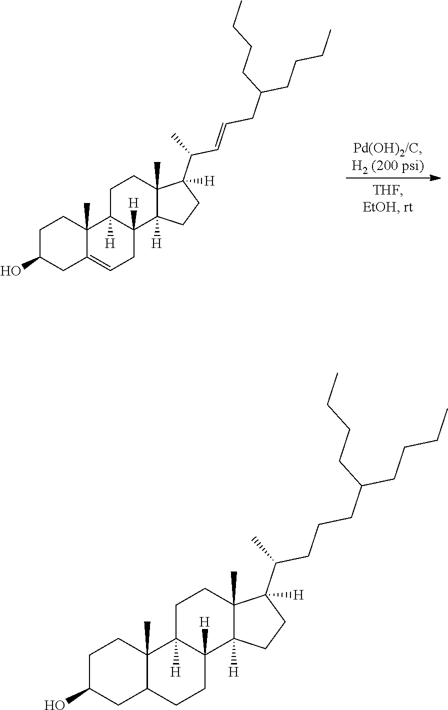

Synthesized according to general procedure B for reduction described above. Sterol 215 (53.2 mg, 113 μmol), Pd(OH)₂/C (15.9 mg, 113 μmol), THF (1.6 mL), and EtOH (2.3 mL). The crude material was purified by silica gel chromatography (0-10-20-40-60% EtOAc:hexanes) to afford the product (47 mg, 88%) as a clear oil. ¹H NMR: (300 MHz, CDCl₃) δ 3.58 (septet, J=6.0 Hz, 1H), 1.96 (ddd, J=12.0, 3.0, 3.0 Hz, 1H), 1.88-0.80 (m, 51H), 0.80 (s, 3H), 0.65 (s, 3H), 0.62 (ddd, J=15.0, 12.0, 6.0 Hz, 1H).

### (3S,8R,9S,10S,13R,14S,17R)-17-((R)-6-Ethyloctan-2-yl)-10,13-dimethylhexadecahydro-1H-cyclopenta[a]phenanthren-3-ol (221)

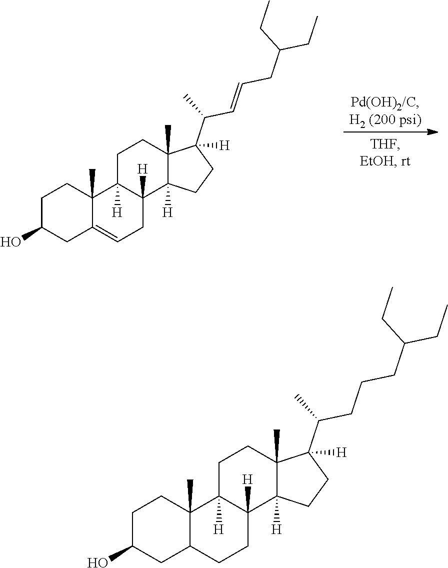

Synthesized according to general procedure B for reduction described above. Sterol 219 (70 mg, 170 μmol), Pd(OH)₂/C (23.8 mg, 170 μmol), THF (2.4 mL), and EtOH (3.4 mL). The crude material was purified by silica gel chromatography (0-10-20-40-60% EtOAc:hexanes) to afford the product (57 mg, 81%) as a white solid. ¹H NMR: (300 MHz, CDCl₃) δ 3.65-3.51 (m, 1H), 1.96 (ddd, J=12.0, 3.0, 3.0 Hz, 1H), 1.87-0.94 (m, 34H), 0.90 (d, J=6.0 Hz, 4H), 0.85 (s, 2H), 0.83 (s, 3H), 0.80 (s, 4H), 0.65 (s, 3H), 0.70-0.56 (m, 1H).

### (3S,8R,9S,10S,13R,14S,17R)-17-((2R)-5,6-Diethyloctan-2-yl)-10,13-dimethylhexadecahydro-1H-cyclopenta[a]phenanthren-3-ol (222)

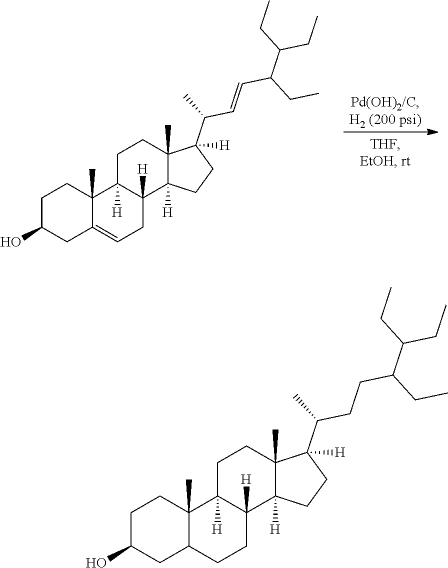

Synthesized according to general procedure B for reduction described above. Sterol 220 (60 mg, 136 μmol), Pd(OH)₂/C (19.1 mg, 136 μmol), THE (1.9 mL), and EtOH (2.7 mL). The crude material was purified by silica gel chromatography (0-10-20-40-60% EtOAc:hexanes) to afford the product (52 mg, 85%) as a white solid. ¹H NMR: (300 MHz, CDCl₃) δ 3.65-3.51 (m, 1H), 1.96 (ddd, J=12.0, 6.0, 3.0 Hz, 1H), 1.89-1.61 (m, 4H), 1.61-1.43 (m, 4H), 1.43-0.81 (m, 39H), 0.80 (m, 3H), 0.64 (s, 3H), 0.62 (ddd, J=15.0, 12.0, 6.0 Hz, 1H).

### Example 26. Synthesis of (3S,8R,9S,10R,13S,14S)-10,13-Dimethyl-17-(((trifluoromethyl)sulfonyl) oxy)-2,3,4,7,8,9,10,11,12,13,14,15-dodecahydro-1H-cyclopenta[a]phenanthren-3-yl acetate (i-26)

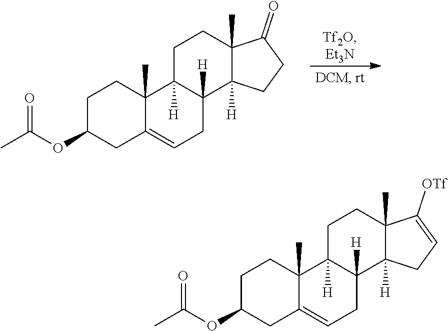

To a stirred solution of dehydroisoandrosterone 3-acetate (5.00 g, 15.1 mmol) in DCM (151 mL) was added triflic anhydride (2.80 mL, 16.6 mmol). The resulting mixture stirred at rt for 5 minutes. A solution of Et₃N (2.11 mL, 15.1 mmol) in DCM (50 mL) was slowly added, and the resulting mixture was allowed to stir at rt for an addition 3.5 hours. The reaction was quenched with water, and the layers were separated. The aqueous layer was extracted with DCM (2×), and the combined organic extracts were washed with brine, dried (MgSO₄), filtered, and concentrated. The crude material was purified by silica gel chromatography (0-5-10-20-40% EtOAc:hexanes) to afford the product (2.87 g, 41%) as a yellow-orange oil. ¹H NMR: (300 MHz, CDCl₃) δ 5.59 (br dd, J=3.0, 3.0 Hz, 1H), 5.39 (br d, J=6.0 Hz, 1H), 4.67-4.53 (m, 1H), 2.41-2.29 (m, 2H), 2.24 (ddd, J=15.0, 6.0, 3.0 Hz, 1H), 2.09-1.95 (m, 2H), 2.03 (s, 3H), 1.92-1.40 (m, 10H), 1.21-1.03 (m, 2H), 1.06 (s, 3H), 1.00 (s, 3H).

### Example 27. General Procedure for Suzuki Coupling

A flame dried flask equipped with a stir bar was charged with the sterol (1 equiv.), the boronic acid (1.1 equiv.), and bis(triphenylphosphine)palladium(II) dichloride (0.1 equiv.). The flask and its contents were vacuum flushed and purged with argon (3×). Then THE (0.15 M) was added followed by a saturated solution of NaHCO₃ (0.5 M) that had been sparged with N₂ for 15 minutes prior to addition. The reaction mixture was heated to 60° C. and stirred for the allotted time indicated below. The reaction was cooled to room temperature, the solvent was removed under vacuum, and the resulting black residue was dissolved in DCM and washed with water. The aqueous layer was extracted with DCM (2×) and the combined organic layers were dried (MgSO₄), filtered, and concentrated. The crude material was purified as indicated below.

### (3S,8R,9S,10R,13S,14S)-10,13-Dimethyl-17-phenyl-2,3,4,7,8,9,10,11,12,13,14,15-dodecahydro-1H-cyclopenta[a]phenanthren-3-yl acetate (i-27a)

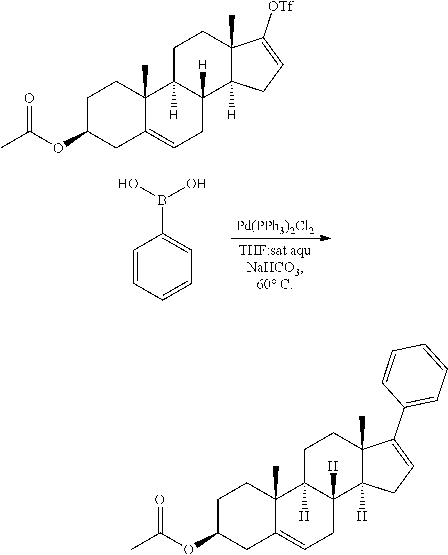

Synthesized according to the general procedure for Suzuki coupling described above. Sterol i-26 (500 mg, 1.08 mmol), phenylboronic acid (145 mg, 1.19 mmol), Pd(PPh₃)₂Cl₂ (75.9 mg, 108 μmol), THE (7.3 mL), and saturated aqueous NaHCO₃ (2.2 mL). The reaction stirred at 60° C. for 3 hours. The crude material was purified by silica gel chromatography (0-20% EtOAc:hexanes) to afford the product (323 mg, 77%) as a white solid. ¹H NMR: (300 MHz, CDCl₃) δ 7.41-7.35 (m, 2H), 7.34-7.19 (m, 3H), 5.92 (dd, J=6.0, 3.0 Hz, 1H), 5.43 (d, J=6.0 Hz, 1H), 4.70-4.56 (m, 1H), 2.43-2.31 (m, 2H), 2.24 (ddd, J=15.0, 6.0, 3.0 Hz, 1H), 2.15-1.98 (m, 3H), 2.04 (s, 3H), 1.93-1.81 (m, 2H), 1.81-1.42 (m, 7H), 1.31-1.02 (m, 2H), 1.09 (s, 3H), 1.07 (s, 3H).

### (3S,8R,9S,10R,13S,14S)-10,13-Dimethyl-17-(p-tolyl)-2,3,4,7,8,9,10,11,12,13,14,15-dodecahydro-1H-cyclopenta[a]phenanthren-3-yl acetate (i-27b)

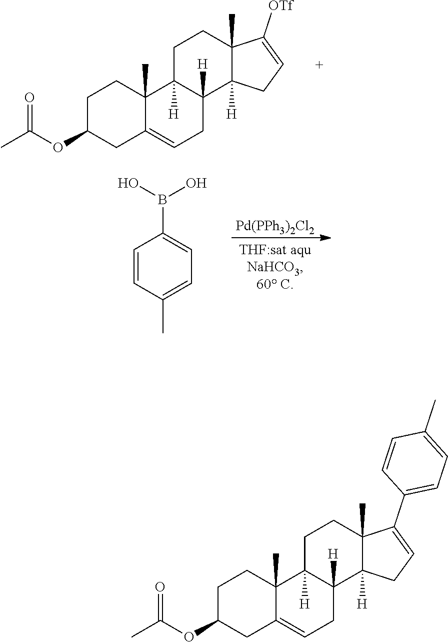

Synthesized according to the general procedure for Suzuki coupling described above. Sterol i-26 (500 mg, 1.08 mmol), p-tolylboronic acid (162 mg, 1.19 mmol), Pd(PPh₃)₂Cl₂ (75.9 mg, 108 μmol), THE (7.3 mL), and saturated aqueous NaHCO₃ (2.2 mL). The reaction stirred at 60° C. for 3 hours. The crude material was purified by silica gel chromatography (0-20% EtOAc:hexanes) to afford the product (341 mg, 78%) as an off-white solid. ¹H NMR: (300 MHz, CDCl₃) δ 7.27 (d, J=9.0 Hz, 2H), 7.11 (d, J=9.0 Hz, 2H), 5.87 (dd, J=6.0, 3.0 Hz, 1H), 5.42 (d, J=6.0 Hz, 1H), 4.69-4.55 (m, 1H), 2.41-2.30 (m, 2H), 2.34 (s, 3H), 2.22 (ddd, J=15.0, 6.0, 3.0 Hz, 1H), 2.13-1.96 (m, 3H), 2.04 (s, 3H), 1.93-1.81 (m, 2H), 1.80-1.42 (m, 7H), 1.29-1.01 (m, 2H), 1.08 (s, 3H), 1.05 (s, 3H).

### (3S,8R,9S,10R,13S,14S)-17-(4-Isopropylphenyl)-10,13-dimethyl-2,3,4,7,8,9,10,11,12,13,14,15-dodecahydro-1H-cyclopenta[a]phenanthren-3-yl acetate (i-27c)

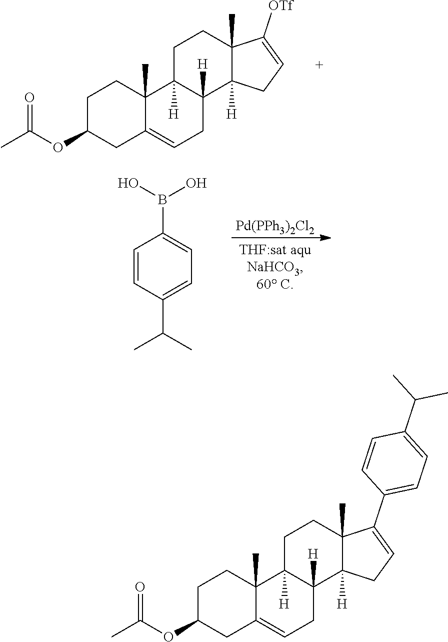

Synthesized according to the general procedure for Suzuki coupling described above. Sterol i-26 (500 mg, 1.08 mmol), 4-isopropylpheylboronic acid (195 mg, 1.19 mmol), Pd(PPh₃)₂Cl₂ (75.9 mg, 108 μmol), THE (7.3 mL), and saturated aqueous NaHCO₃ (2.2 mL). The reaction stirred at 60° C. for 3 hours. The crude material was purified by silica gel chromatography (0-20% EtOAc:hexanes) to afford the product (356 mg, 76%) as a white solid. ¹H NMR: (300 MHz, CDCl₃) δ 7.32 (d, J=9.0 Hz, 2H), 7.17 (d, J=9.0 Hz, 2H), 5.89 (dd, J=3.0, 3.0 Hz, 1H), 5.43 (d, J=6.0 Hz, 1H), 4.70-4.55 (m, 1H), 2.90 (septet, 1H), 2.43-2.31 (m, 2H), 2.22 (ddd, J=12.0, 6.0, 3.0 Hz, 1H), 2.17-1.97 (m, 3H), 2.05 (s, 3H), 1.94-1.82 (m, 2H), 1.80-1.43 (m, 7H), 1.33-1.01 (m, 8H), 1.26 (d, J=6.0 Hz, 3H), 1.08 (d, J=9.0 Hz, 3H).

### (3S,8R,9S,10R,13S,14S)-17-(4-(tert-Butyl)phenyl)-10,13-dimethyl-2,3,4,7,8,9,10,11,12,13,14,15-dodecahydro-1H-cyclopenta[a]phenanthren-3-yl acetate (i-27d)

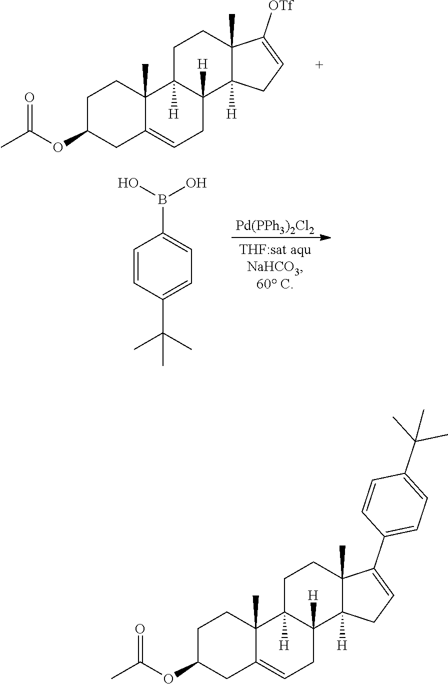

Synthesized according to the general procedure for Suzuki coupling described above. Sterol i-26 (500 mg, 1.08 mmol), 4-tert-butylpheylboronic acid (212 mg, 1.19 mmol), Pd(PPh₃)₂Cl₂ (75.9 mg, 108 μmol), THE (7.3 mL), and saturated aqueous NaHCO₃ (2.2 mL). The reaction stirred at 60° C. for 3 hours. The crude material was purified by silica gel chromatography (0-20% EtOAc:hexanes) to afford the product (378 mg, 78%) as a white solid. ¹H NMR: (300 MHz, CDCl₃) δ 7.33 (s, 4H), 5.90 (dd, J=3.0, 3.0 Hz, 1H), 5.43 (d, J=6.0 Hz, 1H), 4.70-4.55 (m, 1H), 2.43-2.31 (m, 2H), 2.22 (ddd, J=15.0, 6.0, 3.0 Hz, 1H), 2.18-1.96 (m, 3H), 2.04 (s, 3H), 1.94-1.82 (m, 2H), 1.81-1.42 (m, 7H), 1.33 (s, 9H), 1.23-1.03 (m, 2H), 1.09 (s, 3H), 1.07 (s, 3H).

### (3S,8R,9S,10R,13S,14S)-10,13-Dimethyl-17-(m-tolyl)-2,3,4,7,8,9,10,11,12,13,14,15-dodecahydro-1H-cyclopenta[a]phenanthren-3-yl acetate (i-27e)

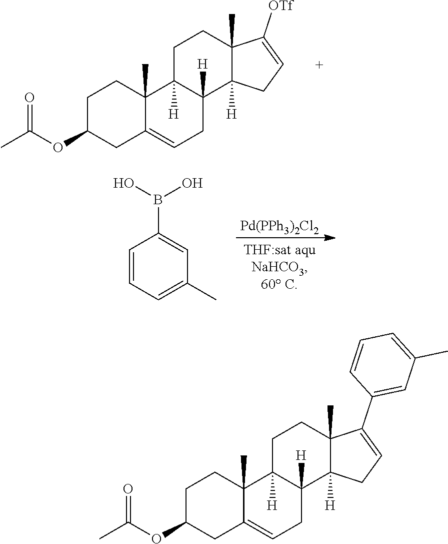

Synthesized according to the general procedure for Suzuki coupling described above. Sterol i-26 (500 mg, 1.08 mmol), m-tolylboronic acid (162 mg, 1.19 mmol), Pd(PPh₃)₂Cl₂ (75.9 mg, 108 μmol), THE (7.3 mL), and saturated aqueous NaHCO₃ (2.2 mL). The reaction stirred at 60° C. for 3 hours. The crude material was purified by silica gel chromatography (0-20% EtOAc:hexanes) to afford the product (336 mg, 77%) as a clear oil. ¹H NMR: (300 MHz, CDCl₃) δ 7.24-7.15 (m, 3H), 7.11-7.01 (m, 1H), 5.90 (dd, J=3.0, 3.0 Hz, 1H), 5.43 (d, J=3.0 Hz, 1H), 4.71-4.56 (m, 1H), 2.43-2.32 (m, 2H), 2.35 (s, 3H), 2.23 (ddd, J=15.0, 6.0, 3.0 Hz, 1H), 2.15-1.98 (m, 3H), 2.05 (s, 3H), 1.95-1.82 (m, 2H), 1.81-1.43 (m, 8H), 1.22-1.03 (m, 2H), 1.10 (s, 3H), 1.07 (s, 3H).

### (3S,8R,9S,10R,13S,14S)-17-(3,5-Dimethylphenyl)-10,13-dimethyl-2,3,4,7,8,9,10,11,12,13,14,15-dodecahydro-1H-cyclopenta[a]phenanthren-3-yl acetate (i-27f)

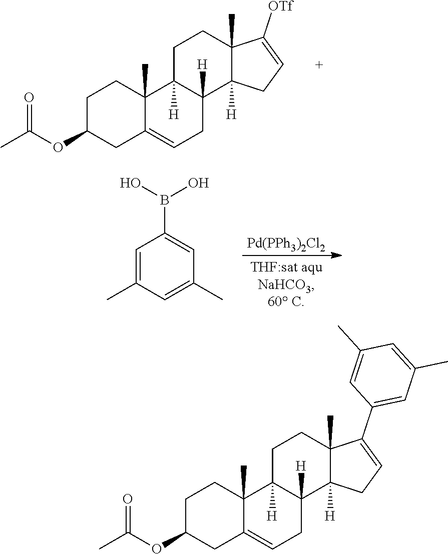

Synthesized according to the general procedure for Suzuki coupling described above. Sterol i-26 (500 mg, 1.08 mmol), 3,5-dimethylphenylboronic acid (178 mg, 1.19 mmol), Pd(PPh₃)₂Cl₂ (75.9 mg, 108 μmol), THE (7.3 mL), and saturated aqueous NaHCO₃ (2.2 mL). The reaction stirred at 60° C. for 3 hours. The crude material was purified by silica gel chromatography (0-20% EtOAc:hexanes) to afford the product (380 mg, 84%) as a clear oil. ¹H NMR: (300 MHz, CDCl₃) δ 7.01 (br s, 2H), 6.90 (br s, 1H), 5.89 (dd, J=6.0, 3.0 Hz, 1H), 5.44 (br d, J=6.0 Hz, 1H), 4.72-4.56 (m, 1H), 2.42-2.29 (m, 2H), 2.32 (s, 6H), 2.23 (ddd, J=15.0, 6.0, 3.0 Hz, 1H), 2.16-1.97 (m, 3H), 2.06 (s, 3H), 1.95-1.83 (m, 2H), 1.81-1.44 (m, 8H), 1.35-1.27 (m, 1H), 1.24-1.04 (m, 2H), 1.10 (s, 3H), 1.07 (s, 3H).

### (3S,8R,9S,10R,13S,14S)-10,13-Dimethyl-17-(o-tolyl)-2,3,4,7,8,9,10,11,12,13,14,15-dodecahydro-1H-cyclopenta[a]phenanthren-3-yl acetate (i-27g)

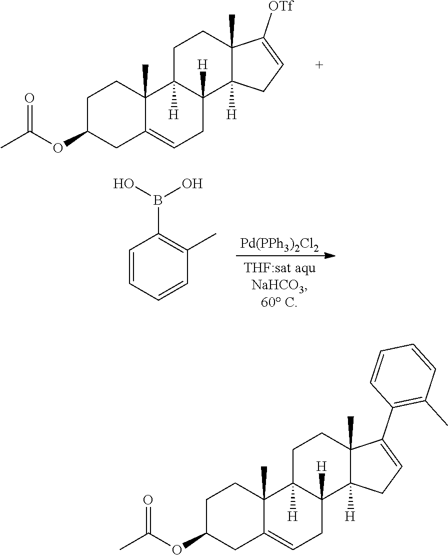

Synthesized according to the general procedure for Suzuki coupling described above. Sterol i-27g (500 mg, 1.08 mmol), o-tolylboronic acid (162 mg, 1.19 mmol), Pd(PPh₃)₂Cl₂ (75.9 mg, 108 μmol), THE (7.3 mL), and saturated aqueous NaHCO₃ (2.2 mL). The reaction stirred at 60° C. for 3 hours. The crude material was purified by silica gel chromatography (0-20% EtOAc:hexanes) to afford the product (375 mg, 86%) as a white solid. ¹H NMR: (300 MHz, CDCl₃) δ 7.25-7.06 (m, 4H), 5.59 (dd, J=3.0, 3.0 Hz, 1H), 5.45 (br d, J=6.0 Hz, 1H), 4.71-4.57 (m, 1H), 2.44-2.25 (m, 3H), 2.32 (s, 3H), 2.17-2.01 (m, 2H), 2.05 (s, 3H), 1.94-1.82 (m, 2H), 1.82-1.70 (m, 2H), 1.69-1.49 (m, 6H), 1.24-1.06 (m, 2H), 1.09 (s, 3H), 0.97 (s, 3H).

### (3S,8R,9S,10R,13S,14S)-17-(2,6-Dimethylphenyl)-10,13-dimethyl-2,3,4,7,8,9,10,11,12,13,14,15-dodecahydro-1H-cyclopenta[a]phenanthren-3-yl acetate (i-27h)

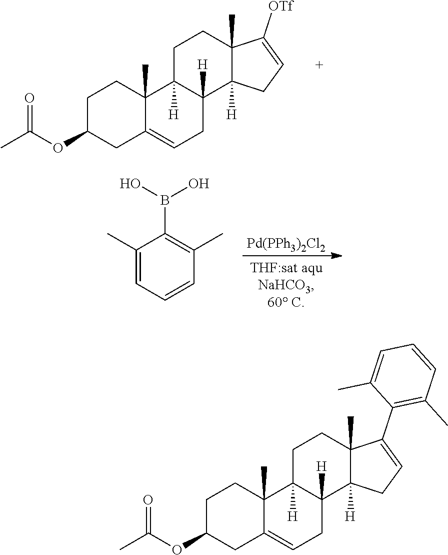

Synthesized according to the general procedure for Suzuki coupling described above. Sterol i-26 (700 mg, 1.51 mmol), 2,6-dimethylphenyl acid (250 mg, 1.67 mmol), Pd(PPh₃)₂Cl₂ (106 mg, 151 μmol), THE (10 mL), and saturated aqueous NaHCO₃ (3.0 mL). The reaction stirred at 60° C. for 60 h. The crude material was purified by silica gel chromatography (0-20% EtOAc:hexanes) to afford the product (80 mg, 13%) as a white solid. ¹H NMR: (300 MHz, CDCl₃) δ 7.11-6.98 (m, 3H), 5.53 (dd, J=3.0, 3.0 Hz, 1H), 5.43 (br d, J=3.0 Hz, 1H), 4.70-4.55 (m, 1H), 2.40-2.25 (m, 2H), 2.29 (s, 3H), 2.27 (s, 3H), 2.23-2.03 (m, 3H), 2.04 (s, 3H), 1.92-1.78 (m, 2H), 1.77-1.42 (m, 8H), 1.21-1.04 (m, 2H), 1.07 (s, 3H), 0.96 (s, 3H).

### Example 28. General Procedure for Acetate Deprotection

A round bottom flask equipped with a stir bar was charged with the sterol (1 equiv.), potassium carbonate (10 equiv.), MeOH (0.03 M), and THE (0.12 M). The resulting mixture was heated to 45° C. and stirred for 1 hour. The reaction was then quenched with a saturated solution of NH₄Cl, layers were separated, and the aqueous layer was extracted with DCM (3×). The combined organic layers were dried over MgSO₄, filtered, and concentrated. The crude material was purified as indicated below.

### (3S,8R,9S,10R,13S,14S)-10,13-Dimethyl-17-phenyl-2,3,4,7,8,9,10,11,12,13,14,15-dodecahydro-1H-cyclopenta[a]phenanthren-3-ol (19)

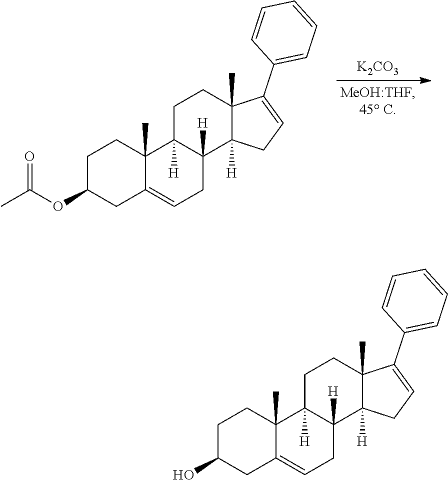

Synthesized according to the general procedure for acetate deprotection described above. Sterol i-27a (323 mg, 827 μmol), K₂CO₃ (1.14 g, 8.27 mmol), MeOH (28 mL), and THE (6.9 mL). The crude material was purified by silica gel chromatography (0-20-40-60-80-100% EtOAc:hexanes) to afford the product (261 mg, 91%) as a white solid. ¹H NMR: (300 MHz, CDCl₃) δ 7.41-7.34 (m, 2H), 7.33-7.19 (m, 3H), 5.92 (dd, J=6.0, 3.0 Hz, 1H), 5.40 (br d, J=6.0 Hz, 1H), 3.61-3.47 (m, 1H), 2.38-2.18 (m, 3H), 2.14-1.99 (m, 3H), 1.90-1.42 (m, 10H), 1.18-1.00 (m, 2H), 1.08 (s, 3H), 1.07 (s, 3H).

### 3S,8R,9S,10R,13S,14S)-10,13-Dimethyl-17-(p-tolyl)-2,3,4,7,8,9,10,11,12,13,14,15-dodecahydro-1H-cyclopenta[a]phenanthren-3-ol (195)

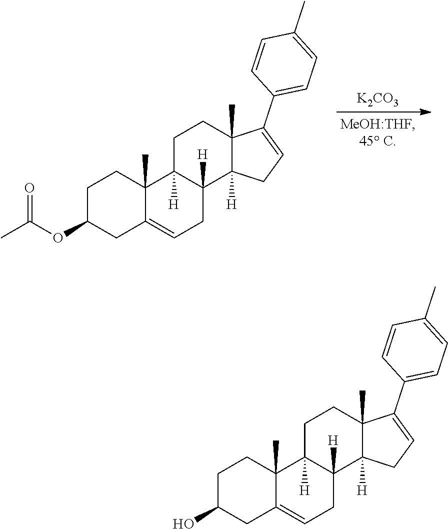

Synthesized according to the general procedure for acetate deprotection described above. Sterol i-27b (341 mg, 843 μmol), K₂CO₃ (1.17 g, 8.43 mmol), MeOH (28 mL), and THE (7.0 mL). The crude material was purified by silica gel chromatography (0-20-40-60-80-100% EtOAc:hexanes) to afford the product (184 mg, 60%) as a white solid. ¹H NMR: (300 MHz, CDCl₃) δ 7.27 (d, J=9.0 Hz, 2H), 7.11 (d, J=9.0 Hz, 2H), 5.87 (dd, J=6.0, 3.0 Hz, 1H), 5.39 (br d, J=3.0 Hz, 1H), 3.61-3.47 (m, 1H), 2.40-2.15 (m, 3H), 2.33 (s, 3H), 2.13-1.97 (m, 3H), 1.90-1.43 (m, 10H), 1.17-1.00 (m, 2H), 1.07 (s, 3H), 1.05 (s, 3H).

### (3S,8R,9S,10R,13S,14S)-17-(4-Isopropylphenyl)-10,13-dimethyl-2,3,4,7,8,9,10,11,12,13,14,15-dodecahydro-1H-cyclopenta[a]phenanthren-3-ol (196)

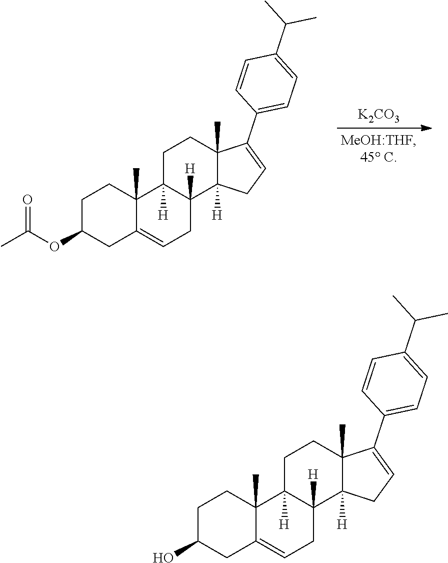

Synthesized according to the general procedure for acetate deprotection described above. Sterol i-27c (356 mg, 823 μmol), K₂CO₃ (1.14 g, 8.23 mmol), MeOH (27 mL), and THE (6.9 mL). The crude material was purified by silica gel chromatography (0-20-40-60-80-100% EtOAc:hexanes) to afford the product (313 mg, 97%) as a white solid. ¹H NMR: (300 MHz, CDCl₃) δ 7.32 (d, J=9.0 Hz, 2H), 7.16 (d, J=9.0 Hz, 2H), 5.89 (dd, J=3.0, 3.0 Hz, 1H), 5.40 (d, J=6.0 Hz, 1H), 3.62-3.47 (m, 1H), 2.89 (septet, 1H), 2.38-1.96 (m, 6H), 1.91-1.41 (m, 10H), 1.26 (d, J=9.0 Hz, 6H), 1.18-0.99 (m, 2H), 1.08 (s, 3H), 1.07 (s, 3H).

### (3S,8R,9S,10R,13S,14S)-17-(4-(tert-Butyl)phenyl)-10,13-dimethyl-2,3,4,7,8,9,10,11,12,13,14,15-dodecahydro-1H-cyclopenta[a]phenanthren-3-ol (197)

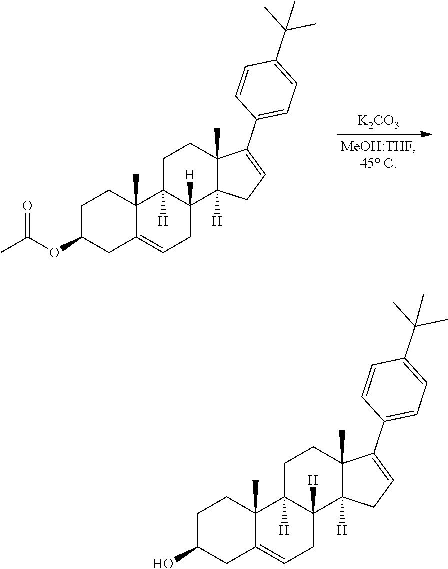

Synthesized according to the general procedure for acetate deprotection described above. Sterol i-27d (378 mg, 823 μmol), K₂CO₃ (1.17 g, 8.46 mmol), MeOH (27 mL), and THE (7.1 mL). The crude material was purified by silica gel chromatography (0-20-40-60-80-100% EtOAc:hexanes) to afford the product (313 mg, 91%) as a white solid. ¹H NMR: (300 MHz, CDCl₃) δ 7.32 (br s, 4H), 5.90 (dd, J=3.0, 3.0 Hz, 1H), 5.40 (d, J=6.0 Hz, 1H), 3.62-3.46 (m, 1H), 2.38-1.96 (m, 6H), 1.90-1.41 (m, 10H), 1.32 (s, 9H), 1.18-0.99 (m, 2H), 1.07 (s, 3H), 1.06 (s, 3H).

### (3S,8R,9S,10R,13S,14S)-10,13-Dimethyl-17-(m-tolyl)-2,3,4,7,8,9,10,11,12,13,14,15-dodecahydro-1H-cyclopenta[a]phenanthren-3-ol (198)

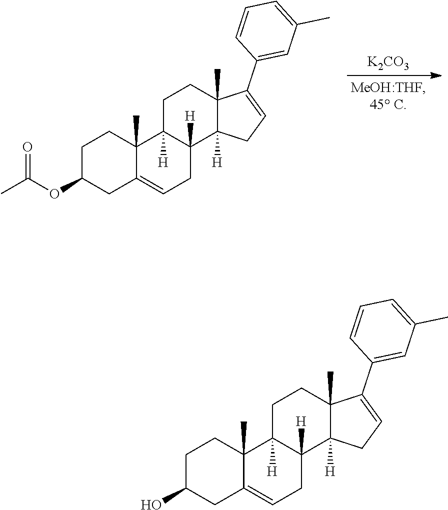

Synthesized according to the general procedure for acetate deprotection described above. Sterol i-27e (336 mg, 831 μmol), K₂CO₃ (1.15 g, 8.31 mmol), MeOH (27 mL), and THE (6.9 mL). The crude material was purified by silica gel chromatography (0-20-40-60-80-100% EtOAc:hexanes) to afford the product (263 mg, 87%) as a white solid. ¹H NMR: (300 MHz, CDCl₃) δ 7.24-7.15 (m, 3H), 7.10-7.02 (m, 1H), 5.90 (dd, J=6.0, 3.0 Hz, 1H), 5.40 (br d, J=6.0 Hz, 1H), 3.62-3.47 (m, 1H), 2.35 (s, 3H), 2.35-2.16 (m, 3H), 2.14-1.97 (m, 3H), 1.90-1.40 (m, 10H), 1.17-0.99 (m, 2H), 1.08 (s, 3H), 1.06 (s, 3H).

### (3S,8R,9S,10R,13S,14S)-17-(3,5-Dimethylphenyl)-10,13-dimethyl-2,3,4,7,8,9,10,11,12,13,14,15-dodecahydro-1H-cyclopenta[a]phenanthren-3-ol (199)

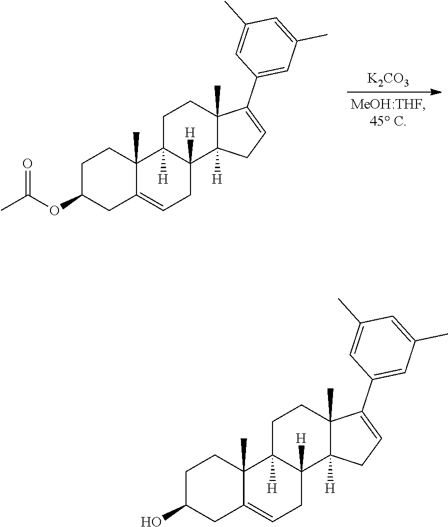

Synthesized according to the general procedure for acetate deprotection described above. Sterol i-27f (380 mg, 908 μmol), K₂CO₃ (1.26 g, 9.08 mmol), MeOH (30 mL), and THE (7.6 mL). The crude material was purified by silica gel chromatography (0-60% EtOAc:hexanes) to afford the product (271 mg, 79%) as a white solid. ¹H NMR: (300 MHz, CDCl₃) δ 6.99 (br s, 2H), 6.89 (br s, 1H), 5.88 (dd, J=6.0, 3.0 Hz, 1H), 5.40 (d, J=6.0 Hz, 1H), 3.61-3.47 (m, 1H), 2.40-2.15 (m, 3H), 2.31 (s, 6H), 2.14-1.96 (m, 3H), 1.91-1.40 (m, 10H), 1.18-1.00 (m, 2H), 1.08 (s, 3H), 1.06 (s, 3H).

### (3S,8R,9S,10R,13S,14S)-10,13-Dimethyl-17-(o-tolyl)-2,3,4,7,8,9,10,11,12,13,14,15-dodecahydro-1H-cyclopentaaLphenanthren-3-ol (20)

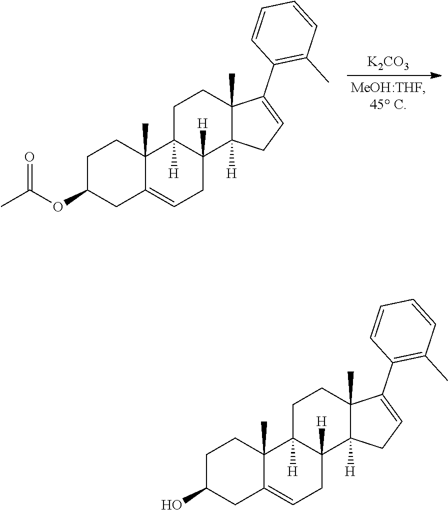

Synthesized according to the general procedure for acetate deprotection described above. Sterol i-27g (375 mg, 927 μmol), K₂CO₃ (1.28 g, 9.27 mmol), MeOH (31 mL), and THE (7.7 mL). The crude material was purified by silica gel chromatography (0-60% EtOAc:hexanes) to afford the product (307 mg, 91%) as a white solid. ¹H NMR: (300 MHz, CDCl₃) δ 7.25-7.05 (m, 4H), 5.59 (dd, J=3.0, 3.0 Hz, 1H), 5.41 (br d, J=6.0 Hz, 1H), 3.62-3.46 (m, 1H), 2.40-2.19 (m, 3H), 2.31 (s, 3H), 2.15-2.04 (m, 2H), 1.97 (br s, 1H), 1.90-1.43 (m, 10H), 1.18-1.04 (m, 2H), 1.07 (s, 3H), 0.96 (s, 3H).

### (3S,8R,9S,10R,13S,14S)-17-(2,6-Dimethylphenyl)-10,13-dimethyl-2,3,4,7,8,9,10,11,12,13,14,15-dodecahydro-1H-cyclopenta[a]phenanthren-3-ol (21)

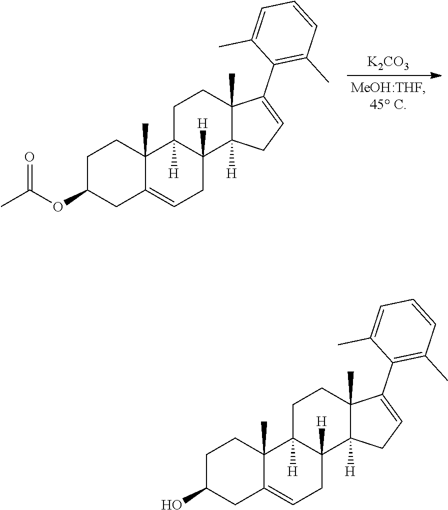

Synthesized according to the general procedure for acetate deprotection described above. Sterol i-27h (135 mg, 323 μmol), K₂CO₃ (446 mg, 3.23 mmol), MeOH (11 mL), and THE (5.4 mL; a 0.06 M amount of THE was used in this case to achieve solubility). The crude material was purified by silica gel chromatography (0-60% EtOAc:hexanes) to afford the product (107 mg, 88%) as a white solid. ¹H NMR: (300 MHz, CDCl₃) δ 7.11-6.96 (m, 3H), 5.53 (dd, J=6.0, 3.0 Hz, 1H), 5.41 (br d, J=6.0 Hz, 1H), 3.62-3.47 (m, 1H), 2.39-2.02 (m, 5H), 2.29 (s, 3H), 2.26 (s, 3H), 1.92-1.41 (m, 11H), 1.17-1.02 (m, 2H), 1.05 (s, 3H), 0.96 (s, 3H).

### Example 29. General Procedure C for Reduction

The sterol (1 equiv.) was added to a steel parr reactor equipped with a stir bar and dissolved in THE (0.07 M). Ethanol (0.05 M) and palladium hydroxide on carbon (1 equiv.) were subsequently added to the reactor. The parr reactor was sealed, evacuated, and refilled with H₂ gas (3×), and the pressure was set to 100 psi. The reaction was stirred at 500 rpm at rt for 18 hours. The vessel was then evacuated, refilled with N₂ gas, and opened. The crude reaction mixture was filtered through a Celite pad. The Celite pad was washed with MeOH and the crude material was concentrated and purified as indicated below.

### (3S,8R,9S,10S,13S,14S,17S)-10,13-Dimethyl-17-phenylhexadecahydro-1H-cyclopenta[a]phenanthren-3-ol (200)

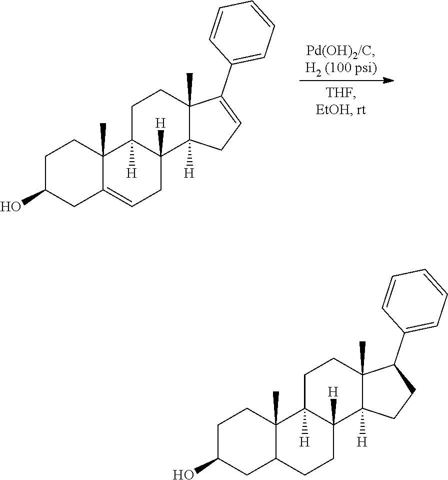

Synthesized according to general procedure C for reduction described above. Sterol 19 (80.0 mg, 230 μmol), Pd(OH)₂/C (32.2 mg, 230 μmol), THF (3.3 mL), and EtOH (4.6 mL). The crude material was purified by silica gel chromatography (0-20-40-60-80% EtOAc:hexanes) to afford the product (62 mg, 77%) as a white solid. ¹H NMR: (300 MHz, CDCl₃) δ 7.32-7.24 (m, 2H), 7.23-7.14 (m, 3H), 3.67-3.53 (m, 1H), 2.67 (dd, J=9.0, 9.0 Hz, 1H), 2.17-2.01 (m, 1H), 2.01-1.86 (m, 1H), 1.85-1.66 (m, 4H), 1.63-1.50 (m, 3H), 1.50-1.06 (m, 11H), 1.05-0.88 (m, 2H), 0.80 (s, 3H), 0.70 (ddd, J=12.0, 12.0, 3.0 Hz, 1H), 0.46 (s, 3H).

### (3S,8R,9S,10S,13S,14S,17S)-10,13-Dimethyl-17-(p-tolyl)hexadecahydro-1H-cyclopenta[a]phenanthren-3-ol (201)

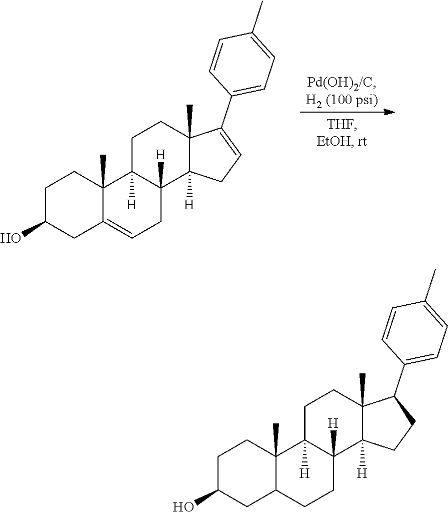

Synthesized according to general procedure C for reduction described above. Sterol 195 (90.0 mg, 248 μmol), Pd(OH)₂/C (34.9 mg, 248 μmol), THF (3.5 mL), and EtOH (5.0 mL). The crude material was purified by silica gel chromatography (0-20-40-60-80% EtOAc:hexanes) to afford the product (53 mg, 58%) as a white solid. ¹H NMR: (300 MHz, CDCl₃) δ 7.09 (s, 4H), 3.67-3.53 (m, 1H), 2.63 (dd, J=9.0, 9.0 Hz, 1H), 2.32 (s, 3H), 2.14-1.87 (m, 2H), 1.86-1.66 (m, 4H), 1.62-1.49 (m, 3H), 1.48-1.07 (m, 11H), 1.05-0.88 (m, 2H), 0.80 (s, 3H), 0.70 (ddd, J=9.0, 9.0, 3.0 Hz, 1H), 0.45 (s, 3H).

### (3S,8R,9S,10S,13S,14S,17S)-17-(4-Isopropylphenyl)-10,13-dimethylhexadecahydro-1H-cyclopenta[a]phenanthren-3-ol (202)

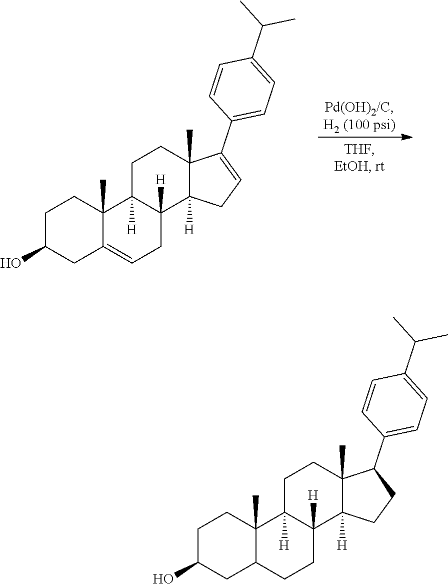

Synthesized according to general procedure C for reduction described above. Sterol 196 (120 mg, 307 μmol), Pd(OH)₂/C (43.1 mg, 307 μmol), THF (4.4 mL), and EtOH (6.1 mL). The crude material was purified by silica gel chromatography (0-20-40-60-80% EtOAc:hexanes) to afford the product (77 mg, 64%) as a white solid. ¹H NMR: (300 MHz, CDCl₃) δ 7.13 (br s, 4H), 3.67-3.52 (m, 1H), 2.88 (septet, 1H), 2.64 (dd, J=9.0, 9.0 Hz, 1H), 2.15-1.86 (m, 2H), 1.86-1.66 (m, 4H), 1.63-1.49 (m, 4H), 1.49-1.07 (m, 10H), 1.25 (d, J=6.0 Hz, 6H), 1.07-0.88 (m, 2H), 0.81 (s, 3H), 0.70 (ddd, J=12.0, 12.0, 6.0 Hz, 1H), 0.46 (s, 3H).

### (3S,8R,9S,10S,13S,14S,17S)-17-(4-(tert-Butyl)phenyl)-10,13-dimethylhexadecahydro-1H-cyclopenta[a]phenanthren-3-ol (203)

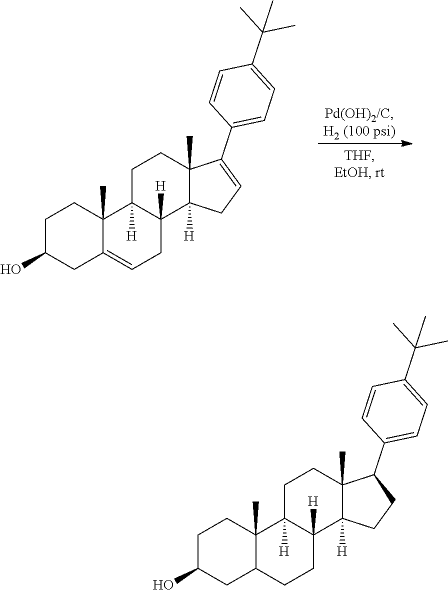

Synthesized according to general procedure C for reduction described above. Sterol 197 (120 mg, 297 μmol), Pd(OH)₂/C (41.6 mg, 297 μmol), THF (4.2 mL), and EtOH (5.9 mL). The crude material was purified by silica gel chromatography (0-20-40-60-80% EtOAc:hexanes) to afford the product (105 mg, 87%) as a white solid. ¹H NMR: (300 MHz, CDCl₃) δ 7.29 (d, J=6.0 Hz, 2H), 7.13 (d, J=9.0 Hz, 2H), 3.67-3.53 (m, 1H), 2.64 (dd, J=9.0, 9.0 Hz, 1H), 2.15-2.00 (m, 1H), 2.00-1.87 (m, 1H), 1.86-1.68 (m, 4H), 1.64-1.48 (m, 4H), 1.48-1.07 (m, 10H), 1.31 (s, 9H), 1.06-0.88 (m, 2H), 0.81 (s, 3H), 0.71 (ddd, J=12.0, 12.0, 6.0 Hz, 1H), 0.47 (s, 3H).

### (3S,8R,9S,10S,13S,14S,17S)-10,13-Dimethyl-17-(m-tolyl)hexadecahydro-1H-cyclopenta[a]phenanthren-3-ol (204)

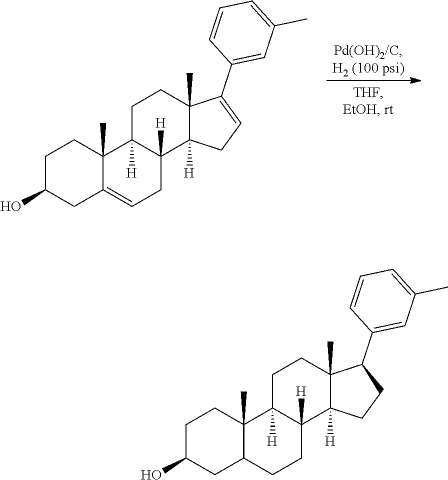

Synthesized according to general procedure C for reduction described above. Sterol 198 (120 mg, 331 μmol), Pd(OH)₂/C (46.5 mg, 331 μmol), THF (4.7 mL), and EtOH (6.6 mL). The crude material was purified by silica gel chromatography (0-20-40-60-80% EtOAc:hexanes) to afford the product (87 mg, 72%) as a white solid. ¹H NMR: (300 MHz, CDCl₃) δ 7.22-7.12 (m, 1H), 7.01 (br d, J=6.0 Hz, 3H), 3.67-3.52 (m, 1H), 2.63 (dd, J=9.0, 9.0 Hz, 1H), 2.34 (s, 3H), 2.16-2.01 (m, 1H), 1.99-1.87 (m, 1H), 1.86-1.67 (m, 4H), 1.66-1.50 (m, 4H), 1.49-1.07 (m, 10H), 1.05-0.89 (m, 2H), 0.81 (s, 3H), 0.71 (ddd, J=12.0, 12.0, 6.0 Hz, 1H), 0.46 (s, 3H).

### (3S,8R,9S,10S,13S,14S,17S)-17-(3,5-Dimethylphenyl)-10,13-dimethylhexadecahydro-1H-cyclopenta[a]phenanthren-3-ol (205)

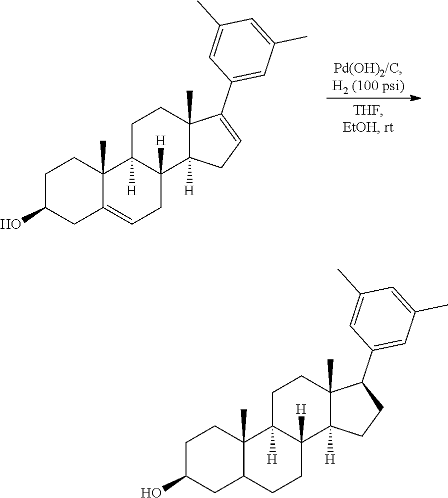

Synthesized according to general procedure C for reduction described above. Sterol 199 (120 mg, 319 μmol), Pd(OH)₂/C (44.7 mg, 319 μmol), THF (4.6 mL), and EtOH (6.4 mL). The crude material was purified by silica gel chromatography (0-20-40-60-80% EtOAc:hexanes) to afford the product (109 mg, 90%) as a clear oil. ¹H NMR: (300 MHz, CDCl₃) δ 6.85 (br s, 1H), 6.82 (br s, 2H), 3.67-3.53 (m, 1H), 2.60 (dd, J=9.0, 9.0 Hz, 1H), 2.31 (s, 6H), 2.15-2.00 (m, 1H), 1.99-1.86 (m, 1H), 1.86-1.68 (m, 4H), 1.64-1.51 (m, 4H), 1.49-1.08 (m, 10H), 1.06-0.90 (m, 2H), 0.82 (s, 3H), 0.71 (ddd, J=12.0, 12.0, 3.0 Hz, 1H), 0.48 (s, 3H).

### (3S,8R,9S,10S,13S,14S,17S)-10,13-Dimethyl-17-(o-tolyl)hexadecahydro-1H-cyclopenta[a]phenanthren-3-ol (206)

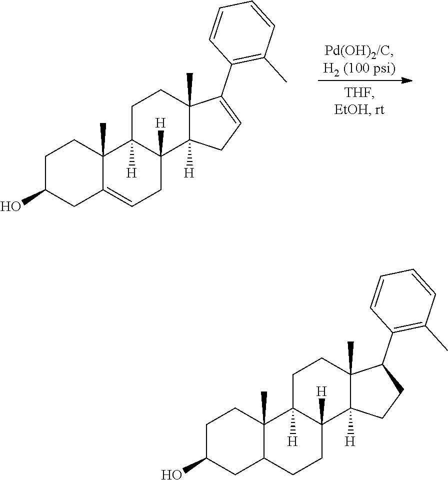

Synthesized according to general procedure C for reduction described above. Sterol 20 (120 mg, 331 μmol), Pd(OH)₂/C (46.5 mg, 331 μmol), THF (4.7 mL), and EtOH (6.6 mL). The crude material was purified by silica gel chromatography (0-20-40-60-80% EtOAc:hexanes) to afford the product (83 mg, 68%) as a white solid. ¹H NMR: (300 MHz, CDCl₃) δ 7.35-7.27 (br m, 1H), 7.19-7.03 (br m, 3H), 3.67-3.52 (m, 1H), 3.06 (dd, J=9.0, 9.0 Hz, 1H), 2.35 (s, 3H), 2.03-1.91 (m, 2H), 1.86-1.65 (m, 4H), 1.64-1.06 (m, 14H), 1.05-0.88 (m, 2H), 0.82 (s, 3H), 0.71 (ddd, J=12.0, 12.0, 3.0 Hz, 1H), 0.62 (s, 3H).

### (3S,8R,9S,10S,13S,14S,17S)-17-(2,6-Dimethylphenyl)-10,13-dimethylhexadecahydro-1H-cyclopenta[a]phenanthren-3-ol (207)

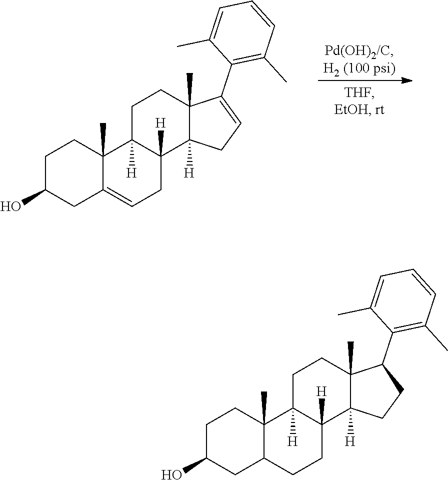

Synthesized according to general procedure C for reduction described above. Sterol 21 (55.0 mg, 146 μmol), Pd(OH)₂/C (20.5 mg, 146 μmol), THE (2.1 mL), and EtOH (2.9 mL). The crude material was purified by silica gel chromatography (0-20-40-60-80% EtOAc:hexanes) to afford the product (45 mg, 81%) as a white solid. ¹H NMR: (300 MHz, CDCl₃) δ 6.99 (br s, 3H), 3.67-3.54 (m, 1H), 3.38 (dd, J=9.0, 6.0 Hz, 1H), 2.45 (br s, 3H), 2.38 (br s, 3H), 2.34-2.19 (m, 1H), 1.96-1.09 (m, 19H), 1.06-0.89 (m, 2H), 0.81 (s, 3H), 0.72 (ddd, J=12.0, 12.0, 6.0 Hz, 1H), 0.70 (s, 3H).

### Example 30. Synthesis of (3S,8S,9S,10R,13S,14S,17S)-10,13-Dimethyl-17-(2-methyl-1,3-dioxolan-2-yl)-2,3,4,7,8,9,10,11,12,13,14,15,16,17-tetradecahydro-1H-cyclopenta[a]phenanthren-3-ol (208)

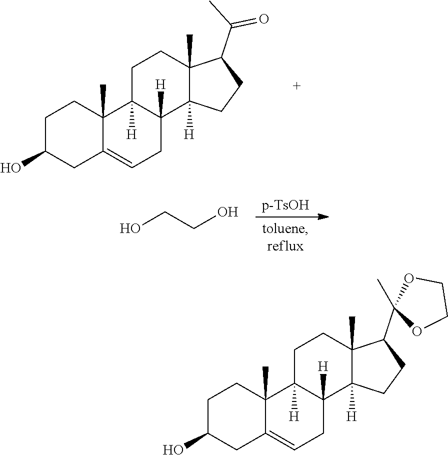

A mixture of pregnenolone (3.00 g, 9.48 mmol), toluene (95 mL), ethylene glycol (636 μL, 11.4 mmol), and p-toluenesulfonic acid (90.1 mg, 474 μmol), in a flask equipped with a Dean-Stark apparatus was heated to reflux overnight. The reaction was cooled to rt and the mixture was diluted with EtOAc and water. The layers were separated, and the organic layer was washed with saturated aqueous NaHCO₃ (2×) and brine (2×). The organic layer was dried (MgSO₄), filtered, and concentrated. The crude material was purified by silica gel chromatography (0-50% EtOAc:hexanes) to afford the product (182 mg, 5%) as a white solid. ¹H NMR: (300 MHz, MeOD) δ 5.34 (br d, J=6.0 Hz, 1H), 4.03-3.79 (m, 4H), 3.46-3.32 (m, 1H), 2.29-2.15 (m, 2H), 2.09 (ddd, J=12.0. 3.0, 3.0 Hz, 1H), 2.03-1.39 (m, 13H), 1.30-0.88 (m, 5H), 1.27 (s, 3H), 1.02 (s, 3H), 0.80 (s, 3H).

### Example 31. Synthesis of (3S,8S,9S,10R,13S,14S,17S)-10,13-Dimethyl-17-(2-methyl-1,3-dioxan-2-yl)-2,3,4,7,8,9,10,11,12,13,14,15,16,17-tetradecahydro-1H-cyclopenta[a]phenanthren-3-ol (209)

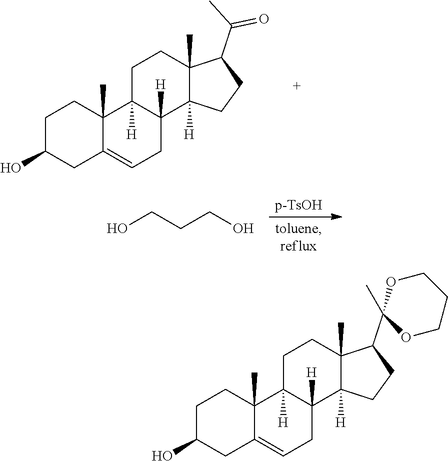

A mixture of pregnenolone (3.00 g, 9.48 mmol), toluene (95 mL), 1,3-propandiol (817 μL, 11.4 mmol), and p-toluenesulfonic acid (90.1 mg, 474 μmol), in a flask equipped with a Dean-Stark apparatus was heated to reflux overnight. The reaction was cooled to rt and the mixture was diluted with EtOAc and water. The layers were separated, and the organic layer was washed with saturated aqueous NaHCO₃ (2×) and brine (2×). The organic layer was dried (MgSO₄), filtered, and concentrated. The crude material was purified by silica gel chromatography (0-50% EtOAc:hexanes) to afford the product (93 mg, 3%) as a white solid. ¹H NMR: (300 MHz, CDCl₃) δ 5.34 (br d, J=6.0 Hz, 1H), 3.97 (dddd, J=12.0, 12.0, 6.0, 3.0 Hz, 2H), 3.89-3.74 (m, 2H), 3.59-3.43 (m, 1H), 2.33-2.10 (m, 3H), 2.06-1.76 (m, 5H), 1.72-0.78 (m, 15H), 1.41 (s, 3H), 1.01 (s, 3H), 0.84 (s, 3H).

### Example 32. Synthesis of (3S,8S,9S,10R,13R,14S,17R)-17-((2R)-5-Hydroxy-5-methylheptan-2-yl)-10,13-dimethyl-2,3,4,7,8,9,10,11,12,13,14,15,16,17-tetradecahydro-1H-cyclopenta[a]phenanthren-3-ol (210)

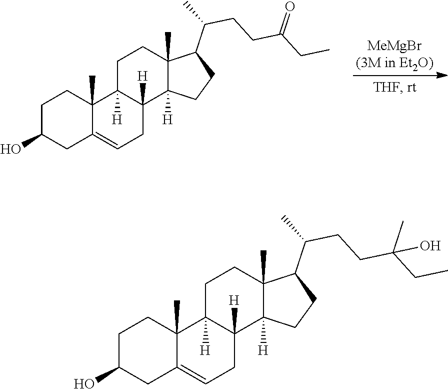

To a solution of the sterol 158 (160 mg, 414 μmol) dissolved in THE (3.6 mL) was added dropwise MeMgBr (3 M in Et₂O, 690 μL, 2.07 mmol) at room temperature. The resulting mixture was allowed to stir at room temperature overnight prior to being quenched with saturated aqueous NH₄Cl. The layers were separated, and the aqueous layer was extracted with EtOAc (2×). The organic extracts were combined, dried (MgSO₄), filtered, and concentrated. The crude material was purified by silica gel chromatography (0-20-40-60-80% EtOAc:hexanes) to afford the product (141 mg, 85%) as a white solid. ¹H NMR: (300 MHz, CDCl₃) δ 5.35 (br d, J=6.0 Hz, 1H), 3.59-3.45 (m, 1H), 2.34-2.16 (m, 2H), 2.06-1.91 (m, 2H), 1.90-1.76 (m, 3H), 1.64-1.22 (m, 15H), 1.22-0.83 (m, 13H), 1.13 (s, 3H), 1.01 (s, 3H), 0.68 (s, 3H).

### Example 33. Synthesis of (3S,8S,9S,10R,13R,14S,17R)-17-((2R)-5-Ethyl-5-hydroxyoctan-2-yl)-10,13-dimethyl-2,3,4,7,8,9,10,11,12,13,14,15,16,17-tetradecahydro-1H-cyclopenta[a]phenanthren-3-ol (211)

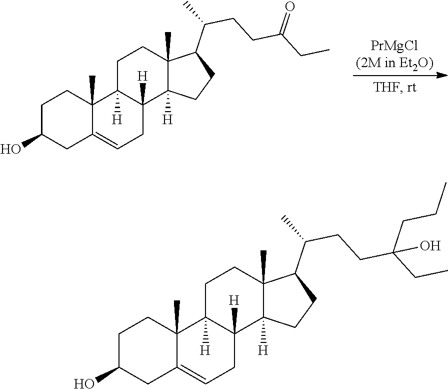

To a solution of the sterol 158 (160 mg, 414 μmol) dissolved in THF (3.6 mL) was added dropwise PrMgCl (2 M in Et₂O, 1.04 mL, 2.07 mmol) at room temperature. The resulting mixture was allowed to stir at room temperature overnight prior to being quenched with saturated aqueous NH₄Cl. The layers were separated, and the aqueous layer was extracted with EtOAc (2×). The organic extracts were combined, dried (MgSO₄), filtered, and concentrated. The crude material was purified by silica gel chromatography (0-20-40-60-80% EtOAc:hexanes) to afford the product (146 mg, 82%) as a white solid. ¹H NMR: (300 MHz, CDCl₃) δ 5.34 (br d, J=6.0 Hz, 1H), 3.59-3.43 (m, 1H), 2.35-2.14 (m, 2H), 2.04-1.91 (m, 2H), 1.91-1.77 (m, 3H), 1.64-1.18 (m, 19H), 1.18-0.79 (m, 16H), 1.00 (s, 3H), 0.67 (s, 3H).

### Example 34. (3S,8R,9S,10S,13R,14S,17R)-17-((2R)-5-Hydroxy-5-methylheptan-2-yl)-10,13-dimethylhexadecahydro-1H-cyclopenta[a]phenanthren-3-ol (212)

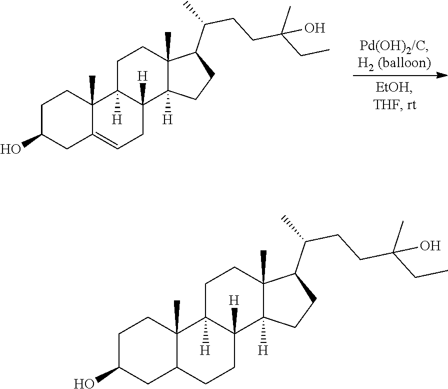

To a flask equipped with a stir bar was added Pd(OH)₂/C (27.9 mg, 199 μmol). The unsaturated sterol 210 (80 mg, 199 μmol) dissolved in THF (2.8 mL) was added to the flask followed by the addition of EtOH (7.1 mL). The flask was sealed with a septum, evacuated, and backfilled with N₂ gas. This process was repeated a total of three times followed by a final evacuation. A H₂ balloon was inserted through the septum, and the resulting reaction was allowed to stir overnight at room temperature. The reaction was then filtered through a Celite pad, and the filtrate was concentrated. The crude material was purified by silica gel chromatography (0-10-20-40-60-80% EtOAc:hexanes) to afford the product (55 mg, 68%) as a white solid. ¹H NMR: (300 MHz, CDCl₃) δ 3.66-3.52 (m, 1H), 1.95 (ddd, J=12.0, 3.0, 3.0 Hz, 1H), 1.90-1.18 (m, 22H), 1.17-0.84 (m, 14H), 1.12 (s, 3H), 0.80 (s, 3H), 0.65 (s, 3H), 0.62 (ddd, J=12.0, 12.0, 6.0 Hz, 1H).

### Example 35. Synthesis of (3S,8R,9S,10S,13R,14S,17R)-17-((2R)-5-Ethyl-5-hydroxyoctan-2-yl)-10,13-dimethylhexadecahydro-1H-cyclopenta[a]phenanthren-3-ol (213)

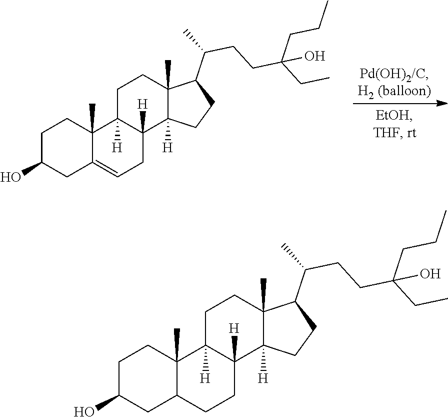

To a flask equipped with a stir bar was added Pd(OH)₂/C (26.1 mg, 186 μmol). The unsaturated sterol 211 (80 mg, 186 μmol) dissolved in THE (2.6 mL) was added to the flask followed by the addition of EtOH (6.6 mL). The flask was sealed with a septum, evacuated, and backfilled with N₂ gas. This process was repeated a total of three times followed by a final evacuation. A H₂ balloon was inserted through the septum, and the resulting reaction was allowed to stir overnight at room temperature. The reaction was then filtered through a Celite pad, and the filtrate was concentrated. The crude material was purified by silica gel chromatography (0-10-20-40-60-80% EtOAc:hexanes) to afford the product (65 mg, 81%) as a white solid. ¹H NMR: (300 MHz, CDCl₃) δ 3.65-3.51 (m, 1H), 1.95 (ddd, J=12.0, 3.0, 3.0 Hz, 1H), 1.89-1.17 (m, 26H), 1.16-0.75 (m, 17H), 0.79 (s, 3H), 0.64 (s, 3H), 0.61 (ddd, J=12.0, 12.0, 3.0 Hz, 1H).

### Example 36. Synthesis of (3S,8R,9S,10R,13S,14S)-10,13-Dimethyl-1,2,3,4,7,8,9,10,11,12,13,14,15,16-tetradecahydrospiro[cyclopenta[a]phenanthrene-17,2′-[1,3]dioxolan]-3-ol (223)

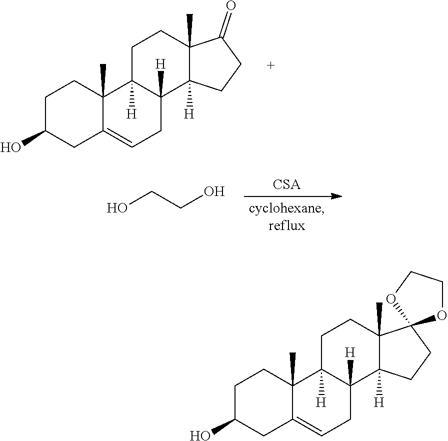

To a solution of dehydroepiandrosterone (1.00 g, 3.47 mmol) in cyclohexane (100 mL) was added ethylene glycol (582 μL, 10.4 mmol) and camphorsulfonic acid (9.7 mg, 42.0 μmol). The resulting mixture was heated to reflux for 4 hours using a Dean-Stark apparatus. The reaction mixture was cooled to rt, and diluted with EtOAc. Layers were separated and the organic layer was washed with saturated aqueous NaHCO₃ and brine. The organic layer was dried (MgSO₄), filtered, and concentrated to afford a crude white solid. The crude material was purified by silica gel chromatography (0-20-40-60-80% EtOAc:hexanes) to afford the product (913 mg, 79%) as a white solid. ¹H NMR: (300 MHz, MeOD) δ 5.35 (br d, J=6.0 Hz, 1H), 3.96-3.79 (m, 4H), 3.47-3.33 (m, 1H), 2.31-2.13 (m, 2H), 2.07-1.19 (m, 16H), 1.11 (dd, J=12.0, 3.0 Hz, 1H), 1.03 (s, 3H), 0.95 (ddd, J=9.0, 9.0, 3.0 Hz, 1H), 0.86 (s, 3H).

### Example 37. Synthesis of (3S,8R,9S,10R,13S,14S)-10,13-Dimethyl-1,2,3,4,7,8,9,10,11,12,13,14,15,16-tetradecahydrospiro[cyclopenta[a]phenanthrene-17,2′-[1,3]dioxan]-3-ol (224)

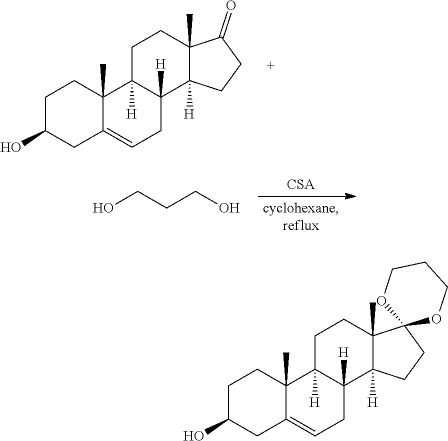

To a solution of dehydroepiandrosterone (1.00 g, 3.47 mmol) in cyclohexane (100 mL) was added 1,3-propandiol (747 μL, 10.4 mmol) and camphorsulfonic acid (9.7 mg, 42.0 μmol). The resulting mixture was heated to reflux for 4 hours using a Dean-Stark apparatus. The reaction mixture was cooled to rt, and diluted with EtOAc. Layers were separated and the organic layer was washed with saturated aqueous NaHCO₃ and brine. The organic layer was dried (MgSO₄), filtered, and concentrated to afford a crude white solid. The crude material was purified by silica gel chromatography (0-80% EtOAc:hexanes) to afford the product (639 mg, 53%) as a white solid. ¹H NMR: (300 MHz, MeOD) δ 5.34 (br d, J=6.0 Hz, 1H), 4.03 (ddd, J=12.0, 12.0, 3.0 Hz, 1H), 3.89-3.76 (m, 3H), 3.46-3.33 (m, 1H), 2.37 (ddd, J=12.0, 9.0, 6.0 Hz, 1H), 2.29-2.13 (m, 2H), 2.07-1.20 (m, 17H), 1.09 (dd, J=12.0, 3.0 Hz, 1H), 1.03 (s, 3H), 0.98-0.86 (m, 1H), 0.79 (s, 3H).

### Example 38. Synthesis of (((3S,8S,9S,10R,13R,14S,17R)-10,13-Dimethyl-17-((R,E)-5-propyloct-5-en-2-yl)-2,3,4,7,8,9,10,11,12,13,14,15,16,17-tetradecahydro-1H-cyclopenta[a]phenanthren-3-yl)oxy)triisopropylsilane (i-38)

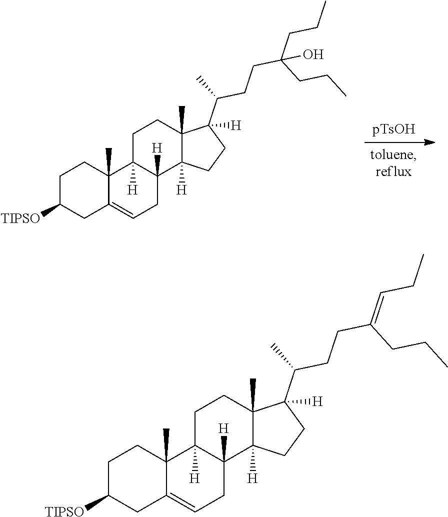

The tertiary alcohol (95.0 mg, 158 μmol) was dissolved in toluene (1 mL), and a catalytic amount of p-toluenesulfonic acid (3.01 mg, 16.0 μmol) was added. The resulting mixture was refluxed overnight. The solution was then cooled to rt and diluted with EtOAc. The organic layer was washed with water, dried (MgSO₄), filtered, and concentrated. The crude material was purified by silica gel chromatography (0-1-2-5-10% EtOAc:hexanes) to afford the product (59.0 mg, 64%) as a clear oil and as a series of regio- and geometric isomers. ¹H NMR: (300 MHz, CDCl₃, reported as seen in spectrum) δ 6.02-5.85 (m, 0.33H), 5.65-5.46 (m, 0.39H), 5.44-5.27 (m, 1.32H), 5.20-5.00 (m, 1.31H), 3.65-3.48 (m, 1H), 2.37-1.67 (m, 17.8H), 1.67-0.80 (m, 61.5H), 0.77-0.61 (m, 4H).

### Example 39. Synthesis of (3S,8S,9S,10R,13R,14S,17R)-10,13-Dimethyl-17-((R,E)-5-propyloct-5-en-2-yl)-2,3,4,7,8,9,10,11,12,13,14,15,16,17-tetradecahydro-1H-cyclopenta[a]phenanthren-3-ol (225)

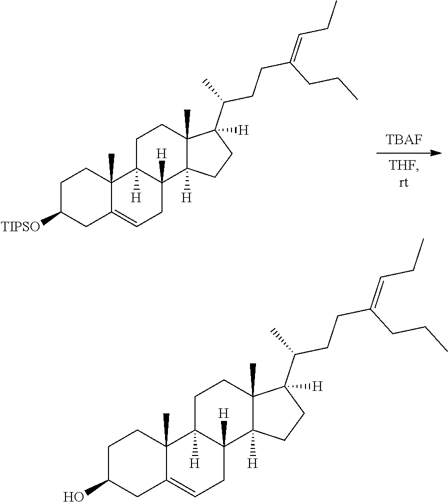

To a vial equipped with a stir bar was added the sterol i-38 (59.0 mg, 101 μmol) and THF (1.0 mL). TBAF (1.0 M in THF, 506 μL, 506 μmol) was added and the resulting mixture was allowed to stir at rt for 2 h. Reaction was then quenched with saturated aqueous NaHCO₃ and the layers were separated. Aqueous layer was extracted with EtOAc (2×) and organic extracts were combined, dried (MgSO₄), filtered, and concentrated. The crude material was purified by silica gel chromatography (10-20-40-60% EtOAc:hexanes) to afford the product (24.0 mg, 56%) as a white solid. ¹H NMR: (300 MHz, CDCl₃) δ 5.35 (br d, J=6.0 Hz, 1H), 5.16-5.02 (br m, 1H), 3.60-3.44 (m, 1H), 2.36-2.16 (m, 2H), 2.13-1.66 (m, 11H), 1.63-1.23 (m, 13H), 1.22-0.81 (m, 18H), 0.68 (s, 3H).

### Example 40. Synthesis of (3S,8R,9S,10S,13R,14S,17R)-10,13-Dimethyl-17-((R)-5-propyloctan-2-yl)hexadecahydro-1H-cyclopenta[a]phenanthren-3-ol (226)

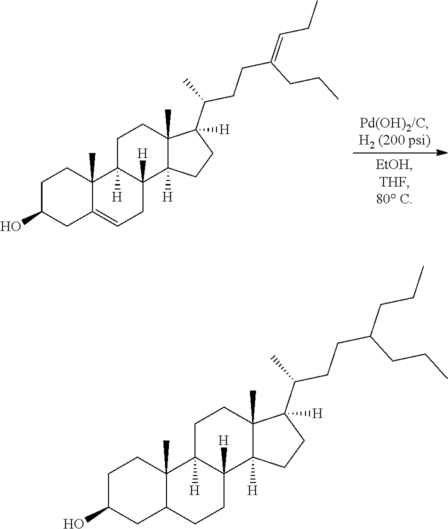

The unsaturated sterol 225 (26.0 mg, 60.9 μmol) was added to a steel parr reactor equipped with a stir bar and dissolved in THF (1 mL). EtOH (2 mL) and palladium hydroxide on carbon (8.6 mg, 60.9 μmol) were subsequently added to the reactor. The parr reactor was sealed, evacuated, and refilled with H₂ gas (3×), and the pressure was set to 200 psi. The reaction was stirred at 500 rpm at 80° C. for 3 h. The vessel was then evacuated, refilled with N₂ gas, and opened. The crude reaction mixture was filtered through a Celite pad. The Celite pad was washed with MeOH and the crude material was concentrated. The crude material was purified by silica gel chromatography (0-10-20-40-70% EtOAc:hexanes) to afford the product (20 mg, 76%) as a white solid. ¹H NMR: (300 MHz, CDCl₃) δ 3.66-3.51 (m, 1H), 1.95 (ddd, J=12.0, 3.0, 3.0 Hz, 1H), 1.88-1.40 (m, 8H), 1.39-0.82 (m, 37H), 0.80 (s, 3H), 0.64 (s, 3H), 0.62 (br ddd, J=15.0, 12.0, 6.0 Hz, 1H).

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

1. A compound having the structure of Formula I: 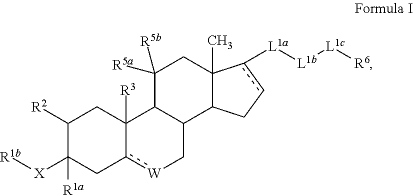
wherein
R^{1a} is H, optionally substituted C₁-C₆ alkyl, optionally substituted C₂-C₆ alkenyl, or optionally substituted C₂-C₆ alkynyl;
X is O or S;
R^{1b} is H, optionally substituted C₁-C₆ alkyl, or 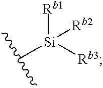
each of R^{b1}, R^{b2}, and R^{b3} is, independently, optionally substituted C₁-C₆ alkyl or optionally substituted C₆-C₁₀ aryl;
R² is H or OR^{A}, wherein R^{A} is H or optionally substituted C₁-C₆ alkyl;
R³ is H or 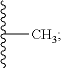
each [symbol] independently represents a single bond or a double bond;
W is CR^{4a} or CR^{4a}R^{4b}, wherein if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form 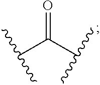 [SMILES: *C(*)=O]
L^{1a} is absent, 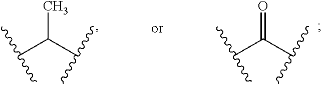
L^{1b} is absent, e 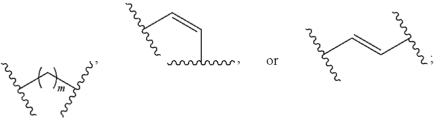
m is 1, 2, or 3;
L^{1c} is absent, 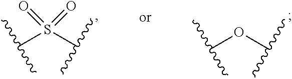
and
R⁶ is optionally substituted C₃-C₂₀ cycloalkyl, optionally substituted C₃-C₂₀ cycloalkenyl, optionally substituted C₆-C₂₀ aryl, optionally substituted C₂-C₁₉ heterocyclyl, or optionally substituted C₂-C₁₉ heteroaryl,
or a pharmaceutically acceptable salt thereof.

2. The compound of claim 1, wherein the compound has the structure of Formula Ia: 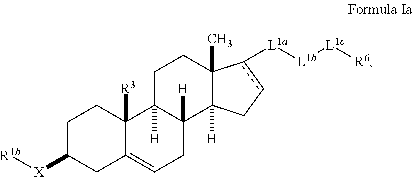
or a pharmaceutically acceptable salt thereof.

3. The compound of claim 1, wherein the compound has the structure of Formula Ib: 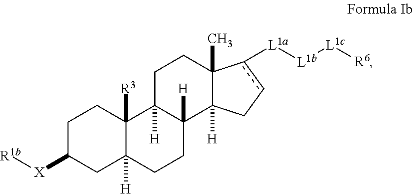
or a pharmaceutically acceptable salt thereof.

4. The compound of claim 1, wherein the compound has the structure of Formula Ic: 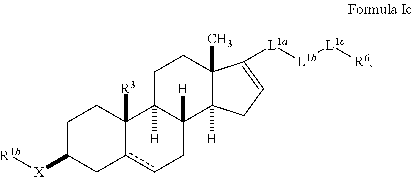
or a pharmaceutically acceptable salt thereof.

5. The compound of claim 1, wherein the compound has the structure of Formula Id: 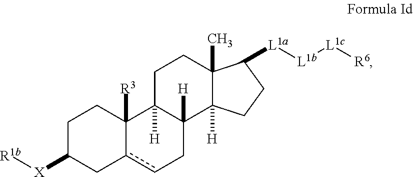
or a pharmaceutically acceptable salt thereof.

6. The compound of any one of claims 1 to 5, wherein L^{1a} is absent.

7. The compound of any one of claims 1 to 5, wherein L^{1a} is

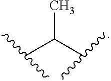 [SMILES: *C(*)C]

8. The compound of any one of claims 1 to 5, wherein L^{1a} is

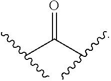 [SMILES: *C(*)=O]

9. The compound of any one of claims 1 to 8, wherein L^{1b} is absent.

10. The compound of any one of claims 1 to 8, wherein L^{1b} is

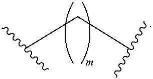

11. The compound of claim 10, wherein m is 1 or 2.

12. The compound of any one of claims 1 to 8, wherein L^{1b} is

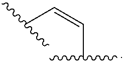 [SMILES: */C=C\*]

13. The compound of any one of claims 1 to 8, wherein L^{1b} is

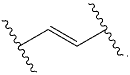 [SMILES: */C=C/*]

14. The compound of any one of claims 1 to 13, wherein L^{1c} is absent.

15. The compound of any one of claims 1 to 13, wherein L^{1c} is

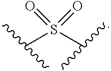 [SMILES: *S(*)(=O)=O]

16. The compound of any one of claims 1 to 13, wherein L^{1c} is

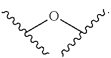 [SMILES: *O*]

17. The compound of any one of claims 1 to 16, wherein R⁶ is optionally substituted C₆-C₂₀ aryl.

18. The compound of claim 17, wherein R⁶ is optionally substituted C₆-C₁₂ aryl.

19. The compound of claim 18, wherein R⁶ is optionally substituted C₆-C₁₀ aryl.

20. The compound of claim 19, wherein R⁶ is 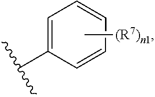
wherein
n1 is 0, 1, 2, 3, 4, or 5; and
each R⁷ is, independently, halo or optionally substituted C₁-C₆ alkyl.

21. The compound of claim 20, wherein each R⁷ is, independently,

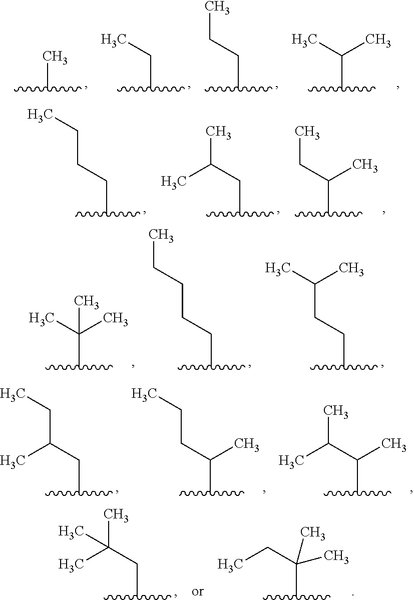

22. The compound of claim 21, wherein n1 is 0, 1, or 2.

23. The compound of claim 22, wherein R⁶ is

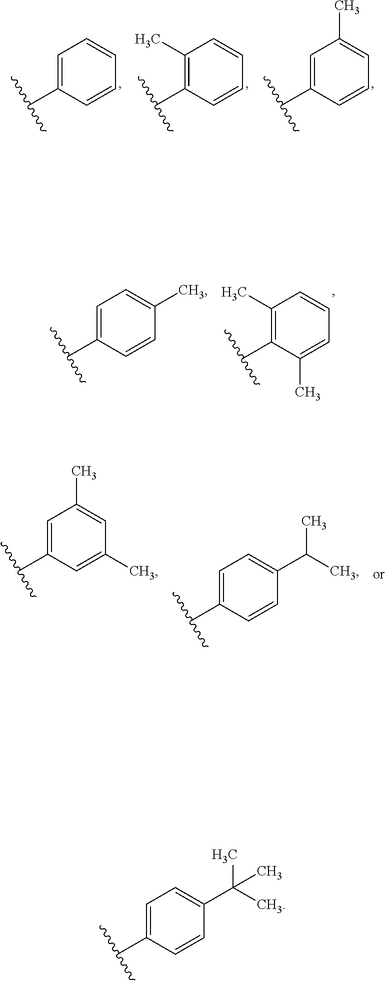

24. The compound of any one of claims 1 to 16, wherein R⁶ is optionally substituted C₃-C₂₀ cycloalkyl.

25. The compound of claim 24, wherein R⁶ is optionally substituted C₃-C₁₂ cycloalkyl.

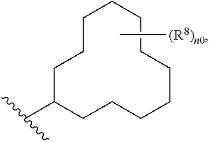

26. The compound of claim 25, wherein R⁶ is, wherein
n0 is 0, 1, 2, 3, 4, 5, 6, 7, 8, 9, 10, 11, 12, 13, 14, 15, 16, 17, 18, 19, 20, 21, 22, or 23; and
each R⁸ is, independently, halo or optionally substituted C₁-C₆ alkyl.

27. The compound of claim 26, wherein each R⁸ is, independently,

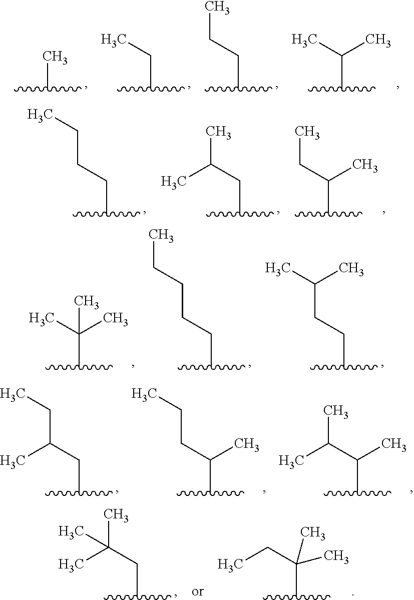

28. The compound of claim 27, wherein n0 is 0, 1, 2, 3, 4, 5, or 6.

29. The compound of claim 28, wherein R⁶ is

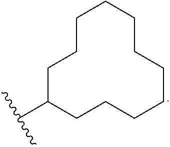 [SMILES: *C1CCCCCCCCCCC1]

30. The compound of claims 1 to 16, wherein R⁶ is optionally substituted C₃-C₁₀ cycloalkyl.

31. The compound of claim 30, wherein R⁶ is optionally substituted C₃-C₁₀ monocycloalkyl.

32. The compound of claim 31, wherein R⁶ is 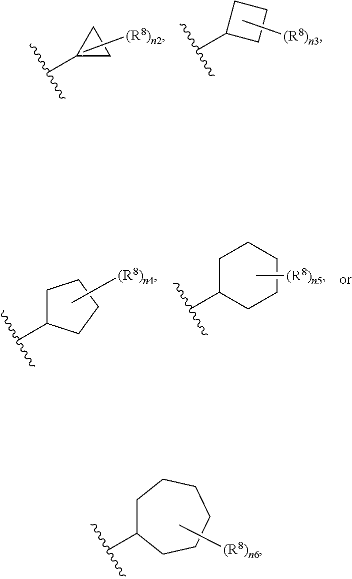
wherein
n2 is 0, 1, 2, 3, 4, or 5;
n3 is 0, 1, 2, 3, 4, 5, 6, or 7;
n4 is 0, 1, 2, 3, 4, 5, 6, 7, 8, or 9;
n5 is 0, 1, 2, 3, 4, 5, 6, 7, 8, 9, 10, or 11;
n6 is 0, 1, 2, 3, 4, 5, 6, 7, 8, 9, 10, 11, 12, or 13; and
each R⁸ is, independently, halo or optionally substituted C₁-C₆ alkyl.

33. The compound of claim 32, wherein each R⁸ is, independently,

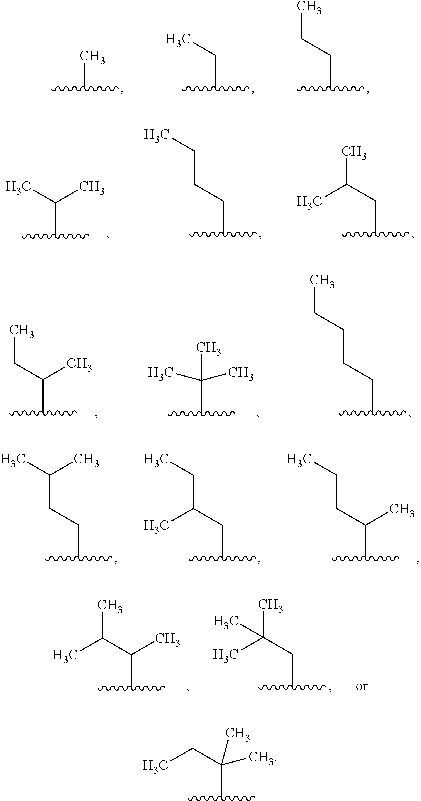

34. The compound of claim 33, wherein n2 is 0 or 1.

35. The compound of claim 34, wherein R⁶ is

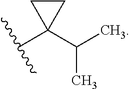 [SMILES: *C1(C(C)C)CC1]

36. The compound of claim 33, wherein n3 is 0 or 1.

37. The compound of claim 36, wherein R⁶ is

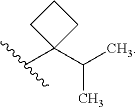 [SMILES: *C1(C(C)C)CCC1]

38. The compound of claim 33, wherein n4 is 0, 1, or 2.

39. The compound of claim 38, wherein R⁶ is

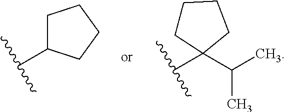

40. The compound of claim 33, wherein n5 is 0, 1, 2, or 3.

41. The compound of claim 40, wherein R⁶ is

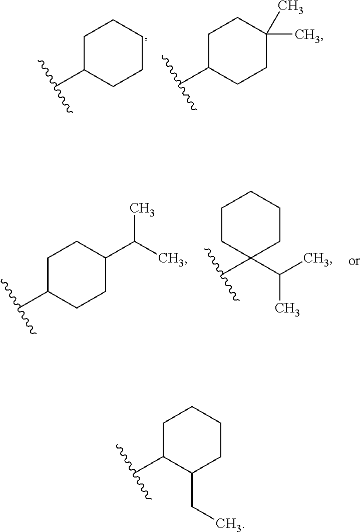

42. The compound of claim 33, wherein n6 is 0, 1, 2, 3, or 4.

43. The compound of claim 42, wherein R⁶ is

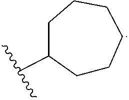 [SMILES: *C1CCCCCC1]

44. The compound of claim 30, wherein R⁶ is optionally substituted C₃-C₁₀ polycycloalkyl.

45. The compound of claim 44, wherein R⁶ is

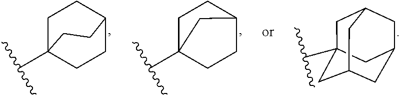 [SMILES: *C12CCC(CC1)CC2 ; *C12CCC(CC1)C2 ; *C12CC3CC(CC(C3)C1)C2]

46. The compound of any one of claims 1 to 16, wherein R⁶ is optionally substituted C₃-C₂₀ cycloalkenyl.

47. The compound of claim 46, wherein R⁶ is optionally substituted C₃-C₁₂ cycloalkenyl.

48. The compound of claim 47, wherein R⁶ is optionally substituted C₃-C₁₀ cycloalkenyl.

49. The compound of claim 48, wherein R⁶ is 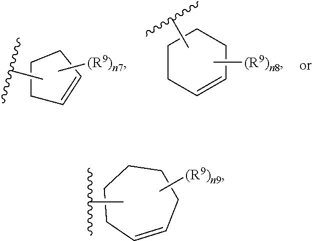
wherein
n7 is 0,1, 2, 3, 4, 5, 6, or 7;
n8 is 0, 1, 2, 3, 4, 5, 6, 7, 8, or 9;
n9 is 0, 1, 2, 3, 4, 5, 6, 7, 8, 9, 10, or 11; and
each R⁹ is, independently, halo or optionally substituted C₁-C₆ alkyl.

50. The compound of claim 49, wherein R⁶ is

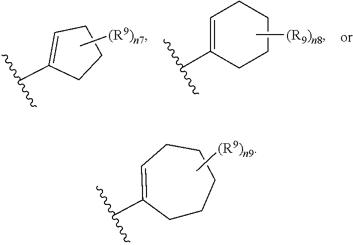

51. The compound of claim 49 or 50, wherein each R⁹ is, independently,

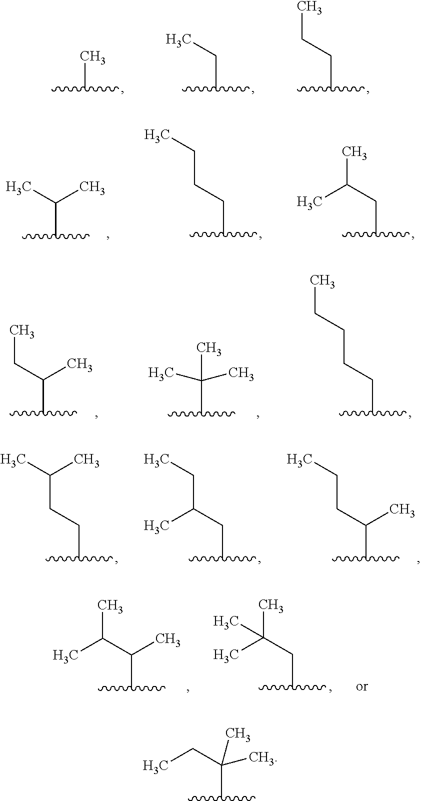

52. The compound of claim 51, wherein n7 is 0, 1, or 2.

53. The compound of claim 52, wherein R⁶ is

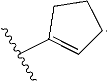 [SMILES: *C1=CCCC1]

54. The compound of claim 51, wherein n8 is 0, 1, 2, or 3.

55. The compound of claim 54, wherein R⁶ is

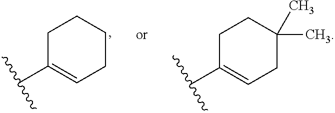

56. The compound of claim 51, wherein n9 is 0, 1, 2, 3, or 4.

57. The compound of claim 56, wherein R⁶ is

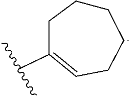 [SMILES: *C1=CCCCCC1]

58. The compound of any one of claims 1 to 16, wherein R⁶ is optionally substituted C₂-C₁₉ heterocyclyl.

59. The compound of claim 58, wherein R⁶ is optionally substituted C₂-C₁ heterocyclyl.

60. The compound of claim 59, wherein R⁶ is optionally substituted C₂-C₉ heterocyclyl.

61. The compound of claim 60, wherein R⁶ is 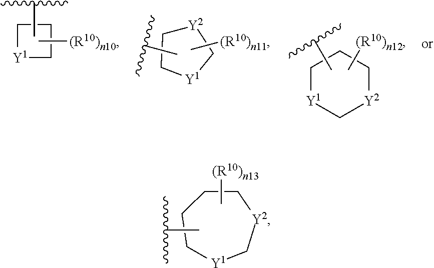
wherein
n10 is 0, 1, 2, 3, 4, or 5;
n11 is 0, 1, 2, 3, 4, 5, 6, or 7;
n12 is 0, 1, 2, 3, 4, 5, 6, 7, or 8;
n13 is 0, 1, 2, 3, 4, 5, 6, 7, 8, or 9;
each R¹⁰ is, independently, halo or optionally substituted C₁-C₆ alkyl; and
each of Y¹ and Y² is, independently, O, S, NR^{B}, or CR^{11a}R^{11b},
wherein R^{B} is H or optionally substituted C₁-C₆ alkyl;
each of R^{11a} and R^{11b} is, independently, H, halo, or optionally substituted C₁-C₆ alkyl; and
if Y² is CR^{11a}R^{11b}, then Y¹ is O, S, or NR^{B}.

62. The compound of claim 61, wherein Y¹ is O.

63. The compound of claim 61 or 62, wherein Y² is O.

64. The compound of claim 61 or 62, wherein Y² is CR^{11a}R^{11b}.

65. The compound of any one of claims 61 to 64, wherein each R¹⁰ is, independently,

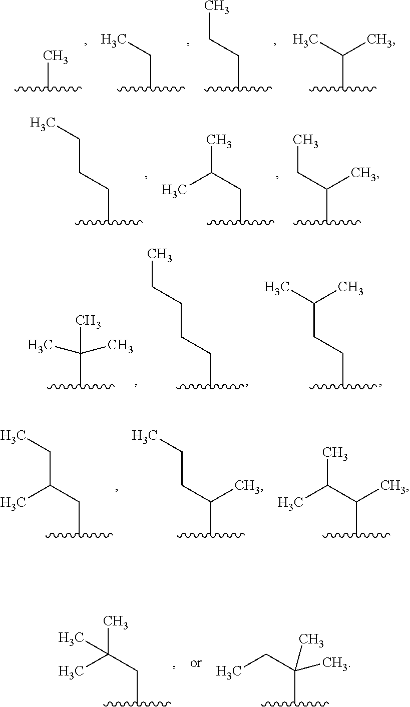

66. The compound of claim 65, wherein n10 is 0 or 1.

67. The compound of claim 66, wherein R⁶ is

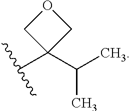 [SMILES: *C1(C(C)C)COC1]

68. The compound of claim 65, wherein n11 is 0, 1, 2, 3, 4, or 5.

69. The compound of claim 68, wherein R⁶ is

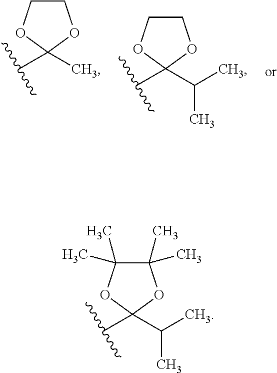

70. The compound of claim 65, wherein n12 is 0, 1, 2, 3, 4, 5, or 6.

71. The compound of claim 70, wherein R⁶ is CH₃, CH₃, or CH₃

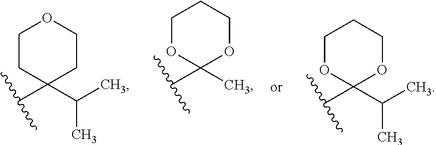

72. The compound of any one of claims 1 to 16, wherein R⁶ is optionally substituted C₂-C₁₉ heteroaryl.

73. The compound of claim 72, wherein R⁶ is optionally substituted C₂-C₁₁ heteroaryl.

74. The compound of claim 73, wherein R⁶ is optionally substituted C₂-C₉ heteroaryl.

75. The compound of claim 74, wherein R⁶ is 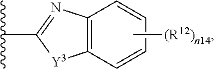
wherein
Y³ is NR^{C}, O, or S;
n14 is 0, 1, 2, 3, or 4;
R^{C} is H or optionally substituted C₁-C₆ alkyl; and
each R¹² is, independently, halo or optionally substituted C₁-C₆ alkyl.

76. The compound of claim 75, wherein each R¹² is, independently,

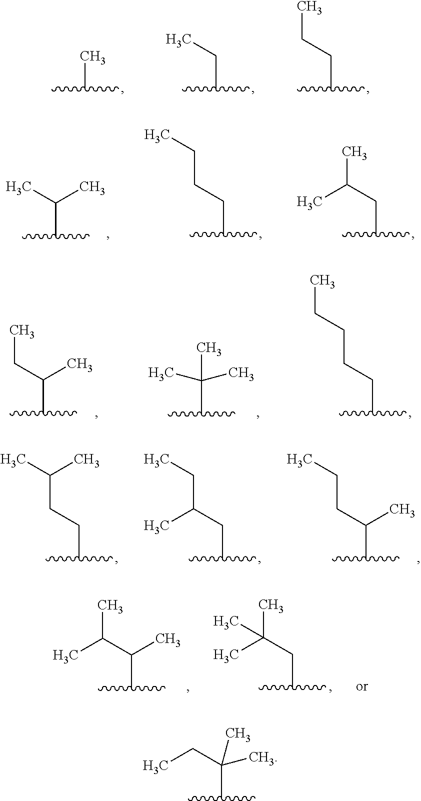

77. The compound of claim 76, wherein n14 is 0, 1, or 2.

78. The compound of any one of claims 75 to 77, wherein Y³ is S.

79. The compound of claim 78, wherein R⁶ is

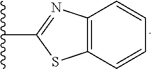 [SMILES: *c1nc2ccccc2s1]

80. The compound of claim any one of claims 75 to 77, wherein Y³ is NR^{C}.

81. The compound of claim 80, wherein R^{C} is H or

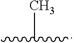

82. The compound of claim 81, wherein R⁶ is

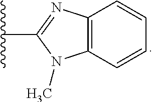 [SMILES: *c1nc2ccccc2n1C]

83. A compound having the structure of Formula II: 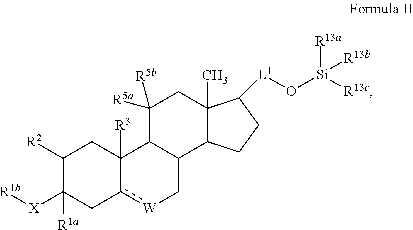
wherein
R^{1a} is H, optionally substituted C₁-C₆ alkyl, optionally substituted C₂-C₆ alkenyl, or optionally substituted C₂-C₆ alkynyl;
X is O or S;
R^{1b} is H or optionally substituted C₁-C₆ alkyl;
R² is H or OR^{A}, where R^{A} is H or optionally substituted C₁-C₆ alkyl;
R³ is H or 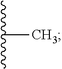 [symbol]
represents a single bond or a double bond;
W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form 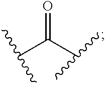 [SMILES: *C(*)=O]
L¹ is optionally substituted C₁-C₆ alkylene; and
each of R^{13a}, R^{13b}, and R^{13c} is, independently, optionally substituted C₁-C₆ alkyl or optionally substituted C₆-C₁₀ aryl,
or a pharmaceutically acceptable salt thereof.

84. The compound of 83, wherein the compound has the structure of Formula IIa: 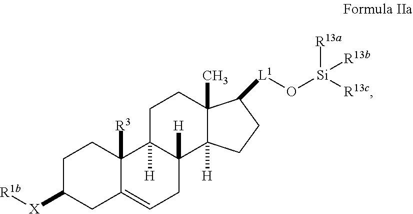
or a pharmaceutically acceptable salt thereof.

85. The compound of 83, wherein the compound has the structure of Formula IIb: 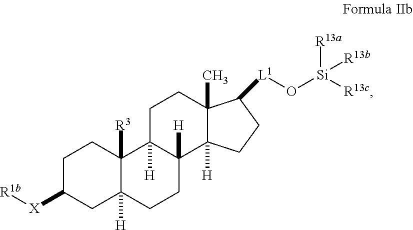
or a pharmaceutically acceptable salt thereof.

86. The compound of any one of claims 83 to 85, wherein each of R^{13a}, R^{13b}, and R^{13c} is, independently,

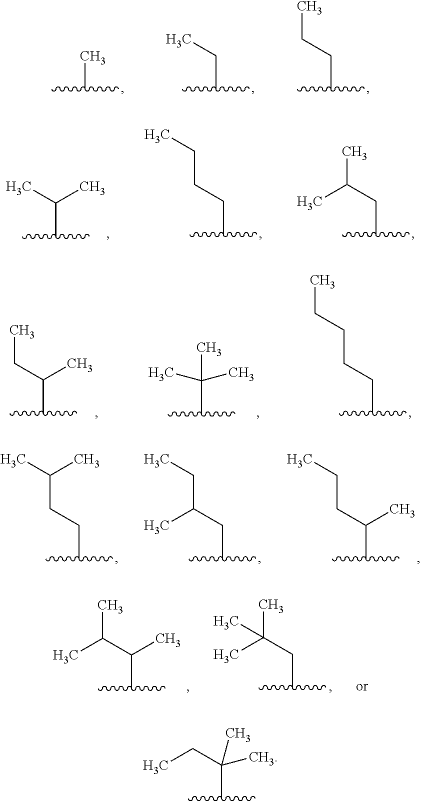

87. A compound having the structure of Formula III: 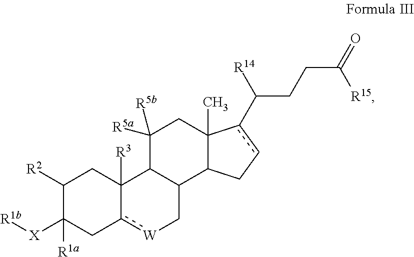
wherein
R^{1a} is H, optionally substituted C₁-C₆ alkyl, optionally substituted C₂-C₆ alkenyl, or optionally substituted C₂-C₆ alkynyl;
X is O or S;
R^{1b} is H or optionally substituted C₁-C₆ alkyl;
R² is H or OR^{A}, where R^{A} is H or optionally substituted C₁-C₆ alkyl;
R³ is H or 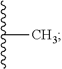
each [symbol] independently represents a single bond or a double bond;
W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, hydroxyl, optionally substituted C₁-C₆ alkyl, —OS(O)₂R^{4c}, where R^{4c} is optionally substituted C₁-C₆ alkyl or optionally substituted C₆-C₁₀ aryl;
each of R^{5a} and R^{5b} is, independently, H or OR^{A}, or R^{5a} and R^{5b}, together with the atom to which each is attached, combine to form 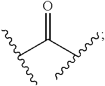 [SMILES: *C(*)=O]
R¹⁴ is H or C₁-C₆ alkyl; and
R¹⁵ is 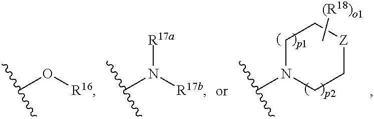
where
R¹⁶ is H or optionally substituted C₁-C₆ alkyl;
R^{17a} is H, optionally substituted C₆-C₁₀ aryl, or optionally substituted C₁-C₆ alkyl;
R^{17b} is H, OR^{17c}, optionally substituted C₆-C₁₀ aryl, or optionally substituted C₁-C₆ alkyl;
R^{17c} is H or optionally substituted C₁-C₆ alkyl;
o1 is 0, 1, 2, 3, 4, 5, 6, 7, or 8;
p1 is 0, 1, or 2;
p2 is 0, 1, or 2;
Z is CH₂, O, S, or NR^{D}, where R^{D} is H or optionally substituted C₁-C₆ alkyl; and
each R¹⁸ is, independently, halo or optionally substituted C₁-C₆ alkyl,
or a pharmaceutically acceptable salt thereof.

88. The compound of claim 87, wherein the compound has the structure of Formula IIIa: 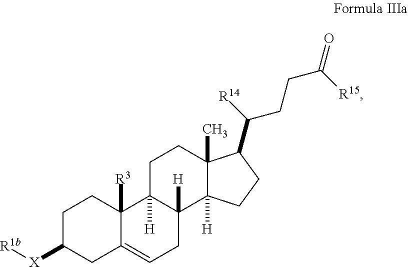
or a pharmaceutically acceptable salt thereof.

89. The compound of claim 87, wherein the compound has the structure of Formula IIIb: 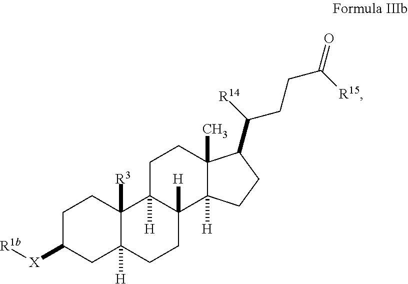
or a pharmaceutically acceptable salt thereof.

90. The compound of any one of claims 87 to 89, wherein R¹⁴ is

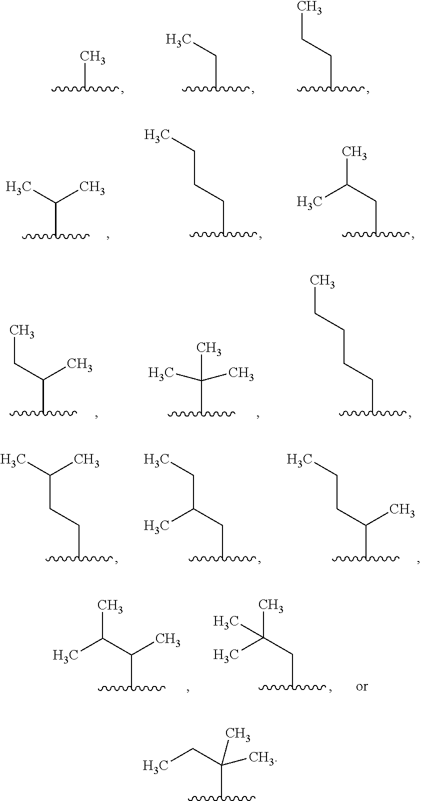

91. The compound of any one of claims 87 to 90, wherein R¹⁴ is

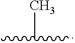

92. The compound of any one of claims 87 to 91, wherein R¹⁵ is

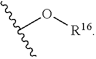

93. The compound of claim 92, wherein R¹⁶ is

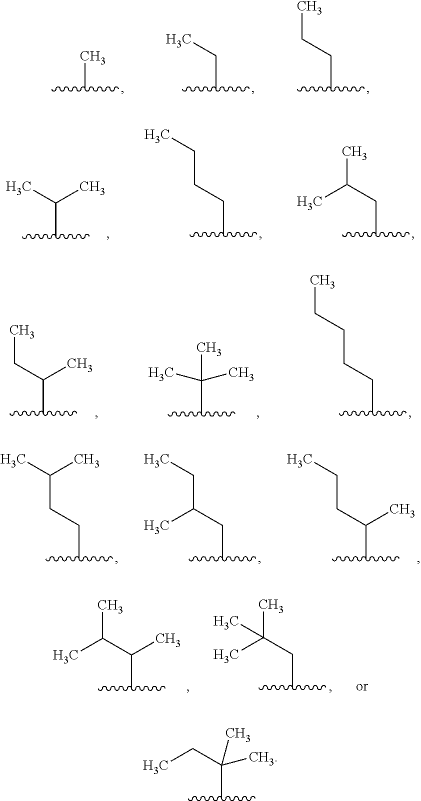

94. The compound of any one of claims 87 to 93, wherein R¹⁵ is

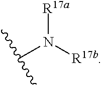

95. The compound of claim 94, wherein R^{17a} is H or optionally substituted C₁-C₆ alkyl.

96. The compound of claim 94 or 95, wherein R^{17b} is H or optionally substituted C₁-C₆ alkyl.

97. The compound of claim 94 or 95, wherein R^{17b} is optionally substituted C₆-C₁₀ aryl.

98. The compound of claim 94 or 95, wherein R^{17b} is OR^{17c}.

99. The compound of any one of claims 87 to 93, wherein R¹⁵ is

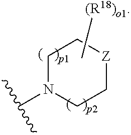

100. The compound of claim 99, wherein each R¹⁸ is, independently,

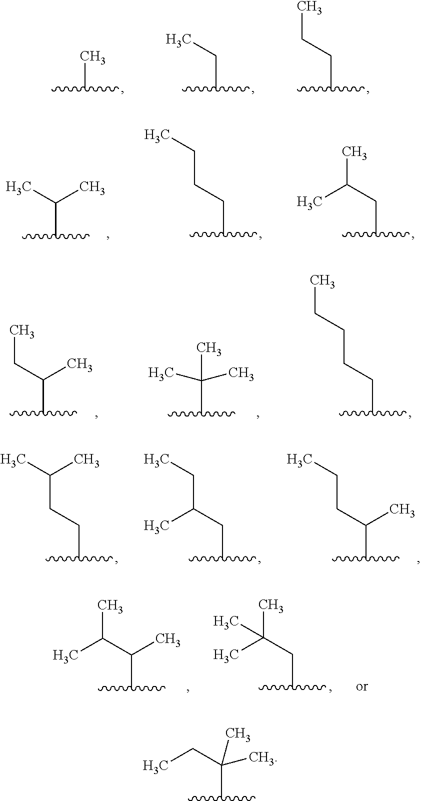

101. The compound of claim 99 or 100, wherein Z is CH₂.

102. The compound of claim 99 or 100, wherein Z is O or NR^{D}.

103. The compound of any one of claims 99 to 102, wherein p1 is 0 or 1.

104. The compound of any one of claims 99 to 103, wherein p2 is 0 or 1.

105. A compound having the structure of Formula IV: 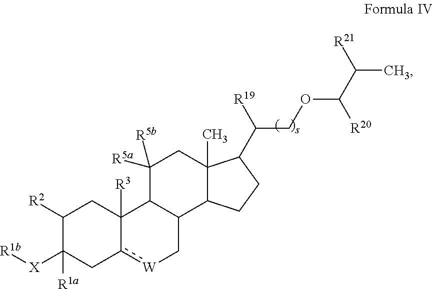
wherein
R^{1a} is H, optionally substituted C₁-C₆ alkyl, optionally substituted C₂-C₆ alkenyl, or optionally substituted C₂-C₆ alkynyl;
X is O or S;
R^{1b} is H or optionally substituted C₁-C₆ alkyl;
R² is H or OR^{A}, where R^{A} is H or optionally substituted C₁-C₆ alkyl;
R³ is H or 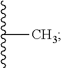 [symbol]
represents a single bond or a double bond;
W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form 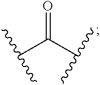 [SMILES: *C(*)=O]
s is 0 or 1;
R¹⁹ is H or C₁-C₆ alkyl;
R²⁰ is C₁-C₆ alkyl; and
R²¹ is H or C₁-C₆ alkyl,
or a pharmaceutically acceptable salt thereof.

106. The compound of claim 105, wherein the compound has the structure of Formula IVa: 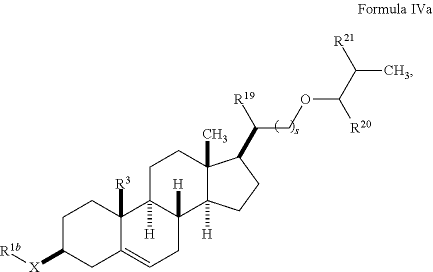
or a pharmaceutically acceptable salt thereof.

107. The compound of claim 105, wherein the compound has the structure of Formula IVb: 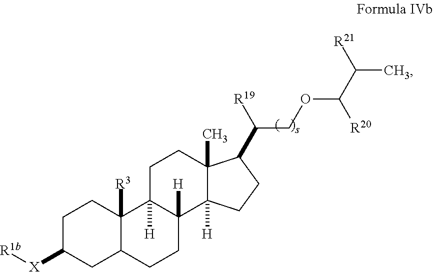
or a pharmaceutically acceptable salt thereof.

108. The compound of any one of claims 105 to 107, wherein each of R¹⁹, R²⁰, and R²¹ is, independently,

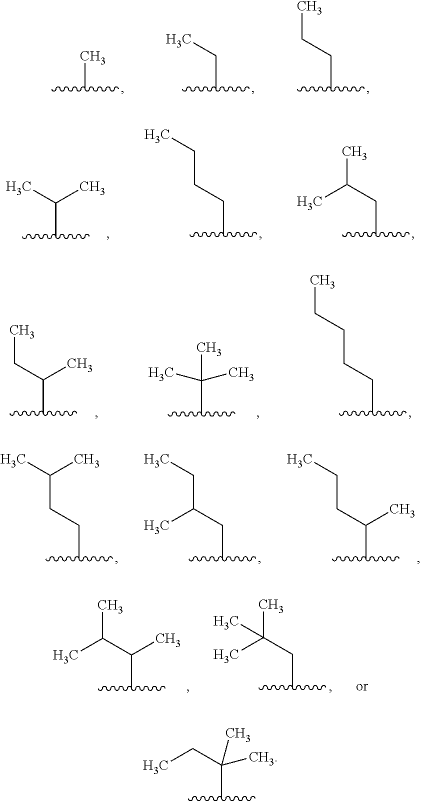

109. A compound having the structure of Formula V: 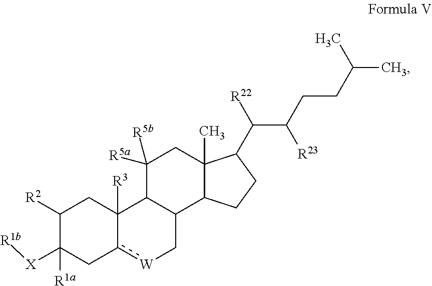
wherein
R^{1a} is H, optionally substituted C₁-C₆ alkyl, optionally substituted C₂-C₆ alkenyl, or optionally substituted C₂-C₆ alkynyl;
X is O or S;
R^{1b} is H or optionally substituted C₁-C₆ alkyl;
R² is H or OR^{A}, wherein R^{A} is H or optionally substituted C₁-C₆ alkyl;
R³ is H or 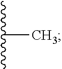 [symbol]
represents a single bond or a double bond;
W is CR^{4a} or CR^{4a}R^{4b}, wherein if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form 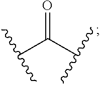 [SMILES: *C(*)=O]
R²² is H or C₁-C₆ alkyl; and
R²³ is halo, hydroxyl, optionally substituted C₁-C₆ alkyl, or optionally substituted C₁-C₆ heteroalkyl,
or a pharmaceutically acceptable salt thereof.

110. The compound of claim 109, wherein the compound has the structure of Formula Va: 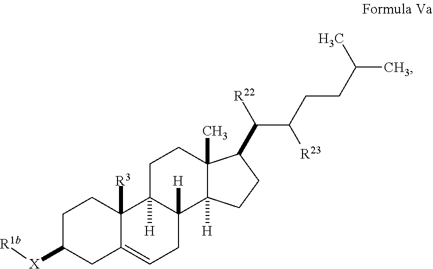
or a pharmaceutically acceptable salt thereof.

111. The compound of claim 109, wherein the compound has the structure of Formula Vb: 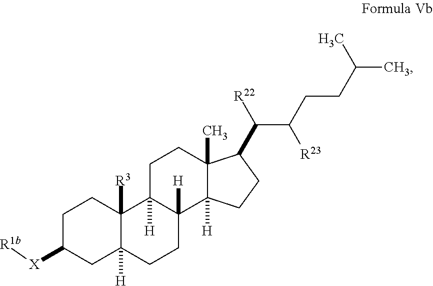
or a pharmaceutically acceptable salt thereof.

112. The compound of any one of claims 109 to 111, wherein each of R²² and R²³ is, independently,

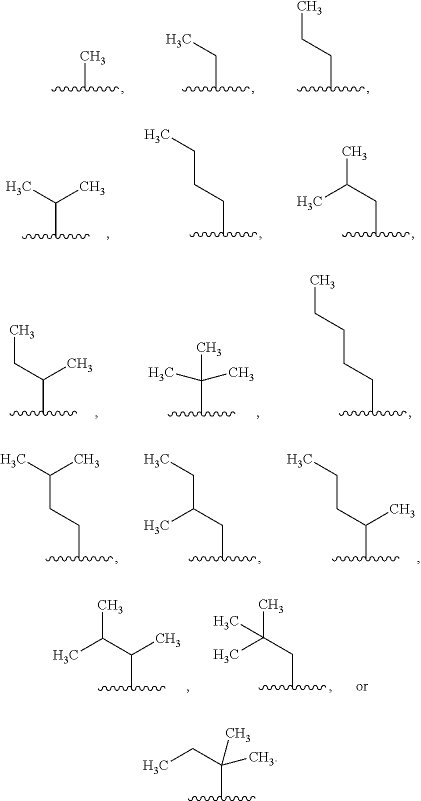

113. A compound having the structure of Formula VI: 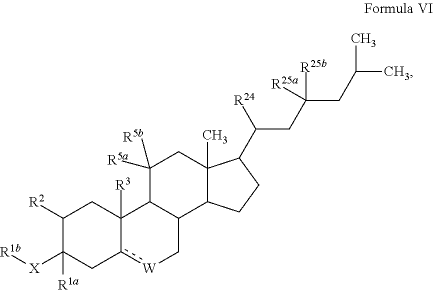
wherein
R^{1a} is H, optionally substituted C₁-C₆ alkyl, optionally substituted C₂-C₆ alkenyl, or optionally substituted C₂-C₆ alkynyl;
X is O or S;
R^{1b} is H or optionally substituted C₁-C₆ alkyl;
R² is H or OR^{A}, wherein R^{A} is H or optionally substituted C₁-C₆ alkyl;
R³ is H or 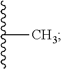 [symbol]
represents a single bond or a double bond;
W is CR^{4a} or CR^{4a}R^{4b}, wherein if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form 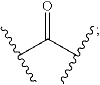 [SMILES: *C(*)=O]
R²⁴ is H or C₁-C₆ alkyl; and
each of R^{25a} and R^{25b} is C₁-C₆ alkyl,
or a pharmaceutically acceptable salt thereof.

114. The compound of claim 113, wherein the compound has the structure of Formula Via: 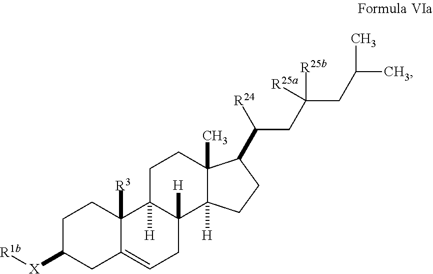
or a pharmaceutically acceptable salt thereof.

115. The compound of claim 113, wherein the compound has the structure of Formula VIb: 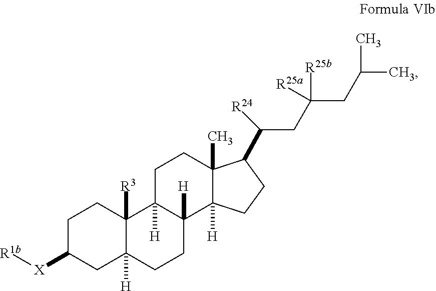
or a pharmaceutically acceptable salt thereof.

116. The compound of any one of claims 113 to 115, wherein each of R²⁴, R^{25a}, and R^{25b} is, independently,

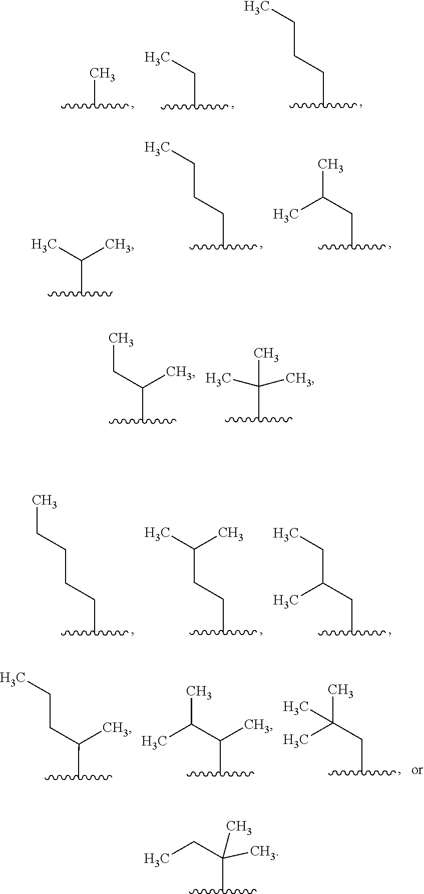

117. A compound having the structure of Formula VII: 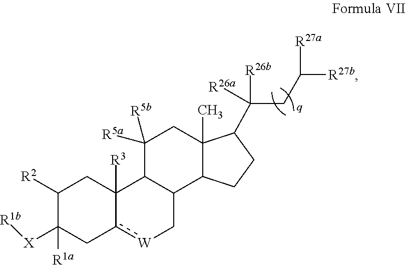
wherein
R^{1a} is H, optionally substituted C₁-C₆ alkyl, optionally substituted C₂-C₆ alkenyl, optionally substituted C₂-C₆ alkynyl, or 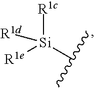
wherein each of R^{1c}, R^{1c}, and R^{1e} is, independently, optionally substituted C₁-C₆ alkyl or optionally substituted C₆-C₁₀ aryl;
X is O or S;
R^{1b} is H or optionally substituted C₁-C₆ alkyl;
R² is H or OR^{A}, wherein R^{A} is H or optionally substituted C₁-C₆ alkyl;
R³ is H or 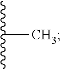 [symbol]
represents a single bond or a double bond;
W is CR^{4a} or CR^{4a}R^{4b}, wherein if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form 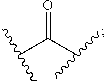 [SMILES: *C(*)=O]
q is 0 or 1;
each of R^{26a} and R^{26b} is, independently, H or optionally substituted C₁-C₆ alkyl, or R^{26a} and R^{26b}, together with the atom to which each is attached, combine to form 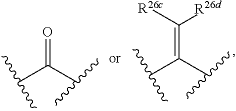
wherein each of R^{26c} and R²⁶ is, independently, H or optionally substituted C₁-C₆ alkyl; and
each of R^{27a} and R^{27b} is H, hydroxyl, or optionally substituted C₁-C₆ alkyl,
or a pharmaceutically acceptable salt thereof.

118. The compound of claim 117, wherein the compound has the structure of Formula VIIa: 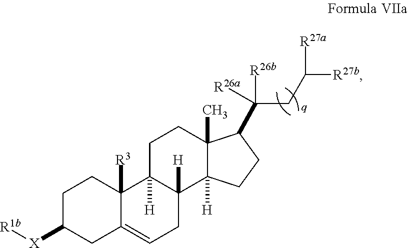
or a pharmaceutically acceptable salt thereof.

119. The compound of claim 117, wherein the compound has the structure of Formula VIIb: 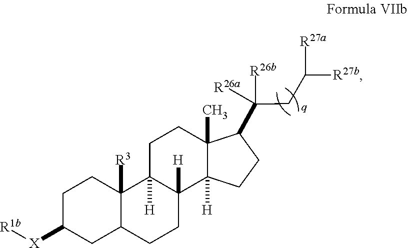
or a pharmaceutically acceptable salt thereof.

120. The compound of any one of claims 117 to 119, wherein each of R²⁶, R^{27a}, and R^{27b} is, independently,

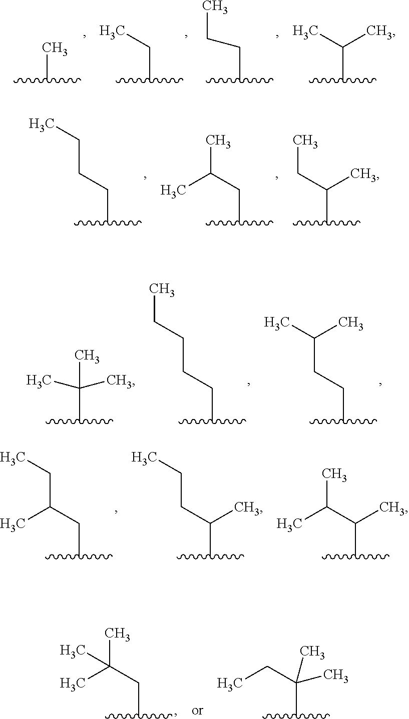

121. A compound having the structure of Formula VIII: 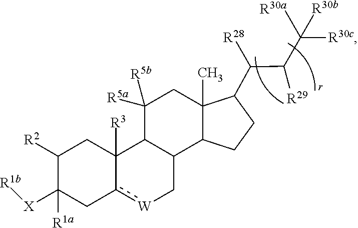
wherein
R^{1a} is H, optionally substituted C₁-C₆ alkyl, optionally substituted C₂-C₆ alkenyl, or optionally substituted C₂-C₆ alkynyl;
X is O or S;
R^{1b} is H or optionally substituted C₁-C₆ alkyl;
R² is H or OR^{A}, where R^{A} is H or optionally substituted C₁-C₆ alkyl;
R³ is H or 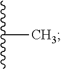 [symbol]
represents a single bond or a double bond;
W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form 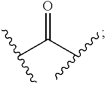 [SMILES: *C(*)=O]
R²⁸ is H or optionally substituted C₁-C₆ alkyl;
r is 1, 2, or 3;
each R²⁹ is, independently, H or optionally substituted C₁-C₆ alkyl; and
each of R^{30a}, R^{30b}, and R^{30c} is C₁-C₆ alkyl,
or a pharmaceutically acceptable salt thereof.

122. The compound of claim 121, wherein the compound has the structure of Formula VIIIa: 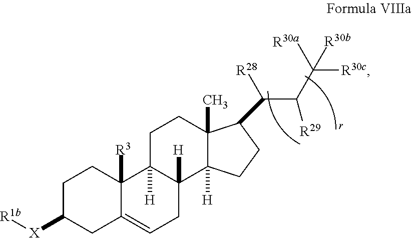
or a pharmaceutically acceptable salt thereof.

123. The compound of claim 121, wherein the compound has the structure of Formula VIIIb: 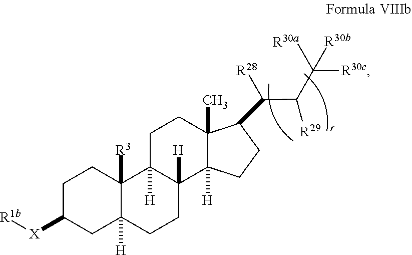
or a pharmaceutically acceptable salt thereof.

124. The compound of any one of claims 121 to 123, wherein each of R²⁸, R^{30a}, R^{30b}, and R^{30c} is independently,

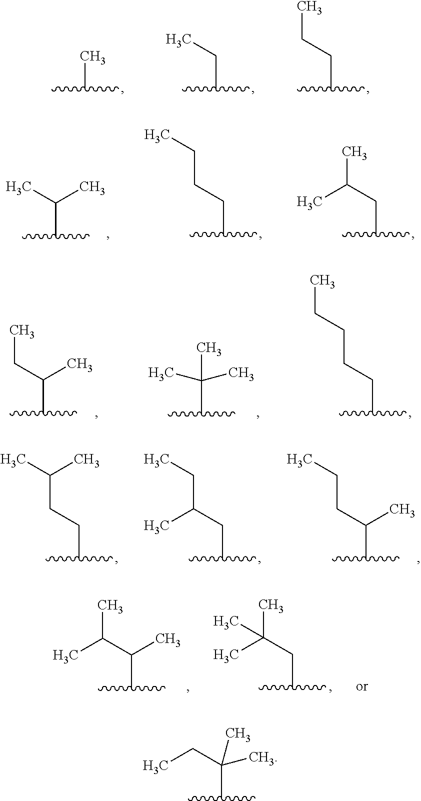

125. The compound of any one of claims 121 to 124, wherein each R²⁹ is, independently, H,

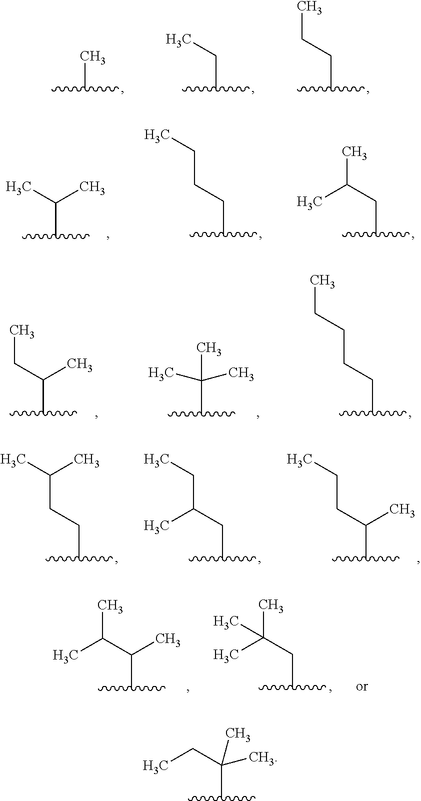

126. A compound having the structure of Formula IX: 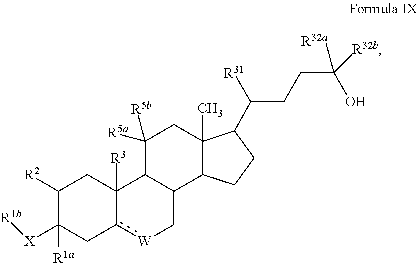
wherein
R^{1a} is H, optionally substituted C₁-C₆ alkyl, optionally substituted C₂-C₆ alkenyl, or optionally substituted C₂-C₆ alkynyl;
X is O or S;
R^{1b} is H or optionally substituted C₁-C₆ alkyl;
R² is H or OR^{A}, where R^{A} is H or optionally substituted C₁-C₆ alkyl;
R³ is H or 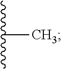 [symbol]
represents a single bond or a double bond;
W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form 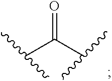 [SMILES: *C(*)=O]
R³¹ is H or C₁-C₆ alkyl; and
each of R^{32a} and R^{32b} is C₁-C₆ alkyl,
or a pharmaceutically acceptable salt thereof.

127. The compound of claim 126, wherein the compound has the structure of Formula IXa: 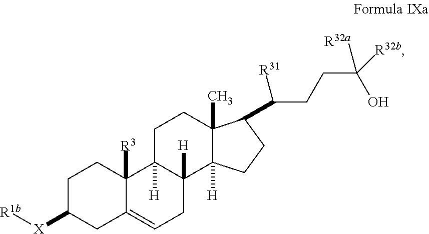
or a pharmaceutically acceptable salt thereof.

128. The compound of claim 126, wherein the compound has the structure of Formula IXb: 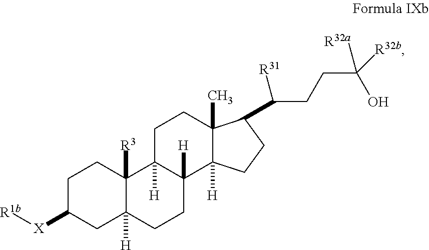
or a pharmaceutically acceptable salt thereof.

129. The compound of any one of claims 126 to 128, wherein each of R³¹, R^{32a}, and R^{32b} is, independently,

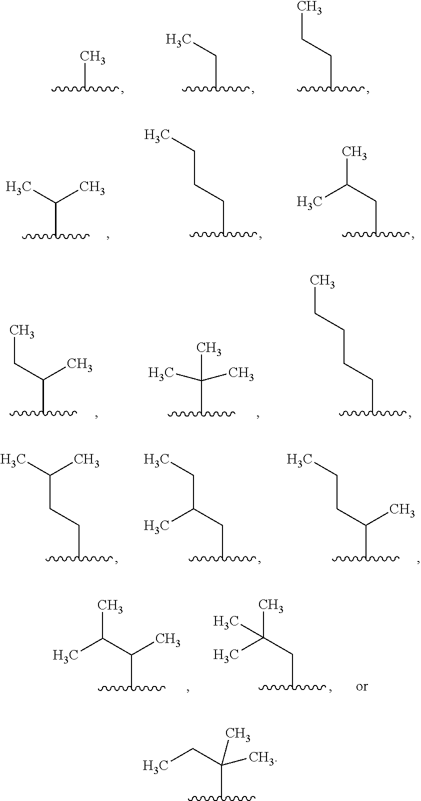

130. A compound having the structure of Formula X: 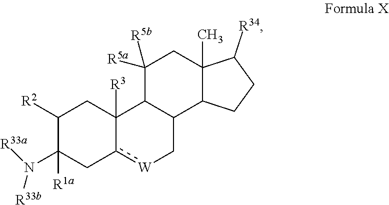
wherein
R^{1a} is H, optionally substituted C₁-C₆ alkyl, optionally substituted C₂-C₆ alkenyl, or optionally substituted C₂-C₆ alkynyl;
X is O or S;
R² is H or OR^{A}, wherein R^{A} is H or optionally substituted C₁-C₆ alkyl;
R³ is H or 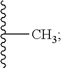 [symbol]
represents a single bond or a double bond;
W is CR^{4a} or CR^{4a}R^{4b}, wherein if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form 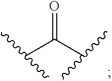 [SMILES: *C(*)=O]
R^{33a} is optionally substituted C₁-C₆ alkyl or 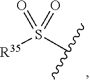
wherein R³⁵ is optionally substituted C₁-C₆ alkyl or optionally substituted C₆-C₁₀ aryl;
R^{33b} is H or optionally substituted C₁-C₆ alkyl; or
R³⁵ and R^{33b}, together with the atom to which each is attached, form an optionally substituted C₃-C₉ heterocyclyl; and
R³⁴ is optionally substituted C₁-C₆ alkyl or optionally substituted C₁-C₆ heteroalkyl,
or a pharmaceutically acceptable salt thereof.

131. The compound of claim 130, wherein the compound has the structure of Formula Xa: 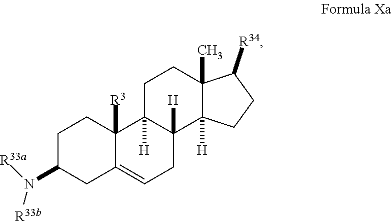
or a pharmaceutically acceptable salt thereof.

132. The compound of claim 130, wherein the compound has the structure of Formula Xb: 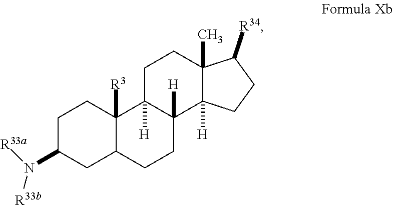
or a pharmaceutically acceptable salt thereof.

133. The compound of any one of claims 130 to 132, wherein R^{33a} is R³⁵

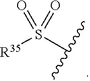

134. The compound of any one of claims 130 to 133, wherein R³⁵ is

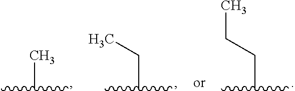

135. The compound of claim 130 or 134, wherein R³⁵ is 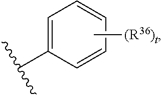
wherein
t is 0, 1, 2, 3, 4, or 5; and
each R³⁶ is, independently, halo, hydroxyl, optionally substituted C₁-C₆ alkyl, or optionally substituted C₁-C₆ heteroalkyl.

136. The compound of any one of claims 130 to 135, wherein R³⁴ is 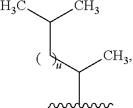
wherein u is 0, 1, 2, 3, or 4.

137. The compound of claim 136, wherein u is 3 or 4.

138. A compound having the structure of Formula XI: 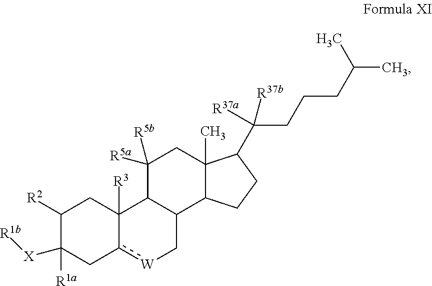
wherein
R^{1a} is H, optionally substituted C₁-C₆ alkyl, optionally substituted C₂-C₆ alkenyl, or optionally substituted C₂-C₆ alkynyl;
X is O or S;
R² is H or OR^{A}, wherein R^{A} is H or optionally substituted C₁-C₆ alkyl;
R₃ is H or 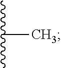 [symbol]
represents a single bond or a double bond;
W is CR^{4a} or CR^{4a}R^{4b}, wherein if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form 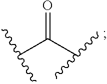 [SMILES: *C(*)=O]
each of R^{37a} and R^{37b} is, independently, optionally substituted C₁-C₆ alkyl, optionally substituted C₁-C₆ heteroalkyl, halo, or hydroxyl,
or a pharmaceutically acceptable salt thereof.

139. The compound of claim 138, wherein the compound has the structure of Formula XIa: 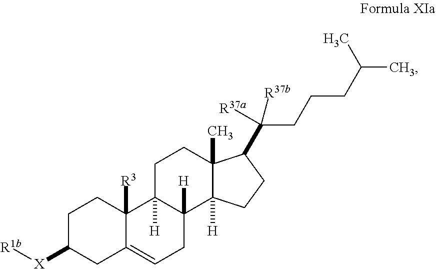
or a pharmaceutically acceptable salt thereof.

140. The compound of claim 138, wherein the compound has the structure of Formula XIb: 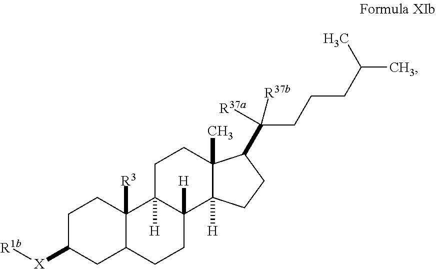
or a pharmaceutically acceptable salt thereof.

141. The compound of any one of claims 138 to 140, wherein R^{37a} is hydroxyl.

142. The compound of any one of claims 138 to 141, wherein R^{37b} is

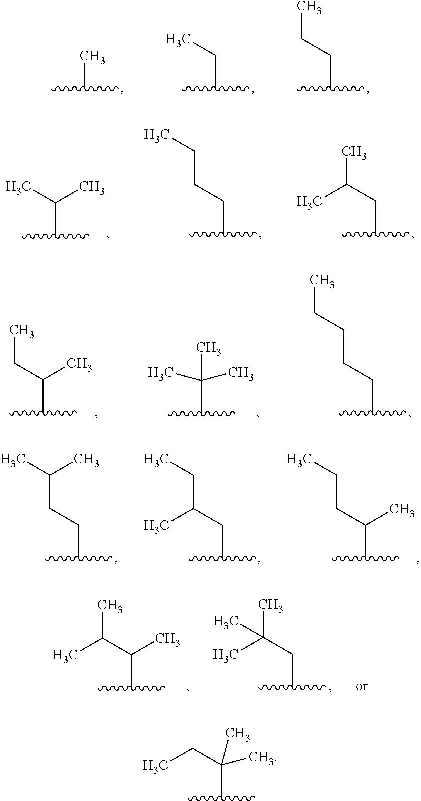

143. A compound having the structure of Formula XII: 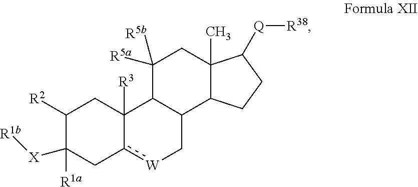
wherein
R^{1a} is H, optionally substituted C₁-C₆ alkyl, optionally substituted C₂-C₆ alkenyl, or optionally substituted C₂-C₆ alkynyl;
X is O or S;
R² is H or OR^{A}, wherein R^{A} is H or optionally substituted C₁-C₆ alkyl;
R³ is H or 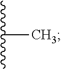 [symbol]
represents a single bond or a double bond;
W is CR^{4a} or CR^{4a}R^{4b}, wherein if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form 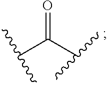 [SMILES: *C(*)=O]
and
Q is O, S, or NR^{E}, wherein R^{E} is H or optionally substituted C₁-C₆ alkyl; and
R³⁸ is optionally substituted C₁-C₆ alkyl,
or a pharmaceutically acceptable salt thereof.

144. The compound of claim 143, wherein the compound has the structure of Formula XIIa: 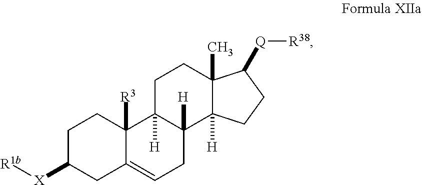
or a pharmaceutically acceptable salt thereof.

145. The compound of claim 143, wherein the compound has the structure of Formula XIIb: 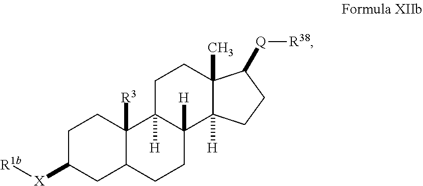
or a pharmaceutically acceptable salt thereof.

146. The compound of any one of claims 143 to 145, wherein Q is NR^{E}.

147. The compound of any one of claims 143 to 146, wherein R^{E} is H or

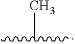

148. The compound of claim 147, wherein R^{E} is

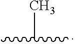

149. The compound of any one of claims 144 to 148, wherein R³⁸ is 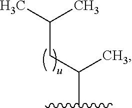
wherein u is 0, 1, 2, 3, or 4.

150. A compound having the structure of Formula XIII: 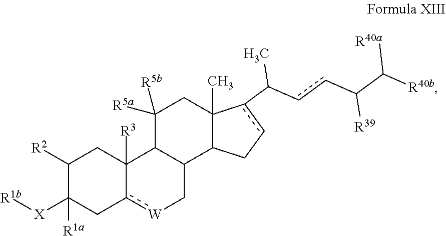
wherein
R^{1a} is H, optionally substituted C₁-C₆ alkyl, optionally substituted C₂-C₆ alkenyl, or optionally substituted C₂-C₆ alkynyl;
X is O or S;
R^{1b} is H, optionally substituted C₁-C₆ alkyl, or 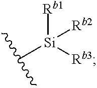
each of R^{b1}, R^{b2}, and R^{b3} is, independently, optionally substituted C₁-C₆ alkyl or optionally substituted C₆-C₁₀ aryl;
R² is H or OR^{A}, where R^{A} is H or optionally substituted C₁-C₆ alkyl;
R³ is H or 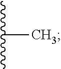
each [symbol] independently represents a single bond or a double bond;
W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form 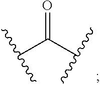 [SMILES: *C(*)=O]
R³⁹ is H or optionally substituted C₂-C₂₀ alkyl;
R^{40a} is optionally substituted C₃-C₂₀ alkyl; and
R^{40b} is optionally substituted C₃-C₂₀ alkyl,
or a pharmaceutically acceptable salt thereof.

151. The compound of claim 150, wherein the compound has the structure of Formula XIIa: 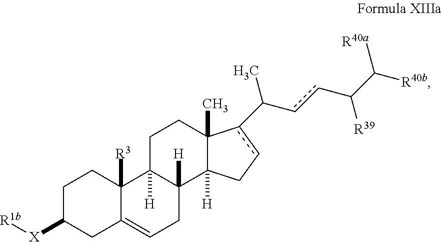
or a pharmaceutically acceptable salt thereof.

152. The compound of claim 150, wherein the compound has the structure of Formula XIIb: 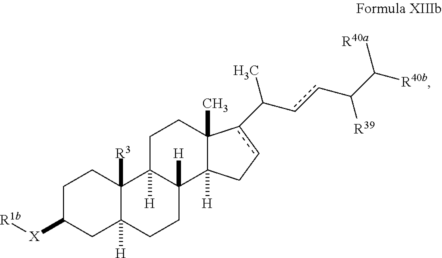
or a pharmaceutically acceptable salt thereof.

153. The compound of claim 150, wherein the compound has the structure of Formula XIIc: 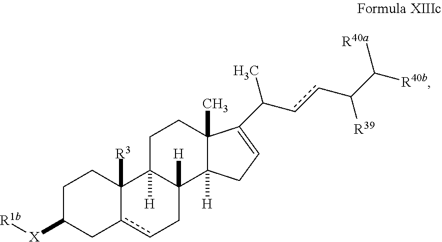
or a pharmaceutically acceptable salt thereof.

154. The compound of claim 150, wherein the compound has the structure of Formula XIId: 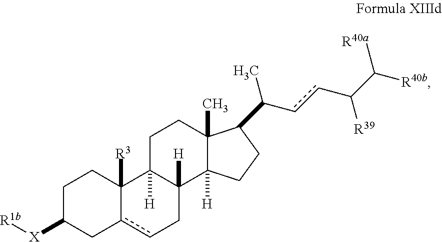
or a pharmaceutically acceptable salt thereof.

155. The compound of any one of claims 150 to 154, wherein R³⁹ is H.

156. The compound of any one of claims 150 to 154, wherein R³⁹ is optionally substituted C₂-C₂₀ alkyl.

157. The compound of claim 156, wherein R³⁹ is optionally substituted C₂-C₁₂ alkyl.

158. The compound of claim 157, wherein R³⁹ is optionally substituted C₂-C₁₀ alkyl.

159. The compound of claim 158, wherein R³⁹ is

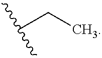 [SMILES: *CC]

160. The compound of any one of claims 150 to 159, wherein R^{40a} is optionally substituted C₃-C₁₂ alkyl.

161. The compound of claim 160, wherein R^{40a} is optionally substituted C₃-C₁₀ alkyl.

162. The compound of claim 161, wherein R^{40a} is

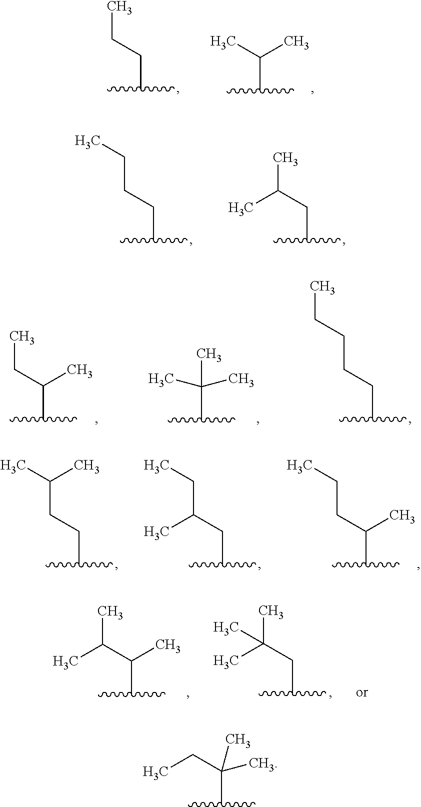

163. The compound of claim 162, wherein R^{40a} is

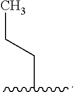 [SMILES: *CCC]

164. The compound of any one of claims 150 to 159, wherein R^{40a} is optionally substituted C₄-C₂₀ alkyl.

165. The compound of claim 164, wherein R^{40a} is

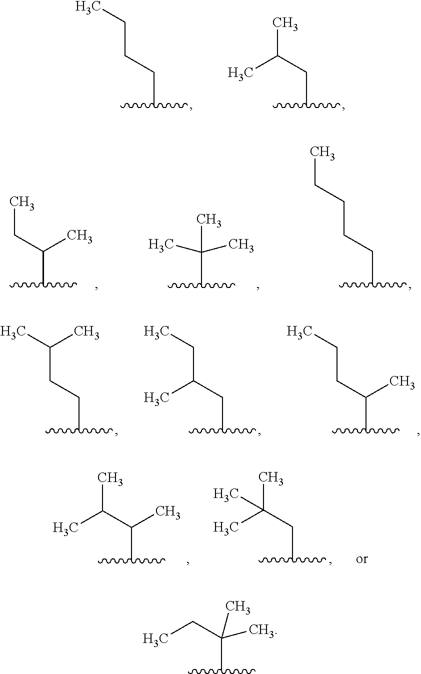

166. The compound of claim 165, wherein R^{40a} is

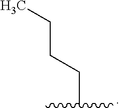 [SMILES: *CCCC]

167. The compound of any one of claims 150 to 166, wherein R^{40a} is

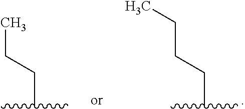

168. The compound of any one of claims 150 to 167, wherein R^{40b} is optionally substituted C₃-C₁₂ alkyl.

169. The compound of claim 168, wherein R^{40b} is optionally substituted C₃-C₁₀ alkyl.

170. The compound of claim 169, wherein R^{40b} is

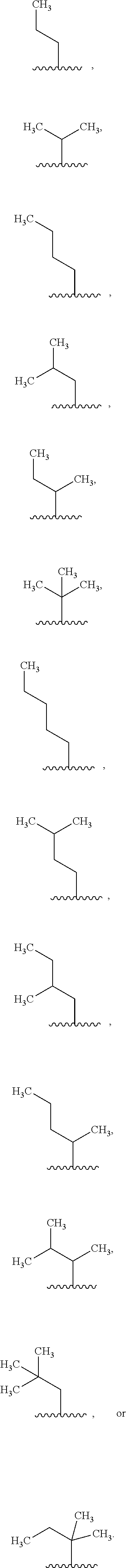

171. The compound of claim 170, wherein R^{40b} is

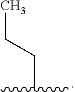 [SMILES: *CCC]

172. The compound of any one of claims 150 to 167, wherein R^{40b} is optionally substituted C₄-C₂₀ alkyl.

173. The compound of claim 172, wherein R^{40b} is

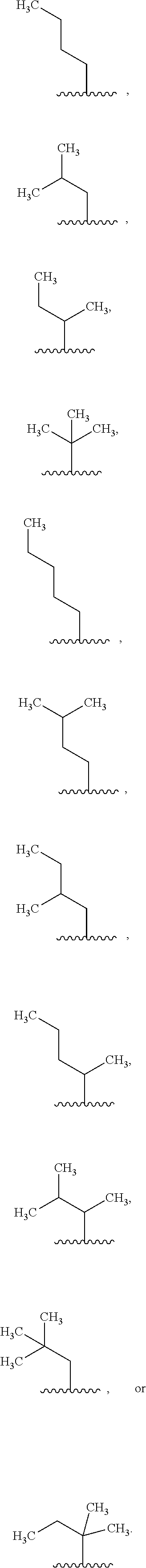

174. The compound of claim 173, wherein R^{40b} is

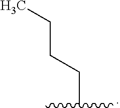 [SMILES: *CCCC]

175. The compound of any one of claims 150 to 174, wherein R^{40b} is

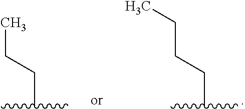

176. The compound of any one of claims 1 to 175, wherein X is O.

177. The compound of any one of claims 1 to 176, wherein R^{1a} is H or optionally substituted C₁-C₆ alkyl.

178. The compound of any one of claims 1 to 177, wherein R^{1a} is H.

179. The compound of any one of claims 1 to 178, wherein R^{1b} is H or optionally substituted C₁-C₆ alkyl.

180. The compound of any one of claims 1 to 179, wherein R^{1b} is H.

181. The compound of any one of claims 1 to 180, wherein R² is H.

182. The compound of any one of claims 1 to 181, wherein R^{4a} is H.

183. The compound of any one of claims 1 to 182, wherein R^{4b} is H.

184. The compound of any one of claims 1 to 183, wherein [symbol] represents a double bond.

185. The compound of any one of claims 1 to 184, wherein R³ is H.

186. The compound of any one of claims 1 to 185, wherein R³ is

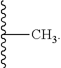

187. The compound of any one of claims 1 to 186, wherein R^{5a} is H.

188. The compound of any one of claims 1 to 187, wherein R^{5b} is H.

189. A compound having the structure of any one of compounds 1-42, 150, 154, 162-165, 169-172, and 184-209 in Table 1, or any pharmaceutically acceptable salt thereof.

190. A compound having the structure of any one of compounds 43-50 and 175-178 in Table 2, or any pharmaceutically acceptable salt thereof.

191. A compound having the structure of any one of compounds 51-67, 149, and 153 in Table 3, or any pharmaceutically acceptable salt thereof.

192. A compound having the structure of any one of compounds 68-73, in Table 4, or any pharmaceutically acceptable salt thereof.

193. A compound having the structure of any one of compounds 74-78 in Table 5, or any pharmaceutically acceptable salt thereof.

194. A compound having the structure of any one of compounds 79 and 80 in Table 6, or any pharmaceutically acceptable salt thereof.

195. A compound having the structure of any one of compounds 81-83, 85-87, 152, and 157 in Table 7, or any pharmaceutically acceptable salt thereof.

196. A compound having the structure of any one of compounds 88-97 in Table 8, or any pharmaceutically acceptable salt thereof.

197. A compound having the structure of any one of compounds 98-105, 180-182, and 210-213 in Table 9, or any pharmaceutically acceptable salt thereof.

198. A compound having the structure of compound 106 in Table 10, or any pharmaceutically acceptable salt thereof.

199. A compound having the structure of any one of compounds 107-108 in Table 11, or any pharmaceutically acceptable salt thereof.

200. A compound having the structure of compound 109 in Table 12, or any pharmaceutically acceptable salt thereof.

201. A compound having the structure of compounds 214-218 in Table 13, or any pharmaceutically acceptable salt thereof.

202. A compound having the structure of any one of compounds 110-130, 155, 156, 160, 161, 166-168, 173, 174, 179, and 219-226 in Table 14, or any pharmaceutically acceptable salt thereof.

203. A lipid nanoparticle comprising:
(i) an ionizable lipid; and
(ii) a structural component,
wherein the structural component comprises a compound of any one of claims 1 to 202 or any one of compounds 131-133 in Table 15.

204. The lipid nanoparticle of claim 203, wherein the lipid nanoparticle further comprises a nucleic acid molecule.

205. A lipid nanoparticle comprising:
(i) an ionizable lipid;
(ii) a structural component;
(iii) optionally, a non-cationic helper lipid;
(iv) optionally, a PEG-lipid; and
(v) a nucleic acid molecule,
wherein the structural component comprises a compound of any one of claims 1 to 202 or any one of compounds 131-133 in Table 15 and optionally a structural lipid component.

206. The lipid nanoparticle of any one of claims 203 to 205, wherein the lipid nanoparticle comprises the compound of any one of claims 1 to 202 or any one of compounds 131-133 in Table 15 in an amount that enhances delivery of the nucleic acid molecule to a cell relative to a lipid nanoparticle lacking the compound.

207. The lipid nanoparticle of any one of claims 203 to 206, wherein the lipid nanoparticle further comprises one or more structural lipids or salts thereof.

208. The lipid nanoparticle of claim 207, wherein the one or more structural lipids is a sterol.

209. The lipid nanoparticle of claim 208, wherein the one or more structural lipids is a phytosterol.

210. The lipid nanoparticle of claim 209, wherein the phytosterol is β-sitosterol, campesterol, stigmasterol, or any combination thereof.

211. The lipid nanoparticle of claim 209 or 210, wherein the one or more structural lipids comprises a mixture of β-sitosterol, campesterol, and stigmasterol.

212. The lipid nanoparticle of claim 211, wherein the one or more structural lipids comprises about 40% of β-sitosterol, about 25% stigmasterol, and about 25% of campesterol.

213. The lipid nanoparticle of claim 211, wherein the one or more structural lipids comprises about 70% of β-sitosterol, about 10% stigmasterol, and about 10% of campesterol.

214. The lipid nanoparticle of claim 208, wherein the one or more structural lipids is a zoosterol.

215. The lipid nanoparticle of claim 214, wherein the zoosterol is cholesterol.

216. The lipid nanoparticle of claim 207, wherein the one or more structural lipids is any one of compounds 84, 134-148, 151, and 159 in Table 16.

217. The lipid nanoparticle of claim 207, wherein the one or more structural lipids is a composition of structural lipids.

218. The lipid nanoparticle of claim 217, wherein the composition of structural lipids is composition 183 in Table 17.

219. The lipid nanoparticle of claim 208, wherein composition 183 includes about 35% to about 45% of compound 141, about 20% to about 30% of compound 140, about 20% to about 30% compound 143, and about 5% to about 15% of compound 148.

220. The lipid nanoparticle of any one of claims 207 to 219, wherein the mol % of the one or more structural lipids is between about 1% and 50% of the mol % of the compound of any one of claims 1 to 202 or any one of compounds 131-133 in Table 15 present in the lipid nanoparticle.

221. The lipid nanoparticle of any one of claims 207 to 219, wherein the mol % of the one or more structural lipids is between about 10% and 40% of the mol % of the compound of any one of claims 1 to 202 or any one of compounds 131-133 in Table 15 present in the lipid nanoparticle.

222. The lipid nanoparticle of any one of claims 207 to 221, wherein the mol % of the one or more structural lipids is between about 20% and 30% of the mol % of the compound of any one of claims 1 to 202 present in the lipid nanoparticle.

223. The lipid nanoparticle of any one of claims 207 to 222, wherein the mol % of the one or more structural lipids is about 30% of the mol % of the compound of any one of claims 1 to 202 present in the lipid nanoparticle.

224. The lipid nanoparticle of any one of claims 203 to 223, wherein the lipid nanoparticle comprises one or more non-cationic helper lipids.

225. The lipid nanoparticle of claim 224, wherein the one or more non-cationic helper lipids is a phospholipid, fatty acid, or any combination thereof.

226. The lipid nanoparticle of claim 225, wherein the phospholipid is a phospholipid that comprises a phosphocholine moiety, a phosphoethanolamine moiety, or a phosphor-1-glycerol moiety.

227. The lipid nanoparticle of claim 225 or 226, wherein the phospholipid is 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, or 1,2-didocosahexaenoyl-sn-glycero-3-phosphocholine.

228. The lipid nanoparticle of claim 227, wherein the phospholipid is DSPC.

229. The lipid nanoparticle of claim 225 or 226, wherein the phospholipid is 1,2-dioleoyl-sn-glycero-3-phosphoethanola mine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, or 1,2-dioleoyl-sn-glycero-3-phospho-rac-(1-glycerol) sodium salt (DOPG).

230. The lipid nanoparticle of claim 225 or 226, wherein the phospholipid is sphingomyelin.

231. The lipid nanoparticle of claim 225, wherein the fatty acid is a long-chain fatty acid.

232. The lipid nanoparticle of claim 231, wherein the fatty acid is palmitic acid, stearic acid, palmitoleic acid, oleic acid, or any combination thereof.

233. The lipid nanoparticle of claim 232, wherein the fatty acid is oleic acid.

234. The lipid nanoparticle of claim 232, wherein the fatty acid is stearic acid.

235. The lipid nanoparticle of any one of claims 203 to 234, wherein the lipid nanoparticle comprises one or more PEG-lipids.

236. The lipid nanoparticle of claim 235, wherein the one or more PEG-lipids is a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, or mixtures thereof.

237. The lipid nanoparticle of claim 235 or 236, wherein the one or more PEG-lipids is PEG-c-DOMG, PEG-DMG, PEG-DLPE, PEG-DMPE, PEG-DPPC, or PEG-DSPE lipid.

238. The lipid nanoparticle of claim 237, wherein the one or more PEG-lipids is PEG-DMG.

239. The lipid nanoparticle of any one of claims 203 to 238, wherein the lipid nanoparticle comprises about 30 mol % to about 60 mol % one or more ionizable lipids, about 0 mol % to about 30 mol % one or more non-cationic helper lipids, about 18.5 mol % to about 48.5 mol % structural component, and about 0 mol % to about 10 mol % one or more PEG-lipids.

240. The lipid nanoparticle of any one of claims 203 to 239, wherein the lipid nanoparticle comprises about 35 mol % to about 55 mol % one or more ionizable lipids, about 5 mol % to about 25 mol % one or more non-cationic helper lipids, about 30 mol % to about 40 mol % structural component, and about 0 mol % to about 10 mol % one or more PEG-lipids.

241. The lipid nanoparticle of any one of claims 203 to 240, wherein the lipid nanoparticle comprises about 50 mol % one or more ionizable lipids, about 10 mol % one or more non-cationic helper lipids, about 38.5 mol % structural component, and about 1.5 mol % one or more PEG-lipids.

242. The lipid nanoparticle of any one of claims 203 to 241, wherein the nucleic acid molecule is RNA or DNA.

243. The lipid nanoparticle of any one of claims 203 to 242, wherein the nucleic acid is DNA.

244. The lipid nanoparticle of claim 243, wherein the nucleic acid molecule is ssDNA.

245. The lipid nanoparticle of claim 243, wherein the nucleic acid is DNA comprising CRISPR.

246. The lipid nanoparticle of any one of claims 203 to 242, wherein the nucleic acid is RNA.

247. The lipid nanoparticle of claim 246, wherein the nucleic acid molecule is a shortmer, an antagomir, an antisense, a ribozyme, a small interfering RNA (siRNA), an asymmetrical interfering RNA (aiRNA), a microRNA (miRNA), a Dicer-substrate RNA (dsRNA), a small hairpin RNA (shRNA), or a messenger RNA (mRNA).

248. The lipid nanoparticle of claim 242 or 247, wherein the nucleic acid molecule is an mRNA.

249. The lipid nanoparticle of claim 248, wherein the mRNA is a modified mRNA comprising one or more modified nucleobases.

250. The lipid nanoparticle of claim 248 or 249, wherein the mRNA comprises one or more of a stem loop, a chain terminating nucleoside, a polyA sequence, a polyadenylation signal, and a 5′ cap structure.

251. The lipid nanoparticle of any one of claims 203 to 250, wherein the structural component comprises a compound of any one of claims 1 to 82 or 176 to 188.

252. The lipid nanoparticle of any one of claims 203 to 250, wherein the structural component comprises a compound of any one of claims 83 to 86 or 176 to 188.

253. The lipid nanoparticle of any one of claims 203 to 250, wherein the structural component comprises a compound of any one of claims 87 to 104 or 176 to 188.

254. The lipid nanoparticle of any one of claims 203 to 250, wherein the structural component comprises a compound of any one of claims 105 to 108 or 176 to 188.

255. The lipid nanoparticle of any one of claims 203 to 250, wherein the structural component comprises a compound of any one of claims 109 to 112 or 176 to 188.

256. The lipid nanoparticle of any one of claims 203 to 250, wherein the structural component comprises a compound of any one of claims 113 to 116 or 176 to 188.

257. The lipid nanoparticle of any one of claims 203 to 250, wherein the structural component comprises a compound of any one of claims 117 to 120 or 176 to 188.

258. The lipid nanoparticle of any one of claims 203 to 250, wherein the structural component comprises a compound of any one of claims 121 to 125 or 176 to 188.

259. The lipid nanoparticle of any one of claims 203 to 250, wherein the structural component comprises a compound of any one of claims 126 to 129 or 176 to 188.

260. The lipid nanoparticle of any one of claims 203 to 250, wherein the structural component comprises a compound of any one of claims 130 to 137 or 176 to 188.

261. The lipid nanoparticle of any one of claims 203 to 250, wherein the structural component comprises a compound of any one of claims 138 to 142 or 176 to 188.

262. The lipid nanoparticle of any one of claims 203 to 250, wherein the structural component comprises a compound of any one of claims 143 to 149 or 176 to 188.

263. The lipid nanoparticle of any one of claims 203 to 250, wherein the structural component comprises a compound of any one of claims 150 to 188.

264. The lipid nanoparticle of any one of claims 203 to 250, wherein the structural component comprises a compound of any one of claims 189-202.

265. The lipid nanoparticle of any one of claims 203 to 250, wherein the lipid nanoparticle further comprises an additional compound of any one of claims 1 to 202
